(19) United States
(12) Patent Application Publication
(10) Pub. No.: US 2026/0139044 A1
(43) Pub. Date: May 21, 2026

(54) TUMOR-TARGETED SPLIT IL2 RECEPTOR AGONISTS

(71) Applicant: Regeneron Pharmaceuticals, Inc., Tarrytown, NY (US)

(72) Inventors: Jiaxi WU, Pleasantville, NY (US); Supriya PATEL, Briarcliff Manor, NY (US); Vidur GARG, White Plains, NY (US); Tong ZHANG, New Rochelle, NY (US); Erica ULLMAN, Yorktown Heights, NY (US); Chia-Yang LIN, Scarsdale, NY (US); Andrew J. MURPHY, Croton-on-Hudson, NY (US); George D. YANCOPOULOS, Yorktown Heights, NY (US)

(73) Assignee: Regeneron Pharmaceuticals, Inc., Tarrytown, NY (US)

(21) Appl. No.: 19/301,816

(22) Filed: Aug. 15, 2025

Related U.S. Application Data
(60) Provisional application No. 63/684,111, filed on Aug. 16, 2024.
(60) Provisional application No. 63/730,246, filed on Dec. 10, 2024.

(51) Int. Cl. C07K 16/22; A61K 38/00; A61P 35/04; C07K 16/28; C07K 16/30

(52) U.S. Cl. CPC C07K 16/22; A61K 38/00; A61P 35/04; C07K 16/2866; C07K 16/3007; C07K 16/3069; C07K 16/3092; C07K 2317/31; C07K 2317/53; C07K 2317/55; C07K 2317/622

(57) ABSTRACT

The present disclosure relates to tumor-targeted split IL2 receptor agonists with improved therapeutic profiles.

### 1. CROSS-REFERENCE TO RELATED APPLICATIONS

This application claims the priority benefit of U.S. provisional application No. 63/684,111, filed Aug. 16, 2024 and U.S. provisional application No. 63/730,246, filed Dec. 10, 2024, the contents of each of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Aug. 13, 2025, is named RGN-054US_SL.xml and is 256,693 bytes in size.

### 3. BACKGROUND

Interleukin 2 (IL-2 or IL2) is a pluripotent cytokine produced primarily by CD4+ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilitates the proliferation and differentiation of B-cells and the synthesis of immunoglobulin by B-cells, and stimulates the generation, proliferation and activation of natural killer (NK) cells (see Waldmann, 2009, Nat Rev Immunol 6:595-601 and Malek, 2008, Annu Rev Immunol 26:453-79).

Due to its pleotropic effects, IL2 is not optimal for inhibiting tumor growth. The use of IL2 as an antineoplastic agent has been limited by the serious toxicities that accompany the doses necessary for a tumor response. Proleukin® (marketed by Prometheus Laboratories, San Diego, Calif.), is a recombinant form of IL2 that is approved for the treatment of metastatic melanoma and metastatic renal cancer, but its side effects are so severe that its use is only recommended in a hospital setting with access to intensive care. Patients receiving high-dose IL2 treatment frequently experience severe cardiovascular, pulmonary, renal, hepatic, gastrointestinal, neurological, cutaneous, haematological and systemic adverse events, which require intensive monitoring and in-patient management. The major side effect of IL2 therapy is vascular leak syndrome (VLS), which leads to the accumulation of interstitial fluid in the lungs and liver resulting in pulmonary edema and liver damage. There is no treatment for VLS other than withdrawal of IL2. Low-dose IL2 regimens have been tested in patients to avoid VLS, however, at the expense of suboptimal therapeutic results. It has been shown that IL2-induced pulmonary edema resulted from direct binding of IL2 to lung endothelial cells, which express low to intermediate levels of functional high affinity IL2 receptors (Krieg et al., 2010, Proc Nat Acad Sci USA 107:11906-11).

A variety of IL2 variants and prodrugs have been generated with the aim of reducing the toxicity of IL2 cancer therapy. However, it has been surprisingly discovered that such molecules have poor therapeutic indices for cancer therapy. For example, the PEGylated IL2 prodrug bempegaldesleukin failed to improve on the therapeutic efficacy of a PD1 checkpoint inhibitor in melanoma patients in phase 3 clinical studies (Mullard, 2022, Nature Reviews Drug Discovery 21:327 (doi: 10.1038/d41573-022-00069-3)).

Thus, there is a need in the art for novel IL2 therapies with improved therapeutic efficacy and safety profiles.

### 4. SUMMARY

The present disclosure provides tumor-targeted split IL2 receptor agonists.

In certain aspects, the tumor-targeted split IL2 receptor agonists address the drawbacks of IL2 therapy (i.e., therapy comprising activation of IL2 signaling in a subject, e.g., IL2 receptor agonist therapy), and are characterized by improved therapeutic profiles by virtue of efficacy and/or improved safety profiles. The tumor-targeted split IL2 receptor agonists of the disclosure typically comprise two components, or a “combination”, formulated in a single formulation or separate formulations, comprising a tumor-targeted IL2Rβ binding molecule and a tumor-targeted IL2Rγ binding molecule. Exemplary tumor-targeted split IL2 receptor agonists are disclosed in Section 6.2 and numbered embodiments 1 to 14 and 29 to 232. Exemplary tumor-targeted IL2Rβ binding molecules are disclosed in Section 6.3 and numbered embodiments 29 to 33, 37 to 196, 205 to 206, and 209 to 214. Exemplary tumor-targeted IL2Rγ binding molecules are disclosed in Section 6.4 and numbered embodiments 29 to 33, 37 to 44, 197 to 204, 207 to 211 and 220 to 222.

The disclosure further provides nucleic acids encoding the tumor-targeted split IL2 receptor agonists of the disclosure and their components. The nucleic acids can be in the form of a single nucleic acid (e.g., a vector encoding all components of the tumor-targeted split IL2 receptor agonists) or a plurality of nucleic acids (e.g., two or more vectors encoding the different components and/or their individual polypeptide chains). The disclosure further provides host cells and cell lines engineered to express the nucleic acids and the tumor-targeted split IL2 receptor agonists of the disclosure. The disclosure further provides methods of producing a tumor-targeted split IL2 receptor agonist of the disclosure. Exemplary nucleic acids, host cells, cell lines, and methods of producing tumor-targeted split IL2 receptor agonists are described in Section 6.10.

The disclosure further provides pharmaceutical compositions comprising the tumor-targeted split IL2 receptor agonists of the disclosure. Exemplary pharmaceutical compositions are described in Section 6.11.

Further provided herein are methods of using the tumor-targeted split IL2 receptor agonists, e.g., for eliciting anti-tumor cytotoxicity and treating cancerous conditions. Exemplary methods are described in Section 6.12 and numbered embodiments 15 to 233, infra.

### 5. BRIEF DESCRIPTION OF THE DRAWINGS

FIGS. 1A and 1B illustrate the IL2 receptor and the common subunits of the IL2 and I-15 receptors. FIG. 1A illustrates the low, intermediate and high affinity IL2 receptor subunits. FIG. 1B illustrates the IL2 and I-15 signaling pathways, which have unique receptor subunits but share common β/γ receptor subunits.

FIGS. 2A-2C show exemplary tumor-targeted IL2Rβ binding molecule structures. FIG. 2A shows a tumor-targeted IL2Rβ binding molecule comprising (a) a tumor targeting moiety in Fab format (e.g., a Fab derived from an antibody against a tumor-associated antigen) connected to the N-terminus of a first Fc domain via a first linker (1), and (b) an IL2Rβ binding moiety in Fab format (e.g., a Fab derived from an antibody against IL2Rβ) connected to the N-terminus of a second Fc domain via a second linker (2). FIG. 2B shows a tumor-targeted IL2Rβ binding molecule comprising (a) a tumor targeting moiety in Fab format (e.g., a Fab derived from an antibody against a tumor-associated antigen) connected to the N-terminus of a first Fc domain via a first linker (1), and (b) an IL2Rβ binding moiety in single domain antibody (sdAb) format (e.g., an sdAb against IL2Rβ) connected to the N-terminus of a second Fc domain via a second linker (2). FIG. 2C shows a tumor-targeted IL2Rβ binding molecule comprising (a) a tumor targeting moiety in sdAb format (e.g., an sdAb against a tumor-associated antigen) connected to the N-terminus of a first Fc domain via a first linker (1), and (b) an IL2Rβ binding moiety in sdAb format (e.g., an sdAb against IL2Rβ) connected to the N-terminus of a second Fc domain via a second linker (2). FIG. 2D shows a tumor-targeted IL2Rβ binding molecule comprising (a) a tumor targeting moiety in scFv format (e.g., an scFv against a tumor-associated antigen) connected to the N-terminus of a first Fc domain via a first linker (1) and an IL2Rβ binding moiety in sdAb format (e.g., an sdAb against IL2Rβ) connected to the N-terminus of a second Fc domain via a second linker (2). FIG. 2E shows a tumor-targeted IL2Rβ binding molecule comprising (a) a tumor targeting moiety in scFv format (e.g., an scFv derived from an antibody against a tumor-associated antigen) connected to the N-terminus of a first Fc domain via a first linker (1) and an IL2Rβ binding moiety in scFv format (e.g., an scFv derived from an antibody against IL2Rβ) connected to the N-terminus of a second Fc domain via a second linker (2). FIG. 2F shows a tumor-targeted IL2Rβ binding molecule comprising (a) a tumor targeting moiety in sdAb format (e.g., an sdAb against a tumor-associated antigen) connected to the N-terminus of a first Fc domain via a first linker (1) and an IL2Rβ binding moiety in scFv format (e.g., an scFv derived from an antibody against IL2Rβ) connected to the N-terminus of a second Fc domain via a second linker (2). FIG. 2G shows a tumor-targeted IL2Rβ binding molecule comprising (a) a tumor targeting moiety in Fab format (e.g., a Fab derived from an antibody against a tumor-associated antigen) connected to the N-terminus of a first Fc domain via a first linker (1) and an IL2Rβ binding moiety in scFv format (e.g., an scFv derived from an antibody against IL2Rβ) connected to the N-terminus of a second Fc domain via a second linker (2). FIG. 2H shows a tumor-targeted IL2Rβ binding molecule comprising (a) a tumor targeting moiety in scFv format (e.g., an scFv derived from an antibody against a tumor-associated antigen) connected to the N-terminus of a first Fc domain via a first linker (1) and an IL2Rβ binding moiety in Fab format (e.g., a Fab derived from an antibody against IL2Rβ) connected to the N-terminus of a second Fc domain via a second linker (2).

FIGS. 3A-3C show exemplary tumor-targeted IL2Rγ binding molecule structures. FIG. 3A shows a tumor-targeted IL2Rγ binding molecule comprising (a) a tumor targeting moiety in Fab format (e.g., a Fab derived from an antibody against a tumor-associated antigen) connected to the N-terminus of a first Fc domain via a first linker (1), and (b) an IL2Rγ binding moiety in Fab format (e.g., a Fab derived from an antibody against IL2Rγ) connected to the N-terminus of a second Fc domain via a second linker (2). FIG. 3B shows a tumor-targeted IL2Rγ binding molecule comprising (a) a tumor targeting moiety in Fab format (e.g., a Fab derived from an antibody against a tumor-associated antigen) connected to the N-terminus of a first Fc domain via a first linker (1), and (b) an IL2Rγ binding moiety in single domain antibody (sdAb) format (e.g., an sdAb against IL2Rγ) connected to the N-terminus of a second Fc domain via a second linker (2). FIG. 3C shows a tumor-targeted IL2Rγ binding molecule comprising (a) a tumor targeting moiety in sdAb format (e.g., an sdAb against a tumor-associated antigen) connected to the N-terminus of a first Fc domain via a first linker (1), and (b) an IL2Rγ binding moiety in sdAb format (e.g., an sdAb against IL2Rγ) connected to the N-terminus of a second Fc domain via a second linker (2). FIG. 3D shows a tumor-targeted I IL2Rγ binding molecule comprising (a) a tumor targeting moiety in scFv format (e.g., an scFv against a tumor-associated antigen) connected to the N-terminus of a first Fc domain via a first linker (1) and an IL2Rγ binding moiety in sdAb format (e.g., an sdAb against IL2Rγ) connected to the N-terminus of a second Fc domain via a second linker (2). FIG. 3E shows a tumor-targeted IL2Rγ binding molecule comprising (a) a tumor targeting moiety in scFv format (e.g., an scFv derived from an antibody against a tumor-associated antigen) connected to the N-terminus of a first Fc domain via a first linker (1) and an IL2Rγ binding moiety in scFv format (e.g., an scFv derived from an antibody against IL2Rγ) connected to the N-terminus of a second Fc domain via a second linker (2). FIG. 3F shows a tumor-targeted IL2Rγ binding molecule comprising (a) a tumor targeting moiety in sdAb format (e.g., an sdAb against a tumor-associated antigen) connected to the N-terminus of a first Fc domain via a first linker (1) and an IL2Rγ binding moiety in scFv format (e.g., an scFv derived from an antibody against IL2Rγ) connected to the N-terminus of a second Fc domain via a second linker (2). FIG. 3G shows a tumor-targeted IL2Rγ binding molecule comprising (a) a tumor targeting moiety in Fab format (e.g., a Fab derived from an antibody against a tumor-associated antigen) connected to the N-terminus of a first Fc domain via a first linker (1) and an IL2Rγ binding moiety in scFv format (e.g., an scFv derived from an antibody against IL2Rγ) connected to the N-terminus of a second Fc domain via a second linker (2). FIG. 3H shows a tumor-targeted IL2Rγ binding molecule comprising (a) a tumor targeting moiety in scFv format (e.g., an scFv derived from an antibody against a tumor-associated antigen) connected to the N-terminus of a first Fc domain via a first linker (1) and an IL2Rγ binding moiety in Fab format (e.g., a Fab derived from an antibody against IL2Rγ) connected to the N-terminus of a second Fc domain via a second linker (2).

FIGS. 4A-4C show exemplary multispecific T-cell engager configurations. FIG. 4A shows a bispecific T-cell engager which has a TAA targeting moiety and a T-cell receptor (e.g., CD3) targeting moiety, both in Fab formats, located N-terminally to an Fc domain. Although the TAA targeting moieties and T-cell receptor complex targeting moieties are illustrated as Fabs, they can be in other formats, e.g., scFvs or other formats described in Sections 6.7.2 and 6.7.3, respectively. FIG. 4B shows a bispecific T-cell engager in a CrossMab^{CH-CL} format, which comprises a domain crossover between the CH1 and CL domains of one of the Fabs. Although the domain crossover is illustrated between the CH1 and CL domains of the T-cell receptor complex targeting moiety, it can be present between the CH1 and CL domains of the TAA targeting moiety instead of or in addition to the domain crossover illustrated here. FIG. 4C shows a bispecific T-cell engager in BiTE format, which comprises a T-cell receptor complex targeting moiety and a TAA targeting moiety, both in scFv format, connected via a linker, e.g., a linker described in Section 6.9.

FIGS. 5A-5F are cartoon illustrations depicting the cell-cell linkage between a tumor cell and a lymphocyte in the presence of combinations of tumor-targeted IL2Rβ and IL2Rγ binding molecules. FIG. 5A illustrates a tumor-targeted split IL2R agonist binding to a tumor cell and a T-cell. Each of the IL2Rβ and IL2Rγ binding molecules binds to a tumor-associated antigen via a Fab domain and to IL2Rβ and to IL2Rγ, respectively, via Fab domains. 1: Tumor-associated antigen (TAA); 2: IL2Rβ; 3: IL2Rγ. FIG. 5B illustrates a tumor-targeted split IL2R agonist binding to a tumor cell and a T-cell. Each of the IL2Rβ and IL2Rγ binding molecules binds to a tumor-associated antigen via a Fab domain and to IL2Rβ and to IL2Rγ, respectively, via sdAb domains. 1: Tumor-associated antigen (TAA); 2: IL2Rβ; 3: IL2Rγ. FIG. 5C illustrates a tumor-targeted split IL2R agonist binding to a tumor cell and a T-cell. Each of the IL2Rβ and IL2Rγ binding molecules binds to a tumor-associated antigen via an sdAb domain and to IL2Rβ and to IL2Rγ, respectively, via sdAb domains. 1: Tumor-associated antigen (TAA); 2: IL2Rβ; 3: IL2Rγ. FIG. 5D illustrates a tumor-targeted split IL2R agonist binding to a tumor cell and a T-cell. Each of the IL2Rβ and IL2Rγ binding molecules binds to a different tumor-associated antigen via a Fab domain and to IL2Rβ and to IL2Rγ, respectively, via Fab domains. 1a: First tumor-associated antigen (TAA); 1b: second tumor-associated antigen (TAA); 2: IL2Rβ; 3: IL2Rγ. FIG. 5E illustrates a tumor-targeted split IL2R agonist binding to a tumor cell and a T-cell. Each of the IL2Rβ and IL2Rγ binding molecules binds to a different tumor-associated antigen via a Fab domain and to IL2Rβ and to IL2Rγ, respectively, via sdAb domains. 1a: First tumor-associated antigen (TAA); 1 b: second tumor-associated antigen (TAA); 2: IL2Rβ; 3: IL2Rγ. FIG. 5F illustrates a tumor-targeted split IL2R agonist binding to a tumor cell and a T-cell. Each of the IL2Rβ and IL2Rγ binding molecules binds to a different tumor-associated antigen via an sdAb domain and to IL2Rβ and to IL2Rγ, respectively, via sdAb domains. 1a: First tumor-associated antigen (TAA); 1b: second tumor-associated antigen (TAA); 2: IL2Rβ; 3: IL2Rγ.

FIGS. 6A-6F are graphs that show signaling reporter activation in YT/STAT5-Luc reporter cells by human IL2 (Proleukin), bispecific IL2Rβ×IL2Rγ binding molecules, and tumor-targeted IL2Rβ and IL2Rγ binding molecules alone or in combinations in the absence or presence of PSMA-expressing cells. FIG. 6A is a graph that shows STAT5-Luc reporter activity in YT cells by tumor-targeted IL2Rβ binding molecule B1×PSMA(5) and IL2Rγ binding molecule G1×PSMA(8) alone or in combination, in the absence of PSMA-expressing cells. FIG. 6B is a graph that shows STAT5-Luc reporter activity in YT cells by tumor-targeted IL2Rβ binding molecule B1×PSMA(5) and IL2Rγ binding molecule G1×PSMA(8) alone or in combination, in the presence of 293/hPSMA cells. FIG. 6C is a graph that shows STAT5-Luc reporter activity in YT cells by tumor-targeted IL2Rβ binding molecule B1×PSMA(5) and IL2Rγ binding molecule G1×PSMA(8) alone or in combination, in the presence of Raji/hPSMA cells. FIG. 6D is a graph that shows STAT5-Luc reporter activity in YT cells by tumor-targeted IL2Rβ binding molecule B2×PSMA(3) and IL2Rγ binding molecule G4×PSMA(8) alone or in combination, in the absence of PSMA-expressing cells. FIG. 6E is a graph that shows STAT5-Luc reporter activity in YT cells by tumor-targeted IL2Rβ binding molecule B2×PSMA(3) and IL2Rγ binding molecule G4×PSMA(8) alone or in combination, in the presence of 293/hPSMA cells. FIG. 6F is a graph that shows STAT5-Luc reporter activity in YT cells of tumor-targeted IL2Rβ binding molecule B2×PSMA(3) and IL2Rγ binding molecule G4×PSMA(8) alone or in combination, in the presence of Raji/hPSMA cells.

FIGS. 7A-7N are graphs that show signaling reporter activation in YT/STAT5-Luc reporter cells by human IL2 (Proleukin), bispecific IL2Rβ×IL2Rγ binding molecules, and tumor-targeted IL2Rβ and IL2Rγ binding molecules alone or in combinations in the absence or presence of endogenous PSMA-expressing cells or MUC16-expressing cells. FIG. 7A is a graph that shows STAT5-Luc reporter activity in YT cells by tumor-targeted IL2Rβ binding molecule B1×PSMA(5) and IL2Rγ binding molecule G1×PSMA(8) alone or in combination, in the absence of PSMA-expressing cells. FIG. 7B is a graph that shows STAT5-Luc reporter activity in YT cells by tumor-targeted IL2Rβ binding molecule B1×PSMA(5) and IL2Rγ binding molecule G1×PSMA(8) alone or in combination, in the presence of 22Rv1 cells. FIG. 7C is a graph that shows STAT5-Luc reporter activity in YT cells by tumor-targeted IL2Rβ binding molecule B1×PSMA(5) and IL2Rγ binding molecule G1×PSMA(8) alone or in combination, in the presence of LNCaP cells. FIG. 7D is a graph that shows STAT5-Luc reporter activity in YT cells by tumor-targeted IL2Rβ binding molecule B2×PSMA(5) and IL2Rγ binding molecule G2×PSMA(8) alone or in combination, in the absence of PSMA-expressing cells. FIG. 7E is a graph that shows STAT5-Luc reporter activity in YT cells by tumor-targeted IL2Rβ binding molecule B2×PSMA(5) and IL2Rγ binding molecule G2×PSMA(8) alone or in combination, in the presence of 22Rv1 cells. FIG. 7F is a graph that shows STAT5-Luc reporter activity in YT cells by tumor-targeted IL2Rβ binding molecule B2×PSMA(5) and IL2Rγ binding molecule G2×PSMA(8) alone or in combination, in the presence of LNCaP cells. FIG. 7G is a graph that shows STAT5-Luc reporter activity in YT cells by tumor-targeted IL2Rβ binding molecule B2×PSMA(5) and IL2Rγ binding molecule G3×PSMA(8) alone or in combination, in the absence of PSMA-expressing cells. FIG. 7H is a graph that shows STAT5-Luc reporter activity in YT cells of tumor-targeted IL2Rβ binding molecule B2×PSMA(5) and IL2Rγ binding molecule G3×PSMA(8) alone or in combination, in the presence of 22Rv1 cells. FIG. 7I is a graph that shows STAT5-Luc reporter activity in YT cells of tumor-targeted IL2Rβ binding molecule B2×PSMA(5) and IL2Rγ binding molecule G3×PSMA(8) alone or in combination, in the presence of LNCaP cells. FIG. 7J is a graph that shows STAT5-Luc activity in YT cells of tumor-targeted IL2Rβ binding molecule B2×PSMA(5) and IL2Rγ binding molecule G4×PSMA(8) alone or in combination, in the absence of PSMA-expressing cells. FIG. 7K is a graph that shows STAT5-Luc reporter activity in YT cells of tumor-targeted IL2Rβ binding molecule B2×PSMA(5) and IL2Rγ binding molecule G4×PSMA(8) alone or in combination, in the presence of 22Rv1 cells. FIG. 7L is a graph that shows STAT5-Luc reporter activity in YT cells of tumor-targeted IL2Rβ binding molecule B2×PSMA(5) and IL2Rγ binding molecule G4×PSMA(8) alone or in combination, in the presence of LNCaP cells. FIG. 7M is a graph that shows STAT5-Luc reporter activity in YT cells by tumor-targeted IL2Rβ binding molecule B1×MUC16(4) and IL2Rγ binding molecule G1×MUC16(9) alone or in combination, in the absence of MUC16-expressing cells. FIG. 7N is a graph that shows STAT5-Luc reporter activity in YT cells by tumor-targeted IL2Rβ binding molecule B1×MUC16(4) and IL2Rγ binding molecule G1×MUC16(9) alone or in combination, in the presence of OVCAR3 cells.

FIGS. 8A-8D are graphs that show the activation of phospho-STAT5 (pSTAT5) signaling in unstimulated resting primary human T-cells upon treatment with tumor-targeted IL2Rβ and IL2Rγ binding molecule combination B1×PSMA(8)+G1×PSMA(3) with or without the presence of target expressing tumor cells. FIG. 8A is a graph that shows activation of phospho-STAT5 (pSTAT5) in CD8+ T-cells. FIG. 8B is a graph that shows activation of phospho-STAT5 (pSTAT5) in CD4+ T-cells. FIG. 8C is a graph that shows activation of phospho-STAT5 (pSTAT5) in Treg cells. FIG. 8D shows the activation of phospho STAT5 (pSTAT5) signaling in pre-activated CD8T cells upon treatment with combination of tumor-targeted B1×PSMA(8)+G1×PSMA(3). Combination non-tumor targeted (NT) antibodies B1×NT+G1×NT was used as a control.

FIGS. 9A-9B show cell killing and IFNγ release by tumor-targeted IL2Rβ and IL2Rγ binding molecules. FIG. 9A shows cell killing by B2×PSMA(8)+G3×PSMA(3). FIG. 9B shows IFNγ release by B2×PSMA(8)+G3×PSMA(3).

FIGS. 10A-10C are graphs that show the signaling reporter activation in YT/STAT5-Luc reporter cells by human IL2 (Proleukin), bispecific IL2Rβ×IL2Rγ binding molecules, and HER2-targeted IL2Rβ and IL2Rγ binding molecules alone or in combinations in the absence or presence of endogenous HER2-expressing cells. FIG. 10A is a graph that shows STAT5-Luc reporter activity by HER2-targeted IL2Rβ binding molecules and HER2-targeted IL2Rγ binding molecules alone or in combination, in the presence of NCI-N87 cells, which express HER2 at relatively high levels. FIG. 10B is a graph that shows STAT5-Luc reporter activity by HER2-targeted IL2Rβ binding molecules and HER2-targeted IL2Rγ binding molecules alone or in combination, in the presence of JIMT-1 cells, which express intermediate levels of HER2. FIG. 10C is a graph that shows STAT5-Luc reporter activity by HER2-targeted IL2Rβ binding molecules and HER2-targeted IL2Rγ binding molecules alone or in combination, in the presence of NCI-N87 cells, which express HER2 at relatively low levels.

FIGS. 11A-11B are graphs that show the signaling reporter activation in YT/STAT5-Luc reporter cells by human IL2 (Proleukin), bispecific IL2Rβ×IL2Rγ binding molecules, and EGFR-targeted IL2Rβ and EGFR- or HER2-targeted IL2Rγ binding molecules alone or in combinations in the absence or presence of endogenous EGFR- and HER2-expressing cells. FIG. 11A is a graph that shows STAT5-Luc reporter activity by EGFR-targeted IL2Rβ binding molecules and EGFR- or HER2-targeted IL2Rγ binding molecules alone or in combination, in the presence of JIMT-1 cells.

FIG. 11B is a graph that shows STAT5-Luc reporter activity by EGFR-targeted IL2Rβ binding molecules and EGFR- or HER2-targeted IL2Rγ binding molecules alone or in combination, in the presence of NCI-H292 cells.

FIG. 12 is a graph that shows the effect of the PSMA-targeting moiety format on STAT5-Luc reporter activity by PSMA-targeted IL2Rβ binding molecules and PSMA-targeted IL2Rγ binding molecules alone or in combination, in the presence of C4-2 cells. The following constructs were evaluated alone or in combinations: PSMA(1)×B, which comprises a PSMA-targeting moiety comprising a PSMA(5) Fab and an IL2Rβ-binding moiety comprising a B1 sdAb; PSMA(2)×G, which comprises a PSMA-targeting moiety comprising a PSMA(8) Fab and an IL2Rβ-binding moiety comprising a G1 sdAb; PSMA(1)scFv×B, which comprises a PSMA-targeting moiety comprising a PSMA(5) scFv and an IL2Rβ-binding moiety comprising a B1 sdAb; and PSMA(2)scFv×G, which comprises a PSMA-targeting moiety comprising a PSMA(8) scFv and an IL2Rβ-binding moiety comprising a G1 sdAb. IL2 (Proleukin) and B×G (B1×G1 bispecific antibody) were used as controls.

FIGS. 13A-13C are graphs that show STAT5-Luc reporter activity by tumor-targeted IL2Rβ binding molecules and tumor-targeted IL2Rγ binding molecules alone or in combination. FIG. 13A is a graph showing STAT5-Luc reporter activity in the presence of EGFR-targeted IL2Rβ binding molecules and EGFR-targeted IL2Rγ binding molecules (alone or in combination) in the presence of A431 cells, which express EGFR. FIG. 13B is a graph showing STAT5-Luc reporter activity in the presence of MSLN-targeted IL2Rβ binding molecules and MSLN-targeted IL2Rγ binding molecules (alone or in combination) in the presence of PEO1 cells, which express MSLN. FIG. 13C is a graph showing STAT5-Luc reporter activity in the presence of STEAP1-targeted IL2Rβ binding molecules and STEAP1-targeted IL2Rγ binding molecules (alone or in combination) in the presence of C4-2 cells, which express STEAP1.

FIGS. 14A-14C show STAT5-Luc reporter activity and target cell killing by combinations of tumor-targeted IL2Rβ binding molecules and tumor-targeted IL2Rγ binding molecules or control constructs. FIG. 14A displays the combinations assessed in FIGS. 14B and 14C. FIG. 14B is a graph showing STAT5-Luc reporter activity in the presence of combinations of PSMA-targeted IL2Rβ binding molecules and PSMA-targeted IL2Rγ binding molecules in the presence of C4-2 tumor cells. FIG. 14C is a graph showing HEK293/MUC/16/PSMA target cell killing by combinations of PSMA-targeted IL2Rβ binding molecules and PSMA-targeted IL2Rγ binding molecules in the presence of constant MUC16×CD3 bispecific antibody.

FIGS. 15A-15F show changes in post-implantation tumor radiance in mice treated with combinations of tumor-targeted IL2Rβ binding molecules and tumor-targeted IL2Rγ binding molecules or control constructs. FIG. 15A is a graph showing average tumor radiance in all groups of mice evaluated. FIG. 15B is a graph showing tumor radiance in individual mice treated with an isotype antibody. FIG. 15C is a graph showing tumor radiance in individual mice treated with MSLN×CD3 bispecific antibody. FIG. 15D is a graph showing tumor radiance in individual mice treated with combination of IL2Rβ×MUC16+IL2Rγ×MUC16. FIG. 15E is a graph showing tumor radiance in individual mice treated with combination of IL2Rβ×MUC16+IL2Rγ×MUC16 in the presence of MSLN×CD3 bispecific antibody. FIG. 15F is a graph showing tumor radiance in individual mice treated with combination of MSLN×CD3 and IL2Rβ×IL2Rγ.

FIG. 16 shows percent changes in body weight (BW) post-implantation in mice treated with combinations of tumor-targeted IL2Rβ binding molecules and tumor-targeted IL2Rγ binding molecules or control constructs.

FIGS. 17A-17C show T cell expansion in mice treated with combinations of tumor-targeted IL2Rβ binding molecules and tumor-targeted IL2Rγ binding molecules or control constructs. FIG. 17A is a graph showing total CD3+ T cells per ml blood of mice treated with combinations of tumor-targeted IL2Rβ binding molecules and tumor-targeted IL2Rγ binding molecules or control constructs. FIG. 17B is a graph showing CD4+ T cells per ml blood of mice treated with combinations of tumor-targeted IL2Rβ binding molecules and tumor-targeted IL2Rγ binding molecules or control constructs. FIG. 17C is a graph showing CD8+ T cells per ml blood of mice treated with combinations of tumor-targeted IL2Rβ binding molecules and tumor-targeted IL2Rγ binding molecules or control constructs.

FIGS. 18A-18D show signaling reporter activation in YT/STAT5-Luc reporter cells by tumor-targeted IL2Rβ and IL2Rγ binding molecules alone or in combinations. FIG. 18A is a graph that shows STAT5-Luc reporter activity in YT cells by tumor-targeted IL2Rβ binding molecule B1×PSMA(5) and IL2Rγ binding molecule G1×PSMA(5) alone or in combination. FIG. 18B is a graph that shows STAT5-Luc reporter activity in YT cells by tumor-targeted IL2Rβ binding molecule B1×PSMA(3) and IL2Rγ binding molecule G1×PSMA(3) alone or in combination. FIG. 18C is a graph that shows STAT5-Luc reporter activity in YT cells by tumor-targeted IL2Rβ binding molecule B1×PSMA(8) and IL2Rγ binding molecule G1×PSMA(8) alone or in combination. FIG. 18A is a graph that shows STAT5-Luc reporter activity in YT cells by tumor-targeted IL2Rβ binding molecule B1×PSMA(5) and IL2Rγ binding molecule G1×PSMA(8) alone or in combination.

FIGS. 19A-19D show signaling reporter activation in YT/STAT5-Luc reporter cells by tumor-targeted IL2Rβ and IL2Rγ binding molecules alone or in competing or non-competing combinations in terms of PSMA-targeting arms. FIG. 19A is a graph that shows STAT5-Luc reporter activity in YT cells by tumor-targeted IL2Rβ binding molecule B1×PSMA(5) and IL2Rγ binding molecule G1×PSMA(3) alone or in combination. FIG. 19B is a graph that shows STAT5-Luc reporter activity in YT cells by tumor-targeted IL2Rβ binding molecule B1×PSMA(5) and IL2Rγ binding molecule G1×PSMA(8) alone or in combination. FIG. 19C is a graph that shows STAT5-Luc reporter activity in YT cells by tumor-targeted IL2Rβ binding molecule B2×PSMA(5) and IL2Rγ binding molecule G4×PSMA(3) alone or in combination. FIG. 19D is a graph that shows STAT5-Luc reporter activity in YT cells by tumor-targeted IL2Rβ binding molecule B2×PSMA(5) and IL2Rγ binding molecule G4×PSMA(8) alone or in combination.

### 6. DETAILED DESCRIPTION

### 6.1. Definitions

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antigen Binding Domain or ABD: The term “antigen binding domain” or “ABD” as used herein refers to the portion of a targeting moiety that is capable of specific, non-covalent, and reversible binding to a target molecule.

Associated: The term “associated” in the context of a protein or protein component (e.g., a tumor-targeted IL2Rβ binding molecule; a tumor-targeted IL2Rγ binding molecule; a targeting moiety such as a Fab) refers to a functional relationship between two amino acid sequences on one or more polypeptide chains. In particular, the term “associated” means that two or more sequences or polypeptide chains are associated with one another, e.g., non-covalently through molecular interactions or covalently through one or more disulfide bridges or chemical cross-linkages, so as to produce a functional protein or protein component. Examples of associations that might be present in a tumor-targeted split IL2 receptor agonist of the disclosure include (but are not limited to) associations between homodimeric or heterodimeric Fc domains in an Fc region, associations between VH and VL regions in a Fab or scFv, associations between CH1 and CL in a Fab, and associations between CH3 and CH3 in a domain substituted Fab.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like.

Complementarity Determining Region or CDR: The terms “complementarity determining region” or “CDR,” as used herein, refer to the sequences of amino acids within antibody variable regions which confer antigen specificity and binding affinity. In general, there are three CDRs in each heavy chain variable region (CDR-H1, CDR-H2, CDR-H3) and three CDRs in each light chain variable region (CDR1-L1, CDR-L2, CDR-L3). Though most naturally occurring antibodies are composed of heavy chains and light chains, camelids (e.g., camels, dromedaries, llamas, and alpacas) and some sharks produce antibodies that consist only of heavy chains. These antibodies bind antigenic epitopes using a single variable domain known as VHH and contain only heavy chain CDRs (CDR-H1, CDR-H2, CDR-H3). Exemplary conventions that can be used to identify the boundaries of CDRs include, e.g., the Kabat definition, the Chothia definition, the ABM definition and the IMGT definition. See, e.g., Kabat, 1991, “Sequences of Proteins of Immunological Interest,” National Institutes of Health, Bethesda, Md. (Kabat numbering scheme); AI-Lazikani et al., 1997, J. Mol. Biol. 273:927-948 (Chothia numbering scheme); Martin et al., 1989, Proc. Natl. Acad. Sci. USA 86:9268-9272 (ABM numbering scheme); and Lefranc et al., 2003, Dev. Comp. Immunol. 27:55-77 (IMGT numbering scheme). Public databases are also available for identifying CDR sequences within an antibody.

EC50: The term “EC50” refers to the half maximal effective concentration of a molecule or combination of molecules (such as a tumor-targeted split IL2 receptor agonist) which induces a response halfway between the baseline and maximum after a specified exposure time. In relation to an antibody, the EC50 essentially represents the concentration of the antibody where 50% of its maximal effect is observed. In relation to a tumor-targeted split IL2 receptor agonist, the EC50 value represents the concentration of both components where 50% of their maximal value is observed.

Effector Function: The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which may be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody may be altered by altering, e.g., enhancing or reducing, the affinity of the antibody for an effector molecule such as an Fc receptor or a complement component. Binding affinity will generally be varied by modifying the effector molecule binding site, and in this case, it is appropriate to locate the site of interest and modify at least part of the site in a suitable way. It is also envisaged that an alteration in the binding site on the antibody for the effector molecule need not alter significantly the overall binding affinity but may alter the geometry of the interaction rendering the effector mechanism ineffective as in non-productive binding. It is further envisaged that an effector function may also be altered by modifying a site not directly involved in effector molecule binding, but otherwise involved in performance of the effector function.

Epitope: An epitope, or antigenic determinant, is a portion of an antigen (e.g., target molecule) recognized by an antibody or other antigen-binding moiety as described herein. An epitope can be linear or conformational.

Fab: The term “Fab” in the context of a targeting moiety of the disclosure refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab.

Fc Domain and Fc Region: The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction.

Host cell: The term “host cell” as used herein refers to cells into which a nucleic acid of the disclosure has been introduced. The terms “host cell” and “recombinant host cell” are used interchangeably herein. It is understood that such terms refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. Typical host cells are eukaryotic host cells, such as mammalian host cells. Exemplary eukaryotic host cells include yeast and mammalian cells, for example vertebrate cells such as a mouse, rat, monkey or human cell line, for example HKB11 cells, PER.C6 cells, HEK cells or CHO cells.

Interleukin-2, IL2: IL2 (also “IL-2”) is a class I cytokine. Human IL2, whose amino acid sequence is found in under GenBank accession number NP 000577.2, is synthesized as a precursor polypeptide of 153 amino acids, from which 20 amino acids are removed to generate mature secreted IL2 (Taniguchi et al., 1983, Nature 302(5906):305-10). An exemplary mature human IL2 has amino acid sequence of SEQ ID NO:4.

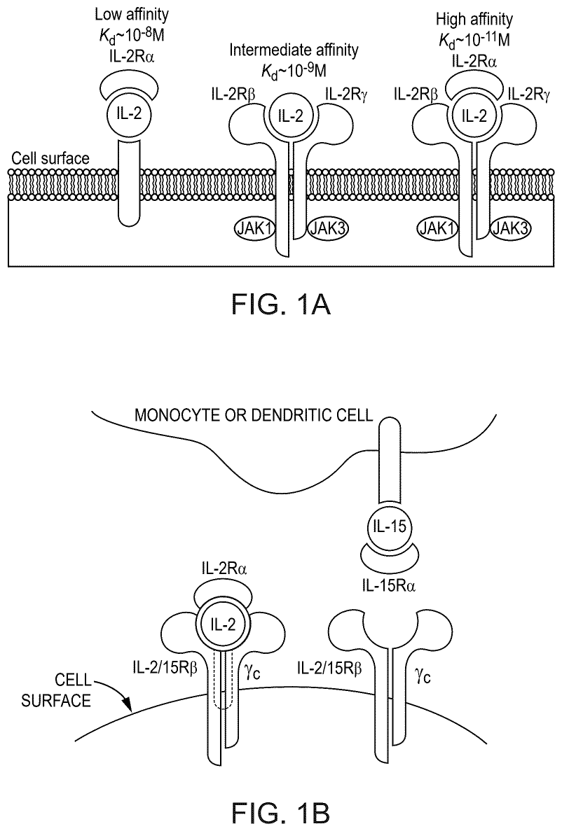

IL2 Receptor: The interleukin-2 receptor (IL2R) is expressed in two different signaling configurations: a dimeric form that consists of IL2Rβ (CD122) and IL2Rγ (CD132) and shows intermediate affinity for IL2 and a trimeric high-affinity form consisting of IL2Rα (CD25), IL2Rβ (CD122) and IL2Rγ (CD132). Cytokine binding induces receptor oligomerization that leads to the juxtaposition of the intracellular domains of IL2Rβ and IL2Rγ, resulting in activation of JAK/TYK kinases associated with the receptor subunits intracellularly. IL2Rα binds specifically to IL2 and is not involved in signal transduction, but increases affinity of the receptor to IL2. IL2Rβ is shared by IL2 and IL15, and IL2Rγ is shared by IL2, IL4, IL7, IL9, IL15, and IL21. IL2Rα and IL2Rβ alone can bind IL-2, whereas IL2Rγ alone does not (see, e.g., Wang et al., 2009, Annu Rev Immunol., 1:29-60 and references cited therein). Exemplary human IL2 receptor sequences are provided as SEQ ID NO:1 (IL2Rα), SEQ ID NO:2 (IL2Rβ), and SEQ ID NO:3 (IL2Rγ). FIG. 1A illustrates the high and intermediate affinity IL2 receptor configurations and FIG. 1B illustrates the common subunits of the IL2 and IL15 receptors.

Operably linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence.

Single Chain Fv or scFv: The term “single chain Fv” or “scFv” as used herein refers to a polypeptide chain comprising the VH and VL domains of antibody, where these domains are present in a single polypeptide chain.

Single Domain Antibody or sdAb: The term “single domain antibody” or “sdAb” as used herein refers to an antibody or antigen binding fragment thereof comprising a single binding domain (e.g., heavy chain variable region) capable of binding a target molecule without pairing with a corresponding CDR-containing polypeptide (e.g., a light chain). An sdAb or sdAb fragment can be derived from a VH, a VHH, or from a non-antibody scaffold protein, for example a designed ankyrin repeat protein (darpin), an avimer, an anticalin/lipocalin, a centyrin or a fynomer. A sdAb typically lacks a CH1 domain and thus cannot associate with a light chain.

Single Domain VH Antibody or sdVH: The term “single domain VH” or “sdVH” as used herein refers to a variable region of an sdAb that is not of camelid or cartilaginous fish origin. An sdVH can be, for example, of human or non-human mammalian origin. A basic sdVH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

Specifically (or selectively) binds: The term “specifically (or selectively) binds” as used herein means that a targeting moiety, e.g., an antibody, or antigen binding domain (“ABD”) thereof, forms a complex with a target molecule that is relatively stable under physiologic conditions. Specific binding can be characterized by a K_{D} of about 5×10⁻²M or less (e.g., less than 5×10⁻²M, less than 10⁻²M, less than 5×10⁻³M, less than 10⁻³M, less than 5×10⁻⁴M, less than 10⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, or less than 10⁻¹⁰M). Methods for determining the binding affinity of an antibody or an antibody fragment, e.g., a tumor-targeted split IL2 receptor agonist or a component targeting moiety, to a target molecule are well known in the art and include, for example, equilibrium dialysis, surface plasmon resonance (e.g., Biacore assays), fluorescent-activated cell sorting (FACS) binding assays and the like. A tumor-targeted split IL2 receptor agonist of the disclosure comprising a targeting moiety or an ABD thereof that specifically binds a target molecule from one species can, however, have cross-reactivity to the target molecule from one or more other species.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Target Molecule: The term “target molecule” as used herein refers to any biological molecule (e.g., protein, carbohydrate, lipid or combination thereof) expressed on a cell surface or in the extracellular matrix that can be specifically bound by a targeting moiety in a tumor-targeted split IL2 receptor agonist of the disclosure. In various embodiments of the tumor-targeted split IL2 receptor agonists of the disclosure, a target molecule can be tumor-associated antigen, IL2Rβ or IL2Rγ.

Targeting Moiety: The term “targeting moiety” as used herein refers to any molecule or binding portion (e.g., an immunoglobulin or an antigen binding fragment) thereof that can bind to a cell surface molecule, e.g., at a site to which a tumor-targeted split IL2 receptor agonist of the disclosure is to be localized. In some embodiments, a targeting moiety binds to a cell surface molecule on tumor cells or on lymphocytes in the tumor microenvironment. The targeting moiety can also have a functional activity in addition to localizing a molecule to a particular site. For example, a targeting moiety in a tumor-targeted split IL2 receptor that is an anti-IL2Rβ or anti-IL2Rγ antibody or an antigen binding portion thereof can modulate (e.g., agonize) IL2 signaling in T-lymphocytes.

Treat, Treatment, Treating: As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more Tumor-targeted split IL2 receptor agonists of the disclosure. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” or “TAA” refers to a molecule (typically a protein, carbohydrate, lipid or some combination thereof) that is expressed on the surface of a cancer cell, either entirely or as a fragment, and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell, for instance, 1-fold overexpression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment, and not synthesized or expressed on the surface of a normal cell. Accordingly, the term “TAA” encompasses antigens that are specific to cancer cells, sometimes known in the art as tumor-specific antigens (“TSAs”).

Tumor-targeted IL2Rβ binding molecule: The term “tumor-targeted IL2Rβ binding molecule” as used herein refers to a molecule comprising a tumor-associated antigen (“TAA”) targeting moiety and an IL2Rβ binding moiety. In some embodiments, the combination of a tumor-targeted IL2Rβ binding molecule and a tumor-targeted IL2Rγ binding molecule results in signaling via the IL2 receptor (e.g., the intermediate affinity IL2 receptor) and/or clustering of IL2Rβ and IL2Rγ receptor subunits. Additionally or alternatively, the combination of a tumor-targeted IL2Rβ binding molecule and a tumor-targeted IL2Rγ binding molecule results in signaling via the IL15 receptor.

Tumor-targeted IL2Rγ binding molecule: The term “tumor-targeted IL2Rγ binding molecule” as used herein refers to a molecule comprising a tumor-associated antigen (“TAA”) targeting moiety and an IL2Rγ binding moiety. In some embodiments, the combination of a tumor-targeted IL2Rβ binding molecule and a tumor-targeted IL2Rγ binding molecule results in signaling via the IL2 receptor (e.g., the intermediate affinity IL2 receptor) and/or clustering of IL2Rβ and IL2Rγ receptor subunits. Additionally or alternatively, the combination of a tumor-targeted IL2Rβ binding molecule and a tumor-targeted IL2Rγ binding molecule results in signaling via the IL15 receptor.

Universal Light Chain: The term “universal light chain” as used herein in the context of a targeting moiety refers to a light chain polypeptide capable of pairing with the heavy chain region of an antibody or antibody fragment, e.g., a targeting moiety, and also capable of pairing with other heavy chain regions. Universal light chains are also known as “common light chains.”

VHH: The term “VHH” refers to a variable region of an antibody consisting of only a heavy chain, e.g., an antibody of camelid or cartilaginous fish origin. A VHH variable region can bind to a target molecule in the absence of a light chain. A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

VH: The term “VH” refers to the variable region of an immunoglobulin heavy chain of an antibody, including the heavy chain of an scFv or a Fab.

VL: The term “VL” refers to the variable region of an immunoglobulin light chain, including the light chain of an scFv or a Fab.

### 6.2. Tumor-Targeted Split IL2 Receptor Agonists

The present disclosure provides tumor-targeted split IL2 receptor agonists. Tumor-targeted split IL2 receptor agonists comprise two components, a tumor-targeted IL2Rβ binding molecule and a tumor-targeted IL2Rγ binding molecule. Thus, a tumor-targeted split IL2 receptor agonist of the disclosure is sometimes referred to herein as a “combination.”

The tumor-targeted IL2Rβ binding molecule and a tumor-targeted IL2Rγ binding molecule each comprise a tumor-targeting moiety, preferably a tumor-associated antigen (“TAA”) targeting moiety. In some embodiments, the tumor-targeted IL2Rβ binding molecule and a tumor-targeted IL2Rγ binding molecule each comprises a TAA targeting moiety, and the TAA targeting moiety of the tumor-targeted IL2Rβ binding molecule and the TAA targeting moiety of the tumor-targeted IL2Rγ binding molecule are typically capable of binding to the same cell, e.g., a tumor cell.

The tumor-targeted IL2Rβ binding molecule further comprises an IL2Rβ binding moiety, and the tumor-targeted IL2Rγ binding molecule further comprises an IL2Rγ binding moiety. The IL2Rβ and IL2Rγ binding moieties can each be a targeting moiety (e.g., an antigen binding fragment of an anti-IL2Rβ or anti-IL2Rγ antibody, respectively). In some embodiments, the IL2Rβ and IL2Rγ binding moieties, upon binding to their respective targets on an IL2 receptor-expressing cell, elicit IL2 receptor signaling, e.g., STAT5 phosphorylation and/or signaling as measured in a reporter assay such as that described in Section 8.1.2 and/or Section 8.1.3.

When the tumor-targeted IL2Rβ binding molecule and tumor-targeted IL2Rγ binding molecule are in proximity of a tumor cell recognized by the TAA targeting moiety and a cell harboring the IL2 receptor such as a cytotoxic T-lymphocyte, the tumor-targeted split IL2 receptor agonist can cross-link the tumor cell and cytotoxic T-lymphocyte, thereby triggering a cytotoxic immune response against the tumor cell.

The use of singular terms, such as “tumor-targeted split IL2 receptor agonist” and “combination” is for convenience only and does not necessitate that the tumor-targeted IL2Rβ binding molecule and tumor-targeted IL2Rγ binding molecule be present in the same composition, but merely that the two components be capable of being used with one another, e.g., to trigger a cytotoxic immune response against a tumor cell.

Examples of tumor-targeted IL2Rβ binding molecules and their components are described in Section 6.3 and subsections thereof.

Examples of tumor-targeted IL2Rγ binding molecules and their components are described in Section 6.4 and subsections thereof.

Suitable TAA targeting moieties for including in the tumor-targeted IL2Rβ binding molecules and tumor-targeted IL2Rγ binding molecules in a tumor-targeted split IL2 receptor agonist are exemplified in Section 6.5.

Suitable formats of the targeting moieties in a tumor-targeted split IL2 receptor agonist (e.g., a TAA targeting moiety, an IL2Rβ targeting moiety, or an IL2Rγ targeting moiety) are disclosed in Section 6.6. In certain aspects, one or more of the targeting moieties are single domain antibodies. In some embodiments, the IL2Rβ targeting moiety is a single domain antibody. In some embodiments, the IL2Rγ targeting moiety is a single domain antibody. In some embodiments, both the IL2Rβ targeting moiety and IL2Rγ targeting moiety are single domain antibodies.

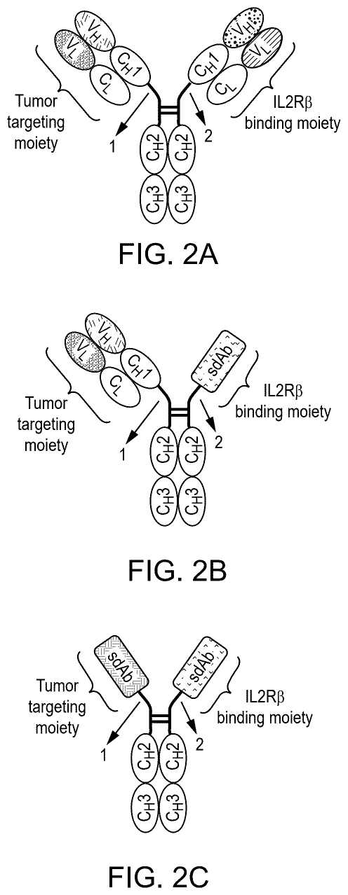

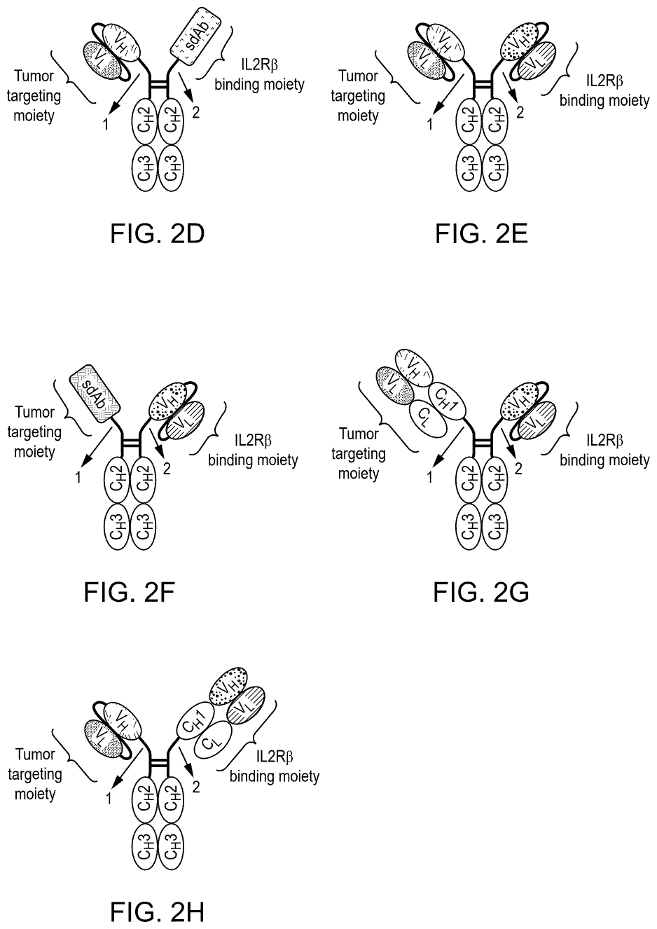

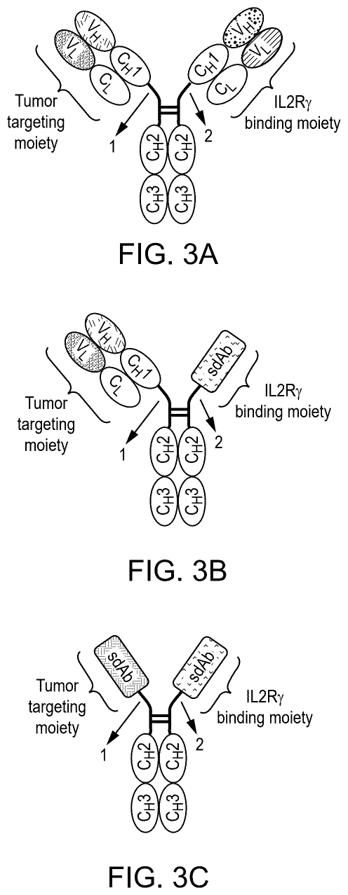

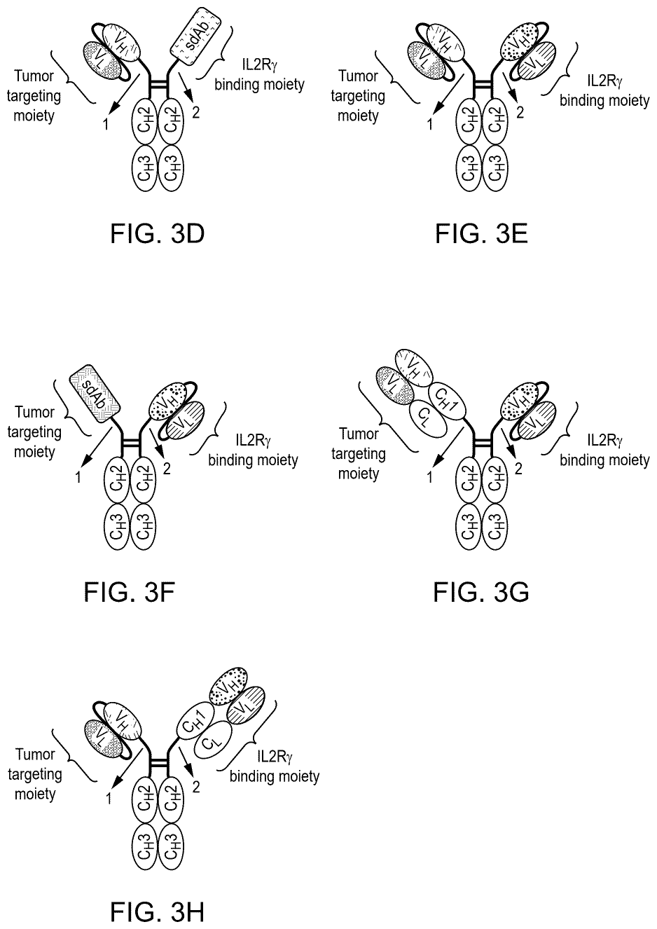

The tumor-targeted IL2Rβ binding molecule and tumor-targeted IL2Rγ binding molecule typically contain Fc domains to which the TAA targeting moieties and the IL2Rβ or IL2Rγ binding moieties are operably linked. Suitable Fc domains are disclosed in Section 6.8. Suitable arrangements of Fc domain, TAA targeting moiety and IL2Rβ binding moiety in a tumor-targeted IL2Rβ binding molecule are disclosed in FIGS. 2A-2H. Suitable arrangements of Fc domain, TAA targeting moiety and IL2Rγ binding moiety in a tumor-targeted IL2Rγ binding molecule are disclosed in FIGS. 3A-3H.

One or more domains in a tumor-targeted IL2Rβ binding molecule and/or a tumor-targeted IL2Rγ binding molecule may be connected to one another via one or more linkers. Suitable linkers are disclosed in Section 6.9.

Nucleic acids encoding, and host cells capable of expressing, a tumor-targeted IL2Rβ binding molecule and/or a tumor-targeted IL2Rγ binding molecule are disclosed in Section 6.10.

Pharmaceutical compositions comprising the tumor-targeted IL2Rβ binding molecules, tumor-targeted IL2Rγ binding molecules and tumor-targeted split IL2 receptor agonists are disclosed in Section 6.11.

Methods of using the tumor-targeted split IL2 receptor agonists, e.g., to treat cancer or elicit anti-cancer immunity, are disclosed in Section 6.12.

### 6.3. Tumor-Targeted IL2Rβ Binding Molecule

### 6.3.1. Tumor-Associated Antigen Targeting Moieties

The tumor-targeted IL2Rβ binding molecule of the tumor-targeted split IL2 receptor agonists of the disclosure comprise a tumor-associated antigen (“TAA”) targeting moiety. Typically, the TAA recognized by the TAA targeting moiety of the tumor-targeted IL2Rβ binding molecule is expressed on the same cancer cell as the TAA recognized by the TAA targeting moiety of the tumor-targeted IL2Rγ binding molecule.

In some embodiments, both TAA targeting moieties recognize the same TAA, whether on the same epitope or on different epitopes. If the TAA targeting moieties recognize different epitopes, they preferably can bind to the cancer cell simultaneously and/or in a non-competing manner (e.g., are non-competing TAA targeting moieties as determined using an antibody cross-competition assay as described in Section 8.1.6). When the TAA(s) recognized by both TAA targeting moieties are expressed on the same cancer cell, the TAA(s) may be the same TAA or different TAAs.

In some embodiments, both TAA targeting moieties recognize different TAAs expressed on the same cancer cell.

Suitable TAA targeting moieties are described in Section 6.5.

### 6.3.2. IL2Rβ Binding Moieties

In some embodiments, the tumor-targeted IL2Rβ binding molecule of the tumor-targeted split IL2 receptor agonists of the disclosure comprise an IL2Rβ targeting moiety as an IL2Rβ binding moiety.

The IL2Rβ targeting moiety typically is or comprises an antigen binding domain of an antibody. The IL2Rβ targeting moiety can be any format, e.g., as disclosed in Section 6.6 or subsections thereof. In some embodiments, the IL2Rβ targeting moiety is a Fab. In some embodiments, the IL2Rβ targeting moiety is an scFv. In some embodiments, the IL2Rβ targeting moiety is a sdAb. In some embodiments, the IL2Rβ targeting moiety (e.g., IL2Rβ targeting sdAb) is an agonistic binder capable of activating IL2 receptor signaling following binding to IL2Rβ expressed on a cell. Such an agonistic IL2Rβ targeting moiety may be capable of activating IL2 receptor signaling alone and/or in combination with an IL2Rγ agonistic binder (e.g., an IL2Rγ binding moiety as described in Section 6.4.2).

In some embodiments, the IL2Rβ targeting moiety is based on an antibody comprising both heavy and light chain variable regions. Exemplary anti-IL2Rβ antibodies comprising both heavy and light chain variable regions are set forth in Table R1 below.

TABLE R1
Exemplary Anti-IL2Rβ Variable Heavy (VH) and Light (VL) Chain Amino Acid
Sequences

| Target or | | |
|---|---|---|
| Description | Reference | Sequence |
| Anti-IL2Rβ | VH: SEQ ID NO: 11 of | VH: |
| antibody | WO 2022/212848 A1; | QVQLQESGPGLVKPSGTLSLTCAVSGGSISSSDWWSWVR |
| F09C | VL: SEQ ID NO: 47 of | QPPGKGLEWIGEIDHSGSTNYNPSLMSRVTISVDKSKNQ |
| | WO 2022/212848 A1 | FSLKLSSVTAADTAVYFCGRGSWELSDAFDIRGQGTLVT |
| | | VSS (SEQ ID NO: 55) |
| | | VL: |
| | | EIVMTQSPATLSVSPGERATLSCRASQSVSSNLAWYQQK |
| | | PGQAPRLLIYGASTRATGIPARFSGSGSGTEFTLTISSL |
| | | QSEDFAVYYCQQYNNWPWTFGQGTKVEIK (SEQ ID |
| | | NO: 56) |
| Anti-IL2Rβ | VH: SEQ ID NO: 12 of | VH: |
| antibody | WO 2022/212848 A1; | QVQLQESGPGLVKSSETLSLTCTVSGGSISSSDWWSWVR |
| F09G | VL: SEQ ID NO: 47 of | QPPGKGLEWIGEIDHSGSTNYNPSLMSRVTISVDKSKNQ |
| | WO 2022/212848 A1 | FSLKLSSVTAADTAVYFCARGSWELTDAFDIRGQGTLVT |
| | | VSS (SEQ ID NO: 57) |
| | | VL: |
| | | EIVMTQSPATLSVSPGERATLSCRASQSVSSNLAWYQQK |
| | | PGQAPRLLIYGASTRATGIPARFSGSGSGTEFTLTISSL |
| | | QSEDFAVYYCQQYNNWPWTFGQGTKVEIK (SEQ ID |
| | | NO: 56) |
| Anti-IL2Rβ | VH: SEQ ID NO: 13 of | VH: |
| antibody | WO 2022/212848 A1; | QVQLQESSPGLVKPSETLSLTCTVSGGSISSSNWWSWVR |
| F09K | VL: SEQ ID NO: 47 of | QPPGKGLEWIGEISHSGSTNYNPSLKSRVTISVDKSKNQ |
| | WO 2022/212848 A1 | FSLRLSSVTAADTAVYFCGRGSWELTDAFDIRGQGTLVT |
| | | VSS (SEQ ID NO: 58) |
| | | VL: |
| | | EIVMTQSPATLSVSPGERATLSCRASQSVSSNLAWYQQK |
| | | PGQAPRLLIYGASTRATGIPARFSGSGSGTEFTLTISSL |
| | | QSEDFAVYYCQQYNNWPWTFGQGTKVEIK (SEQ ID |
| | | NO: 56) |
| Anti-IL2Rβ | VH: SEQ ID NO: 14 of | VH: |
| antibody | WO 2022/212848 A1; | QVQLVESGGGVVQPGRSLRLSCAASGFTFSSYGMHWVRQ |
| F18E | VL: SEQ ID NO: 47 of | APGKEREWVAVISYDGSNKYYTDSVKGRFTISRDNSKNT |
| | WO 2022/212848 A1 | LYLEMNSLRAEDTAVYYCARDLDYDVLTGDPVGGFDIWG |
| | | QGTLVTVSS (SEQ ID NO: 59) |
| | | VL: |
| | | EIVMTQSPATLSVSPGERATLSCRASQSVSSNLAWYQQK |
| | | PGQAPRLLIYGASTRATGIPARFSGSGSGTEFTLTISSL |
| | | QSEDFAVYYCQQYNNWPWTFGQGTKVEIK (SEQ ID |
| | | NO: 56) |
| Anti-IL2Rβ | VH: SEQ ID NO: 35 of | VH: |
| antibody | WO 2017/021540 A1; | EVQLVQSGAEVKKPGSSVKVSCKASGYTFTNYYMHWVRQ |
| P2C4 | VL: SEQ ID NO: 1 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 60) |
| | | VL: |
| | | QSALTQPASVSGSPGQSIAISCTGTSSDIGHYDFVSWYQ |
| | | QHPGTAPKLIIYDINNRPSGISNRFSGSKSDNMASLTIS |
| | | GLQPEDEADYYCSAYTSSDTLVFGGGTKLT (SEQ ID |
| | | NO: 61) |
| Anti-IL2Rβ | VH: SEQ ID NO: 39 of | VH: |
| antibody | WO 2017/021540 A1; | EVQLVQSGTEVKKPGASVKVSCKASGYTFTTYAMHWVRQ |
| P2H7 | VL: SEQ ID NO: 5 of | APGQSLEWMGWINTGNGNTKYSQNFQGRVTMTRDTSIST |
| | WO 2017/021540 A1 | AYMELSRLRSDDTAVYYCARDLGQLERLYFWGQGTLVTV |
| | | SS (SEQ ID NO: 62) |
| | | VL: |
| | | DIQMTQSPSTLSASVGDRVTLSCRAGQAISSWLAWYQQK |
| | | PGKAPKLLIYKASNLESGVPSRFSGGGSGAEFTLTISSL |
| | | QPDDFATYYCQQYQSYPYTFGQGTKLEIR (SEQ ID |
| | | NO: 63) |
| Anti-IL2Rβ | VH: SEQ ID NO: 43 of | VH: |
| antibody | WO 2017/021540 A1; | HVQLVETGGGLVQPGGSLRLSCAASGFTFSSYAMSWVRQ |
| P2D12 | VL: SEQ ID NO: 9 of | APGKGLEWVSAISGSGGSTYYADSVKGRFTISRDNSKNT |
| | WO 2017/021540 A1 | LYLQMNSLRAEDTAVYYCARDLGDYWGQGTLVTVSS |
| | | (SEQ ID NO: 64) |
| | | VL: |
| | | DIQLTQSPSSLSASVGDRVTITCQASQDIGNYLNWYQLK |
| | | PGKAPKLLIYDASNLETGVPSRFSGSGSGTDFTFTISSL |
| | | QPEDIATYYCLQLYDYPLTFGGGTKVEIK (SEQ ID |
| | | NO: 65) |
| Anti-IL2Rβ | VH: SEQ ID NO: 47 of | VH: |
| antibody | WO 2017/021540 A1; | QVQLQQWGAGLLKPSETLSLTCAVYGGSFSGYYWSWIRQ |
| P1G11 | VL: SEQ ID NO: 13 of | PPGKGLEWIGEINHSGSTNYNPSLKSRVTISVDTSKNQF |
| | WO 2017/021540 A1 | SLKLSSVTAADTAVYYCARSSSGDAFDIWGQGTMVTVSS |
| | | (SEQ ID NO: 66) |
| | | VL: |
| | | NFMLTQPHSVSESPGKTVTISCTRSSGSIASNYVQWYQQ |
| | | RPGSSPTTVIFDDNQRPTGVPDRFSAAIDTSSSSASLTI |
| | | SGLTAEDEADYYCQSSHSTAVVFGGGTKLTVL (SEQ |
| | | ID NO: 67) |
| Anti-IL2Rβ | VH: SEQ ID NO: 35 of | VH: |
| antibody | WO 2017/021540 A1; | EVQLVQSGAEVKKPGSSVKVSCKASGYTFTNYYMHWVRQ |
| P2C4_A4 | VL: SEQ ID NO: 17 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 60) |
| | | VL: |
| | | QSALTQPASVSGSPGQSIAISCTGTSSDIGDYDFVSWYQ |
| | | QHPGTAPKLIIYDINNRPSGISNRFSGSKSDNMASLTIS |
| | | GLQPEDEADYYCSAYTSSDTLVFGGGTKLT (SEQ ID |
| | | NO: 68) |
| Anti-IL2Rβ | VH: SEQ ID NO: 51 of | VH: |
| antibody | WO 2017/021540 A1; | EVQLVQSGAEVKKPGSSVKVSCKASGYTFTNYYMHWVRQ |
| P2C4_A9 | VL: SEQ ID NO: 1 of | APGQGLEWMGAIMPSRGGTSYPQKFQGRVTMTGDTSTST |
| | WO 2017/021540 A1 | VYMELSSLRSEDTAVYYCARGEYYYDSSGYYNWGQGTLV |
| | | TVSS (SEQ ID NO: 69) |
| | | VL: |
| | | QSALTQPASVSGSPGQSIAISCTGTSSDIGHYDFVSWYQ |
| | | QHPGTAPKLIIYDINNRPSGISNRFSGSKSDNMASLTIS |
| | | GLQPEDEADYYCSAYTSSDTLVFGGGTKLT (SEQ ID |
| | | NO: 61) |
| Anti-IL2Rβ | VH: SEQ ID NO: 53 of | VH: |
| antibody | WO 2017/021540 A1; | EVQLVQSGAEVKKPGSSVKVSCKASGYTFTNYYIHWVRQ |
| P2C4_B6 | VL: SEQ ID NO: 23 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 70) |
| | | VL: |
| | | QSALTQPASVSGSPGQSIAISCTGTSSDIGHYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 71) |
| Anti-IL2Rβ | VH: SEQ ID NO: 53 of | VH: |
| antibody | WO 2017/021540 A1; | EVQLVQSGAEVKKPGSSVKVSCKASGYTFTNYYIHWVRQ |
| P2C4_E9 | VL: SEQ ID NO: 31 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 70) |
| | | VL: |
| | | QSALTQPASVSGSPGQSIAISCTGTSSDIGHYDFVSWYQ |
| | | QHPGTAPKLIIYDINNRASGISNRFSGSKSDNMASLTIS |
| | | GLQPEDEADYYCSAYTSSDTVVFGGGTKLT (SEQ ID |
| | | NO: 72) |
| Anti-IL2Rβ | VH: SEQ ID NO: 35 of | VH: |
| antibody | WO 2017/021540 A1; | EVQLVQSGAEVKKPGSSVKVSCKASGYTFTNYYMHWVRQ |
| P2C4_B1 | VL: SEQ ID NO: 19 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 60) |
| | | VL: |
| | | QSALTQPASVSGSPGQSIAISCTGTSSDIGHYDFVSWYQ |
| | | QHPGTAPKLIIYDNNNRPSGISNRFSGSKSDNMASLTIS |
| | | GLQPEDEADYYCSAYTSSDTLVFGGGTKLT (SEQ ID |
| | | NO: 73) |
| Anti-IL2Rβ | VH: SEQ ID NO: 35 of | VH: |
| antibody | WO 2017/021540 A1; | EVQLVQSGAEVKKPGSSVKVSCKASGYTFTNYYMHWVRQ |
| P2C4_E7 | VL: SEQ ID NO: 30 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 60) |
| | | VL: |
| | | QSALTQPASVSGSPGQSIAISCTGTSSDIGHYDFVSWYQ |
| | | QHPGTAPKLIIYDINNRPSGISNRFSGSKSDDMASLTIS |
| | | GLQPEDEADYYCSAYTSSDTVVFGGGTKLT (SEQ ID |
| | | NO: 74) |
| Anti-IL2Rβ | VH: SEQ ID NO: 55 of | VH: |
| antibody | WO 2017/021540 A1; | EVQLVQSGAEVKKPGSSVKVSCKASGYTFTNYYMHWVRQ |
| P2C4_B8 | VL: SEQ ID NO: 23 of | PPGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 75) |
| | | VL: |
| | | QSALTQPASVSGSPGQSIAISCTGTSSDIGHYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 71) |
| Anti-IL2Rβ | VH: SEQ ID NO: 35 of | VH: |
| antibody | WO 2017/021540 A1; | EVQLVQSGAEVKKPGSSVKVSCKASGYTFTNYYMHWVRQ |
| P2C4_B5 | VL: SEQ ID NO: 21 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 60) |
| | | VL: |
| | | QSALTQPASVSGSPGQSITISCTGTSSDIGHYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 76) |
| Anti-IL2Rβ | VH: SEQ ID NO: 56 of | VH: |
| antibody | WO 2017/021540 A1; | EVQLVQSGAEVKKPGSTVKVSCKASGYTFTNYYMHWVRQ |
| P2C4_B12 | VL: SEQ ID NO: 24 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 77) |
| | | VL: |
| | | QSALTQPASVSGSPGQSIAISCTGTSSDIGHYDFISWYQ |
| | | QHPGTAPKLIIYDFNNRPSGISNRFSGSKSDNMASLTIS |
| | | GLQPEDEADYYCSAYTSSDTLVFGGGTKLT (SEQ ID |
| | | NO: 78) |
| Anti-IL2Rβ | VH: SEQ ID NO: 35 of | VH: |
| antibody | WO 2017/021540 A1; | EVQLVQSGAEVKKPGSSVKVSCKASGYTFTNYYMHWVRQ |
| P2C4_C4 | VL: SEQ ID NO: 27 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 60) |
| | | VL: |
| | | QSALTQPASVSGSPGQSIAISCTGTSSDIGHYDFVSWYQ |
| | | QHPGTAPKLIIYDNNNRPSGISNRFSGSKSDNMASLTIS |
| | | GLQPEDEADYYCSAYTSSDTVVFGGGTKLT (SEQ ID |
| | | NO: 79) |
| Anti-IL2Rβ | VH: SEQ ID NO: 35 of | VH: |
| antibody | WO 2017/021540 A1; | EVQLVQSGAEVKKPGSSVKVSCKASGYTFTNYYMHWVRQ |
| P2C4_C7 | VL: SEQ ID NO: 28 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 60) |
| | | VL: |
| | | QSALTQPASVSGSPGQSIVISCTGTSSDIGHYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 80) |
| Anti-IL2Rβ | VH: SEQ ID NO35: of | VH: |
| antibody | WO 2017/021540 A1; | EVQLVQSGAEVKKPGSSVKVSCKASGYTFTNYYMHWVRQ |
| P2C4_E6 | VL: SEQ ID NO: 29 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 60) |
| | | VL: |
| | | QSALTQPASVSGSPGQSIAISCTGTSSDIGDYDFVSWYQ |
| | | QHPGTAPKLIIYDINNRPSGISNRFSGSKSDNMASLIIS |
| | | GLQPEDEADYYCSAYTSSDTLVFGGGTKLT (SEQ ID |
| | | NO: 81) |
| Anti-IL2Rβ | VH: SEQ ID NO: 35 of | VH: |
| antibody | WO 2017/021540 A1; | EVQLVQSGAEVKKPGSSVKVSCKASGYTFTNYYMHWVRQ |
| P2C4_F8 | VL: SEQ ID NO: 33 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 60) |
| | | VL: |
| | | QSALTQPASVSGNPGQSIAISCTGTSSDIGHYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 82) |
| Anti-IL2Rβ | VH: SEQ ID NO: 61 of | VH: |
| antibody | WO 2017/021540 A1; | EVQLVQSGAEVKKPGSSVKVSCKASGYTFTNYYMHWVRQ |
| P2C4_F11 | VL: SEQ ID NO: 34 of | APGQGLEWMGAIMPSRGGTSYPQKFQGRVTMTGDTSTST |
| | WO 2017/021540 A1 | VYMELSSLRSEDTAMYYCARGEYYYDSSGYYYWGQGTLV |
| | | TVSS (SEQ ID NO: 83) |
| | | VL: |
| | | QSTLTQPASVSGSPGQSITISCTGTSSDIGHYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 84) |
| Anti-IL2Rβ | VH: SEQ ID NO: 62 of | VH: |
| antibody | WO 2017/021540 A1; | EVQLVQSGAEVKKPGSSVKVSCKASGYTFTNYYMHWVRQ |
| P2C4_G2 | VL: SEQ ID NO: 23 of | APGQGLEWMGAIMPSRGGTSYPQKFQGRVTMTGDTSTST |
| | WO 2017/021540 A1 | VYMELSSLRTEDTAVYYCARGEYYYDSSGYYYWGQGTLV |
| | | TVSS (SEQ ID NO: 85) |
| | | VL: |
| | | QSALTQPASVSGSPGQSIAISCTGTSSDIGHYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 71) |
| Anti-IL2Rβ | VH: SEQ ID NO: 63 of | VH: |
| antibody | WO 2017/021540 A1; | EVQLVQSGAEVKKPGSSVKVSCKASGYTFTNYYMHWVRQ |
| P2C4_G11 | VL: SEQ ID NO: 23 of | APGQGLEWMGAIMPSRGGTSYPQKFQGRVTMTGDTSTST |
| | WO 2017/021540 A1 | VYMELSNLRSEDTAVYYCARGEYYYDSSGYYYWGQGTLV |
| | | TVSS (SEQ ID NO: 86) |
| | | VL: |
| | | QSALTQPASVSGSPGQSIAISCTGTSSDIGHYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 71) |
| Anti-IL2Rβ | VH: SEQ ID NO: 64 of | VH: |
| antibody | WO 2017/021540 A1; | EVQLVQSGAEVKKPGSSVKVSCKASGYTFTNYYMHWVRQ |
| P2C4_H1 | VL: SEQ ID NO: 23 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | NVSS (SEQ ID NO: 87) |
| | | VL: |
| | | QSALTQPASVSGSPGQSIAISCTGTSSDIGHYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 71) |
| Anti-IL2Rβ | VH: SEQ ID NO: 65 of | VH: |
| antibody | WO 2017/021540 A1; | EVQLVQSGAEVKKPGSSVKVSCKASGYTFSNYYMHWVRQ |
| P2C4_H2 | VL: SEQ ID NO: 23 of | APGQGLEWIGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 88) |
| | | VL: |
| | | QSALTQPASVSGSPGQSIAISCTGTSSDIGHYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 71) |
| Anti-IL2Rβ | VH: SEQ ID NO: 66 of | VH: |
| antibody | WO 2017/021540 A1; | EVQLVQSGAEVKKPGSSVKVSCKATGYTFTNYYMHWVRQ |
| P2C4_H3 | VL: SEQ ID NO: 23 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTLV |
| | | TVSS (SEQ ID NO: 89) |
| | | VL: |
| | | QSALTQPASVSGSPGQSIAISCTGTSSDIGHYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 71) |
| Anti-IL2Rβ | VH: SEQ ID NO: 150 of | VH: |
| antibody | WO 2017/021540 A1; | EVQLVQSGAEVKKPGSSVKVSCKASGYTFTNYYMHWVRQ |
| P2C4_C1D10 | VL: SEQ ID NO: 148 of | APGQGLEWMGAIMPSRGGTSYPQKFQGRVTMTGDTSTST |
| | WO 2017/021540 A1 | VYMELSSLRSEDTAVYYCARGEYYYDSSGYYYWGQGTPV |
| | | TVSS (SEQ ID NO: 90) |
| | | VL: |
| | | QSALTQPASVSGSPGQSIAISCTGTSSDIGDYDFVSWYQ |
| | | QHPGTAPKLIIYDINNRPSGISNRFSGSKSDNMASLTIS |
| | | GLQPEDEADYYCSAYTSSDTVVFGGGTKLT (SEQ ID |
| | | NO: 91) |
| Anti-IL2Rβ | VH: SEQ ID NO: 151 of | VH: |
| antibody | WO 2017/021540 A1; | EVQLVQSGAEVKKPGASVKVSCKASGYTFTNYYMHWVRQ |
| P2C4_FW2 | VL: SEQ ID NO: 149 of | APGQGLEWMGAIMPSRGGTSYPQKFQGRVTITADKSTST |
| | WO 2017/021540 A1 | AYMELSSLRSEDTAVYYCARGEYYYDSSGYYYWGQGTLV |
| | | TVSS (SEQ ID NO: 92) |
| | | VL: |
| | | QSVLTQPPSVSGAPGQRVTISCTGTSSDIGHYDFVSWYQ |
| | | QLPGTAPKLLIYDINNRPSGVPDRFSGSKSGTSASLAIT |
| | | GLQAEDEADYYCSAYTSSDTLVFGGGTKLT (SEQ ID |
| | | NO: 93) |
| Anti-IL2Rβ | VH: SEQ ID NO: 161 of | VH: |
| antibody | US 2023/0295348 A1; | EVQLVQSGTEVKKPGASVKVSCKASGYTFTTYAMHWVRQ |
| P2H7 | VL: SEQ ID NO: 162 of | APGQSLEWMGWINTGNGNTKYSQNFQGRVTMTRDTSIST |
| | US 2023/0295348 A1 | AYMELSRLRSDDTAVYYCARDLGQLERLYFWGQGTLVTV |
| | | SS (SEQ ID NO: 62) |
| | | VL: |
| | | DIQMTQSPSTLSASVGDRVTLSCRAGQAISSWLAWYQQK |
| | | PGKAPKLLIYKASNLESGVPSRFSGGGSGAEFTLTISSL |
| | | QPDDFATYYCQQYQSYPYTFGQGTKLEIR (SEQ ID |
| | | NO: 63) |
| Anti-IL2Rβ | VH: SEQ ID NO: 169 of | VH: |
| antibody | US 2023/0295348 A1; | HVQLVETGGGLVQPGGSLRLSCAASGFTFSSYAMSWVRQ |
| P2D12 | VL: SEQ ID NO: 170 of | APGKGLEWVSAISGSGGSTYYADSVKGRFTISRDNSKNT |
| | US 2023/0295348 A1 | LYLQMNSLRAEDTAVYYCARDLGDYWGQGTLVTVSS |
| | | (SEQ ID NO: 64) |
| | | VL: |
| | | DIQLTQSPSSLSASVGDRVTITCQASQDIGNYLNWYQLK |
| | | PGKAPKLLIYDASNLETGVPSRFSGSGSGTDFTFTISSL |
| | | QPEDIATYYCLQLYDYPLTFGGGTKVEIK (SEQ ID |
| | | NO: 65) |
| Anti-IL2Rβ | VH: SEQ ID NO: 175 of | VH: |
| antibody | US 2023/0295348 A1; | QVQLQQWGAGLLKPSETLSLTCAVYGGSFSGYYWSWIRQ |
| P1G11 | VL: SEQ ID NO: 176 of | PPGKGLEWIGEINHSGSTNYNPSLKSRVTISVDTSKNQF |
| | US 2023/0295348 A1 | SLKLSSVTAADTAVYYCARSSSGDAFDIWGQGTMVTVSS |
| | | (SEQ ID NO: 66) |
| | | VL: |
| | | NFMLTQPHSVSESPGKTVTISCTRSSGSIASNYVQWYQQ |
| | | RPGSSPTTVIFDDNQRPTGVPDRFSAAIDTSSSSASLTI |
| | | SGLTAEDEADYYCQSSHSTAVVFGGGTKLTVL (SEQ |
| | | ID NO: 67) |
| Anti-IL2Rβ | VH: SEQ ID NO: 4 of US | VH: |
| antibody | 2023/0295348 A1; VL: | EVQLLESGGGLVQPGGSLRLSCAASGFTFSSYAMSWVRQ |
| AL1 | SEQ ID NO: 42 of US | APGKGLEWVSAISGSGGSTYYADSVKGRFTISRDNSKNT |
| | 2023/0295348 A1 | LYLQMNSLRAEDTAVYYCAKSYGAFDYWGQGTLVTVSS |
| | | (SEQ ID NO: 167) |
| | | VL: |
| | | DIQMTQSPSSLSASVGDRVTITCQASQDISNYLNWYQQK |
| | | PGKAPKLLIYDASNLETGVPSRFSGSGSGTDFTFTISSL |
| | | QPEDIATYYCQQDANDPRTFGQGTKVEIK (SEQ ID |
| | | NO: 168) |
| Anti-IL2Rβ | VH: SEQ ID NO: 4 of US | VH: |
| antibody | 2023/0295348 A1; VL: | EVQLLESGGGLVQPGGSLRLSCAASGFTFSSYAMSWVRQ |
| AL2 | SEQ ID NO: 47 of US | APGKGLEWVSAISGSGGSTYYADSVKGRFTISRDNSKNT |
| | 2023/0295348 A1 | LYLQMNSLRAEDTAVYYCAKSYGAFDYWGQGTLVTVSS |
| | | (SEQ ID NO: 167) |
| | | VL: |
| | | DIQMTQSPSSLSASVGDRVTITCQASQDIGTYLNWYQQK |
| | | PGKAPKLLIYEASTLETGVPSRFSGSGSGTDFTFTISSL |
| | | QPEDIATYYCQQDGARDDYATFGQGTKVEIK (SEQ ID |
| | | NO: 169) |
| Anti-IL2Rβ | VH: SEQ ID NO: 4 of US | VH: |
| antibody | 2023/0295348 A1; VL: | EVQLLESGGGLVQPGGSLRLSCAASGFTFSSYAMSWVRQ |
| AL3 | SEQ ID NO: 52 of US | APGKGLEWVSAISGSGGSTYYADSVKGRFTISRDNSKNT |
| | 2023/0295348 A1 | LYLQMNSLRAEDTAVYYCAKSYGAFDYWGQGTLVTVSS |
| | | (SEQ ID NO: 167) |
| | | VL: |
| | | DIQMTQSPSSLSASVGDRVTITCQASQDIDDYLNWYQQK |
| | | PGKAPKLLIYDASNLQSGVPSRFSGSGSGTDFTLTISSL |
| | | QPEDFATYYCQQDSMDPRTFGQGTKVEIK (SEQ ID |
| | | NO: 170) |
| Anti-IL2Rβ | VH: SEQ ID NO: 4 of US | VH: |
| antibody | 2023/0295348 A1; VL: | EVQLLESGGGLVQPGGSLRLSCAASGFTFSSYAMSWVRQ |
| AL4 | SEQ ID NO: 57 of US | APGKGLEWVSAISGSGGSTYYADSVKGRFTISRDNSKNT |
| | 2023/0295348 A1 | LYLQMNSLRAEDTAVYYCAKSYGAFDYWGQGTLVTVSS |
| | | (SEQ ID NO: 167) |
| | | VL: |
| | | DIQMTQSPSSLSASVGDRVTITCRASQSIDEYLNWYQQK |
| | | PGKAPKLLIYEASKLQSGVPSRFSGSGSGTDFTLTISSL |
| | | QPEDFATYYCQQDGAMDTYATFGQGTKVEIK (SEQ ID |
| | | NO: 171) |
| Anti-IL2Rβ | VH: SEQ ID NO: 4 of US | VH: |
| antibody | 2023/0295348 A1; VL: | EVQLLESGGGLVQPGGSLRLSCAASGFTFSSYAMSWVRQ |
| AL5 | SEQ ID NO: 62 of US | APGKGLEWVSAISGSGGSTYYADSVKGRFTISRDNSKNT |
| | 2023/0295348 A1 | LYLQMNSLRAEDTAVYYCAKSYGAFDYWGQGTLVTVSS |
| | | (SEQ ID NO: 167) |
| | | VL: |
| | | EIVLTQSPATLSLSPGERATLSCRASQSVDEYLAWYQQK |
| | | PGQAPRLLIYDASERATGIPARFSGSGSGTDFTLTISSL |
| | | EPEDFAVYYCQQDATDPRTFGQGTKVEIK (SEQ ID |
| | | NO: 172) |

In some aspects, the IL2Rβ targeting moiety competes with an antibody set forth in Table R1 for binding to IL2Rβ. In further aspects, the IL2Rβ targeting moiety comprises CDRs having CDR sequences of an antibody set forth in Table R1. In some embodiments, the targeting moiety comprises all 6 CDR sequences of an antibody set forth in Table R1. In other embodiments, the IL2Rβ targeting moiety comprises at least the heavy chain CDR sequences (CDR-H1, CDR-H2, CDR-H3) of such antibody and the light chain CDR sequences of a universal light chain. In further aspects, an IL2Rβ targeting moiety comprises a VH comprising the amino acid sequence of the VH of an antibody set forth in Table R1. In some embodiments, the IL2Rβ targeting moiety further comprises a VL comprising the amino acid sequence of the VL of the antibody set forth above in Table R1. In other embodiments, the IL2Rβ targeting moiety further comprises a universal light chain VL sequence.

In some embodiments, the IL2Rβ targeting moieties are based on the exemplary anti-IL2Rβ single domain antibodies or antibody sequences set forth in Table R2 below.

TABLE R2
Exemplary Anti-IL2Rβ Single Domain Antibody (sdAb) Amino Acid Sequences

| Target or | | | SEQ |
|---|---|---|---|
| Description | Reference | Sequence | ID NO |
| IL2RB_F09C | SEQ ID NO: 11 | QVQLQESGPGLVKPSGTLSLTCAVSGGSISSSDWW | 55 |
| | of WO | SWVRQPPGKGLEWIGEIDHSGSTNYNPSLMSRVTI | |
| | 2022/212848 A1 | SVDKSKNQFSLKLSSVTAADTAVYFCGRGSWELSD | |
| | | AFDIRGQGTLVTVSS | |
| IL2RB_F09G | SEQ ID NO: 12 | QVQLQESGPGLVKSSETLSLTCTVSGGSISSSDWW | 57 |
| | of WO | SWVRQPPGKGLEWIGEIDHSGSTNYNPSLMSRVTI | |
| | 2022/212848 A1 | SVDKSKNQFSLKLSSVTAADTAVYFCARGSWELTD | |
| | | AFDIRGQGTLVTVSS | |
| IL2RB_F09K | SEQ ID NO: 13 | QVQLQESSPGLVKPSETLSLTCTVSGGSISSSNWW | 58 |
| | of WO | SWVRQPPGKGLEWIGEISHSGSTNYNPSLKSRVTI | |
| | 2022/212848 A1 | SVDKSKNQFSLRLSSVTAADTAVYFCGRGSWELTD | |
| | | AFDIRGQGTLVTVSS | |
| IL2RB_F18E | SEQ ID NO: 14 | QVQLVESGGGVVQPGRSLRLSCAASGFTFSSYGMH | 59 |
| | of WO | WVRQAPGKEREWVAVISYDGSNKYYTDSVKGRFTI | |
| | 2022/212848 A1 | SRDNSKNTLYLEMNSLRAEDTAVYYCARDLDYDVL | |
| | | TGDPVGGFDIWGQGTLVTVSS | |
| 14-MP02C03 | SEQ ID NO: 14 | EVQLVESGGGLVQTGGSLRLSCAASGSQFINDVMG | 94 |
| | of WO | WYRQVPGKQRELVADMDDTGSTEYADSVKGRFTIL | |
| | 2023/067194 A1 | RDSVKNTAYLQMSNLKPEDTGVYYCKAGLWIKGRH | |
| | | FDYWGQGTQVTVSS | |
| 15-MP02E06 | SEQ ID NO: 15 | QVQLVESGGGSVQPGGSLRLSCAASGFTFSNYAMS | 95 |
| | of WO | WVRQAPGKGLEWVASITGFGRGTDYADSVKGRFTI | |
| | 2023/067194 A1 | SRDNAEDTLYLQMNSLKPEDTAVYYCAKYSSSTYY | |
| | | PPTPARGRDYRGQGTQVTVSS | |
| 16-MP02B08 | SEQ ID NO: 16 | EVQLVESGGGLVQAGGSLRLSCAASGRAIENYPVG | 96 |
| | of WO | WFRQAPGKEREFVAAITWISGSTLYADSVKGRFTI | |
| | 2023/067194 A1 | SRDNAKNTVYLQMSSLKPEDTALYYCAAALKTITR | |
| | | GQNDYSYWGQGTQVTVSS | |
| 17-MP02C09 | SEQ ID NO: 17 | QVQLQESGGGLVQAGGSLRLSCVASGSVSSINGMA | 97 |
| | of WO | WYRQGADNQRVLVAAISRVGNTAYGDSVKGRFTIS | |
| | 2023/067194 A1 | RQNARNTVYLQMNSLKPEDTAVYYCNADSWGGDDY | |
| | | WGQGTQVTVSS | |
| 18-MP02C03 | SEQ ID NO: 18 | QVQLVESGGGLVQPGGSLRLSCAISGGTLDSYGIG | 98 |
| | of WO | WVRQAPGKQREGVSCMSRSDDRTYYADSVKGRFTI | |
| | 2023/067194 A1 | SKDSAKNTVYLQMTSLKPEDTAVYYCAAVDAYGCS | |
| | | LVQPTTYDFWGLGTQVTVSS | |
| 19-MP02F08 | SEQ ID NO: 19 | EVQLVESGGGLVQTGGSLRLSCAASGGTFSRDAMA | 99 |
| | of WO | WFRQVPGKEREFVALISWSGATTNYADSVKGRFAI | |
| | 2023/067194 A1 | SRDNGKNTVYLQMNRLKPADTAIYYCAADRRPMGS | |
| | | RSYFEPTEYDDWGQGTQVTVSS | |
| 20-MP02F10 | SEQ ID NO: 20 | EVQLVESGGGLVQAGGSLRLSCAASGRDFSSYAMG | 100 |
| | of WO | WFRQAPGKEREFVVAITWTKRSTDFPDSVKGRFTI | |
| | 2023/067194 A1 | SRDNAKNTVYLDMNSLKPEDTAVYYCASARGLPVT | |
| | | PLGDIIYWGEGTLVTVSS | |
| 21-MP03A 12 | SEQ ID NO: 21 | EVQLVESGGGLVQAGGSLRLSCAASGRTFSINAMG | 101 |
| | of WO | WFRQAPGKEREFVAAISRSGGSTVYVDGVKGRFTI | |
| | 2023/067194 A1 | SRDNAKNTVYLQMNSLEPEDTAVYYCAATMAVGWT | |
| | | TRWRTADFDSWGQGTQVTVSS | |
| 22-MP03F12 | SEQ ID NO: 22 | EVQLVESGGGLVQAGGSLRLSCAASGSIFSINAMA | 102 |
| | of WO | WFRQVPGMERELVAAISRDGGASVYRDSVKGRFTI | |
| | 2023/067194 A1 | SRDNSKNTVYLQMNTLKPEDTAIYVCAATRAIGWT | |
| | | ARWITTDFDFWGQGTQVTVSS | |
| 23-MP06F03 | SEQ ID NO: 23 | QVQLVESGGGLVQAGGSLRLSCAVSGDVFVRYTMA | 103 |
| | of WO | WFRQAPGKEREFVASVTDSGRTTDYVHSVKGRFTV | |
| | 2023/067194 A1 | SRDNAKNTVYLQMNNLKPEDTAVYYCAANTDYFQI | |
| | | KSLDANTWGQGTQVTVSS | |
| 24-MP06E05 | SEQ ID NO: 24 | QVQLVESGGELVQGGASLRLSCAASGRTFSNANMA | 104 |
| | of WO | WFRQAPEKEREFVALITWSSGSTLYADSVKGRFTI | |
| | 2023/067194 A1 | SRDNARKMVYLQMNSLKPEDTAVYYCAADGPPYSG | |
| | | TYYRYDTYDYWGQGTQVTVSS | |
| 25-MP06F05 | SEQ ID NO: 25 | QVQLVESGGGLVQTGDSLRLSCAASGRSLDTTYIA | 105 |
| | of WO | WFRQAPGKERDFLAYISPRFSHTWYADSVKGRFTI | |
| | 2023/067194 A1 | SRNIAKRTVDLEMNSLEPEDTAVYYCAAREHSGST | |
| | | AWEHYDHWGQGTQVTVSS | |
| 26-MP06A07 | SEQ ID NO: 26 | QVQLQESGGGLVQAGGSLRLSCAASGDVFVRYTMA | 106 |
| | of WO | WFRQAPGKEREFVASVTDSGRTTEYVDSVKGRFTV | |
| | 2023/067194 A1 | SRDNAKNTAYLQMNNLKPEDTAIYYCAANTDYFQI | |
| | | RSLDLNTWGQGTQVTVSS | |
| βNb1 | Sequence | QVQLQESGGGSVQAGGSLRLSCVTSGYTYSSANMA | 107 |
| | disclosed in FIG. | WFRQAPGKEREGVAIITPSGRATTYADSVKGRFTI | |
| | S1 of Yen et al., | SRDNAANTLYLQMNSLKPEDTAMYYCAADTPPYSG | |
| | 2023, Cell. | LWYAERTYNYWGQGTQVTVSS | |
| | 185(8): 1414- | | |
| | 1430.e19 | | |
| βNb3 | Sequence | QVQLQESGGGSVQAGGSLRLSCTASGFTFDDEDMG | 108 |
| | disclosed in FIG. | WYRQAPGNECELVSSIGSLGRRYYADSVKDRFAIS | |
| | S1 of Yen et al., | QDNAKNTVYLQMNSLKPEDTAVYYCAATKGGSWLD | |
| | 2023, Cell. | SILASCQGAFGYWGQGTQVTVSS | |
| | 185(8): 1414- | | |
| | 1430.e19 | | |
| βNb4 | Sequence | QVQLQESGGGSVQAGGSLRLSCAASGSTSCSSVMR | 109 |
| | disclosed in FIG. | WYRQAPGKEREFVSSINSDRRTVYADSVKGRFTIS | |
| | S1 of Yen et al., | QDNAKSTLYLQMNSLKAEDTATYYCQRELYGDSWC | |
| | 2023, Cell. | QGNYWGQGTQVTVSS | |
| | 185(8): 1414- | | |
| | 1430.e19 | | |
| βNb6 | Sequence | QVQLQESGGGSVQAGGSLRLSCAASSYTISSVCMG | 110 |
| | disclosed in FIG. | WFRQAPGKEREGVAGIAPDGSTGYGDSVKGRFTIS | |
| | S1 of Yen et al., | KDNAKNTLYLQMNSLKPEDTAMYYCAAASPGRCFL | |
| | 2023, Cell. | PRTALEPALYYNWGQGTQVTVSS | |
| | 185(8): 1414- | | |
| | 1430.e19 | | |
| hIL3Rb-VHH- | SEQ ID NO: 1 of | QVQLQESGGGSVQAGGSLRLSCVGSGYTYDTSDMS | 173 |
| 1 | U.S. | WYRQAPGKEREFVSDIDSGDWAAYADAVKGRFTIS | |
| | 20230/272090 | RDNAKKTVYLQMNSLEPEDTAMYYCKASYWKWGKL | |
| | A1 | NNFWGPGTQVTVSS | |
| hIL3Rb-VHH- | SEQ ID NO: 5 of | QVQLQESGGGLVQPGGSLRLSCVASGFTFSNYWIF | 174 |
| 2 | U.S. | WVRQAAGKGLEWLSTSNTGGDTTKYADSVKGRFTI | |
| | 20230/272090 | SRDSAKNTEYLQMNSLKPEDTAVYYCETGRCARSG | |
| | A1 | GYQGTQVTVSS | |
| hIL3Rb-VHH- | SEQ ID NO: 9 of | QVQLQESGGGLVQPGGSLKLSCAASGFRFSNYGMS | 175 |
| 3 | U.S. | WVRQAPGEGLEWVSYINGDGSRTHYADSVKGRFTI | |
| | 20230/272090 | SRDNAKNTLYLQLNSLKTEDTAMYYCEKGLSRDGW | |
| | A1 | SLSAASRGQGTQVTVSS | |
| hIL3Rb-VHH- | SEQ ID NO: 13 | QVQLQESGGGSVQTGGSLRLSCAVSGYTTYSFNYM | 176 |
| 4 | of U.S. | GWFRQAPGKEREGVAVIYTGGGSTLYADSVKGRFT | |
| | 20230/272090 | ISQDNAKNTVYLQMNSLKPEDTAMYYCAADDQRFA | |
| | A1 | SPLYAYFGYWGQGTQVTVSS | |
| hIL3Rb-VHH- | SEQ ID NO: 17 | QVQLQESGGGSVQVGGSLRLSCATSGDTKSIRCMG | 177 |
| 5 | of U.S. | WFRQTPGKEREGIAAIDREGFATYADSVYDRFTIA | |
| | 20230/272090 | QDNAQNTLYLEMNALKPEDTAMYYCAAQNMCRVVR | |
| | A1 | GAMTGVDYWGKGTQVTVSS | |
| hIL3Rb-VHH- | SEQ ID NO: 21 | QVQLQESGGGSVQAGGSLRLSCAASEYTASRYCMA | 178 |
| 6 | of U.S. | WFRQAPGKEREGVAAIHPGGGTTYYADSVKGRFSI | |
| | 20230/272090 | SQDSADNTLYLQMNSLKPEDTAMYYCAAGSLWVPF | |
| | A1 | GDRCAANYWGQGTQVTVSS | |
| hIL3Rb-VHH- | SEQ ID NO: 25 | QVQLQESGGGSVQAGGSLRLSCAASGYEYCRIHMT | 179 |
| 7 | of U.S. | WYRQGPGKEREFVSSIGSDGRKTYANSVTGRFTIS | |
| | 20230/272090 | RDNANHTVYLQMNSLSPEDTAMYYCKTEYLYGLGC | |
| | A1 | PDGSAYWGQGTQVTVSS | |
| hIL3Rb-VHH- | SEQ ID NO: 29 | QVQLQESGGGSVQVGGSLKLSCAASGYTYSSYYCM | 180 |
| 8 | of U.S. | GWFRQAPGKEREGVAAIDSDGSTSYADSVKGRFTI | |
| | 20230/272090 | SQDDAKNTLYLQMNSLKPEDTAMYYCAASYEVVDC | |
| | A1 | YPSGYGQDYWGKGTQVTVSS | |

In some aspects, the IL2RR targeting moiety competes with an antibody set forth above in Table R2 for binding to IL2RR. In further aspects, the IL2RR targeting moiety comprises CDRs having CDR sequences of an antibody set forth in Table R2. In some embodiments, the IL2RR targeting moiety comprises all 3 CDR sequences of an antibody set forth in Table R2. In further aspects, an IL2Rγ targeting moiety comprises a VH (e.g., a VHH or sdVH) comprising the amino acid sequence of the VH of an antibody set forth in Table R2.

In some embodiments, the IL2RR targeting moiety binds an epitope at similar proximity to cell membrane as the IL2Rγ targeting of the tumor-targeted split IL2 receptor agonist. In some embodiments, if the IL2Rγ targeting moiety binds to the D1 domain of IL2Rγ, then the IL2RR targeting moiety binds to 02 domain of IL2RR.

### 6.4. Tumor-Targeted IL2Rγ Binding Molecule

### 6.4.1. Tumor-Associated Antigen Targeting Moieties

The tumor-targeted IL2Rγ binding molecule of the tumor-targeted split IL2 receptor agonists of the disclosure comprise a tumor-associated antigen (“TAA”) targeting moiety. Typically, the TAA recognized by the TAA targeting moiety of the tumor-targeted IL2Rγ binding molecule is expressed on the same cancer cell as the TAA recognized by the TAA targeting moiety of the tumor-targeted IL2Rβ binding molecule.

In some embodiments, both TAA targeting moieties recognize the same TAA, whether on the same epitope or on different epitopes. If the TAA targeting moieties recognize different epitopes, they preferably can bind to the cancer cell simultaneously and/or in a non-competing manner (e.g., are non-competing TAA targeting moieties as determined using an antibody cross-competition assay as described in Section 8.1.6). Both TAA targeting moieties recognize TAAs expressed on the same cancer cell, which may be the same TAA or different TAAs.

In some embodiments, both TAA targeting moieties recognize different TAAs expressed on the same cancer cell.

Suitable TAA targeting moieties are described in Section 6.5.

### 6.4.2. IL2Rγ Binding Moieties

In some embodiments, the tumor-targeted IL2Rγ binding molecule of the tumor-targeted split IL2 receptor agonists of the disclosure comprise an IL2Rγ targeting moiety as an IL2Rγ binding moiety.

The IL2Rγ targeting moiety typically is or comprises an antigen binding domain of an antibody. The IL2Rγ targeting moiety can be any format, e.g., as disclosed in Section 6.6 or subsections thereof. In some embodiments, the IL2Rγ targeting moiety is a Fab. In some embodiments, the IL2Rγ targeting moiety is an scFv. In some embodiments, the IL2Rγ targeting moiety is a sdAb. In some embodiments, the IL2Rγ targeting moiety (e.g., IL2Rγ targeting sdAb) is an agonistic binder capable of activating IL2 receptor signaling following binding to IL2Rγ expressed on a cell. Such an agonistic IL2Rγ targeting moiety may be capable of activating IL2 receptor signaling alone and/or in combination with an IL2Rβ agonistic binder (e.g., an IL2Rβ binding moiety as described in Section 6.3.2).

In some embodiments, the IL2Rγ targeting moiety is based on an antibody comprising both heavy and light chain variable regions. Exemplary anti-IL2Rγ antibodies are set forth in Table R3 below.

TABLE R3
Exemplary Anti-IL2Rγ Variable Heavy (VH) and Light (VL) Chain Amino Acid Sequences

| Target or | | |
|---|---|---|
| Description | Reference | Sequence |
| Anti-IL2Rγ | VH: SEQ ID NO: 22 of | VH: |
| antibody | WO 2022/212848 A1 | QVQLVESGGGLVKPGGSLRLSCAASGFTFSDYYMSWIRQ |
| F16A | VL: SEQ ID NO: 47 of | APGKGLEWVSSISSSGDTIYYADSVQGRFTLSRDNAENS |
| | WO 2022/212848 A1 | LFLQMNSLRAEDTAVYYCARGDAVSITGDYRGQGTLVTV |
| | | SS (SEQ ID NO: 111) |
| | | VL: |
| | | EIVMTQSPATLSVSPGERATLSCRASQSVSSNLAWYQQK |
| | | PGQAPRLLIYGASTRATGIPARFSGSGSGTEFTLTISSL |
| | | QSEDFAVYYCQQYNNWPWTFGQGTKVEIK (SEQ ID |
| | | NO: 56) |
| Anti-IL2Rγ | VH: SEQ ID NO: 23 of | VH: |
| antibody | WO 2022/212848 A1 | QVQLVESGGGLVKPGGSLRLSCAASGFTFSDYYMSWIRQ |
| F16B | VL: SEQ ID NO: 47 of | APGKGLEWVSYISSSGSTIYYADSVKGRFTISRDNAKNS |
| | WO 2022/212848 A1 | LYLQMNSLRAEDTAVYYCARGDAVSITGDYRGQGTLVTV |
| | | SS (SEQ ID NO: 112) |
| | | VL: |
| | | EIVMTQSPATLSVSPGERATLSCRASQSVSSNLAWYQQK |
| | | PGQAPRLLIYGASTRATGIPARFSGSGSGTEFTLTISSL |
| | | QSEDFAVYYCQQYNNWPWTFGQGTKVEIK (SEQ ID |
| | | NO: 56) |
| Anti-IL2Rγ | VH: SEQ ID NO: 24 of | VH: |
| antibody | WO 2022/212848 A1 | QVQLVESGGGLVKPGGSLRLSCAASGFTFNDYYMSWIRQ |
| F16C | VL: SEQ ID NO: 47 of | APGKGLEWVSHISSSGSTIYYADSVKGRFTVSRDNANNS |
| | WO 2022/212848 A1 | LYLQMHSLRAEDTAVYYCARGDAVSITGDYRGQGTLVTV |
| | | SS (SEQ ID NO: 113) |
| | | VL: |
| | | EIVMTQSPATLSVSPGERATLSCRASQSVSSNLAWYQQK |
| | | PGQAPRLLIYGASTRATGIPARFSGSGSGTEFTLTISSL |
| | | QSEDFAVYYCQQYNNWPWTFGQGTKVEIK (SEQ ID |
| | | NO: 56) |
| Anti-IL2Rγ | VH: SEQ ID NO: 25 of | VH: |
| antibody | WO 2022/212848 A1 | QVQLVESGGDLVKPGGSLRLSCAASGFTFSDYYMSWLRQ |
| F18A | VL: SEQ ID NO: 47 of | APGKELEWVSHISSSGTTTYYADSVEGRFTITRDNAKNS |
| | WO 2022/212848 A1 | LYLQMNSLRAEDTAVYYCARGAAVAPGFDSRGQGTLVTV |
| | | SS (SEQ ID NO: 114) |
| | | VL: |
| | | EIVMTQSPATLSVSPGERATLSCRASQSVSSNLAWYQQK |
| | | PGQAPRLLIYGASTRATGIPARFSGSGSGTEFTLTISSL |
| | | QSEDFAVYYCQQYNNWPWTFGQGTKVEIK (SEQ ID |
| | | NO: 56) |
| Anti-IL2Rγ | VH: SEQ ID NO: 76 of | VH: |
| antibody | WO 2017/021540 A1; | QVQLQQWGAGLLKPSETLSLTCAVYGGSFSGYYWSWIRQ |
| P1A3 | VL: SEQ ID NO: 67 of | PPGKGLEWIGEINHSGSTNYNPSLKSRATISVDTSKNQF |
| | WO 2017/021540 A1 | SLKLSSVTAADTAVYYCATSPGGYSGGYFQHWGQGTLVT |
| | | VSS (SEQ ID NO: 115) |
| | | VL: |
| | | DVVMTQSPLSLPVTPGEPASISCRSSQSLLHSNGYNYLD |
| | | WYLQKPGQSPQLLIYLGSNRDSGVPDRFSGSGSGTDFTL |
| | | KISRVEAEDVGVYYCMQGTHWPWTFGQGTKVEIK (SEQ |
| | | ID NO: 116) |
| Anti-IL2Rγ | VH: SEQ ID NO: 82 of | VH: |
| antibody | WO 2017/021540 A1; | QVQLQQWGAGLLKPSETLSLTCAVYGGSFSGYYWSWIRQ |
| P1A3_B3 | VL: SEQ ID NO: 67 of | PPGKGLEWIGEINHFGSTNYNPSLKSRATISVDTSKNQF |
| | WO 2017/021540 A1 | SLKLSSVTAADTAVYYCATSPGGYSGGYFQHWGQGTLVT |
| | | VSS (SEQ ID NO: 117) |
| | | VL: |
| | | DVVMTQSPLSLPVTPGEPASISCRSSQSLLHSNGYNYLD |
| | | WYLQKPGQSPQLLIYLGSNRDSGVPDRFSGSGSGTDFTL |
| | | KISRVEAEDVGVYYCMQGTHWPWTFGQGTKVEIK (SEQ |
| | | ID NO: 116) |
| Anti-IL2Rγ | VH: SEQ ID NO: 84 of | VH: |
| antibody | WO 2017/021540 A1; | QVQLQQWGAGMLKPSETLSLTCAVYGGSFSGYYWSWIRQ |
| P1A3_E8 | VL: SEQ ID NO: 67 of | PPGKGLEWIGEINHFGSTNYNPSLKSRATISVDTSKNQF |
| | WO 2017/021540 A1 | SLKLSSVTAADTAVYYCATSPGGYSGGYFQHWGQGTLVT |
| | | VSS (SEQ ID NO: 118) |
| | | VL: |
| | | DVVMTQSPLSLPVTPGEPASISCRSSQSLLHSNGYNYLD |
| | | WYLQKPGQSPQLLIYLGSNRDSGVPDRFSGSGSGTDFTL |
| | | KISRVEAEDVGVYYCMQGTHWPWTFGQGTKVEIK (SEQ |
| | | ID NO: 116) |
| Anti-IL2Rγ | VH: SEQ ID NO: 78 of | VH: |
| antibody | WO 2017/021540 A1; | QVQLQESGPGLVKPSETLSLTCTVSGGSISSSSYYWGWI |
| P2B9 | VL: SEQ ID NO: 71 of | RQPPGKGLEWIGSIYYSGSTYYNPSLKSRVTISVDTSKN |
| | WO 2017/021540 A1 | QFSLKLSSVTAADTAVYYCAGDILTGYALDYWGQGTLVT |
| | | VSS (SEQ ID NO: 119) |
| | | VL: |
| | | SYELTQPPSMSVSPGQTARITCSGDALPKQFAFWYQQKP |
| | | GQAPVLVIYKDTERPSGIPERFSGSSSGTTVTLTITGVQ |
| | | AEDEADYYCQSPDSSGTVEVFGGGTKLTVL (SEQ ID |
| | | NO: 120) |
| Anti-IL2Rγ | VH: SEQ ID NO: 82 of | VH: |
| antibody | WO 2017/021540 A1; | QVQLQQWGAGLLKPSETLSLTCAVYGGSFSGYYWSWIRQ |
| P1A3_B4 | VL: SEQ ID NO: 75 of | PPGKGLEWIGEINHFGSTNYNPSLKSRATISVDTSKNQF |
| | WO 2017/021540 A1 | SLKLSSVTAADTAVYYCATSPGGYSGGYFQHWGQGTLVT |
| | | VSS (SEQ ID NO: 117) |
| | | VL: |
| | | DVVMTQSPLSLPVTPGESVSISCRSSQSLLHSNGYNYLD |
| | | WYLQKPGQSPQLLIYLGSNRDSGVPDRFSGSGSGTDFTL |
| | | KISRVEAEDVGVYYCMQGTHWPWTFGQGTKVEIK (SEQ |
| | | ID NO: 121) |
| Anti-IL2Rγ | VH: SEQ ID NO: 153 of | VH: |
| antibody | WO 2017/021540 A1; | EVQLVESGGGLVQPGGSLRLSCAASGGSFSGYYWSWVRQ |
| P1A3_FW2 | VL: SEQ ID NO: 152 of | APGKGLEWVSEINHSGSTNYNPSLKSRFTISRDNSKNTL |
| | WO 2017/021540 A1 | YLQMNSLRAEDTAVYYCARSPGGYSGGYFQHWGQGTLVT |
| | | VSS (SEQ ID NO: 122) |
| | | VL: |
| | | DIQMTQSPSSLSASVGDRVTITCRSSQSLLHSNGYNYLD |
| | | WYQQKPGKAPKLLIYLGSNRDSGVPSRFSGSGSGTDFTL |
| | | TISSLQPEDFATYYCMQGTHWPWTFGQGTKVEIK (SEQ |
| | | ID NO: 123) |
| Anti-IL2Rγ | VH: SEQ ID NO: 2 of | VH: |
| antibody | WO 2020/160242 A1; | QVQLVQSGAEVKKPGASVRVSCKASGYTFTDYDIHWVRQ |
| | VL: SEQ ID NO: 10 of | APGHGLEWMGWINPNSGGTNYAQKFQGRVTMTRDTSIST |
| | WO 2020/160242 A1 | VYMDLSRLRSDDTAVYYCARADYSSSYYYYGMDVWGQGT |
| | | TVTVSS (SEQ ID NO: 181) |
| | | VL: |
| | | DIVMTQSPDSLAVSLGERATINCKSSQSVLYSSKNKNYL |
| | | SWYQQKPGQPPKLLIYWASTREFGVPDRFSGRGSGTDFT |
| | | LTISSLQAEDVAVYYCQQYYTTPYTFGQGTKLEIK |
| | | (SEQ ID NO: 182) |
| Anti-IL2Rγ | VH: SEQ ID NO: 22 of | VH: |
| antibody | WO 2020/160242 A1; | QVQLVESGGGVVQPGRSLRLSCTASGFTFRSYDMYWVRQ |
| | VL: SEQ ID NO: 30 of | APGKGLEWVSVITYDGNNKYYADSVKGRFTISRDNSKNT |
| | WO 2020/160242 A1 | LFLQMSSLRPEDTAVYYCAKRGLIWVGESFDYWGQGTLV |
| | | TVSS (SEQ ID NO: 183) |
| | | VL: |
| | | DIQMTQSPSTLSASVGDRVTITCRASQSINSWLAWYQQK |
| | | PGKAPNLLIYKASSLESGVPSRFSGSGSGTEFTLTISSL |
| | | QPDDFATYYCQQYKSYSWTFGQGTKVEIK (SEQ ID |
| | | NO: 184) |
| Anti-IL2Rγ | VH: SEQ ID NO: 42 of | VH: |
| antibody | WO 2020/160242 A1; | QVQLVESGGGVVQPGRSLRLSCAASGENFRNFGMHWVRQ |
| | VL: SEQ ID NO: 50 of | APGKGLEWVAGILYDGSSKYYADSVKDRFTISRDNSKNT |
| | WO 2020/160242 A1 | LFLQMNSLRAEDTAMYYCAKEEDTAMVPFDSWGPGTLVT |
| | | VSS (SEQ ID NO: 185) |
| | | VL: |
| | | DIQLTQSPSFLSASVGDRVTITCWASQGISSYLAWYQQK |
| | | PGKAPTLLIYAASTLQSGVPSRFSGSGSGTEFTLTISSL |
| | | QPEDFASYYCQQLKSYPLTFGGGTKVEIK (SEQ ID |
| | | NO: 186) |
| Anti-IL2Rγ | VH: SEQ ID NO: 62 of | VH: |
| antibody | WO 2020/160242 A1; | QVQLQESGPGLVKPSQTLSLTCTVSGGSISSGGYYWSWI |
| | VL: SEQ ID NO: 70 of | RQHPGKGLEWIGFIYYSGKTYYNPSLKSRLTISVDTSKS |
| | WO 2020/160242 A1 | QFSLKLRSVTAADTAVYYCARLGYTNSAGWFDPWGQGTL |
| | | VTVSS (SEQ ID NO: 187) |
| | | VL: |
| | | DIQMTQSPSSLSASVGDRVTITCRASQSISSYLNWYQQK |
| | | PGKAPNLLIYAASSLQSGVPSRFSGSGSGTDFTLTISSL |
| | | QPEDLATYYCQQSYTTPFTFGPGTKVDIK (SEQ ID |
| | | NO: 188) |
| Anti-IL2Rγ | VH: SEQ ID NO: 81 of | VH: |
| antibody | WO 2020/160242 A1; | EVQLVESGGGLVKPGGSLRLSCAASGFTFSTAWMSWVRQ |
| | VL: SEQ ID NO: 89 of | SPGRGLEWVGRMKSKTDGGTTFYAAPVKGRFTISRDDSK |
| | WO 2020/160242 A1 | NTLYLQMNSLKTEDTAVYYCTTGLVPAFYKYYGVDVWGQ |
| | | GTTVTVSS (SEQ ID NO: 189) |
| | | VL: |
| | | DIQMTQSPSSLSASVGDRITITCQASQDITNYLNWYQQK |
| | | PGKAPNLLIYDASNLVTGVPSRFSGSGSGTDFTFTILSL |
| | | QPEDIATYYCQQYDSLLTFGPGTKVDIK (SEQ ID |
| | | NO: 190) |
| Anti-IL2Rγ | VH: SEQ ID NO: 101 of | VH: |
| antibody | WO 2020/160242 A1; | EVQLVESGGGLVQPGGSLRLSCAASGFTFNNYAMHWVRQ |
| | VL: SEQ ID NO: 109 of | APGKGLEYVSSISSSGGSTYYEDSVKGRFTISRDNSKNT |
| | WO 2020/160242 A1 | LYLQMGSLRAEDMAVYYCARSFYGSGTYYDTFDMWGQGT |
| | | MVTVSS (SEQ ID NO: 191) |
| | | VL: |
| | | DIQMTQSPSSLSASIGDRVTITCRASQSISRYLNWYQQK |
| | | PGKAPKLLIYAASSLQSGVPSRFSASGSGTDFTLTISSL |
| | | QPEDFATYYCQQSYSTPFTFGQGTKLEIK (SEQ ID |
| | | NO: 192) |
| Anti-IL2Rγ | VH: SEQ ID NO: 119 of | VH: |
| antibody | WO 2020/160242 A1; | QVQLVESGGDLVKPGGSLRLSCATSGFTFSDFYMTWIRQ |
| | VL: SEQ ID NO: 127 of | APGKGLEWISYISNSGSIVKYADSVKGRFTISRDNAKNS |
| | WO 2020/160242 A1 | LYLQMNSLRAEDTAIYYCARFYGDRWGQGTLVTVSS |
| | | (SEQ ID NO: 193) |
| | | VL: |
| | | DIQLTQSPSFLSASVGDRVTITCWASQGISTFLAWYQQK |
| | | PGKAPKLLIYAASTLQSGVPSRFSGSGSGTDFTLTISSL |
| | | QPEDFATYHCQQLNNYPWTFGQGTKVEIK (SEQ ID |
| | | NO: 194) |
| Anti-IL2Rγ | VH: SEQ ID NO: 138 of | VH: |
| antibody | WO 2020/160242 A1; | QVQLVESGGGLVKPGGSLRLSCEASGFTFNDFYMTWIRQ |
| | VL: SEQ ID NO: 146 of | APGKGLEWIAYISKSGDKMRYADSVKGRFSTSRDNAKNS |
| | WO 2020/160242 A1 | LSLQMNSLRAEDTAVYYCARFYGDIWGQGTLVTVSS |
| | | (SEQ ID NO: 195) |
| | | VL: |
| | | DIQLTQSPSFLSASVGDRVTITCWASQDISSFLVWYQQK |
| | | PGKAPNLLIYAASALQSGVPSRFSGSGSGTEFTLTISSL |
| | | QPEDFASYYCEQLNNYPWTFGQGTKVEIK (SEQ ID |
| | | NO: 196) |
| Anti-IL2Rγ | VH: SEQ ID NO: 156 of | VH: |
| antibody | WO 2020/160242 A1; | EVQLVESGGRLVQPGGSLRLSCEASGFTFSNYGMTWVRQ |
| | VL: SEQ ID NO: 164 of | APGKGLEWVSVISGSDNRKYYAESVKGRFTISRDNSKNT |
| | WO 2020/160242 A1 | LYLQMNSLRAEDTAVYYCAKLGYSRSSKDFYYGMDVWGQ |
| | | GTTVTVSS (SEQ ID NO: 197) |
| | | VL: |
| | | DIVMTQSPDSLAVSLGERATINCKSSQSVLYNSNNRNYL |
| | | VWYQQKPGQSPKLLIYWASTRESGVPDRFSGSGSGTDFT |
| | | LTISSLQAEDVAVYYCQQYYNVPYTFGQGTKLEIK |
| | | (SEQ ID NO: 198) |
| Anti-IL2Rγ | VH: SEQ ID NO: 174 of | VH: |
| antibody | WO 2020/160242 A1; | EVQLVESGGGVVRPGGSLRLSCAASGFTFDDYGMSWVRQ |
| | VL: SEQ ID NO: 182 of | APGKGLEWISSINRNGGSADYADSVKGRFTISRDNAKNS |
| | WO 2020/160242 A1 | LFLQMSSLRAEDTALYHCASGEFRFDYWGQGTLVTVSS |
| | | (SEQ ID NO: 199) |
| | | VL: |
| | | DIQMTQSPSSLSASVGDRVTITCRASQSISSYLNWYQQK |
| | | PGKAPKLLIYAASSLQSGVPSRFSGSGSGTDFTLTISSL |
| | | QPEDFATYYCQQSYSTPPITFGQGTRLEIK (SEQ ID |
| | | NO: 43) |
| Anti-IL2Rγ | VH: SEQ ID NO: 190 of | VH: |
| antibody | WO 2020/160242 A1; | EVQLVESGGGLVQPGRSLRLSCAASGFTLEDYAMHWVRQ |
| | VL: SEQ ID NO: 182 of | APGKGLEWVSGISWNRGSTGYADSVKGRFTISRDNAKNS |
| | WO 2020/160242 A1 | LYLQMTSLRAEDTALYYCAKGFYSMDVWGQGTTVTVSS |
| | | (SEQ ID NO: 200) |
| | | VL: |
| | | DIQMTQSPSSLSASVGDRVTITCRASQSISSYLNWYQQK |
| | | PGKAPKLLIYAASSLQSGVPSRFSGSGSGTDFTLTISSL |
| | | QPEDFATYYCQQSYSTPPITFGQGTRLEIK (SEQ ID |
| | | NO: 43) |
| Anti-IL2Rγ | VH: SEQ ID NO: 200 of | VH: |
| antibody | WO 2020/160242 A1; | QVQLQQSGPGLVKPSQTLSLTCAISGDSVSSNIAAWNWI |
| | VL: SEQ ID NO: 182 of | RLSPSRGLEWLGRTFFRSTWFYDYSLSVKGRITINPDTS |
| | WO 2020/160242 A1 | KNQFSLHLNSVTPEDAAVYYCARTGRRWSLDYWGQGTLV |
| | | TVSS (SEQ ID NO: 201) |
| | | VL: |
| | | DIQMTQSPSSLSASVGDRVTITCRASQSISSYLNWYQQK |
| | | PGKAPKLLIYAASSLQSGVPSRFSGSGSGTDFTLTISSL |
| | | QPEDFATYYCQQSYSTPPITFGQGTRLEIK (SEQ ID |
| | | NO: 43) |
| Anti-IL2Rγ | VH: SEQ ID NO: 210 of | VH: |
| antibody | WO 2020/160242 A1; | EVQLVESGGGVVRPGGSLRLSCATSGFTFDDYGMSWVRQ |
| | VL: SEQ ID NO: 182 of | VPGKGLEWVSSVNRNGGTTDYADSVKGRFTISRDNAKRS |
| | WO 2020/160242 A1 | LFLQMNSLRAEDTALYHCATGELFFDYWGQGTLVTVSS |
| | | (SEQ ID NO: 202) |
| | | VL: |
| | | DIQMTQSPSSLSASVGDRVTITCRASQSISSYLNWYQQK |
| | | PGKAPKLLIYAASSLQSGVPSRFSGSGSGTDFTLTISSL |
| | | QPEDFATYYCQQSYSTPPITFGQGTRLEIK (SEQ ID |
| | | NO: 43) |
| Anti-IL2Rγ | VH: SEQ ID NO: 218 of | VH: |
| antibody | WO 2020/160242 A1; | QVQLVQSGAEVKKPGASVKVSCKASGYTFTGHYMHWVRQ |
| | VL: SEQ ID NO: 226 of | APGQGLEWMGWIYPHSGHTNYAKRFQGRVTMTRDTSITT |
| | WO 2020/160242 A1 | AYMELIRLRSDDTAVYYCARRSGRSWYFDLWGRGTLVTV |
| | | SS (SEQ ID NO: 203) |
| | | VL: |
| | | EIVLTQSPGTLSLSPGERATLSCRASQSVSSSYLAWYQQ |
| | | KPGQAPRLLIYGASSRATGIPDRFSGSGSGTDFTLTISR |
| | | LEPEDFAVYYCQQYGSSPWTFGQGTKVEIK (SEQ ID |
| | | NO: 204) |
| Anti-IL2Rγ | VH: SEQ ID NO: 238 of | VH: |
| antibody | WO 2020/160242 A1; | EVQLVESGGGLVQPGGSLGLSCAASGFTFSNYAMSWVRQ |
| | VL: SEQ ID NO: 246 of | APGKGLEWVSAVSGGGGGTYYADSVKGRFTISRDNSKNT |
| | WO 2020/160242 A1 | VLLQMNSLRAEDTAVYYCARGRTGGLDYWGPGTLVTVSS |
| | | (SEQ ID NO: 205) |
| | | VL: |
| | | DVVMTQSPLSLPVIFGQPASISCRSSQSLVDSDGNTYLN |
| | | WLQQRPGQSPRRLIYEVSNRDSGVPDRFSGSGSGTDFTL |
| | | TISRVEAEDVGIYYCMQGTRWPPTFGGGTKVEIK (SEQ |
| | | ID NO: 206) |
| Anti-IL2Rγ | VH: SEQ ID NO: 258 of | VH: |
| antibody | WO 2020/160242 A1; | EVQLVESGGGVVRPGGSLRLSCAASGFIFDDYDMSWVRQ |
| | VL: SEQ ID NO: 266 of | PPGRGLEWVSGIDWFGGTRGYADSMKGRFTISRDNAKNS |
| | WO 2020/160242 A1 | LYLQMNSLRVEDTAFYYCARGGAIVGAVTPFDYWGQGTL |
| | | VTVSS (SEQ ID NO: 207) |
| | | VL: |
| | | DIQMTQSPSSLSASVGNRVTLSCRASQSINTYLSWYQQR |
| | | PGKAPKLLIYAASSLQSGVPSRFSGSGAGTDFTLTISSL |
| | | QPEDFATYYCQQSYSAPLTFGGGTKVEIK (SEQ ID |
| | | NO: 208) |
| Anti-IL2Rγ | VH: SEQ ID NO: 276 of | VH: |
| antibody | WO 2020/160242 A1; | QLQLQESGPGLVKPSETLSLTCTVSGGSISIKNYYWGWI |
| | VL: SEQ ID NO: 182 of | RQPPGKGLEWIGSIYYSGTTYYNPSLKSRVTISVDTSKN |
| | WO 2020/160242 A1 | QFSLKLSSVTAADTAVYHCARHGYSYGHGWFDPWGQGTL |
| | | VTVSS (SEQ ID NO: 209) |
| | | VL: |
| | | DIQMTQSPSSLSASVGDRVTITCRASQSISSYLNWYQQK |
| | | PGKAPKLLIYAASSLQSGVPSRFSGSGSGTDFTLTISSL |
| | | QPEDFATYYCQQSYSTPPITFGQGTRLEIK (SEQ ID |
| | | NO: 43) |
| Anti-IL2Rγ | VH: SEQ ID NO: 286 of | VH: |
| antibody | WO 2020/160242 A1; | QVQLQQSGPGLVKPSQTLSLTCDISGDSVSSNIATWNWI |
| | VL: SEQ ID NO: 182 of | RQSPSRGLEWLGRTYYRSKWYKDYAVSVKSRITINPDTS |
| | WO 2020/160242 A1 | KNQFSLQVNSVTPEDTAVYYCARMTGPRYYFEYWGQGTL |
| | | VTVSS (SEQ ID NO: 210) |
| | | VL: |
| | | DIQMTQSPSSLSASVGDRVTITCRASQSISSYLNWYQQK |
| | | PGKAPKLLIYAASSLQSGVPSRFSGSGSGTDFTLTISSL |
| | | QPEDFATYYCQQSYSTPPITFGQGTRLEIK (SEQ ID |
| | | NO: 43) |
| Anti-IL2Rγ | VH: SEQ ID NO: 296 of | VH: |
| antibody | WO 2020/160242 A1; | EVQLVESGGGVVRPGGSLRLSCAASGFTFDDFDMSWVRQ |
| | VL: SEQ ID NO: 304 of | GPGKGLEWVSGINWHGSSTGYADSVKGRFTISRDNAKNS |
| | WO 2020/160242 A1 | LYLQMSSLRAEDTALYHCVRGGTIVGATTPLDYWGQGTL |
| | | VTVSS (SEQ ID NO: 211) |
| | | VL: |
| | | DIQMTQSPSSLSASVGDRVTMTCRASRTISSYLSWYQQK |
| | | SGKVPNLLIFGASSLQSGVPSRFSASGSGTDFTLIISSL |
| | | QPEDFATYYCQQSYSSPLTFGGGTKVEIK (SEQ ID |
| | | NO: 212) |
| Anti-IL2Rγ | VH: SEQ ID NO: 315 of | VH: |
| antibody | WO 2020/160242 A1; | EVQLVESGGDLVQPGGSLRLSCTASGFIFRNYAMNWVRQ |
| | VL: SEQ ID NO: 323 of | APGKGLEWLSGILGSNDNTYYVDSVKGRFTISRDNSRNT |
| | WO 2020/160242 A1 | LYLQMNSLRAEDSAVYYCAKGDAGGFDYWGQGTLVTVSS |
| | | (SEQ ID NO: 213) |
| | | VL: |
| | | DVVMTQSPLSLPVILGQPASISCRSSQSLVSSDGNTYLN |
| | | WFQQRPGQSPRRLIYKVSNRDSGVPDRFSGSGSGTDFTL |
| | | KISRVEAEDVGAYYCMQGSYWPPTFGQGTKLEIK (SEQ |
| | | ID NO: 214) |
| Anti-IL2Rγ | VH: SEQ ID NO: 335 of | VH: |
| antibody | WO 2020/160242 A1; | QVQLVESGGGVVKPGGSLRLSCAASGFTFSNSGIHWVRQ |
| | VL: SEQ ID NO: 182 of | APGKGLEWVALISYAGSNKYYADSVKGRFTISRDNSKNT |
| | WO 2020/160242 A1 | LSLQMNSLRAEDTAVYYCAKEVWTGTYDSFDMWGRGTMV |
| | | TVSS (SEQ ID NO: 215) |
| | | VL: |
| | | DIQMTQSPSSLSASVGDRVTITCRASQSISSYLNWYQQK |
| | | PGKAPKLLIYAASSLQSGVPSRFSGSGSGTDFTLTISSL |
| | | QPEDFATYYCQQSYSTPPITFGQGTRLEIK (SEQ ID |
| | | NO: 43) |
| Anti-IL2Rγ | VH: SEQ ID NO: 345 of | VH: |
| antibody | WO 2020/160242 A1; | EVQLVESGGGLVQPGGSLRLSCAASGFIFSSYEMHWVRQ |
| | VL: SEQ ID NO: 353 of | APGKGLEWISYISSSGTTIYYADSVKGRFTISRDNAKNS |
| | WO 2020/160242 A1 | LYLHMNSLRAEDTAVYYCTRARITGTFDVFDIWGQGTMV |
| | | TVSS (SEQ ID NO: 216) |
| | | VL: |
| | | DIQMTQSPSSLSASVGDRVTITCRASQSISSYLNWYQQK |
| | | PGKAPKLLIFAASNLQSGVPSRFSGSRSGTDFTLTISSL |
| | | QPEDFATYYCQQNYNIPYTFGQGTKLEIK (SEQ ID |
| | | NO: 217) |
| Anti-IL2Rγ | VH: SEQ ID NO: 361 of | VH: |
| antibody | WO 2020/160242 A1; | QVQLQESGPGLVKPSQTLSLTCTVSGGSITSGGYYWSWI |
| | VL: SEQ ID NO: 368 of | RQYPGQGLEWIGYIYYSGKTYYNPSFTSRITISVDTSKK |
| | WO 2020/160242 A1 | QFSLKMSSVTAADTAVYYCARAGFTSSNGWFDPWGQGTL |
| | | VTVSS (SEQ ID NO: 218) |
| | | VL: |
| | | DIQMTQSPSSLSASVGDRVTITCRASQNIRSYLNWYQQK |
| | | PGKAPKLLIYSASSLQSGVPSRFSGSGSGTDFTLTISSL |
| | | QPEDFPTYYCQQTYSSPWTFGPGTKVEIK (SEQ ID |
| | | NO: 219) |
| Anti-IL2Rγ | VH: SEQ ID NO: 177 of | VH: |
| antibody | U.S. 2023/0295348 A1; | QVQLQQWGAGLLKPSETLSLTCAVYGGSFSGYYWSWIRQ |
| P1A3 B3 | VL: SEQ ID NO: 178 of | PPGKGLEWIGEINHFGSTNYNPSLKSRATISVDTSKNQF |
| | U.S. 2023/0295348 A1 | SLKLSSVTAADTAVYYCATSPGGYSGGYFQHWGQGTLVT |
| | | VSS (SEQ ID NO: 117) |
| | | VL: |
| | | DVVMTQSPLSLPVTPGEPASISCRSSQSLLHSNGYNYLD |
| | | WYLQKPGQSPQLLIYLGSNRDSGVPDRFSGSGSGTDFTL |
| | | KISRVEAEDVGVYYCMQGTHWPWTFGQGTKVEIK (SEQ |
| | | ID NO: 116) |
| Anti-IL2Rγ | VH: SEQ ID NO: 179 of | VH: |
| antibody | U.S. 2023/0295348 A1; | QVQLQQWGAGMLKPSETLSLTCAVYGGSFSGYYWSWIRQ |
| P1A3 E8 | VL: SEQ ID NO: 180 of | PPGKGLEWIGEINHFGSTNYNPSLKSRATISVDTSKNQF |
| | U.S. 2023/0295348 A1 | SLKLSSVTAADTAVYYCATSPGGYSGGYFQHWGQGTLVT |
| | | VSS (SEQ ID NO: 118) |
| | | VL: |
| | | DVVMTQSPLSLPVTPGEPASISCRSSQSLLHSNGYNYLD |
| | | WYLQKPGQSPQLLIYLGSNRDSGVPDRFSGSGSGTDFTL |
| | | KISRVEAEDVGVYYCMQGTHWPWTFGQGTKVEIK (SEQ |
| | | ID NO: 116) |
| Anti-IL2Rγ | VH: SEQ ID NO: 181 of | VH: |
| antibody | U.S. 2023/0295348 A1; | QVQLQQWGAGLLKPSETLSLTCAVYGGSFSGYYWSWIRQ |
| P1A3 E9 | VL: SEQ ID NO: 182 of | PPGKGLEWIGEINHFGSTNYNPSLKSRATISVDTSKNQF |
| | U.S. 2023/0295348 A1 | SLKLSSVTAADTAVYYCATSPGGYSGGYFQHWGQGTLVT |
| | | VSS (SEQ ID NO: 117) |
| | | VL: |
| | | DVVMTQSPLSLPVTPGEPASISCRSSQSLLHSNGYNYLD |
| | | WYLQKPGQSPQLLIYLGSNRDSGVPDRFSGSGSGTDFTL |
| | | KISRVEAEDVGVYYCMQGTHWPWTFGQGTKVEIK (SEQ |
| | | ID NO: 116) |
| Anti-IL2Rγ | VH: SEQ ID NO: 183 of | VH: |
| antibody | U.S. 2023/0295348 A1; | QVQLQQWGAGLLKPSETLSLTCAVYGGSFSGYYWSWIRQ |
| P1A3 B4 | VL: SEQ ID NO: 184 of | PPGKGLEWIGEINHFGSTNYNPSLKSRATISVDTSKNQF |
| | U.S. 2023/0295348 A1 | SLKLSSVTAADTAVYYCATSPGGYSGGYFQHWGQGTLVT |
| | | VSS (SEQ ID NO: 117) |
| | | VL: |
| | | DVVMTQSPLSLPVTPGESVSISCRSSQSLLHSNGYNYLD |
| | | WYLQKPGQSPQLLIYLGSNRDSGVPDRFSGSGSGTDFTL |
| | | KISRVEAEDVGVYYCMQGTHWPWTFGQGTKVEIK (SEQ |
| | | ID NO: 121) |
| Anti-IL2Rγ | VH: SEQ ID NO: 4 of U.S. | VH: |
| antibody | 2023/0295348 A1; | EVQLLESGGGLVQPGGSLRLSCAASGFTFSSYAMSWVRQ |
| AM1 | VL: SEQ ID NO: 67 of | APGKGLEWVSAISGSGGSTYYADSVKGRFTISRDNSKNT |
| | U.S. 2023/0295348 A1 | LYLQMNSLRAEDTAVYYCAKSYGAFDYWGQGTLVTVSS |
| | | (SEQ ID NO: 167) |
| | | VL: |
| | | DIQMTQSPSSLSASVGDRVTITCRASQSIYYYLNWYQQK |
| | | PGKAPKLLIYDASALQSGVPSRFSGSGSGTDFTLTISSL |
| | | QPEDFATYYCQQIDFTAGSITFGQGTKVEIK (SEQ ID |
| | | NO: 220) |
| Anti-IL2Rγ | VH: SEQ ID NO: 4 of U.S. | VH: |
| antibody | 2023/0295348 A1; | EVQLLESGGGLVQPGGSLRLSCAASGFTFSSYAMSWVRQ |
| AM2 | VL: SEQ ID NO: 72 of | APGKGLEWVSAISGSGGSTYYADSVKGRFTISRDNSKNT |
| | U.S. 2023/0295348 A1 | LYLQMNSLRAEDTAVYYCAKSYGAFDYWGQGTLVTVSS |
| | | (SEQ ID NO: 167) |
| | | VL: |
| | | DIQLTQSPSFLSASVGDRVTITCRASQTIDAPLRWYQQK |
| | | PGKAPKLLIYLTSSLQSGVPSRFSGSGSGTEFTLTISSL |
| | | QPEDFATYYCQQGYAAGPSTFGQGTKVEIK (SEQ ID |
| | | NO: 221) |
| Anti-IL2Rγ | VH: SEQ ID NO: 4 of U.S. | VH: |
| antibody | 2023/0295348 A1; | EVQLLESGGGLVQPGGSLRLSCAASGFTFSSYAMSWVRQ |
| AM3 | VL: SEQ ID NO: 77 of | APGKGLEWVSAISGSGGSTYYADSVKGRFTISRDNSKNT |
| | U.S. 2023/0295348 A1 | LYLQMNSLRAEDTAVYYCAKSYGAFDYWGQGTLVTVSS |
| | | (SEQ ID NO: 167) |
| | | VL: |
| | | DIQMTQSPSTLSASVGDTVTITCRASHYITTWLAWYQQK |
| | | PGKAPKLLIYDVSSLESGVPSRFRGRGSGTEFTLTISSL |
| | | QPDDFATYYCQQYESYSPTFGQGTKVEIK (SEQ ID |
| | | NO: 222) |
| Anti-IL2Rγ | VH: SEQ ID NO: 4 of U.S. | VH: |
| antibody | 2023/0295348 A1; | EVQLLESGGGLVQPGGSLRLSCAASGFTFSSYAMSWVRQ |
| AM4 | VL: SEQ ID NO: 82 of | APGKGLEWVSAISGSGGSTYYADSVKGRFTISRDNSKNT |
| | U.S. 2023/0295348 A1 | LYLQMNSLRAEDTAVYYCAKSYGAFDYWGQGTLVTVSS |
| | | (SEQ ID NO: 167) |
| | | VL: |
| | | DIQMTQSPSTLSASVGDRVTITCRASQTIYGPLNWYQQK |
| | | PGKAPKLLIYSTSYLESGVPSRFSGSGSGTEFTLTISSL |
| | | QPDDFATYYCQQAGYASAPTFGQGTKVEIK (SEQ ID |
| | | NO: 223) |
| Anti-IL2Rγ | VH: SEQ ID NO: 4 of U.S. | VH: |
| antibody | 2023/0295348 A1; | EVQLLESGGGLVQPGGSLRLSCAASGFTFSSYAMSWVRQ |
| AM5 | VL: SEQ ID NO: 87 of | APGKGLEWVSAISGSGGSTYYADSVKGRFTISRDNSKNT |
| | U.S. 2023/0295348 A1 | LYLQMNSLRAEDTAVYYCAKSYGAFDYWGQGTLVTVSS |
| | | (SEQ ID NO: 167) |
| | | VL: |
| | | DIVMTQSPDSLAVSLGERATINCKSSQSVLYSEVAYTAL |
| | | AWYQQKPGQPPKLLIYATSTRESGVPDRFSGSGSGTDFT |
| | | LTISSLQAEDVAVYYCQQGYGHPTFGQGTKVEIK (SEQ |
| | | ID NO: 224) |
| Anti-IL2Rγ | VH: SEQ ID NO: 4 of U.S. | VH: |
| antibody | 2023/0295348 A1; | EVQLLESGGGLVQPGGSLRLSCAASGFTFSSYAMSWVRQ |
| AM6 | VL: SEQ ID NO: 92 of | APGKGLEWVSAISGSGGSTYYADSVKGRFTISRDNSKNT |
| | U.S. 2023/0295348 A1 | LYLQMNSLRAEDTAVYYCAKSYGAFDYWGQGTLVTVSS |
| | | (SEQ ID NO: 167) |
| | | VL: |
| | | DIVMTQSPDSLAVSLGERATINCKSSQSVLYDDFGNANL |
| | | AWYQQKPGQPPKLLIYYGSYRESGVPDRFSGSGSGTDFT |
| | | LTISSLQAEDVAVYYCQQVDVGLAITFGQGTKVEIK |
| | | (SEQ ID NO: 225) |
| Anti-IL2Rγ | VH: SEQ ID NO: 4 of U.S. | VH: |
| antibody | 2023/0295348 A1; | EVQLLESGGGLVQPGGSLRLSCAASGFTFSSYAMSWVRQ |
| AM7 | VL: SEQ ID NO: 97 of | APGKGLEWVSAISGSGGSTYYADSVKGRFTISRDNSKNT |
| | U.S. 2023/0295348 A1 | LYLQMNSLRAEDTAVYYCAKSYGAFDYWGQGTLVTVSS |
| | | (SEQ ID NO: 167) |
| | | VL: |
| | | DIQLTQSPSFLSASVGDRVTITCRASQDIGIELAWYQQK |
| | | PGKAPKLLIYFESHLQSGVPSRFSGSGSGTEFTLTISSL |
| | | QPEDFATYYCQQIRIDPTFGQGTKVEIK (SEQ ID |
| | | NO: 226) |
| Anti-IL2Rγ | VH: SEQ ID NO: 4 of U.S. | VH: |
| antibody | 2023/0295348 A1; | EVQLLESGGGLVQPGGSLRLSCAASGFTFSSYAMSWVRQ |
| AM8 | VL: SEQ ID NO: 102 of | APGKGLEWVSAISGSGGSTYYADSVKGRFTISRDNSKNT |
| | U.S. 2023/0295348 A1 | LYLQMNSLRAEDTAVYYCAKSYGAFDYWGQGTLVTVSS |
| | | (SEQ ID NO: 167) |
| | | VL: |
| | | EIVLTQSPGTLSLSPGERATLSCRASQDVATRGLAWYQQ |
| | | KPGQAPRLLIYGASSRATGIPDRFSGSGSGTDFTLTISR |
| | | LEPEDFAVYYCQQYELEHPATFGQGTKVEIK (SEQ ID |
| | | NO: 227) |
| Anti-IL2Rγ | VH: SEQ ID NO: 4 of U.S. | VH: |
| antibody | 2023/0295348 A1; | EVQLLESGGGLVQPGGSLRLSCAASGFTFSSYAMSWVRQ |
| AM9 | VL: SEQ ID NO: 107 of | APGKGLEWVSAISGSGGSTYYADSVKGRFTISRDNSKNT |
| | U.S. 2023/0295348 A1 | LYLQMNSLRAEDTAVYYCAKSYGAFDYWGQGTLVTVSS |
| | | (SEQ ID NO: 167) |
| | | VL: |
| | | DIQMTQSPSSLSASVGDRVTITCQASQDIAGYLNWYQQK |
| | | PGKAPKLLIYTASTLETGVPSRFSGSGSGTDFTFTISSL |
| | | QPEDIATYYCQQWAFGPVTFGQGTKVEIK (SEQ ID |
| | | NO: 228) |
| Anti-IL2Rγ | VH: SEQ ID NO: 4 of U.S. | VH: |
| antibody | 2023/0295348 A1; | EVQLLESGGGLVQPGGSLRLSCAASGFTFSSYAMSWVRQ |
| AM10 | VL: SEQ ID NO: 112 of | APGKGLEWVSAISGSGGSTYYADSVKGRFTISRDNSKNT |
| | U.S. 2023/0295348 A1 | LYLQMNSLRAEDTAVYYCAKSYGAFDYWGQGTLVTVSS |
| | | (SEQ ID NO: 167) |
| | | VL: |
| | | EIVLTQSPATLSLSPGERATLSCRASQSVFANLNWYQQK |
| | | PGQAPRLLIYDSSGRATGIPARFSGSGSGTDFTLTISSL |
| | | EPEDFAVYYCQQGFGPSLTFGQGTKVEIK (SEQ ID |
| | | NO: 229) |
| Anti-IL2Rγ | VH: SEQ ID NO: 4 of U.S. | VH: |
| antibody | 2023/0295348 A1; | EVQLLESGGGLVQPGGSLRLSCAASGFTFSSYAMSWVRQ |
| AM11 | VL: SEQ ID NO: 117 of | APGKGLEWVSAISGSGGSTYYADSVKGRFTISRDNSKNT |
| | U.S. 2023/0295348 A1 | LYLQMNSLRAEDTAVYYCAKSYGAFDYWGQGTLVTVSS |
| | | (SEQ ID NO: 167) |
| | | VL: |
| | | EIVLTQSPGTLSLSPGERATLSCRASQNVNHNFLTWYQQ |
| | | KPGQAPRLLIYSASARATGIPDRFSGSGSGTDFTLTISR |
| | | LEPEDFAVYYCQQFNYAPLTFGQGTKVEIK (SEQ ID |
| | | NO: 230) |

In some aspects, the IL2Rγ targeting moiety competes with an antibody set forth in Table R3 for binding to IL2Rγ. In further aspects, the IL2Rγ targeting moiety comprises CDRs having CDR sequences of an antibody set forth in Table R3. In some embodiments, the targeting moiety comprises all 6 CDR sequences of an antibody set forth in Table R3. In other embodiments, the targeting moiety comprises at least the heavy chain CDR sequences (CDR-H1, CDR-H2, CDR-H3) of such antibody and the light chain CDR sequences of a universal light chain. In further aspects, an IL2Rγ targeting moiety comprises a VH comprising the amino acid sequence of the VH of an antibody set forth in Table R3. In some embodiments, the IL2Rγ targeting moiety further comprises a VL comprising the amino acid sequence of the VL of an antibody set forth above in Table R3. In other embodiments, the IL2Rγ targeting moiety further comprises a universal light chain VL sequence.

In some embodiments, the IL2Rγ targeting moieties are based on the exemplary anti-IL2Rγ single domain antibodies or antibody sequences set forth in Table R4 below.

TABLE R4
Exemplary Anti-IL2Rγ Single Domain Antibody (sdAb) Amino Acid Sequences

| Target or | | | SEQ |
|---|---|---|---|
| Description | Reference | Sequence | ID NO |
| IL2RG_F16A | SEQ ID NO: 22 | QVQLVESGGGLVKPGGSLRLSCAASGFTFSDYYMS | 111 |
| | of WO | WIRQAPGKGLEWVSSISSSGDTIYYADSVQGRFTL | |
| | 2022/212848 A1 | SRDNAENSLFLQMNSLRAEDTAVYYCARGDAVSIT | |
| | | GDYRGQGTLVTVSS | |
| IL2RG_F16B | SEQ ID NO: 23 | QVQLVESGGGLVKPGGSLRLSCAASGFTFSDYYMS | 112 |
| | of WO | WIRQAPGKGLEWVSYISSSGSTIYYADSVKGRFTI | |
| | 2022/212848 A1 | SRDNAKNSLYLQMNSLRAEDTAVYYCARGDAVSIT | |
| | | GDYRGQGTLVTVSS | |
| IL2RG_F16C | SEQ ID NO: 24 | QVQLVESGGGLVKPGGSLRLSCAASGFTFNDYYMS | 113 |
| | of WO | WIRQAPGKGLEWVSHISSSGSTIYYADSVKGRFTV | |
| | 2022/212848 A1 | SRDNANNSLYLQMHSLRAEDTAVYYCARGDAVSIT | |
| | | GDYRGQGTLVTVSS | |
| IL2RG_F18A | SEQ ID NO: 25 | QVQLVESGGDLVKPGGSLRLSCAASGFTFSDYYMS | 114 |
| | of WO | WLRQAPGKELEWVSHISSSGTTTYYADSVEGRFTI | |
| | 2022/212848 A1 | TRDNAKNSLYLQMNSLRAEDTAVYYCARGAAVAPG | |
| | | FDSRGQGTLVTVSS | |
| 27-MP04G01 | SEQ ID NO: 27 | QVQLVESGGGLVQAGGSLTLSCAAPGRTFGTDVVG | 124 |
| | of WO | WFRQAPGKEREFVASISRSGDGIYYDDSVKGRFTI | |
| | 2023/067194 A1 | SRNNAWNTVNLQMNSLKVEDTAVYYCAAGDGWSTY | |
| | | DYWGQGTQVTVSS | |
| 28-MP04D02 | SEQ ID NO: 28 | QVQLVESGGGLVQAGGSLRLSCAASGRTLSRYAMG | 125 |
| | of WO | WFRQAPGKEREFVTANSWGGDTYYADSVQGRFTFS | |
| | 2023/067194 A1 | RDNAKNTVYLQMNSLQPEDTAVYYCAAAPTSFATT | |
| | | AYSGSNSYAYWGQGTQVTVSS | |
| 29-MP04H02 | SEQ ID NO: 29 | QVQLVESGGGLVQAGGSLRLACVASGLTFDNYYMG | 126 |
| | of WO | WFRQAPGKEREFVAGIIWNGDHTAYADSIKGRFTI | |
| | 2023/067194 A1 | SRDNAKNTAYLRMNSLKPEDTAVYYCAATFWIERA | |
| | | TTPDIGQYAYWGQGTQVTVSS | |
| 30-MP04C03 | SEQ ID NO: 30 | EVQLVESGGGWVQDGGSLRLSCALSGRTFVRGIMG | 127 |
| | of WO | WFRQAPGKEREFVARIIWHINSTRYADSVKGRFTI | |
| | 2023/067194 A1 | SRDSAKNTMYLQMDSLRPEDTAVYYCAARDRYGSG | |
| | | NSLSPSAYDYWGQGTQVTVSS | |
| 31-MP04E03 | SEQ ID NO: 31 | QVQLVESGGGLVQAGGSLRLSCTGYGGAFTGYALG | 128 |
| | of WO | WFRQAPGKEREFVARINWSGSFTYYASSVKGRFTI | |
| | 2023/067194 A1 | SRDNAKNTMYLQMNNLKPEDTAVYYCAADNPSTLA | |
| | | TDYDNWGQGTQVTVSS | |
| 32-MP04A08 | SEQ ID NO: 32 | QVQLVESGGGLVQAGGSLRLSCAASGRTFGSTAVG | 129 |
| | of WO | WFRQVPGKEREFVSAINRSGSATTYADSVKGRFTI | |
| | 2023/067194 A1 | SRDNAKNTVYLQMNSLTPEDTGVYYCAADSLPYGR | |
| | | PYYFQRSAGEYDYWGQGTQVTVSS | |
| 33-MP04C09 | SEQ ID NO: 33 | QLQLVESGGGLVQAGGSLRLSCAASGPTFSRVAVG | 130 |
| | of WO | WFRQAPGKEREFVAAVNRPATMTKYADSVKGRFTV | |
| | 2023/067194 A1 | SRDNAKNTVDLQMNSMKPEDTAVYYCAADSVPYGR | |
| | | PYYWQTSAGDYDYWGQGTQVTVSS | |
| 34-MP04A12 | SEQ ID NO: 34 | QVQLVESGGGLVQAGSSLRLSCAASGRTLSRLAMG | 131 |
| | of WO | WFRQAPGKEREFVAVNSWGGDTFYADSVEGRFTYS | |
| | 2023/067194 A1 | RDNAKSAVYLQMNSLQPEDTAVYYCAAAPTSFATT | |
| | | AYSSSNSYAYWGQGAQVTVSS | |
| 35-MP07G01 | SEQ ID NO: 35 | QVQLQESGGGLVQGGGSLRLSCAASGGIFSSYAMG | 132 |
| | of WO | WFRQAPGKEREFVAAISRSGRSTNYADSVKGRFTI | |
| | 2023/067194 A1 | SRDNAKSTVYLQMNSLKPEETAVYYCAAGRYYNSA | |
| | | YDPSPGDFGSWGHGTQVTVSS | |
| 36-MP07F02 | SEQ ID NO: 36 | QVQLVESGGGLVQAGGSLRLSCAASGRTLSRYAMG | 133 |
| | of WO | WFRQAPGSEREFVAASSWGGDTFYADSVEGRFTFS | |
| | 2023/067194 A1 | RDNAKNAVYLQMNSLQPEDTAAYYCAAAPTSFPTT | |
| | | AYSSSNSYAYWGQGTQVTVSS | |
| 37-MP07F09 | SEQ ID NO: 37 | QVQLVESGGGLVQAGGSLRLSCAASGRTLSRYAMG | 134 |
| | of WO | WFRQAPGKEREYVAIDSWGGDTFYADSVEGRFTFS | |
| | 2023/067194 A1 | RDNAKNEVYLQMNSLQPEDTAVYYCAGAPTSFATT | |
| | | AYSSSNSYRYWGQGTQVTVSS | |
| 38-MP07A11 | SEQ ID NO: 38 | QVQLVESGGGLVQAGGSLRLSCAASGRSLSRDAMG | 135 |
| | of WO | WFRQAPGKEREFVAVMSWGGDTFYTDSVEGRFTFS | |
| | 2023/067194 A1 | RDNAKNAVYLEMNDLQPEDTAVYYCAAAPTSFATT | |
| | | AYSSSNSYSYWGRGTQVTVSS | |
| YNb3 | Sequence | QVQLQESGGGSVQAGGSLRLSCAASGYTYSKNWYM | 136 |
| | disclosed in FIG. | GWFRQTPGKEREGVAVIAYDDWPTYADSVKGRFTI | |
| | S1 of Yen et al., | SKDNTKNTLYLQMNSLKPEDTAMYYCAARQLGGDY | |
| | 2023, Cell. | CYFPNLSRFCYNYWGQGTQVTVSS | |
| | 185(8): 1414- | | |
| | 1430.e19 | | |
| YNb4 | Sequence | QVQLQESGGGSVQAGGSLRLSCTASGFTFNEANHM | 137 |
| | disclosed in FIG. | GWYRQAPGNECELVSTISSDGTTYYPDSVKGRFTI | |
| | S1 of Yen et al., | SQDNAKKTAFLQMNSLKPEDTAVYYCAADQSRRGS | |
| | 2023, Cell. | LCLGQGTQVTVSS | |
| | 185(8): 1414- | | |
| | 1430.e19 | | |
| YNb6 | Sequence | QVQLQESGGGSVQAGGSLRLSCAASGYTYRDYYMG | 138 |
| | disclosed in FIG. | WFRQAPGREREGVASIYTRGSREGSTRYSSSVEGR | |
| | S1 of Yen et al., | FTITLDTAKNTLYLQMNSLKPEDTAMYYCAADDRT | |
| | 2023, Cell. | WLPRVQLGGPRENEYNYWGQGTQVTVSS | |
| | 185(8): 1414- | | |
| | 1430.e19 | | |
| hIL2Rg_VHH- | SEQ ID NO: 1 of | QVQLQESGGGSVQAGGSLRLSCAASGFTFDDSDMG | 231 |
| 1 | U.S. | WYRQAPGNECDLVSTISSDGSTYYADSVKGRFTIS | |
| | 2023/0272089 A1 | QDNAKNTVYLQMDSVKPEDTAVYYCAADFMIAIQA | |
| | | PGAGCWGQGTQVTVSS | |
| hIL2Rg_VHH- | SEQ ID NO: 5 of | QVQLQESGGGSVPAGGSLKLSCAASGFSFSSYPMT | 232 |
| 2 | U.S. | WARQAPGKGLEWVSTIASDGGSTAYAASVEGRFTI | |
| | 2023/0272089 A1 | SRDNAKSTLYLQLNSLKTEDTAMYYCTKGYGDGTP | |
| | | APGQGTQVTVSS | |
| hIL2Rg_VHH- | SEQ ID NO: 9 of | QVQLQESGGGSVQTGGSLRLSCTASGFTFDDREMN | 233 |
| 3 | U.S. | WYRQAPGNECELVSTISSDGSTYYADSVKGRFTIS | |
| | 2023/0272089 A1 | QDNAKNTVYLQMDSVKPEDTAVYYCAADFMIAIQA | |
| | | PGAGCWGQGTQVTVSS | |
| hIL2Rg_VHH- | SEQ ID NO: 13 of | QVQLQESGGGSVQAGGSLRLSCTASGFTFDDSDMG | 234 |
| 4 | U.S. | WYRQAPGNECELVSTISSDGNTYYTDSVKGRFTIS | |
| | 2023/0272089 A1 | QDNAKNTVYLQMNSLGPEDTAVYYCAAEPRGYYSN | |
| | | YGGRRECNYWGQGTQVTVSS | |
| hIL2Rg_VHH- | SEQ ID NO: 17 of | QVQLQESGGGSVQAGGSLRLSCAASGFSFSSYPMT | 235 |
| 5 | U.S. | WARQAPGKGLEWVSTIASDGGSTAYAASVEGRFTI | |
| | 2023/0272089 A1 | SRDNAKSTLYLQLNSLKTEDTAMYYCTKGYGDGTP | |
| | | APGQGTQVTVSS | |
| hIL2Rg_VHH- | SEQ ID NO: 21 of | QVQLQESGGGAVQAGGSLRLSCAASGFTFSNAHMS | 236 |
| 6 | U.S. | WVRQAPGKGREWISSIYSGGSTWYADSVKGRFTIS | |
| | 2023/0272089 A1 | RDNSKNTLYLQLNSLKTEDTAMYYCAENRLHYYSD | |
| | | DDSLRGQGTQVTVSS | |
| hIL2Rg_VHH- | SEQ ID NO: 25 of | QVQLQESGGGLVQPGGSLRLSCAASGFTFDDREMN | 237 |
| 7 | U.S. | WYRQAPGNECELVSTISSDGSTYYADSVKGRFTIS | |
| | 2023/0272089 A1 | QDNAKNTVYLQMDSVKPEDTAVYYCAADFMIAIQA | |
| | | PGAGCWGQGTQVTVSS | |
| hIL2Rg_VHH- | SEQ ID NO: 29 of | QVQLQESGGGSVQAGGSLRLSCVASGYTFSSYCMG | 238 |
| 8 | U.S. | WFRQAPGKEREGVAALGGGSTYYADSVKGRFTISQ | |
| | 2023/0272089 A1 | DNAKNTLYLQMNSLKPEDTAMYYCAAAWVACLEFG | |
| | | GSWYDLARYKHWGQGTQVTVSS | |
| hIL2Rg_VHH- | SEQ ID NO: 33 of | QVQLQESGGGSVQAGGSLRLSCTASGFTFDDSDMG | 239 |
| 9 | U.S. | WYRQAPGGECELVTISSDGSTYYADSVKGRFTISQ | |
| | 2023/0272089 A1 | DNAKNTVYLQMNSLKPEDTAVYYCAAEPRGYYSNY | |
| | | GGRRECNYWGQGTQVTVSS | |
| hIL2Rg_VHH- | SEQ ID NO: 37 of | QVQLQESGGGSVQAGGSLRLSCAASGSIYSSAYIG | 240 |
| 10 | U.S. | WFRQAPGKKREGVAGIYTRDGSTAYADSVKGRFTI | |
| | 2023/0272089 A1 | SQDSAKKTVYLQMNSLKPEDTAMYYCAAGRRTKSY | |
| | | VYIFRPEEYNYWGQGTQVTVSS | |
| hIL2Rg_VHH- | SEQ ID NO: 41 of | QVQLQESGGGSVQAGGSLRLSCAASGFTFSSAHMS | 241 |
| 11 | U.S. | WVRQAPGKGREWIASIYSGGGTFYADSVKGRFTIS | |
| | 2023/0272089 A1 | RDNAKNTLYLQLNSLKTEDTAMYYCATNRLHYYSD | |
| | | DDSLRGQGTQVTVSS | |
| hIL2Rg_VHH- | SEQ ID NO: 45 of | QVQLQESGGGSVQAGGSLRLSCAASGFTFSNAHMS | 242 |
| 12 | U.S. | WVRQAPGKGREWISSIYSGGSTWYADSVKGRFTIS | |
| | 2023/0272089 A1 | RDNSKNTLYLQLNSLKTEDTAMYYCAENRLHYYSD | |
| | | DDSLRGQGTQVTVSS | |
| hIL2Rg_VHH- | SEQ ID NO: 49 of | QVQLQESGGGSVQAGGSLRLSCTASRFIFDDSDMG | 243 |
| 13 | U.S. | WYRQAPGNECELVSTISSDGSTYYADSVKGRFTIS | |
| | 2023/0272089 A1 | RDNAKNTVYLQMNSLKPEDTAVYYCAAEPRGYYSN | |
| | | YGGRRECNYWGQGTQVTVSS | |
| hIL2Rg_VHH- | SEQ ID NO: 53 of | QVQLQESGGGSVQAGGSLKLSCTVSGFTADDSDMG | 244 |
| 14 | U.S. | WYRQGPGNECELVTISSDGSTYYADSVKGRFTISQ | |
| | 2023/0272089 A1 | DNAKNTVYLQMNSLKPEDTAVYYCAAEPRGYYSNY | |
| | | GGRRECNYWGQGTQVTVSS | |
| hIL2Rg_VHH- | SEQ ID NO: 57 of | QVQLQESGGGLVQPGGSLRLSCAASGFTFSSAHMS | 245 |
| 15 | U.S. | WVRQAPGKGREWIASIYSGGGTFYADSVKGRFTIS | |
| | 2023/0272089 A1 | RDNAKNTLYLQLNSLKAEDTAMYYCATNRLHYYSD | |
| | | DDSLRGQGTQVTVSS | |
| hIL2Rg_VHH- | SEQ ID NO: 61 of | QVQLQESGGGLVQPGGSLRLSCVASGFTFSNAHMS | 246 |
| 16 | U.S. | WVRQAPGKGREWISSIYSGGSTWYADSVKGRFTIS | |
| | 2023/0272089 A1 | RDNSKNTLYLQLNSLKTEDTAMYYCAENRLHYYSD | |
| | | DDSLRGQGTQVTVSS | |
| hIL2Rg_VHH- | SEQ ID NO: 65 of | QVQLQESGGGLVQPGGSLRLSCAASGFTFSNAHMS | 247 |
| 17 | U.S. | WVRQAPGKGREWISSIYSGGSTWYADSVKGRFTIS | |
| | 2023/0272089 A1 | RDNSKNTLYLQLNSLKTEDTAMYYCAENRLHYYSD | |
| | | DDSLRGQGTQVTVSS | |
| hIL2Rg_VHH- | SEQ ID NO: 69 of | QVQLQESGGGLVQPGGSLRLSCAASGFTFSSYPMT | 248 |
| 18 | U.S. | WARQAPGKGLEWVSTIASDGGSTAYAASVEGRFTI | |
| | 2023/0272089 A1 | SRDNAKSTLYLQLNSLKTEDTAMYYCTKGYGDGTP | |
| | | APGQGTQVTVSS | |
| hIL2Rg_VHH- | SEQ ID NO: 73 of | QVQLQESGGGSVQAGGSLRLSCTASGFTFDDREMN | 249 |
| 19 | U.S. | WYRQAPGNECELVSTISSDGSTYYADSVKGRFTIS | |
| | 2023/0272089 A1 | QDNAKNTVYLQMDSVKPEDTAVYYCAADFMIAIQA | |
| | | PGAGCWGQGTQVTVSS | |
| hIL2Rg_VHH- | SEQ ID NO: 77 of | QVQLQESGGGSVQAGGSLRLSCTASGFTFDDSDMG | 250 |
| 20 | U.S. | WYRQAPGNECELVSTISSDGSTYYADSVKGRFTIS | |
| | 2023/0272089 A1 | QDNAKNTVYLQMNSLKPEDTAVYYCAAEPRGYYSN | |
| | | YGGRRECNYWGQGTQVTVSS | |
| hIL2Rg_VHH- | SEQ ID NO: 81 of | QVQLQESGGGSVQAGGSLRLSCVASGYTSCMGWFR | 25 |
| 21 | U.S. | QAPGKEREAVATIYTRGRSIYYADSVKGRFTISQD | |
| | 2023/0272089 A1 | NAKNTLYLQMNSLKPEDIAMYSCAAGGYSWSAGCE | |
| | | FNYWGQGTQVTVSS | |
| hIL2Rg_VHH- | SEQ ID NO: 85 of | QVQLQESGGGLVQPGGSLRLSCTASGFSFSSYPMT | 252 |
| 22 | U.S. | WARQAPGKGLEWVSTIASDGGSTAYAASVEGRFTI | |
| | 2023/0272089 A1 | SRDNAKSTLYLQLNSLKTEDTAMYYCTKGYGDGTP | |
| | | APGQGTQVTVSS | |
| hIL2Rg_VHH- | SEQ ID NO: 89 of | QVQLQESGGGLVQPGGSLRLSCAASGFSFSSYPMT | 253 |
| 23 | U.S. | WARQAPGKGLEWVSTIASDGGSTAYAASVEGRFTI | |
| | 2023/0272089 A1 | SRDNAKSTLYLQLNSLKTEDTAMYYCTKGYGDGTP | |
| | | APGQGTQVTVSS | |

In some aspects, the IL2Rγ targeting moiety competes with an antibody set forth above in Table R4, for binding to the IL2Rγ. In further aspects, the IL2Rγ targeting moiety comprises CDRs having CDR sequences of an antibody set forth in Table R4. In some embodiments, the IL2Rγ targeting moiety comprises all 3 CDR sequences of the antibody set forth in Table R4. In further aspects, an IL2Rγ targeting moiety comprises a VH (e.g., a VHH or sdVH) comprising the amino acid sequence of the VH of an antibody set forth in Table R4.

In some embodiments, the IL2Rγ targeting moiety binds an epitope at similar proximity to cell membrane as the IL2Rβ targeting of the tumor-targeted split IL2 receptor agonist. In some embodiments, if the IL2Rβ targeting moiety binds to the D2 domain of IL2Rβ, then the IL2Rγ targeting moiety binds to D1 domain of IL2Rγ.

### 6.5. Tumor-Associated Antigen Targeting Moieties

The tumor-targeted IL2Rβ binding molecule and the tumor-targeted IL2Rγ of the tumor-targeted split IL2 receptor agonists of the disclosure both comprise a tumor-associated antigen (“TAA”) targeting moiety. Typically, the TAA recognized by the TAA targeting moiety of the tumor-targeted IL2Rβ binding molecule and the TAA recognized by the TAA targeting moiety of the tumor-targeted IL2Rγ binding molecule are both expressed on the same cancer cell and may be the same TAA or different TAAs. If the TAA targeting moiety of the tumor-targeted IL2Rβ binding molecule and the TAA targeting moiety of the tumor-targeted IL2Rγ binding molecule bind to the same TAA, in some embodiments the TAA targeting moiety of the tumor-targeted IL2Rβ binding molecule and the TAA targeting moiety of the tumor-targeted IL2Rγ binding molecule bind to the TAA in a non-competing fashion such that both the tumor-targeted IL2Rβ binding molecule and the tumor-targeted IL2Rγ binding molecule can bind to the same cell concurrently. In some embodiments, the TAA targeting moiety of the tumor-targeted IL2Rβ binding molecule and the TAA targeting moiety of the tumor-targeted IL2Rγ binding molecule bind to the same epitope of the TAA. In some embodiments, the TAA targeting moiety of the tumor-targeted IL2Rβ binding molecule and the TAA targeting moiety of the tumor-targeted IL2Rγ binding molecule compete for binding to the TAA (e.g., are competing TAA targeting moieties as determined using an antibody cross-competition assay as described in Section 8.1.6). In some embodiments, the TAA targeting moiety of the tumor-targeted IL2Rβ binding molecule and the TAA targeting moiety of the tumor-targeted IL2Rγ binding molecule bind to the TAA in a partially-competing fashion TAA (e.g., are partially-competing TAA targeting moieties as determined using an antibody cross-competition assay as described in Section 8.1.6). In some embodiments, the TAA targeting moiety of the tumor-targeted IL2Rβ binding molecule and the TAA targeting moiety of the tumor-targeted IL2Rγ binding molecule bind to the TAA in a non-competing fashion TAA (e.g., are non-competing TAA targeting moieties as determined using an antibody cross-competition assay as described in Section 8.1.6).

Without being bound by theory, the inventors believe that the incorporation of TAA targeting moieties that bind to the same tumor cell in both the tumor-targeted IL2Rβ binding molecule and the tumor-targeted IL2Rγ binding molecule permits the delivery of high concentrations of IL2 into the tumor microenvironment while engaging tumor reactive lymphocytes, resulting in enhancement of the cytotoxic response against tumor cells with a concomitant reduction of systemic exposure.

Suitable TAA targeting moiety formats are described in Section 6.6. The TAA targeting moiety is preferably an antigen binding domain, for example an antibody or an antigen-binding portion of an antibody, e.g., a Fab, as described in Section 6.7.1, an scFv, as described in Section 6.7.2, or a single domain antibody, as described in Section 6.7.3.

Exemplary target molecules recognized by the TAA targeting moieties of the tumor-targeted IL2Rβ binding molecule and/or the tumor-targeted IL2Rγ binding molecule are melanotransferrin (MELTF or CD228), Fibroblast Activation Protein (FAP), the A1 domain of Tenascin-C(TNC A1), the A2 domain of Tenascin-C(TNC A2), the Extra Domain B of Fibronectin (EDB), the Melanoma-associated Chondroitin Sulfate Proteoglycan (MCSP), MART-1/Melan-A, gp100, Dipeptidyl peptidase IV (DPPIV), adenosine deaminase-binding protein (ADAbp), cyclophilin b, colorectal associated antigen (CRC)-C017-1A/GA733, Carcinoembryonic Antigen (CEA) and its immunogenic epitopes CAP-1 and CAP-2, etv6, aml1, prostate-specific membrane antigen (PSMA), T-cell receptor/CD3-zeta chain, GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, Her3, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD20, MCSP, PDGFβR (β-platelet-derived growth factor receptor), ErbB2 epithelial cell adhesion molecule (EpCAM), EGFR variant III (EGFRvIII), CD19, disialoganglioside GD2, ductal-epithelial mucine, gp36, TAG-72, glioma-associated antigen, β-human chorionic gonadotropin, alphafetoprotein (AFP), lectin-reactive AFP, thyroglobulin, MN-CA IX, human telomerase reverse transcriptase, RU1, RU2 (AS), intestinal carboxyl esterase, mut hsp70-2, M-CSF, PAP, LAGA-1a, prostein, survivin and telomerase, prostate-carcinoma tumor antigen-1 (PCTA-1), ELF2M, neutrophil elastase, ephrin B2, insulin growth factor (IGF1)-I, IGF-II, IGFI receptor, 5T4, ROR1, Nkp30, NKG2D, tumor stromal antigens, CA166-9, the extra domain A (EDA) and extra domain B (EDB) of fibronectin and the A1 domain of tenascin-C (TnC A1).

In some embodiments, the target molecule recognized by the TAA targeting moiety of the tumor-targeted IL2Rβ binding molecule and/or the tumor-targeted IL2Rγ binding molecule is BCMA.

In some embodiments, the target molecule recognized by the TAA targeting moiety of the tumor-targeted IL2Rβ binding molecule and/or the tumor-targeted IL2Rγ binding molecule is CD20. In some embodiments, the target molecule recognized by the TAA targeting moiety of the tumor-targeted IL2Rβ binding molecule and/or the tumor-targeted IL2Rγ binding molecule is EGFR. In some embodiments, the target molecule recognized by the TAA targeting moiety of the tumor-targeted IL2Rβ binding molecule and/or the tumor-targeted IL2Rγ binding molecule is PSMA. In some embodiments, the target molecule recognized by the TAA targeting moiety of the tumor-targeted IL2Rβ binding molecule and/or the tumor-targeted IL2Rγ binding molecule is CA9. In some embodiments, the target molecule recognized by the TAA targeting moiety of the tumor-targeted IL2Rβ binding molecule and/or the tumor-targeted IL2Rγ binding molecule is MSLN. In some embodiments, the target molecule recognized by the TAA targeting moiety of the tumor-targeted IL2Rβ binding molecule and/or the tumor-targeted IL2Rγ binding molecule is EPCAM. In some embodiments, the target molecule recognized by the TAA targeting moiety of the tumor-targeted IL2Rβ binding molecule and/or the tumor-targeted IL2Rγ binding molecule is B7H3. In some embodiments, the target molecule recognized by the TAA targeting moiety of the tumor-targeted IL2Rβ binding molecule and/or the tumor-targeted IL2Rγ binding molecule is HER2/HER3. In some embodiments, the target molecule recognized by the TAA targeting moiety of the tumor-targeted IL2Rβ binding molecule and/or the tumor-targeted IL2Rγ binding molecule is STEAP1. In some embodiments, the target molecule recognized by the TAA targeting moiety of the tumor-targeted IL2Rβ binding molecule and/or the tumor-targeted IL2Rγ binding molecule is CEACAM5.

In some embodiments, the targeting moieties target the exemplary target molecules set forth in Table T1 below, which provides references to exemplary antibodies or antibody sequences upon which the targeting moiety can be based.

TABLE T1
Exemplary Target Molecules

| Target | Antibody Name and/or Binding Sequences |
|---|---|
| 5T4 | GEN1044 |
| Activin Receptor Type II | Bimagrumab |
| (ACVR2) | VH: SEQ ID NOs: 107, 109 of U.S. Pat. No. 8,388,968 B2 |
| | VL: SEQ ID NOs: 93, 95 of U.S. Pat. No. 8,388,968 B2 |
| B7-H3 | Obrindatamab (MGD009) |
| B7-H3 (CD276) | Enoblituzumab (MGA271) |
| B7-H3 (CD276) | MGC018 |
| B7-H3 (CD276) | MGA012 |
| B7-H3 (CD276) | 8H9 |
| B7-H3 (CD276) | VH: the VH sequence of the heavy chain of SEQ ID NO: 21, 26 or |
| | 31 of U.S. 2021/0171641 A1. |
| | VL: the VL sequence of the light chain of SEQ ID NO: 20, 22 or |
| | 30 of U.S. 2021/0171641 A1. |
| B7-H3 (CD276) | VH: the VH sequence of the heavy chain of SEQ ID NO: 21, 29 or |
| | 37 of U.S. 2019/0002563 A1. |
| | VL: the VL sequence of the light chain of SEQ ID NO: 17, 25 or |
| | 33 of U.S. 2019/0002563 A1. |
| B7-H3 (CD276) | VH: the VH sequence of the heavy chain of SEQ ID NO: 146, 147 |
| | or 148 of U.S. Pat. No. 10,640,563. |
| | VL: the VL sequence of the light chain of SEQ ID NO: 143, 144 or |
| | 145 of U.S. Pat. No. 10,640,563. |
| BCMA | VH: the VH sequence of the heavy chain of SEQ ID NO. 126 of |
| | U.S. 2021/0206865 A1 |
| | VL: the VL sequence of the light chain of SEQ ID NO. 129 or |
| | SEQ ID NO. 132 of U.S. 2021/0206865 A1 |
| CA125 (MUC16) | Igobumab |
| CA125 | OvaRex ™ (oregobumab) |
| Cadherin | The antibodies described in US Pub. No. U.S. 2006/0039915. |
| N-cadherin | An antibody that binds to the amino acid sequence of SEQ ID |
| | NO: 10, 17 or 18 of US Pub. No. U.S. 2010/0278821. |
| CD19 | Blincyto ™ (blinatumomab) |
| CD19 | SGN-CD19A |
| CD20 | Bexxar ™ (tositumomab) |
| | VH: the VH sequence of the heavy chain of SEQ ID NO: 124 of |
| | US Patent Pub. U.S. 2017/0002060 A1 |
| | VL: the VL sequence of the light chain of SEQ ID NO: 125 of |
| | US Patent Pub. U.S. 2017/0002060 A1 |
| CD20 | Zevalin ™ (ibritumomab tiuxetan) |
| | VH: SEQ ID NO: 9 of U.S. Pat. No. 5,736, 137 |
| | VL: SEQ ID NO: 6 of U.S. Pat. No. 5,736, 137 |
| CD20 | Rituxan ™ (rituximab) |
| | VH: SEQ ID NO: 9 of U.S. Pat. No. 5,736, 137 |
| | VL: SEQ ID NO: 6 of U.S. Pat. No. 5,736, 137 |
| CD20 | Ocrevus ™ (ocrelizumab) |
| CD20 | Okaratuzumab |
| CD20 | Arzerra ™ (ofatumumab) |
| | VH: SEQ ID NO: 2 of U.S. Pat. No. 8,529,902 |
| | VL: SEQ ID NO: 4 of U.S. Pat. No. 8,529,902 |
| CD20 | Gazyva ™ (obinutuzumab) |
| CD20 | VH: SEQ ID NO: 4 of U.S. 2021/0206870 A1 |
| | VL of SEQ ID NO: 6 of U.S. 2021/0206870 A1 |
| CD20 | Epcoritamab |
| CD22 | Belimumab |
| CD22 | Epratuzumab |
| CD22 | Besponsa ™ (inotuzumab ozogamicin) |
| CD22 | Lumoxiti ™ (moxetumumab pasudox) |
| CD22 | pinatuzumab vedotin |
| CD25 | Zenapax ™ (daclizumab) |
| | VH: SEQ ID NO: 9 of U.S. Pat. No. 7,060,269 |
| | VL: SEQ ID NO: 10 of U.S. Pat. No. 7,060,269 |
| CD30 | Adcetris ™ (brentuximab vedotin) |
| | VH: SEQ ID NO: 2 of U.S. Pat. No. 7,090,843 |
| | VL: SEQ ID NO: 10 of U.S. Pat. No. 7,090,843 |
| CD33 | Myelotarg ™ (gemtuzumab) |
| | Sequence in Man Sung, et al., 1993, Molecular immunology |
| | 30: 1361-1367 |
| CD33 | Lintuzumab |
| CD38 | Darzalex ™ (daratumumab) |
| CD44v6 | vibatuzumab mertansine |
| CD52 | Campath ™ (alemtuzumab) |
| | VH: SEQ ID NO: 1 of US Patent Pub. U.S. 2017/0002060 A1 |
| | VL: SEQ ID NO: 2 of US Patent Pub. U.S. 2017/0002060 A1 |
| CD70 | Blenrep ™ (borsetuzumab mafodotin) |
| CD123 | Flotetuzumab |
| CD221 | Tepezza ™ (teprotumumab) |
| CEA | Hybri-Ceaker ® (altumomab pentetate) |
| CEA | Scintimun ™ (besilesomab) |
| CEA | CEA-CIDE ™ (labetuzumab)) |
| CEA | CEA-Scan ™ (arcitumomab) |
| CEA | hMN-15 |
| | CDR-H1, CDR-H2 and CDR-H3 sequences of SEQ ID NOs: 4-6 |
| | of U.S. Pat. No. 8,771,690 B2 |
| | CDR-L1, CDR-L2 and CDR-L3 sequences of SEQ ID NOs: 1-3 of |
| | U.S. Pat. No. 8,771,690 B2 |
| CEA | CEA binding portion of RO6958688/RG7802 from clinical trial |
| | NCT02324257 |
| CEA | Cibisatamab |
| CEA | CEA binding portion of MEDI-565/MT110/AMG211 from clinical |
| | trials NCT01284231 and NCT02291614 |
| | VH: SEQ ID NO: 49 or 51 of PCT Publication No. WO |
| | 2013/012414 A1 |
| | VL: SEQ ID NO: 48 of PCT Publication No. WO 2013/012414 A1. |
| CEA | Rabetuzumab |
| CEA | Atezolizumab |
| CEA | Cibisatamab |
| CEA | MEDI-565 (AMG211, MT111) |
| CEA | RO6958688 |
| CEA | VH: SEQ ID No. 9 described in WO2022/048883A1 |
| | VL: SEQ ID No. 10 described in WO2022/048883A1 |
| CLDN18.2 | AMG910 |
| DLL3 | AMG757 |
| EGFR | Erbitux ™ (cetuximab) |
| | VH: SEQ ID NO: 11 of U.S. Pat. No. 6,217,866 |
| | VL: SEQ ID NO: 13 of U.S. Pat. No. 6,217,866 |
| EGFR | Vectibix ™ (panitumumab) |
| | VH: SEQ ID NO: 37 of U.S. Pat. No. 6,235,883 |
| | VL: SEQ ID NO: 38 of U.S. Pat. No. 6,235,883 |
| EGFR | Zalutumumab |
| | VH: SEQ ID NO: 64 of WO 2018/140831 A2 |
| | VL: SEQ ID NO: 69 of WO 2018/140831 A2 |
| EGFR | mapatumumab |
| EGFR | Matuzumab |
| EGFR | Nimotuzumab |
| | VH: SEQ ID NO: 51 of WO 2018/140831 A2 |
| | VL: SEQ ID NO: 56 of WO 2018/140831 A2 |
| EGFR | ICR62 |
| EGFR | mAb 528 |
| EGFR | CH806 |
| EGFRv3 | AMG596 |
| EGFRv3 | AMG404 |
| EpCAM | Panorex ™ (edrecolomab) |
| | VH: SEQ ID NO: 129 of WO 2018/140831 A2 |
| | VL: SEQ ID NO: 134 of WO 2018/140831 A2 |
| EpCAM | Adecatumumab |
| | VH: SEQ ID NO: 142 of WO 2018/140831 A2 |
| | VL: SEQ ID NO: 147 of WO 2018/140831 A2 |
| EpCAM | tucotuzumab celmoleukin |
| EpCAM | citatuzumab bogatox |
| EpCAM | EP1629013 B1 |
| | VH: SEQ ID NOs: 80, 84, 88, 92 or 96 |
| | VL: SEQ ID NOs: 82, 86, 90, 94 or 98 |
| EpCAM | G8.8 |
| | HC: SEQ ID NO: 4 of US Patent Pub. No. U.S. 2020/0317806 A1 |
| | HL: SEQ ID NO: 3 of US Patent Pub. No. U.S. 2020/0317806 A1 |
| EpCAM | VH: SEQ ID NOs: 17-22 of WO 2021/211510 A2. |
| | VL: SEQ ID NO: 15-16 of WO 2021/211510 A2. |
| EpCAM | Removab ™(catumaxomab) |
| EpCAM | Vicineum ™ (oportuzumab monatox) |
| EpCAM | M701 |
| GD2 | 3F8 |
| | ReoPro ™ (abiciximab) |
| gpA33 | MGD007 |
| GPC3 | ERY974 |
| GUCY2C | PF-07062119 |
| Her2 | Herceptin ™ (trastuzumab) |
| Her2 | Aldesleukin (proleukine) |
| Her2 | Sargramustim (leukine) |
| Her2 | M802 |
| Her2 | Runimotamab (BTRC4017A, R07227780) |
| Her2 | ISB1302 |
| Her2-neu | Perjeta ™ (pertuzumab) |
| | VH: SEQ ID NO: 16 of WO 2013/096812 A1. |
| | VL: SEQ ID NO: 15 of WO 2013/096812 A1. |
| Her2-neu | Rexomun ™ (ertumaxomab) |
| Integrinα4 | Tysabri ™ (natalizumab) |
| | VH: SEQ ID NOs: 11-13 of U.S. Pat. No. 5,840,299 |
| | VL: SEQ ID NOs: 7-8 of U.S. Pat. No. 5,840,299 |
| Integrinα4 β7 | Entyvio ™ (vedolizumab) |
| | HC: SEQ ID NO: 2 of US Patent Pub. U.S. 2012/0282249. |
| | LC: SEQ ID NO: 4 of US Patent Pub. U.S. 2012/0282249. |
| Integrinα5 β1 | VH: SEQ ID NO: 2 of European Patent No. 1 755 659. |
| | VL: SEQ ID NO: 4 of European Patent No. 1 755 659. |
| Integrin β1 | VH: SEQ ID NO: 2, 6, 8, 10, 12, 14, 29-43 or 91-100 of |
| | U.S. Patent Pub. U.S. 2022/0089744. |
| | VL: SEQ ID NO: 4, 16, 18, 20, 22, 44-57 or 107-116 of |
| | U.S. Patent Pub. U.S. 2022/0089744. |
| Mesothelin | Amatuximab |
| Mesothelin | HPN536 |
| MUC1 | civatuzumab tetraxetane |
| MUC1 | Pankomab ™ (gatipotuzumab) |
| MUC1 | Femtumumab |
| MUC1 | Cantuzumab ravtansine |
| MUC16 (CA125) | Anti-MUC16 antibodies having VH and VL sequences having the |
| | amino acid sequences of any one of the following SEQ ID NO: |
| | pairs from U.S. 2018/0118848A1: 18/26; 82/858; 98/170 |
| MUC17 | AMG199 |
| Nectin-4 | Enfortumab (ASP7465, ASG-22CE, ASG-22ME) |
| | VH: SEQ ID NO: 3 of PCT Pub. WO 2021/151984. |
| | VL: SEQ ID NO: 4 of PCT Pub. WO 2021/151984. |
| Nectin-4 | SBT290 |
| Nectin-4 | VH: SEQ ID NO: 1 of U.S. Pat. No. 11,274, 160. |
| | VL: SEQ ID NO: 2 of U.S. Pat. No. 11,274, 160. |
| Phosphatidylserine | (bavituximab) |
| PSCA | GEM3PSCA |
| PSMA | huJ591 |
| PSMA | Anti-PSMA antibodies having VH and VL sequences having the |
| | amino acid sequences of any one of the following SEQ ID NO: |
| | pairs from WO 2017/023761A1: 2/1642; 10/1642; 18/1642; |
| | 26/1642; 34/1642; 42/1642; 50/1642; 58/1642; 66/1642; 74/1642; |
| | 82/1642; 90/1642; 98/1642; 106/1642; 1 14/1642; 122/130; and |
| | 138/146. |
| PSMA | An antibody such as: PSMA 3.7, PSMA 3.8, PSMA 3.9, PSMA |
| | 3.11, PSMA 5.4, PSMA 7.1, PSMA 7.3, PSMA 10.3, PSMA 1.8.3, |
| | PSMA A3.1.3, PSMA A3.3.1, Abgenix 4.248.2, Abgenix 4.360.3, |
| | Abgenix 4.7.1, Abgenix 4.4.1, Abgenix 4.177.3, Abgenix 4.16.1, |
| | Abgenix 4.22.3, Abgenix 4.28.3, Abgenix 4.40.2, Abgenix 4.48.3, |
| | Abgenix 4.49.1, Abgenix 4.209.3, Abgemx 4.219.3, Abgenix |
| | 4.288.1, Abgenix 4.333.1, Abgemx 4.54.1, Abgenix 4.153.1, |
| | Abgenix 4.232.3, Abgenix 4.292.3, Abgenix 4.304.1, Abgenix |
| | 4.78.1 and Abgenix 4.152.1 described in WO2003034903A2 |
| | A hybridoma cell line such as: PSMA 3.7 (PTA-3257), PSMA 3.8, |
| | PSMA 3.9 (PTA- 3258), PSMA 3.11 (PTA-3269), PSMA 5.4 |
| | (PTA-3268), PSMA 7.1 (PTA-3292), PSMA 7.3 (PTA-3293), |
| | PSMA 10.3 (PTA-3247) , PSMA 1.8.3 (PTA-3906), PSMA A3.1.3 |
| | (PTA- 3904), PSMA A3.3.1 (PTA-3905), Abgenix 4.248.2 (PTA- |
| | 4427), Abgenix 4.360.3 (PTA- 4428), Abgenix 4.7.1 (PTA-4429), |
| | Abgenix 4.4.1 (PTA-4556), Abgenix 4.177.3 (PTA-4557), |
| | Abgenix 4.16.1 (PTA-4357), Abgenix 4.22.3 (PTA-4358), |
| | Abgenix 4.28.3 (PTA-4359), Abgenix 4.40.2 (PTA-4360), |
| | Abgenix 4.48.3 (PTA-4361), Abgenix 4.49.1 (PTA-4362), |
| | Abgenix 4.209.3 (PTA-4365), Abgenix 4.219.3 (PTA-4366), |
| | Abgenix 4.288.1 (PTA-4367), Abgenix 4.333.1 (PTA-4368), |
| | Abgenix 4.54.1 (PTA-4363), Abgenix 4.153.1 (PTA-4388), |
| | Abgenix 4.232.3 (PTA-4389), Abgenix 4.292.3 (PTA-4390), |
| | Abgenix 4.304.1 (PTA-4391), Abgenix 4.78.1 (PTA-4652), and |
| | Abgemx 4.152.1(PTA-4653) described in WO 2003/034903A2. |
| | VH of SEQ ID NOs: 2-7 described in WO 2003/034903A2 |
| | VL of SEQ ID NOs: 8-13 described in WO 2003/034903A2 |
| PSMA | VH: SEQ ID NOs: 225, 239, 253, 267, 281, 295, 309, 323, 337, |
| | 351, 365, 379, 393, 407, 421, 435, 449, 463, 477, 491, 505, 519, |
| | 533, 547, 561, 575, 589, 603 or 617 described in WO |
| | 2011/121110A1. |
| | VL SEQ ID NOs: 230, 244, 258, 272, 286, 300, 314, 328, 342, |
| | 356, 370, 384, 398, 412, 426, 440, 454, 468, 482, 496, 510, 524, |
| | 538, 552, 566, 580, 594, 608 or 622 described in WO |
| | 2011/121110A1. |
| | VH and VL SEQ ID Nos: 235, 249, 263, 277, 291, 305, 319, 333, |
| | 347, 361, 375, 389, 403, 417, 431, 445, 459, 473, 487, 501, 515, |
| | 529, 543, 557, 571, 585, 599, 613 or 627 described in WO |
| | 2011/121110A1. |
| PSMA | An anti-PMSA antibody having a VL amino acid sequence of any |
| | one of SEQ ID NOs: 229-312 of U.S. 2022/0119525 A1 and a VH |
| | of SEQ ID NO: 217 of U.S. 2022/0119525 A1. |
| PSMA | ES414 |
| PSMA | BAY2010112 (pasotuxizumab) |
| PSMA | CCW702 |
| PSMA | JNJ-63898081 |
| PSMA | CC-1 |
| PSMA | Acapatamab |
| PSMA | HPN424 |
| RAAG12 | RAV12 |
| SLAMF7 | Empliciti ™ (elotuzumab) |
| SSTR2 | XmAb ®18087 |
| STEAP1 | VHCDR1 SEQ ID NOs: 14, 33, 182, 184 or 185 described in |
| | U.S.20210179731A1. |
| | VHCDR2 SEQ ID NOs: 15, 21, 34, 182, 184 or 185 described in |
| | U.S.20210179731A1. |
| | VHCDR3 SEQ ID NOs: 16 and 35 described in U.S.20210179731A1. |
| | VH SEQ ID NOs: 182 or 184 described in U.S.20210179731A1. |
| | VLCDR1 SEQ ID NOs: 11 or 30 described in U.S.20210179731A1. |
| | VLCDR2 SEQ ID NOs: 12 or 31 described in U.S.20210179731A1. |
| | VLCDR3 SEQ ID NOs: 13 or 32 described in U.S.20210179731A1. |
| | VL SEQ ID NOs: 183 or 186 described in U.S.20210179731A1. |
| STEAP1 | AMG509 |
| STEAP2 | Anti-STEAP 2 antibodies having CDR-H1, CDR-H2, CDR-H3, |
| | CDR-L1, CDR-L2 and CDR-L3 sequences selected from SEQ ID |
| | NOS: (1) 4-6-8-12-14-16; (2) 20-22-24-28-30-32; (3) 36-38-40- |
| | 44-46-48; (4) 52-54-56-60-62-64; (5) 68-70-72-60-62-64; (6) 76- |
| | 78-80-60-62-64; (7) 84-86-88-60-62-64; (8) 92-94-96-60-62-64; |
| | (9) 100-102-104-60-62-64; (10) 108-110-112-116-118-120; (11) |
| | 124-126-128-132-134-136; (12) 140-142-144-148-150-152; (13) |
| | 156-158-160-164-166-168; (14) 172-174-176-180-182-184; (15) |
| | 188-190-192-196-198-200; (16) 204-206-208-212-214-216; (17) |
| | 220-222-224-228-230-232; (18) 236-238-240-244-246-248; (19) |
| | 252-254-256-260-262-264; (20) 268-270-272-276-278-280; (21) |
| | 284-286-288-292-294-296; (22) 300-302-304-308-310-312; (23) |
| | 316-318-320-324-326-328; (24) 332-334-336-340-342-344; (25) |
| | 348-350-352-356-358-360; (26) 364-366-368-372-374-376; and |
| | (27) 380-382-384-388-390-392 of U.S. Pat. No. 10,772,972 B2. |
| | Anti-STEAP 2 antibodies having (a) a VH comprising the amino |
| | acid of any one of SEQ ID NOs: 2, 18, 34, 50, 66, 74, 82, 90, 98, |
| | 106, 122, 138, 154, 170, 186, 202, 218, 234, 250, 266, 282, 298, |
| | 314, 330, 346, 362, and 378 of U.S. Pat. No. 10,772,972 B2; |
| | and (b) a VL comprising the amino acid sequence of any one of |
| | SEQ ID NOs: 10; 26; 42; 58; 114; 130; 146; 162; 178; 194; 210; |
| | 226, 242; 258; 274; 290; 306; 322; 338; 354; 370; and 386 of U.S. |
| | Pat. No. 10,772,972 B2. |
| | Anti-STEAP 2 antibodies having a VH/VL pair comprising the |
| | amino acid sequences of any of the following pairs of SEQ ID |
| | NOs of U.S. Pat. No. 10,772,972 B2: 2/10; 18/26; 34/42; 50/58; |
| | 66/58; 74/58; 82/58; 90/58; 98/58; 106/114; 122/130; 138/146; |
| | 154/162; 170/178; 186/194; 202/210; 218/226; 234/242; 250/258; |
| | 266/274; 282/290; 298/306; 314/322; 330/338; 346/354; 362/370; |
| | and 378/386. |
| Syndecan-1 (CD 138) | The B-B4 antibody described in Wijdenes et al. (1996) Br. J. |
| | Haematol., 94: 318-323 |
| Syndecan-4 | The amino acid sequence of amino acids 93 and 121 of SEQ ID |
| | NO: 1 or the amino acid sequence of amino acids 92 and 122 of |
| | SEQ ID NO: 2 described in European Patent Pub. EP 2 603 236. |
| TNFR | Enbrel ™ (etanercept) |

In some aspects, the TAA targeting moiety competes with an antibody set forth in Table T1 for binding to the target molecule. In further aspects, the TAA targeting moiety comprises CDRs having CDR sequences of an antibody set forth in Table T1. In some embodiments, the targeting moiety comprises all 6 CDR sequences of the antibody set forth in Table T1. In other embodiments, the targeting moiety comprises at least the heavy chain CDR sequences (CDR-H1, CDR-H2, CDR-H3) of such antibody and the light chain CDR sequences of a universal light chain. In further aspects, a targeting moiety comprises a VH comprising the amino acid sequence of the VH of an antibody set forth in Table T1. In some embodiments, the targeting moiety further comprises a VL comprising the amino acid sequence of the VL of the antibody set forth in Table T1. In other embodiments, the targeting moiety further comprises a universal light chain VL sequence.

In some embodiments, the targeting moieties target the exemplary target molecules set forth in Table T2 below, which provides references to exemplary single domain antibodies or antibody sequences upon which the targeting moiety can be based.

TABLE T2
Exemplary Single Domain Antibody (sdAb) Amino Acid Sequences

| | | | SEQ |
|---|---|---|---|
| Target | Reference | Sequence | ID NO |
| B7H3 | SEQ ID NO: 1 of | HVQLVESGGGLVQPGRSLRLSCAASGFTFSSYWMYWVR | 139 |
| | PCT Publication No. | QTPGKGLEWVSTINRDGSATWYADSVKGRFTISRDNAK | |
| | WO 2021/247794 | NTGYLQMNSLEPDDTAVYYCVSDPDNYSSDEMVPYWGQ | |
| | A2 | GTQVTVSS | |
| B7H3 | SEQ ID NO: 2 of | QVQLVESGGGLVQPGGSLRLSCAASGFTFSSYWMYWVR | 140 |
| | PCT Publication No. | QTPGKGLEWVSTINRDGSATWYADSVKGRFTISRDNAK | |
| | WO 2021/247794 | NTGYLQMNSLKPDDTAVYYCVSDPDNYSSDEMVPYWGQ | |
| | A2 | GTQVTVSS | |
| B7H3 | SEQ ID NO: 3 of | XVQLVESGGGLVQPGXSLRLSCAASGFTFSSYWMYWVR | 141 |
| | PCT Publication No. | QTPGKGLEWVSTINRDGSATWYADSVKGRFTISRDNAK | |
| | WO 2021/247794 | NTGYLQMNSLXPDDTAVYYCVSDPDNYSSDEMVPYWGQ | |
| | A2 | GTQVTVSS | |
| CA9 | SEQ ID NO: 1 of | QVQLVESGGGLVQAGGSLRLSCAASGFTFDDWAIGWFR | 142 |
| | PCT Publication No. | QAPGKEREGVSCISKRHGTTHYADSVKGRFTISSDNAK | |
| | WO 2022/157714 | NTVYLRMNGLKPEDTAVYYCAASSWGSCTVATMRDVDR | |
| | A1 | YDYDYWGQGTQVTVSS | |
| CEACAM | Jancewicz et al, | QVKLEESGGGLVQAGGSLRLSCRTSGRTNSVYTMGWFR | 143 |
| | 2024, Cancer | QAPGKEREFVAQIMWGAGTNTHYADSVKGRFTISRDSA | |
| | Immunol | ESTVYLQMNSLKPEDTAVYYCAANRGIPIAGRQYDYWG | |
| | Immunother. | QGTQVTVSS | |
| | 73(2): 30 | | |
| EpCAM | SEQ ID NO: 1 of | DVQLVESGGGSVQSGGSLRLSCAASGYTYRRYYMGWFR | 144 |
| | PCT Publication No. | QAPGEQREGVAVINNDGRTNYADSVKGRFRISRDNAEN | |
| | WO 2023/044991 | TLHLEMNSLKPEDTAMYYCAATGNILPPMTAVPPLGRQ | |
| | A1 | WYPYWGRGTLVTVSS | |
| EpCAM | SEQ ID NO: 2 of | HVQLVESGGGSVQSGGSLRLSCAASGYAVKNCMGWFRQ | 145 |
| | PCT Publication No. | APGKEREGVAVINRNGITTYADSVKGRFTISQDKDKNT | |
| | WO 2023/044991 | LDLQMNSLKPEDTAMYYCAATPTLLTIPARFLCDVRNP | |
| | A1 | SGFTDWGQGTLVTVSS | |
| EpCAM | SEQ ID NO: 3 of | QVQLVESGGGSVQAGGSLRLSCVVSAYSAYTYKTMCMG | 146 |
| | PCT Publication No. | WFRQAPGKEREGVAAIYRGGLNTYYADSVKGRFIISRD | |
| | WO 2023/044991 | NAESTMYLQMNSLKPEDTAMYYCAADWLRGDDCNIGAN | |
| | A1 | FDYWGQGTQVTVSS | |
| EpCAM | SEQ ID NO: 4 of | QVQLVESGGGSVQAGGSLRLSCVATGFTISRKCMGWFR | 147 |
| | PCT Publication No. | EAPGKKREVIATINTGSSSPYYADGVKGRFTISQDNAK | |
| | WO 2023/044991 | NTVYLQMNSLKPEDTAMYYCAATKGVVVGTGYCGGPYV | |
| | A1 | ERPNSAYWGQGTQVTVSS | |
| EpCAM | SEQ ID NO: 5 of | DVQLVESGGGSVQAGRSLRLSCELSDYTWSTVCMGWFR | 148 |
| | PCT Publication No. | QAPGKEREGVAVIYTRSGGTTYADSAKGRFTISRDNAK | |
| | WO 2023/044991 | DTLYLQMDSLKPEDTAMYYCAAGPLYDGRCTYRSPAFH | |
| | A1 | YWGQGTQVTVSS | |
| EpCAM | SEQ ID NO: 6 of | DVQLVESGGGSAQAGGSLRLSCAASGPTSSLRTMGWFR | 149 |
| | PCT Publication No. | QASGKERERVAVIWDGRTTDYDDSVQDRFTISQDNAKS | |
| | WO 2023/044991 | TVYLQMNTLKPEDTAMYYCAASPRIVPFASTYFQHWGQ | |
| | A1 | GTQVTVSS | |
| EpCAM | SEQ ID NO: 7 of | HVQLVESGGGSVQAGGSLKLSCAASGSIFSGSIFSRCG | 150 |
| | PCT Publication No. | MRWYRQAPGKERELVSSTSKDGFTSYTDSVKGRFTISQ | |
| | WO 2023/044991 | DNANNTLYLQMSSLKTEDTAVYSCAAICAVGGYSLSTY | |
| | A1 | TYWGQGTQVTVSS | |
| EpCAM | SEQ ID NO: 8 of | EVOLVESGGDSVQAGGSLRLSCAASGYSPGSYCMGWFR | 151 |
| | PCT Publication No. | QAPGKERERVAIIESRGTVTYVDSVKGRFTISKDNAKN | |
| | WO 2023/044991 | TLYLQMNSLKPEDTAMYYCAASRPWSGVRCLHDKYDYW | |
| | A1 | GQGTQVTVSS | |
| EpCAM | SEQ ID NO: 9 of | HVQLVESGGGSVQSGGSLRLSCAVSGYAYSSLAWFRQA | 152 |
| | PCT Publication No. | PGKEREGVAALLTAIGGPTRTTYADSVKGRLAISQDHA | |
| | WO 2023/044991 | KNTLYLQMSSLKPEDTAMYYCAAGRPAGTPRWLLLAPR | |
| | A1 | DYNYWGQGTQVTVSS | |
| HER2 | SEQ ID NO: 7 of | QVQLQESGGGSVQAGGSLKLTCAASGYIFNSCGMGWYR | 153 |
| | PCT Publication No. | QSPGRERELVSRISGDGDTWHKESVKGRFTISQDNVKK | |
| | WO 2016/016021 | TLYLQMNSLKPEDTAVYFCAVCYNLETYWGQGTQVTVS | |
| | A1 | S | |
| HER2 | SEQ ID NO: 8 of | QVQLQESGGGLVQPGGSLRLSCAASGFIFSNDAMTWVR | 154 |
| | PCT Publication No. | QAPGKGLEWVSSINWSGTHTNYADSVKGRFTISRDNAK | |
| | WO 2016/016021 | RTLYLQMNSLKDEDTALYYCVTGYGVTKTPTGQGTQVT | |
| | A1 | VSS | |
| HER3 | SEQ ID NO: 265 of | QVQLVQSGGGLVQAGGSLSLSCAFSGRTFSMYTMGWFR | 155 |
| | PCT Publication No. | QAPGKEREFVAANRGRGLSPDIADSVNGRFTISRDNAK | |
| | WO 2021/188736 | NTLYLQMDSLKPEDTAVYYCAADLQYGSSWPQRSSAEY | |
| | A1 | DYWGQGTTVTVSS | |
| MSLN | SEQ ID NO: 1 of | QVQLVQSGGGLVHPGGSLRLSCAASGIDLSLYRMRWYR | 156 |
| | U.S. Publication No. | QAPGKERDLVALITDDGTSYYEDSVKGRFTITRDNPSN | |
| | U.S. 2018/0002439 | KVFLQMNSLKPEDTAVYYCNAETPLSPVNYWGQGTQVT | |
| | A1 | VS | |
| MSLN | SEQ ID NO: 2 of | QVQLVQSGGGLVQAGGSLRLSCAPSGSIFGIRTMDWYR | 157 |
| | U.S. Publication No. | QAPGKERELVARITMDGRVFHADSVKGRFSGSRDGASN | |
| | U.S. 2018/0002439 | AVYLQMNSLKPDDTAVYYCRYSGLTSREDYWGPGTQVT | |
| | A1 | VSS | |
| MSLN | SEQ ID NO: 97 of | QVQLVQSGGGLVHPGGSLRLSCAASGIDLSLYRMRWYR | 156 |
| | PCT Publication No. | QAPGKERDLVALITDDGTSYYEDSVKGRFTITRDNPSN | |
| | WO 2020/023888A2 | KVFLQMNSLKPEDTAVYYCNAETPLSPVNYWGQGTQVT | |
| | | VS | |
| MSLN | SEQ ID NO: 98 of | QVQLVQSGGGLVQAGGSLRLSCAPSGSIFGIRTMDWYR | 157 |
| | PCT Publication No. | QAPGKERELVARITMDGRVFHADSVKGRFSGSRDGASN | |
| | WO 2020/023888A2 | AVYLQMNSLKPDDTAVYYCRYSGLTSREDYWGPGTQVT | |
| | | VSS | |
| MUC16 | SEQ ID NO: 15 of | QVQLQESGGGLVQAGGSLRLSCAASGRTVSSLFMGWFR | 158 |
| | PCT Publication No. | QAPGKERELVAAISRYSLYTYYADSVKGRFTISADNAK | |
| | WO 2020/023888 | NAVYLQMNSLKPEDTAVYYCASKLEYTSNDYDSWGQGT | |
| | A2 | QVTVSS | |
| MUC16 | SEQ ID NO: 20 of | QVQLQESGGGLVQAGDSLRLSCAASGRAVSSLFMGWFR | 159 |
| | PCT Publication No. | RAPGKERELVAAISRYSLYTYYADSVKGRFTISADNAK | |
| | WO 2020/023888 | NAVYLQMNSLKPEDTAVYYCASKLEYTSNDYDSWGQGT | |
| | A2 | QVTVSS | |
| MUC16 | SEQ ID NO: 25 of | QVQLQESGGGLVQAGDSLRLSCAASGRTVSSLFMGWFR | 160 |
| | PCT Publication No. | RAPGKERELVAAISRYSLYTYYADSVKGRFTISADNAK | |
| | WO 2020/023888 | NAVYLQMNSLKPEDTAVYYCASKLEYTSNDYDSWGQGT | |
| | A2 | QVTVSS | |
| MUC16 | SEQ ID NO: 30 of | QVQLQESGGGLVQPGDSMRLSCAAEGDSLDGYVVGWFR | 161 |
| | PCT Publication No. | QAPGKERQGVSSISGDGSMRYVADSVKGRFTISRDNAK | |
| | WO 2020/023888 | NTVYLQMIDLKPEDTGVYYCAADPPTWDYWGQGTQVTV | |
| | A2 | SS | |
| MUC16 | SEQ ID NO: 35 of | QVQLQESGGGLVQPGGSLRLSCAASGRTVSSLFMGWFR | 162 |
| | PCT Publication No. | RAPGKERELVAAISRYSLYTYYADSVKGRFTISADNAK | |
| | WO 2020/023888 | NAVYLQMNSLKPEDTAVYYCASKLEYTSNDYDSWGQGT | |
| | A2 | QVTVSS | |
| MUC16 | SEQ ID NO: 40 of | QVQLQESGGGLVQAGESLRLSCAASGRTVSSLFMGWFR | 163 |
| | PCT Publication No. | RAPGKERELVAAISRYSLYTYYADSVKGRFTISADNAK | |
| | WO 2020/023888 | NAVYLQMNSLKPEDTAVYYCASKLEYTSNDYDSWGQGT | |
| | A2 | QVTVSS | |
| PSMA | Xing et al., 2021 Int. | EVQLVESGGGLVQPGGSLTLSCAASRFMISEYSMHWVR | 164 |
| | J Mol Sci. | QAPGKGLEWVSTINPAGTTDYAESVKGRFTISRDNAKN | |
| | 22(11): 5501 | TLYLQMNSLKPEDTAVYY CDGYGYRGQGTQVTVSS | |
| PSMA | SEQ ID NO: 38 of | QLQLVESGGGLVHAGGSLRLSCAASGSTFSINAIGWYR | 165 |
| | PCT Publication No. | QAPGKQRELVAALSSGGSKNYADSVKGRFTISRDNAKN | |
| | WO 2022/234473 | TVYLQMNRLKPEDTAVYYCNAEIYYSDGVDDGYRGMDY | |
| | A1 | WGKGTQVTVSS | |
| PSMA | SEQ ID NO: 42 of | EVQVVESGGGLVQTGGSLRLSCAASGPPLSSYAVAWFR | 166 |
| | PCT Publication No. | QTPGKEREFVAAISWSGSNTYYADSVKGRFTISKDNAK | |
| | WO 2022/234473 | NTVLVYLQMNSLKPEDTAVYYCAADRRGGPLSDYEWED | |
| | A1 | EYADWGQGTQVTVSS | |

In some aspects, the TAA targeting moiety competes with an antibody set forth above in Table T2, for binding to the target molecule. In further aspects, the TAA targeting moiety comprises CDRs having CDR sequences of an antibody set forth in Table T2. In some embodiments, the targeting moiety comprises all 3 CDR sequences of the antibody set forth in Table T2. In further aspects, a targeting moiety comprises a VH (e.g., a VHH or sdVH) comprising the amino acid sequence of the VH of an antibody set forth in Table T2.

Additional target molecules that can be targeted by the IL2 receptor agonists are disclosed in Table I below and in, e.g., Hafeez et al., 2020, Molecules 25:4764, doi: 10.3390/molecules25204764, particularly in Table 1. Table 1 of Hafeez et al. is incorporated by reference in its entirety herein.

### 6.5.1. PSMA Targeting Moieties

In certain aspects, a TAA targeting moiety of the tumor-targeted split IL2 receptor agonists of the disclosure is a PSMA targeting moiety. In some embodiments, the PSMA targeting moiety is or comprises an antigen-binding domain from an anti-PSMA antibody.

Exemplary anti-PSMA antibodies or antibody sequences are set forth in Tables P1 and P2 below, upon which the TAA targeting moiety can be based.

TABLE P1
Exemplary anti-PSMA Sequences

| Target | Antibody Name or Binding Sequences |
|---|---|
| PSMA | VH: |
| | QVQLVESGGGVVQPGRSLRLSCAASGFTFSSYGMH |
| | WVRQAPGKGLEWVAFMSYDGSNKFYSDSVKGRFTI |
| | SRDNSRKMLFLQMNNLRAEDTAVYYCARDQYYDFL |
| | TDHGVFDYWGQGTLVTVSS |
| | (SEQ ID NO: 254) |
| | VL: |
| | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPWTFGQGTKV |
| | EIK |
| | (SEQ ID NO: 204) |
| PSMA | VH: |
| | QVQLVESGGGVVQPGRSLRLSCAASGFTFSSYGMH |
| | WVRQAPGKGLEWVAVISYAGNNKYYADSVKGRFTV |
| | SRDNSKKTLYLQMNSLRSEDTAVYYCAKDSYYDFL |
| | TDPDVLDIWGQGTMVTVSS |
| | (SEQ ID NO: 255) |
| | VL: |
| | DIQMTQSPSSLSASVGDRVTITCRASQSISSYLNW |
| | YQQKPGKAPKLLIYAASSLQSGVPSRFSGSGSGTD |
| | FTLTISSLQPEDFATYYCQQSYSTPPITFGQGTRL |
| | EIK |
| | (SEQ ID NO: 43) |

In some aspects, the PSMA targeting moiety competes with an antibody set forth in Table P1 for binding to PSMA. In further aspects, the PSMA targeting moiety comprises CDRs having CDR sequences of an anti-PSMA antibody set forth in Table P1. In some embodiments, the PSMA targeting moiety comprises all 6 CDR sequences of an anti-PSMA antibody set forth in Table P1. In other embodiments, the PSMA targeting moiety comprises at least the heavy chain CDR sequences (CDR-H1, CDR-H2, CDR-H3) of an anti-PSMA antibody set forth in Table P1 and the light chain CDR sequences of a universal light chain. In further aspects, the PSMA targeting moiety comprises a VH comprising the amino acid sequence of the VH of an anti-PSMA antibody set forth in Table P1. In some embodiments, the PSMA targeting moiety further comprises a VL comprising the amino acid sequence of the VL of an anti-PSMA antibody set forth in Table P1. In other embodiments, the PSMA targeting moiety further comprises a universal light chain VL sequence.

TABLE P2
Exemplary anti-PSMA Sequences

| Target | Antibody Name or Binding Sequences |
|---|---|
| PSMA | huJ591 |
| PSMA | Anti-PSMA antibodies having VH and VL sequences having the amino acid |
| | sequences of any one of the following SEQ ID NO: pairs from WO |
| | 2017/023761A1: 2/1642; 10/1642; 18/1642; 26/1642; 34/1642; 42/1642; |
| | 50/1642; 58/1642; 66/1642; 74/1642; 82/1642; 90/1642; 98/1642; 106/1642; |
| | 1 14/1642; 122/130; and 138/146. |
| PSMA | An antibody such as: PSMA 3.7, PSMA 3.8, PSMA 3.9, PSMA 3.11, PSMA |
| | 5.4, PSMA 7.1, PSMA 7.3, PSMA 10.3, PSMA 1.8.3, PSMA A3.1.3, PSMA |
| | A3.3.1, Abgenix 4.248.2, Abgenix 4.360.3, Abgenix 4.7.1, Abgenix 4.4.1, |
| | Abgenix 4.177.3, Abgenix 4.16.1, Abgenix 4.22.3, Abgenix 4.28.3, Abgenix |
| | 4.40.2, Abgenix 4.48.3, Abgenix 4.49.1, Abgenix 4.209.3, Abgenix 4.219.3, |
| | Abgenix 4.288.1, Abgenix 4.333.1, Abgenix 4.54.1, Abgenix 4.153.1, |
| | Abgenix 4.232.3, Abgenix 4.292.3, Abgenix 4.304.1, Abgenix 4.78.1 and |
| | Abgenix 4.152.1 described in WO2003034903A2 |
| | A hybridoma cell line such as: PSMA 3.7 (PTA-3257), PSMA 3.8, PSMA 3.9 |
| | (PTA- 3258), PSMA 3.11 (PTA-3269), PSMA 5.4 (PTA-3268), PSMA 7.1 |
| | (PTA-3292), PSMA 7.3 (PTA-3293), PSMA 10.3 (PTA-3247) , PSMA 1.8.3 |
| | (PTA-3906), PSMA A3.1.3 (PTA- 3904), PSMA A3.3.1 (PTA-3905), Abgenix |
| | 4.248.2 (PTA-4427), Abgenix 4.360.3 (PTA- 4428), Abgenix 4.7.1 (PTA- |
| | 4429), Abgenix 4.4.1 (PTA-4556), Abgenix 4.177.3 (PTA-4557), Abgenix |
| | 4.16.1 (PTA-4357), Abgenix 4.22.3 (PTA-4358), Abgenix 4.28.3 (PTA- |
| | 4359), Abgenix 4.40.2 (PTA-4360), Abgenix 4.48.3 (PTA-4361), Abgenix |
| | 4.49.1 (PTA-4362), Abgenix 4.209.3 (PTA-4365), Abgenix 4.219.3 (PTA- |
| | 4366), Abgenix 4.288.1 (PTA-4367), Abgenix 4.333.1 (PTA-4368), Abgenix |
| | 4.54.1 (PTA-4363), Abgenix 4.153.1 (PTA-4388), Abgenix 4.232.3 (PTA- |
| | 4389), Abgenix 4.292.3 (PTA-4390), Abgenix 4.304.1 (PTA-4391), Abgenix |
| | 4.78.1 (PTA-4652), and Abgenix 4.152.1 (PTA-4653) described in WO |
| | 2003/034903A2. |
| | VH of SEQ ID Nos: 2-7 described in WO 2003/034903A2 |
| | VL of SEQ ID Nos: 8-13 described in WO 2003/034903A2 |
| PSMA | VH: SEQ ID Nos: 225, 239, 253, 267, 281, 295, 309, 323, 337, 351, 365, |
| | 379, 393, 407, 421, 435, 449, 463, 477, 491, 505, 519, 533, 547, 561, 575, |
| | 589, 603 or 617 described in WO 2011/121110A1. |
| | VL SEQ ID Nos: 230, 244, 258, 272, 286, 300, 314, 328, 342, 356, 370, |
| | 384, 398, 412, 426, 440, 454, 468, 482, 496, 510, 524, 538, 552, 566, 580, |
| | 594, 608 or 622 described in WO 2011/121110A1. |
| | VH and VL SEQ ID Nos: 235, 249, 263, 277, 291, 305, 319, 333, 347, 361, |
| | 375, 389, 403, 417, 431, 445, 459, 473, 487, 501, 515, 529, 543, 557, 571, |
| | 585, 599, 613 or 627 described in WO 2011/121110A1. |
| PSMA | An anti-PSMA antibody having a VL amino acid sequence of any one of |
| | SEQ ID Nos: 229-312 of U.S. 2022/0119525 A1 and a VH of SEQ ID NO: |
| | 217 of U.S. 2022/0119525 A1. |
| PSMA | ES414 |
| PSMA | BAY2010112 (pasotuxizumab) |
| PSMA | CCW702 |
| PSMA | JNJ-63898081 |
| PSMA | CC-1 |
| PSMA | Acapatamab |
| PSMA | HPN424 |

In some aspects, the PSMA targeting moiety competes with an antibody set forth in Table P2 for binding to PSMA. In further aspects, the PSMA targeting moiety comprises CDRs having CDR sequences of an anti-PSMA antibody set forth in Table P2. In some embodiments, the PSMA targeting moiety comprises all 6 CDR sequences of an anti-PSMA antibody set forth in Table P2. In other embodiments, the PSMA targeting moiety comprises at least the heavy chain CDR sequences (CDR-HI, CDR-H2, CDR-H3) of an anti-PSMA antibody set forth in Table P2 and the light chain CDR sequences of a universal light chain. In further aspects, the PSMA targeting moiety comprises a VH comprising the amino acid sequence of the VH of an anti-PSMA antibody set forth in Table P2. In some embodiments, the PSMA targeting moiety further comprises a VL comprising the amino acid sequence of the VL of an anti-PSMA antibody set forth in Table P2. In other embodiments, the PSMA targeting moiety further comprises a universal light chain VL sequence.

TABLE P3
Exemplary anti-PSMA sdAb Sequences

| Target | Antibody Name or Binding Sequences |
|---|---|
| PSMA | Anti-PSMA sdAb having the following sequence, as disclosed |
| | in Xing et al., 2021 Int. J Mol Sci. 22(11): 5501: |
| | EVQLVESGGGLVQPGGSLTLSCAASRFMISEYSMHWVRQAPGKGLEWVSTINPAGT |
| | TDYAESVKGRFTISRDNAKNTLYLQMNSLKPEDTAVYY (SEQ ID NO: 256) |
| PSMA | Anti-PSMA sdAb (VHH) having SEQ ID NO: 38 of PCT |
| | Publication No. WO 2022/234473 A1: |
| | QLQLVESGGGLVHAGGSLRLSCAASGSTFSINAIGWYRQAPGKQRELVAALSSGGS |
| | KNYADSVKGRFTISRDNAKNTVYLQMNRLKPEDTAVYYCNAEIYYSDGVDDGYRGM |
| | DYWGKGTQVTVSS (SEQ ID NO: 165) |
| PSMA | Anti-PSMA sdAb (VHH) having SEQ ID NO: 42 of PCT |
| | Publication No. WO 2022/234473 A1: |
| | EVQVVESGGGLVQTGGSLRLSCAASGPPLSSYAVAWFRQTPGKEREFVAAISWSGS |
| | NTYYADSVKGRFTISKDNAKNTVLVYLQMNSLKPEDTAVYYCAADRRGGPLSDYEW |
| | EDEYADWGQGTQVTVSS (SEQ ID NO: 166) |

In some aspects, the PSMA targeting moiety competes with a sdAb set forth in Table P3 for binding to PSMA. In further aspects, the PSMA targeting moiety comprises CDRs having CDR sequences of an anti-PSMA sdAb set forth in Table P3. In some embodiments, the PSMA targeting moiety comprises the CDR3 sequence of an anti-PSMA sdAb set forth in Table P3. In some embodiments, the PSMA targeting moiety comprises all 3 CDR sequences of an anti-PSMA sdAb set forth in Table P3.

### 6.5.2. MUC16 Targeting Moieties

In certain aspects, a TAA targeting moiety of the tumor-targeted split IL2 receptor agonists of the disclosure is a MUC16 targeting moiety. In some embodiments, the MUC16 targeting moiety is or comprises an antigen-binding domain from an anti-MUC16 antibody.

Exemplary anti-MUC16 antibodies or antibody sequences are set forth in Tables M1 and M2 below, upon which the TAA targeting moiety can be based.

TABLE M1
Exemplary anti-MUC16 Sequences

| Target | Antibody Name or Binding Sequences |
|---|---|
| MUC16 (CA125) | Anti-MUC16 antibodies having VH and VL sequences having the amino |
| | acid sequences of any one of the following SEQ ID NO: pairs from U.S. |
| | 2018/0118848A1: 18/26; 82/858; 98/170 |

In some aspects, the MUC16 targeting moiety competes with an antibody set forth in Table M1 for binding to MUC16. In further aspects, the MUC16 targeting moiety comprises CDRs having CDR sequences of an anti-MUC16 antibody set forth in Table M1. In some embodiments, the MUC16 targeting moiety comprises all 6 CDR sequences of an anti-MUC16 antibody set forth in Table M1. In other embodiments, the MUC16 targeting moiety comprises at least the heavy chain CDR sequences (CDR-H1, CDR-H2, CDR-H3) of an anti-MUC16 antibody set forth in Table M1 and the light chain CDR sequences of a universal light chain. In further aspects, the MUC16 targeting moiety comprises a VH comprising the amino acid sequence of the VH of an anti-MUC16 antibody set forth in Table M1. In some embodiments, the MUC16 targeting moiety further comprises a VL comprising the amino acid sequence of the VL of an anti-MUC16 antibody set forth in Table M1. In other embodiments, the MUC16 targeting moiety further comprises a universal light chain VL sequence.

TABLE M2
Exemplary anti-MUC16 Sequences

| Target | Antibody Name or Binding Sequences |
|---|---|
| MUC16 | Anti-MUC16 antibodies having VH and VL sequences having |
| | the amino acid sequences of any one of the following |
| | SEQ ID NO: pairs from WO 2016/149368: 1/2; 21/22; |
| | 41/42; 61/62; 81/82; 101/102 |
| MUC16 | abagovomab |
| MUC16 | sofituzumab |

In some aspects, the MUC16 targeting moiety competes with an antibody set forth in Table M2 for binding to MUC16. In further aspects, the MUC16 targeting moiety comprises CDRs having CDR sequences of an anti-MUC16 antibody set forth in Table M2. In some embodiments, the MUC16 targeting moiety comprises all 6 CDR sequences of an anti-MUC16 antibody set forth in Table M2. In other embodiments, the MUC16 targeting moiety comprises at least the heavy chain CDR sequences (CDR-H1, CDR-H2, CDR-H3) of an anti-MUC16 antibody set forth in Table M2 and the light chain CDR sequences of a universal light chain. In further aspects, the MUC16 targeting moiety comprises a VH comprising the amino acid sequence of the VH of an anti-MUC16 antibody set forth in Table M2. In some embodiments, the MUC16 targeting moiety further comprises a VL comprising the amino acid sequence of the VL of an anti-MUC16 antibody set forth in Table M2. In other embodiments, the MUC16 targeting moiety further comprises a universal light chain VL sequence.

TABLE M3
Exemplary anti-MUC16 sdAb Sequences

| Target | Antibody Name or Binding Sequences |
|---|---|
| MUC16 | SEQ ID NO: 15 of PCT Publication No. WO 2020/023888 A2 |
| MUC16 | SEQ ID NO: 25 of PCT Publication No. WO 2020/023888 A2 |
| MUC16 | SEQ ID NO: 30 of PCT Publication No. WO 2020/023888 A2 |
| MUC16 | SEQ ID NO: 35 of PCT Publication No. WO 2020/023888 A2 |
| MUC16 | SEQ ID NO: 40 of PCT Publication No. WO 2020/023888 A2 |

In some aspects, the MUC16 targeting moiety competes with a sdAb set forth in Table M3 for binding to MUC16. In further aspects, the MUC16 targeting moiety comprises ODRs having ODR sequences of an anti-MUCE6 sdAb set forth in Table M3. In some embodiments, the MU16 targeting moiety comprises the ODR3 sequence of an anti-MUC16 sdAb set forth in Table M3. In some embodiments, the MUC16 targeting moiety comprises all 3 ODR sequences of an anti-MUC16 sdAb set forth in Table M3.

### 6.5.3. HER2 Targeting Moieties

In certain aspects, a TAA targeting moiety of the tumor-targeted split IL2 receptor agonists of the disclosure is a HER2 targeting moiety. In some embodiments, the HER2 targeting moiety is or comprises an antigen-binding domain from an anti-HER2 antibody.

Exemplary anti-HER2 antibodies or antibody sequences are set forth in Tables H1 and H2 below, upon which the TAA targeting moiety can be based.

TABLE H1
Exemplary anti-HER2 Sequences

| Target | Antibody Name or Binding Sequences |
|---|---|
| HER2 | Anti-HER2 antibodies having VH and VL sequences having |
| | the amino acid sequences of any one of the following SEQ ID |
| | NO: pairs from PCT Patent Publication No. WO 2021/174113 |
| | A1: 2/18; 10/18; 32/18; 40/18 |

In some aspects, the HER2 targeting moiety competes with an antibody set forth in Table H1 for binding to HER2. In further aspects, the HER2 targeting moiety comprises CDRs having CDR sequences of an anti-HER2 antibody set forth in Table H1. In some embodiments, the HER2 targeting moiety comprises all 6 CDR sequences of an anti-HER2 antibody set forth in Table H1. In other embodiments, the HER2 targeting moiety comprises at least the heavy chain CDR sequences (CDR-H1, CDR-H2, CDR-H3) of an anti-HER2 antibody set forth in Table H1 and the light chain CDR sequences of a universal light chain. In further aspects, the HER2 targeting moiety comprises a VH comprising the amino acid sequence of the VH of an anti-HER2 antibody set forth in Table H1. In some embodiments, the HER2 targeting moiety further comprises a VL comprising the amino acid sequence of the VL of an anti-HER2 antibody set forth in Table H1. In other embodiments, the HER2 targeting moiety further comprises a universal light chain VL sequence.

TABLE H2
Exemplary anti-HER2 Sequences

| Target | Antibody Name or Binding Sequences |
|---|---|
| HER2 | Herceptin ™ (trastuzumab) |
| HER2 | Aldesleukin (proleukine) |
| HER2 | Sargramustim (Leucine) |
| HER2 | M802 |
| HER2 | Runimotamab (BTRC4017A, R07227780) |
| HER2 | ISB1302 |

In some aspects, the HER2 targeting moiety competes with an antibody set forth in Table H2 for binding to HER2. In further aspects, the HER2 targeting moiety comprises CDRs having CDR sequences of an anti-HER2 antibody set forth in Table H2. In some embodiments, the HER2 targeting moiety comprises all 6 CDR sequences of an anti-HER2 antibody set forth in Table H2. In other embodiments, the HER2 targeting moiety comprises at least the heavy chain CDR sequences (CDR-H1, CDR-H2, CDR-H3) of an anti-HER2 antibody set forth in Table H2 and the light chain CDR sequences of a universal light chain. In further aspects, the HER2 targeting moiety comprises a VH comprising the amino acid sequence of the VH of an anti-HER2 antibody set forth in Table H2. In some embodiments, the HER2 targeting moiety further comprises a VL comprising the amino acid sequence of the VL of an anti-HER2 antibody set forth in Table H2. In other embodiments, the HER2 targeting moiety further comprises a universal light chain VL sequence.

TABLE H3
Exemplary anti-HER2 sdAb Sequences

| Target | Antibody Name or Binding Sequences |
|---|---|
| Her2 | SEQ ID NO: 7 of PCT Publication No. WO 2016/016021 A1 |
| Her2 | SEQ ID NO: 8 of PCT Publication No. WO 2016/016021 A1 |

In some aspects, the HER2 targeting moiety competes with a sdAb set forth in Table H3 for binding to HER2. In further aspects, the HER2 targeting moiety comprises CDRs having CDR sequences of an anti-HER2 sdAb set forth in Table H3. In some embodiments, the HER2 targeting moiety comprises the CDR3 sequence of an anti-HER2 sdAb set forth in Table H3. In some embodiments, the HER2 targeting moiety comprises all 3 CDR sequences of an anti-HER2 sdAb set forth in Table H3.

### 6.5.4. EGFR Targeting Moieties

In certain aspects, a TAA targeting moiety of the tumor-targeted split IL2 receptor agonists of the disclosure is an EGFR targeting moiety. In some embodiments, the EGFR targeting moiety is or comprises an antigen-binding domain from an anti-EGFR antibody.

Exemplary anti-EGFR antibodies or antibody sequences are set forth in Tables B1 and B2 below, upon which the TAA targeting moiety can be based.

TABLE B1
Exemplary anti-EGFR Sequences

| Target | Antibody Name or Binding Sequences |
|---|---|
| EGFR | Anti-EGFR antibodies having VH and VL sequences having |
| | the amino acid sequences of any one of the following SEQ |
| | ID NO: pairs from U.S. Pat. No. 9,789,184: |
| | 2/10; 18/26; 34/42; 50/58; 66/74; 82/90; 98/106; 114/122; |
| | 130/138; 146/154; 162/170; 178/186; 194/202; 210/218; |
| | 226/234; 242/250; 258/266; 274/282; 290/298; 306/314; |
| | 322/330; 338/346; 354/362; 370/378. |

In some aspects, the EGFR targeting moiety competes with an antibody set forth in Table B1 for binding to EGFR. In further aspects, the EGFR targeting moiety comprises CDRs having CDR sequences of an anti-EGFR antibody set forth in Table B1. In some embodiments, the EGFR targeting moiety comprises all 6 CDR sequences of an anti-EGFR antibody set forth in Table B1. In other embodiments, the EGFR targeting moiety comprises at least the heavy chain CDR sequences (CDR-H1, CDR-H2, CDR-H3) of an anti-EGFR antibody set forth in Table B1 and the light chain CDR sequences of a universal light chain. In further aspects, the EGFR targeting moiety comprises a VH comprising the amino acid sequence of the VH of an anti-EGFR antibody set forth in Table B1. In some embodiments, the EGFR targeting moiety further comprises a VL comprising the amino acid sequence of the VL of an anti-EGFR antibody set forth in Table 1B1. In other embodiments, the EGFR targeting moiety further comprises a universal light chain VL sequence.

TABLE B2
Exemplary anti-EGFR Sequences

| Target | Antibody Name or Binding Sequences |
|---|---|
| EGFR | Erbitux ™ (cetuximab) |
| | VH: SEQ ID NO: 11 of U.S. Pat. No. 6,217,866 |
| | VL: SEQ ID NO: 13 of U.S. Pat. No. 6,217,866 |
| EGFR | Vectibix ™ (panitumumab) |
| | VH: SEQ ID NO: 37 of U.S. Pat. No. 6,235,883 |
| | VL: SEQ ID NO: 38 of U.S. Pat. No. 6,235,883 |
| EGFR | Zalutumumab |
| | VH: SEQ ID NO: 64 of WO 2018/140831 A2 |
| | VL: SEQ ID NO: 69 of WO 2018/140831 A2 |
| EGFR | Mapatumumab |
| EGFR | Matuzumab |
| EGFR | Nimotuzumab |
| | VH: SEQ ID NO: 51 of WO 2018/140831 A2 |
| | VL: SEQ ID NO: 56 of WO 2018/140831 A2 |
| EGFR | ICR62 |
| EGFR | mAb 528 |
| EGFR | CH806 |
| EGFRv3 | AMG596 |
| EGFRv3 | AMG404 |
| EGFR/CD64 | MDX-447 |
| EGFR | Becotatug |
| EGFR | pimurutamab |
| EGFR | demupitamab |
| EGFR | depatuxizumab |
| EGFR | futuximab |
| EGFR | imgatuzumab |
| EGFR | laprituximab |
| EGFR | losatuxizumab |
| EGFR | modotuximab |
| EGFR | necitumumab |
| EGFR | serclutamab |
| EGFR | tomuzotuximab |
| EGFR | zalutumumab. |

In some aspects, the EGFR targeting moiety competes with an antibody set forth in Table B2 for binding to EGFR. In further aspects, the EGFR targeting moiety comprises CDRs having CDR sequences of an anti-EGFR antibody set forth in Table B2. In some embodiments, the EGFR targeting moiety comprises all 6 CDR sequences of an anti-EGFR antibody set forth in Table B2. In other embodiments, the EGFR targeting moiety comprises at least the heavy chain CDR sequences (CDR-H1, CDR-H2, CDR-H3) of an anti-EGFR antibody set forth in Table B2 and the light chain CDR sequences of a universal light chain. In further aspects, the EGFR targeting moiety comprises a VH comprising the amino acid sequence of the VH of an anti-EGFR antibody set forth in Table B2. In some embodiments, the EGFR targeting moiety further comprises a VL comprising the amino acid sequence of the VL of an anti-EGFR antibody set forth in Table B2. In other embodiments, the EGFR targeting moiety further comprises a universal light chain VL sequence.

TABLE B3
Exemplary anti-EGFR sdAb Sequences

| Target | Antibody Name or Binding Sequences |
|---|---|
| EGFR | SEQ ID NO: 34, 35, 36, 37, 38, 39, 40, 41, 42, 43, 44, 45, |
| | 46, 47, or 48 of U.S. Patent Publication No. US 2024/0156870 |
| | A1. |
| EGFR | SEQ ID NO: 15, 17, 19, 21, 23, 25, 27, 29, 31, 33, or 35 of |
| | PCT Patent Publication No. WO 2008/141449 A1. |

In some aspects, the EGFR targeting moiety competes with a sdAb set forth in Table B3 for binding to EGFR. In further aspects, the EGFR targeting moiety comprises CDRs having CDR sequences of an anti-EGFR sdAb set forth in Table B3. In some embodiments, the EGFR targeting moiety comprises the CDR3 sequence of an anti-EGFR sdAb set forth in Table B3. In some embodiments, the EGFR targeting moiety comprises all 3 CDR sequences of an anti-EGFR sdAb set forth in Table B3.

### 6.5.5. MSLN Targeting Moieties

In certain aspects, a TAA targeting moiety of the tumor-targeted split IL2 receptor agonists of the disclosure is a MSLN targeting moiety. In some embodiments, the MSLN targeting moiety is or comprises an antigen-binding domain from an anti-MSLN antibody.

Exemplary anti-MSLN antibodies or antibody sequences are set forth in Table L1 below, upon which the TAA targeting moiety can be based.

TABLE L1
Exemplary anti-MSLN Sequences

| Target | Antibody Name or Binding Sequences |
|---|---|
| MSLN | amatuximab |
| MSLN | anetumab |
| MSLN | misitatug |

In some aspects, the MSLN targeting moiety competes with an antibody set forth in Table L1 for binding to MSLN. In further aspects, the MSLN targeting moiety comprises CDRs having CDR sequences of an anti-MSLN antibody set forth in Table L. In some embodiments, the MSLN targeting moiety comprises all 6 CDR sequences of an anti-MSLN antibody set forth in Table L. In other embodiments, the MSLN targeting moiety comprises at least the heavy chain CR sequences (CSR-H, CDR-H2, CDR-H3) of an anti-MSLN antibody set forth in Table L1 and the light chain CDR sequences of a universal light chain. In further aspects, the MSLN targeting moiety comprises a VH comprising the amino acid sequence of the VH of an anti-MSLN antibody set forth in Table L11. In some embodiments, the MSLN targeting moiety further comprises a VL comprising the amino acid sequence of the VL of an anti-MSLN antibody set forth in Table L1. In other embodiments, the MSLN targeting moiety further comprises a universal light chain VL sequence.

TABLE L2
Exemplary anti-MSLN sdAb Sequences

| Target | Antibody Name or Binding Sequences |
|---|---|
| MSLN | SEQ ID NO: 1 of U.S. Publication No. US 2018/0002439 A1 |
| MSLN | SEQ ID NO: 2 of U.S. Publication No. US 2018/0002439 A1 |
| MSLN | SEQ ID NO: 97 of PCT Publication No. WO 2020/023888A2 |
| MSLN | SEQ ID NO: 98 of PCT Publication No. WO 2020/023888A2 |

In some aspects, the MSLN targeting moiety competes with a sdAb set forth in Table L2 for binding to MSLN. In further aspects, the MSLN Targeting moiety comprises CDRs having CDR sequences of an anti-MSLN sdAb set forth in Table 1L2. In some embodiments, the MSLN targeting moiety comprises the CDR3 sequence of an anti-MSLN sdAb set forth in Table 1L2. In some embodiments, the MSLN targeting moiety comprises all 3 CDR sequences of an anti-MSLN sdAb set forth in Table 1L2.

### 6.5.6. STEAP1 Targeting Moieties

In certain aspects, a TAA targeting moiety of the tumor-targeted split IL2 receptor agonists of the disclosure is a STEAP1 targeting moiety. In some embodiments, the STEAP1 targeting moiety is or comprises an antigen-binding domain from an anti-STEAP1 antibody.

Exemplary anti-STEAP1 antibodies or antibody sequences are set forth in Table A1 below, upon which the TAA targeting moiety can be based.

TABLE A1
Exemplary anti-STEAP1 Sequences

| Target | Antibody Name or Binding Sequences |
|---|---|
| STEAP1 | VHCDR1 SEQ ID Nos: 14, 33, 182, 184 or 185 described |
| | in US20210179731A1. |
| | VHCDR2 SEQ ID Nos: 15, 21, 34, 182, 184 or 185 |
| | described in US20210179731A1. |
| | VHCDR3 SEQ ID Nos: 16 and 35 described in |
| | US20210179731A1. |
| | VH SEQ ID Nos: 182 or 184 described in |
| | US20210179731A1. |
| | VLCDR1 SEQ ID Nos: 11 or 30 described in |
| | US20210179731A1. |
| | VLCDR2 SEQ ID Nos: 12 or 31 described in |
| | US20210179731A1. |
| | VLCDR3 SEQ ID Nos: 13 or 32 described in |
| | US20210179731A1. |
| | VL SEQ ID Nos: 183 or 186 described in |
| | US20210179731A1. |
| STEAP1 | AMG509 |
| STEAP1 | vandortuzumab |

In some aspects, the STEAP1 targeting moiety competes with an antibody set forth in Table A1 for binding to STEAP1. In further aspects, the STEAP1 targeting moiety comprises CDRs having CDR sequences of an anti-STEAP1 antibody set forth in Table A1. In some embodiments, the STEAP1 targeting moiety comprises all 6 CDR sequences of an anti-STEAP1 antibody set forth in Table A1. In other embodiments, the STEAP1 targeting moiety comprises at least the heavy chain CDR sequences (CDR-H1, CDR-H2, CDR-H3) of an anti-STEAP1 antibody set forth in Table A1 and the light chain CDR sequences of a universal light chain. In further aspects, the STEAP1 targeting moiety comprises a VH comprising the amino acid sequence of the VH of an anti-STEAP1 antibody set forth in Table A1. In some embodiments, the STEAP1 targeting moiety further comprises a VL comprising the amino acid sequence of the VL of an anti-STEAP1 antibody set forth in Table A1. In other embodiments, the STEAP1 targeting moiety further comprises a universal light chain VL sequence.

### 6.6. Multispecific T-Cell Engagers

Aspects of the present disclosure are directed to combinations comprising (a) a tumor-targeted split IL2 receptor agonist (comprising a tumor-targeted IL2Rβ binding molecule and a tumor-targeted IL2Rγ binding molecule) and (b) a multispecific T-cell engager, as well as to methods comprising administration of a tumor-targeted split IL2 receptor agonist and a multispecific T-cell engager simultaneously, sequentially or separately. The tumor-targeted IL2Rβ binding molecule and the tumor-targeted IL2Rγ binding molecule of the tumor-targeted split IL2 receptor agonist can be in the same or separate compositions, and the multispecific T-cell engager can be in the same or separate composition as the tumor-targeted IL2Rβ binding molecule and the tumor-targeted IL2Rγ binding molecule.

As used herein, a “multispecific T-cell engager” describes a molecule comprising: (a) at least one TAA targeting moiety (e.g., as described in Section 6.5); and (b) at least one T-cell receptor (TCR) complex targeting moiety (e.g., as described below). In general, “multispecific T-cell engager,” as used herein, describes a molecule that does not comprise either an IL2Rβ targeting moiety or an IL2Rγ targeting moiety. Also disclosed are pharmaceutical compositions comprising such multispecific T-cell engagers, in some cases together also comprising a tumor-targeted IL2Rβ binding molecule and/or a tumor-targeted IL2Rγ binding molecule. Further disclosed are methods for use of such multispecific T-cell engagers in treatment of cancer in combination with a tumor-targeted split IL2 receptor agonist of the disclosure.

Suitable targeting moiety formats (useful for both the TAA targeting moiety and the TCR complex targeting moiety) are described in Section 6.7. The targeting moiety is preferably an antigen binding domain, e.g., a Fab, as described in Section 6.7.1, an scFv, as described in Section 6.7.2, or a single domain antibody, as described in Section 6.7.3.

Example TAA targeting moieties which may be included in a multispecific T-cell engager disclosed herein are described in Section 6.5. In some embodiments, the TAA targeting moiety of a multispecific T-cell engager targets the same TAA as one or both TAA targeting moieties of the tumor-targeted split IL2 receptor agonist. In some embodiments, the TAA targeting moiety of a multispecific T-cell engager targets a different TAA from both TAA targeting moieties of the tumor-targeted split IL2 receptor agonist. In some embodiments, the TAA targeting moiety of a multispecific T-cell engager targets the same TAA as the TAA targeting moiety of the tumor-targeted IL2Rβ receptor agonist. In some embodiments, the TAA targeting moiety of a multispecific T-cell engager targets the same TAA as the TAA targeting moiety of the tumor-targeted IL2Rγ receptor agonist. In some embodiments, the TAA targeting moiety of a multispecific T-cell engager targets a different TAA as the TAA targeting moiety of the tumor-targeted IL2Rβ receptor agonist. In some embodiments, the TAA targeting moiety of a multispecific T-cell engager targets a different TAA as the TAA targeting moiety of the tumor-targeted IL2Rγ receptor agonist.

Certain example tumor-associated antigens and associated antibodies (or antibody sequences) are provided in Tables T1, T2, P1-P3, M1-M3, H1-H3, B1-B3, L1, L2, and A1. In some embodiments, a multispecific T-cell engager comprises a TAA targeting moiety that specifically binds to a TAA of Table T1. In some embodiments, the TAA is BCMA. In some embodiments, the TAA is CD19. In some embodiments, the TAA is CD20. In some embodiments, the TAA is CD22. In some embodiments, the TAA is EGFR. In some embodiments, the TAA is PSMA. In some embodiments, the TAA is MUC16. In some embodiments, the TAA is CA9. In some embodiments, the TAA is MSLN. In some embodiments, the TAA is EPCAM. In some embodiments, the TAA is B7H3. In some embodiments, the TAA is HER2/HER3 (e.g., HER2). In some embodiments, the TAA is STEAP1. In some embodiments, the TAA is CEACAM5.

A multispecific T-cell engager comprises, in addition to a TAA targeting moiety, a T-cell receptor (TCR) complex targeting moiety. The TCR complex targeting moiety generally binds to any component of the TCR complex. Example targets for a TCR complex targeting moiety of the disclosure include, but are not limited to, CD3 and the T-cell receptor (e.g., TCRαβ or TCRγδ). In some embodiments, the target for the TCR complex targeting moiety is CD3. In some embodiments, the target for the TCR complex targeting moiety is the T-cell receptor (e.g., TCRαβ or TCRγδ). The epitope of the TCR complex targeting moiety can be an individual polypeptide (e.g., CD3 epsilon) or a multimeric component of a protein complex (e.g., the TCRαβ dimer or the TCRγδ dimer of the T-cell receptor complex).

In particular embodiments, a TCR complex targeting moiety of the present disclosure is a CD3 targeting moiety and/or a TCR targeting moiety. A CD3 targeting moiety may be or comprise an antigen-binding domain from an anti-CD3 antibody. A TCR targeting moiety may be or comprise an antigen-binding domain from an anti-TCR antibody.

Exemplary anti-CD3 and anti-TCR antibodies or antibody sequences are set forth in Table G below, upon which the TCR complex targeting moiety can be based.

TABLE G
Exemplary Anti-CD3 and Anti-TCR Antibodies

| Target | Antibody Name and/or Binding Sequences |
|---|---|
| CD3 | The CD3-binding portion of Catumaxomab |
| CD3 | The CD3-binding portion of ertumaxomab |
| CD3 | The CD3-binding portion of anti-PSMA/ |
| | anti-CD3 antibodies described in |
| | WO2011121110A1 |
| CD3 | Anti-CD3 antibody sequences in US 10266593B2 |
| CD3 | Anti-CD3 antibody sequences in US 8846042B2 |
| CD3 | Anti-CD3 antibody sequences US 2016/0355600 |
| CD3 | Anti-CD3 antibody sequences in WO 2014/110601 |
| CD3 | Anti-CD3 antibody sequences in WO 2014/145806 |
| CD3 | Anti-CD3 antibody sequences in U.S. Pat. No. 10,066,015 |
| CD3 | Anti-CD3 antibody sequences in WO 2019/034580 |
| CD3 | Anti-CD3 antibody sequences in WO 2014/056783 |
| CD3 | Anti-CD3 antibody sequences in WO 2013/055809 A1 |
| CD3 | Anti-CD3 antibody sequences in U.S. Pat. No. 10,066,016 |
| CD3 | Anti-CD3 antibody sequences in US 2010/0150918 |
| CD3 | The CD3-binding portion of MT110 |
| CD3 | The CD3-binding portion of Acapatamab (AMG160) |
| CD3 | The CD3-binding portion of AMG199 |
| CD3 | The CD3-binding portion of AMG330 |
| CD3 | The CD3-binding portion of AMG427 (Emirodatamab) |
| CD3 | The CD3-binding portion of AMG562 |
| CD3 | The CD3-binding portion of AMG596 |
| CD3 | The CD3-binding portion of AMG673 |
| CD3 | The CD3-binding portion of AMG701 (Pavurutamab) |
| CD3 | The CD3-binding portion of Tarlatamab (AMG757) |
| CD3 | The CD3-binding portion of AMG910 (Gresonitamab) |
| CD3 | The CD3-binding portion of BAY2010112 (Pasotuxizumab) |
| CD3 | The CD3-binding portion of AMG420 |
| CD3 | The CD3-binding portion of AMG424 |
| CD3 | The CD3-binding portion of AMG509 |
| CD3 | The CD3-binding portion of AMV564 |
| CD3 | The CD3-binding portion of APVO436 |
| CD3 | The CD3-binding portion of Alnuctamab (CC-93269; BMS-986349) |
| CD3 | The CD3-binding portion of ERY974 |
| CD3 | The CD3-binding portion of A-319 |
| CD3 | The CD3-binding portion of GEM333 |
| CD3 | The CD3-binding portion of GEM3PSCA |
| CD3 | The CD3-binding portion of Cevostamab |
| CD3 | The CD3-binding portion of Runimotamab |
| CD3 | The CD3-binding portion of GEN1044 |
| CD3 | Epcoritamab (GEN3013) |
| CD3 | The CD3-binding portion of HPN424 |
| CD3 | The CD3-binding portion of ISB1302 |
| CD3 | The CD3-binding portion of ISB1342 |
| CD3 | The CD3-binding portion of IGM-2323 |
| CD3 | The CD3-binding portion of IMC-F106C |
| CD3 | The CD3-binding portion of IMC-C103C |
| CD3 | The CD3-binding portion of IMCnyeso |
| CD3 | The CD3-binding portion of JNJ-63709178 |
| CD3 | The CD3-binding portion of JNJ-63898081 (JNJ-081) |
| CD3 | The CD3-binding portion of Teclistamab |
| CD3 | The CD3-binding portion of Talquetamab (JNJ-64407564) |
| CD3 | The CD3-binding portion of JNJ-67571244 |
| CD3 | The CD3-binding portion of MGD007 |
| CD3 | The CD3-binding portion of Orlotamab (MGD009) |
| CD3 | The CD3-binding portion of Duvortuxizumab |
| CD3 | The CD3-binding portion of Flotetuzumab (MGD006) |
| CD3 | The CD3-binding portion of MCLA-117 |
| CD3 | The CD3-binding portion of PF-06671008 |
| CD3 | The CD3-binding portion of Elranatamab |
| CD3 | The CD3-binding portion of Odronextamab |
| CD3 | The CD3-binding portion of REGN5458 |
| CD3 | The CD3-binding portion of REGN5459 |
| CD3 | The CD3-binding portion of REGN4018 |
| CD3 | The CD3-binding portion of Glofitamab (RO7082859) |
| CD3 | The CD3-binding portion of RO6958688 (RG7802) |
| CD3 | The CD3-binding portion of SAR440234 |
| CD3 | The CD3-binding portion of TNB-383B |
| CD3 | The CD3-binding portion of M802 |
| CD3 | The CD3-binding portion of Xmab 13676 |
| CD3 | The CD3-binding portion of Xmab18087 |
| CD3 | The CD3-binding portion of Vibecotamab (XmAb14045) |
| CD3 | The CD3-binding portion of Nivatrotamab (Hu3F8-BsAb) |
| CD3 | Anti-CD3 antibody sequences in US20190211100 |
| CD3 | Anti-CD3 antibody sequences in EP1629011B |
| CD3 | VH of SEQ ID NOS. 90 and 98 disclosed in US 2021/0206865 A1 |
| | CDR-H1 of SEQ ID NO: 92 and 100 disclosed in US 2021/0206865 A1 |
| | CDR-H2 of SEQ ID NO: 94 and 102 disclosed in US 2021/0206865 A1 |
| | CDR-H3 of SEQ ID NO: 96 and 104 disclosed in US 2021/0206865 A1 |
| | HC of SEQ ID NO: 127 or SEQ ID NO: 128 disclosed in US 2021/0206865 A1 |
| | LC of SEQ ID NO: 129 or SEQ ID NO: 132 disclosed in US 2021/0206865 A1 |
| CD3 | Anti-CD3 Heavy chain of SEQ ID NO: 2 disclosed in US 2021/0206870 A1 |
| | Anti-CD3 VH SEQ ID NO: 5 disclosed in US 2021/0206870 A1 |
| | Anti-CD3 VL of SEQ ID NO: 6 disclosed in US 2021/0206870 A1 |
| | Anti-CD3 CDR-H1 of SEQ ID NO: 10 disclosed in US 2021/0206870 A1 |
| | Anti-CD3 CDR-H2 of SEQ ID NO: 11 disclosed in US 2021/0206870 A1 |
| | Anti-CD3 CDR-H3 of SEQ ID NO: 12 disclosed in US 2021/0206870 A1 |
| CD3 | Anti-CD3 VH of SEQ ID NO: 92, 102, 112, 122, 132, 142, 156, 166, 176, 186, 196 |
| | or 206 disclosed in US 2022/0119525 A1 |
| | Anti-CD3 CDR-H1 of SEQ ID NO: 93, 103, 113, 123, 133, 143, 157, 167, 177, |
| | 187, 197 or 207 disclosed in US 2022/0119525 A1 |
| | Anti-CD3 CDR-H2 of SEQ ID NO: 94, 104, 114, 124, 134, 144, 158, 168, 178, |
| | 188, 198 or 208 disclosed in US 2022/0119525 A1 |
| | Anti-CD3 CDR-H3 of SEQ ID NO: 95, 105, 115, 125, 135, 145, 159, 169, 179, |
| | 189, 199 or 209 disclosed in US 2022/0119525 A1 |
| | Anti-CD3 VL of SEQ ID NO: 96, 106, 116, 126, 136, 146, 152, 162, 172, 182, 192 |
| | or 202 disclosed in US 2022/0119525 A1 |
| | Anti-CD3 CDR-L1 of SEQ ID NO: 97, 107, 117, 127, 137, 147, 153, 163, 173, |
| | 183, 193 or 203 disclosed in US 2022/0119525 A1 |
| | Anti-CD3 CDR-L2 of SEQ ID NO. 98, 108, 118, 128, 138, 148, 154, 164, 174, |
| | 184, 194 or 204 disclosed in US 2022/0119525 A1 |
| | Anti-CD3 CDR-L3 of SEQ ID NO. 99, 109, 119, 129, 139, 149, 155, 165, 175, |
| | 185, 195 or 205 disclosed in US 2022/0119525 A1 |
| CD3 | L2K |
| CD3 | A2J |
| CD3 | 6G12 |
| CD3 | 1A4 |
| CD3 | OKT3 (Ortho Kung T3; Muromonab-CD3) |
| CD3 | Teplizumab (PRV-031; MGA03) |
| CD3 | Otelixizumab (TRX4) |
| CD3 | Anti-CD3 VH of SEQ ID NO: 2, 18, 34, 50, 66, 82, 98, 114, 130, 146, 162, 178, |
| | 194, 210, 226, 242, 258, 274, 290, 306, 322, 338, 354, 370, 386, 402, 418, 434, |
| | 450, 466, 482, 498, 514, 530, 546, 562, 578, 594, 610, 626, 642, 658, 674, 690, |
| | 706, 722, 738, 754, 770, 786, 802, 818, 834, 850, 866, 882, 898, 914, 930, 946, |
| | 962, 978, 994, 1010, 1026, 1042, 1050, 1058, 1066, 1074, 1082, 1090, 1098, |
| | 1106, 1114, 1122, 1130, 1138, 1146, 1154, 1162, 1170, 1178, 1186, 1194, 1202, |
| | 1210, 1218, or 1226 disclosed in U.S. Pat. No. 9,657,102 B2 |
| | Anti-CD3 CDR-H1 of SEQ ID NO: 4, 20, 36, 52, 68, 84, 100, 116, 132, 148, 164, |
| | 180, 196, 212, 228, 244, 260, 276, 292, 308, 324, 340, 356, 372, 388, 404, 420, |
| | 436, 452, 468, 484, 500, 516, 532, 548, 564, 580, 596, 612, 628, 644, 660, 676, |
| | 692, 708, 724, 740, 756, 772, 788, 804, 820, 836, 852, 868, 884, 900, 916, 932, |
| | 948, 964, 980, 996, 1012, 1028, 1044, 1052, 1060, 1068, 1076, 1084, 1092, |
| | 1100, 1108, 1116, 1124, 1132, 1140, 1148, 1156, 1164, 1172, 1180, 1188, 1196, |
| | 1204, 1212, or 1220, 1228 disclosed in U.S. Pat. No. 9,657,102 B2 |
| | Anti-CD3 CDR-H2 of SEQ ID NO: 6, 22, 38, 54, 70, 86, 102, 118, 134, 150, 166, |
| | 182, 198, 214, 230, 246, 262, 278, 294, 310, 326, 342, 358, 374, 390, 406, 422, |
| | 438, 454, 470, 486, 502, 518, 534, 550, 566, 582, 598, 614, 630, 646, 662, 678, |
| | 694, 710, 726, 742, 758, 774, 790, 806, 822, 838, 854, 870, 886, 902, 918, 934, |
| | 950, 966, 982, 998, 1014, 1030, 1046, 1054, 1062, 1070, 1078, 1086, 1094, |
| | 1102, 1110, 1118, 1126, 1134, 1142, 1150, 1158, 1166, 1174, 1182, 1190, 1198, |
| | 1206, 1214, or 1222, 1230 disclosed in U.S. Pat. No. 9,657,102 B2 |
| | Anti-CD3 CDR-H3 of SEQ ID NO: 8, 24, 40, 56, 72, 88, 104, 120, 136, 152, 168, |
| | 184, 200, 216, 232, 248, 264, 280, 296, 312, 328, 344, 360, 376, 392, 408, 424, |
| | 440, 456, 472, 488, 504, 520, 536, 552, 568, 584, 600, 616, 632, 648, 664, 680, |
| | 696, 712, 728, 744, 460, 776, 792, 808, 824, 840, 856, 872, 888, 904, 920, 936, |
| | 952, 968, 984, 1000, 1016, 1032, 1048, 1056, 1064, 1072, 1080, 1088, 1096, |
| | 1104, 1112, 1120, 1128, 1136, 1144, 1152, 1160, 1168, 1176, 1184, 1192, 1200, |
| | 1208, 1216, or 1224, 1232 disclosed in U.S. Pat. No. 9,657,102 B2 |
| | Anti-CD3 VL of SEQ ID NO: 10, 26, 42, 58, 74, 90, 106, 122, 138, 154, 170, 186, |
| | 202, 218, 234, 250, 266, 282, 298, 314, 330, 346, 362, 378, 394, 410, 426, 442, |
| | 458, 474, 490, 506, 522, 538, 554, 570, 586, 602, 618, 634, 650, 666, 682, 698, |
| | 714, 730, 746, 762, 778, 794, 810, 826, 842, 858, 874, 890, 906, 922, 938, 954, |
| | 970, 986, 1002, 1018, 1034, 1234, 1234, 1234, 1234, 1234, 1234, 1234, 1234, |
| | 1234, 1234, 1234, 1234, 1234, 1234, 1234, 1234, 1234, 1234, 1234, 1234, 1234, |
| | 1234, or 1234, 1234 disclosed in U.S. Pat. No. 9,657,102 B2 |
| | Anti-CD3 CDR-L1 of SEQ ID NO: 12, 28, 44, 60, 76, 92, 108, 124, 140, 156, 172, |
| | 188, 204, 220, 236, 252, 268, 284, 300, 316, 332, 348, 364, 380, 396, 412, 428, |
| | 444, 460, 476, 492, 508, 524, 540, 556, 572, 588, 604, 620, 636, 652, 668, 684, |
| | 700, 716, 732, 748, 764, 780, 796, 812, 828, 844, 860, 876, 892, 908, 924, 940, |
| | 956, 972, 988, 1004, 1020, 1036, 1236, 1236, 1236, 1236, 1236, 1236, 1236, |
| | 1236, 1236, 1236, 1236, 1236, 1236, 1236, 1236, 1236, 1236, 1236, 1236, 1236, |
| | 1236, 1236, or 1236, 1236 disclosed in U.S. Pat. No. 9,657,102 B2 |
| | Anti-CD3 CDR-L2 of SEQ ID NO. 14, 30, 46, 62, 78, 94, 110, 126, 142, 158, 174, |
| | 190, 206, 222, 238, 254, 270, 286, 302, 318, 334, 350, 366, 382, 398, 414, 430, |
| | 446, 462, 478, 494, 510, 526, 542, 558, 574, 590, 606, 622, 638, 654, 670, 686, |
| | 702, 718, 734, 750, 766, 782, 798, 814, 830, 846, 862, 878, 894, 910, 926, 942, |
| | 958, 974, 990, 1006, 1022, 1038, 1238, 1238, 1238, 1238, 1238, 1238, 1238, |
| | 1238, 1238, 1238, 1238, 1238, 1238, 1238, 1238, 1238, 1238, 1238, 1238, 1238, |
| | 1238, 1238, or 1238, 1238 disclosed in U.S. Pat. No. 9,657,102 B2 |
| | Anti-CD3 CDR-L3 of SEQ ID NO. 16, 32, 48, 64, 80, 96, 112, 128, 144, 160, 176, |
| | 192, 208, 224, 240, 256, 272, 288, 304, 320, 336, 352, 368, 384, 400, 416, 432, |
| | 448, 464, 480, 496, 512, 528, 544, 560, 576, 592, 608, 624, 640, 656, 672, 688, |
| | 704, 720, 736, 752, 768, 784, 800, 816, 832, 848, 864, 880, 896, 912, 928, 944, |
| | 960, 976, 992, 1008, 1024, 1040, 1240, 1240, 1240, 1240, 1240, 1240, 1240, |
| | 1240, 1240, 1240, 1240, 1240, 1240, 1240, 1240, 1240, 1240, 1240, 1240, 1240, |
| | 1240, 1240, or 1240, 1240 disclosed in U.S. Pat. No. 9,657,102 B2 |
| | (see also U.S. Pat. No. 9,657,102 B2 at Table 1, incorporated herein by reference) |
| CD3 | VH: |
| | EVQLVESGGGLVQPGRSLRLSCAASGFTFADYTMHWVRQAPGKGLEWVSDIS |
| | WNSGSIAYADSVKGRFTISRDNAKNSLYLQMNSLRTEDTAFYYCAKDSRGYG |
| | HYKYLGLDVWGQGTTVTVSS (SEQ ID NO: 42) |
| | VL: |
| | DIQMTQSPSSLSASVGDRVTITCRASQSISSYLNWYQQKPGKAPKLLIYAASSL |
| | QSGVPSRFSGSGSGTDFTLTISSLQPEDFATYYCQQSYSTPPITFGQGTRLEIK |
| | (SEQ ID NO: 43) |
| CD3 | VH: |
| | EVQLVESGGGLVQPGRSLRLSCAASGFTFDDYSMHWVRQAPGKGLEWVSGIS |
| | WNSGSKGYADSVKGRFTISRDNAKNSLYLQMNSLRAEDTALYYCAKYGSGYGK |
| | FYHYGLDVWGQGTTVTVSS (SEQ ID NO: 44) |
| | VL: |
| | DIQMTQSPSSLSASVGDRVTITCRASQSISSYLNWYQQKPGKAPKLLIYAASSL |
| | QSGVPSRFSGSGSGTDFTLTISSLQPEDFATYYCQQSYSTPPITFGQGTRLEIK |
| | (SEQ ID NO: 43) |
| CD3 | VH: |
| | EVQLVESGGGLVQPGRSLRLSCAASGFTFDDYSMHWVRQAPGKGLEWVSGIS |
| | WNSGSIGYADSVKGRFTISRDNAKNSLYLQMNSLRAEDTALYYCAKDGSGYGK |
| | FYYYGMDVWGQGTTVTVSS (SEQ ID NO: 45) |
| | VL: |
| | DIQMTQSPSSLSASVGDRVTITCRASQSISSYLNWYQQKPGKAPKLLIYAASSL |
| | QSGVPSRFSGSGSGTDFTLTISSLQPEDFATYYCQQSYSTPPITFGQGTRLEIK |
| | (SEQ ID NO: 43) |
| CD3 | VH: |
| | EVQLVESGGGLVQPGRSLRLSCAASGFTFDDYSMHWVRQAPGKGLEWVSGIS |
| | WNSGSIGYADSVKGRFTISRDNAKNSLYLQMNSLRAEDTALYYCAKYGSGYGK |
| | FYYYGMDVWGQGTTVTVSS (SEQ ID NO: 46) |
| | VL: |
| | DIQMTQSPSSLSASVGDRVTITCRASQSISSYLNWYQQKPGKAPKLLIYAASSL |
| | QSGVPSRFSGSGSGTDFTLTISSLQPEDFATYYCQQSYSTPPITFGQGTRLEIK |
| | (SEQ ID NO: 43) |
| TCRαβ | BMA031 sequences disclosed in US 2012/0034221 |
| TCRγδ | 6TCS1 antibody disclosed in U.S. Pat. No. 5,980,892 |

In some aspects, the TCR complex targeting moiety competes with an antibody set forth in Table G for binding to the target (e.g., CD3 or a T-cell receptor). In further aspects, the TCR complex targeting moiety comprises CDRs having CDR sequences of an antibody set forth in Table G. In some embodiments, the TCR complex targeting moiety comprises all 6 CDR sequences of an antibody set forth in Table G. In other embodiments, the TCR complex targeting moiety comprises at least the heavy chain CDR sequences (CDR-H1, CDR-H2, CDR-H3) or an antibody set forth in Table G and the light chain CDR sequences of a universal light chain. In further aspects, a TCR complex targeting moiety comprises a VH comprising the amino acid sequence of the VH of an antibody set forth in Table G. In some embodiments, the TCR complex targeting moiety further comprises a VL comprising the amino acid sequence of the VL of an antibody set forth in Table G. In other embodiments, the TCR complex targeting moiety further comprises a universal light chain VL sequence.

In some embodiments, the multispecific T-cell engager is a bispecific T-cell engager. Certain example bispecific T-cell engagers are provided in Table K. In some embodiments, a bispecific T-cell engager useful in combination with a tumor-targeted split IL2 receptor agonist of the disclosure is a bispecific T-cell engager of Table K. In some embodiments, a bispecific T-cell engager comprises one or more CDR, VH, and/or VL sequences from a bispecific T-cell engager of Table K.

TABLE K
Exemplary Bispecific T-Cell Engagers

| Targets | Name and/or Binding Sequences |
|---|---|
| CD3 × BCMA | Bispecific antibodies bsAb25441D and bsAb25442D described in US |
| | 2022/0306758 A1 and US 2021/0206865 A1 |
| CD3 × CD20 | Bispecific antibodies BS3/20-001, BS3/20-002, BS3/20-003, BS3/20-004, |
| | BS3/20-005, BS3/20-007, and BS3/20-009 described in US 2018/0215823 |
| | A1 |
| CD3 × CD20 | Bispecific antibodies Antibody 1 and Antibody 2 described in US |
| | 20180194841 A1 |
| CD3 × MUC16 | Bispecific antibody BSMUC16/CD3-001 described in US 2020/0399371 A1 |
| CD3 × PSMA | Bispecific antibody PSMA/CD3-005 described in US 2020/0399372 A1 |
| CD3 × CD33 | Bispecific antibodies mAb2 G1 C-LC DANAPA IgG1, mAb2 D5 N-LC |
| | DANAPA IgG1 and mAb2 D5 C-LC DANAPA IgG1 described in US |
| | 2019/0153096A1 |
| CD3 × CLEC12A | Bispecific antibody 5196 × 4327 DM-Fc bsAb described in WO 2017/010874 |
| | A1 |
| CD3 × PSMA | Bispecific antibodies BSPSMA/CD3-001, BSPSMA/CD3-002, BSPSMA/CD3- |
| | 003, BSPSMA/CD3-200, BSPSMA/CD3-300, BSPSMA/CD3-400, |
| | BSPSMA/CD3-004, BSPSMA/CD3-800, BSPSMA/CD3-900, BSPSMA/CD3- |
| | 1000, BSPSMA/CD3-1100, BSPSMA/CD3-1200, BSPSMA/CD3-1300, |
| | BSPSMA/CD3-1400, BSPSMA/CD3-1500, BSPSMA/CD3-1600, |
| | BSPSMA/CD3-1700, BSPSMA/CD3-1800, BSPSMA/CD3-1900, |
| | BSPSMA/CD3-005, BSPSMA/CD3-2100 described in US 2021/0403595 A1 |
| CD3 × BCMA | Bispecific antibodies BCMB72, BC3B7, BC3B8, BC3B9, BC3B10, BC3B11, |
| | BC3B12 described in WO 2017/031104 A1 |
| CD3 × EpCAM | Catumaxomab, MT110 |
| CD3 × EpCAM | MT110 |
| CD3 × HER2/neu | Ertumaxomab |
| CD3 × HER2 | ISB1302 |
| CD3 × HER2 | Runimotamab |
| CD3 × HER2 | M802 |
| CD3 × PSMA | Acapatamab |
| CD3 × PSMA | BAY2010112 (Pasotuxizumab) |
| CD3 × PSMA | JNJ-63898081 (JNJ-081) |
| CD3 × MUC17 | AMG199 |
| CD3 × CD33 | AMG330 |
| CD3 × CD33 | AMG673 |
| CD3 × CD33 | AMV564 |
| CD3 × CD33 | GEM333 |
| CD3 × CD33 | JNJ-67571244 |
| CD3 × FLT3 | AMG427 (Emirodatamab) |
| CD3 × CD19 | AMG562 |
| CD3 × CD19 | A-319 |
| CD3 × CD19 | Duvortuxizumab |
| CD3 × EGFRvIII | AMG596 |
| CD3 × BCMA | Alnuctamab (CC-93269, BMS-986349) |
| CD3 × BCMA | AMG701 (Pavurutamab) |
| CD3 × BCMA | AMG420 |
| CD3 × BCMA | Teclistamab |
| CD3 × BCMA | Elranatamab |
| CD3 × BCMA | REGN5458 |
| CD3 × BCMA | REGN5459 |
| CD3 × BCMA | TNB-383B |
| CD3 × NY-ESO-1 | IMCnyeso |
| CD3 × MAGE-A4 | IMC-C103C |
| CD3 × PRAME | IMC-F106C |
| CD3 × 5T4 | GEN1044 |
| CD3 × DLL3 | AMG757 |
| CD3 × CLDN18.2 | AMG910 (Gresonitamab) |
| CD3 × GPC3 | ERY974 |
| CD3 × gpA33 | MGD007 |
| CD3 × B7-H3 | Orlotamab (MGD007) |
| CD3 × SSTR2 | XmAb-18087 |
| CD3 × PSCA | GEM3PSCA |
| CD3 × CD38 | AMG424 |
| CD3 × CD38 | ISB1342 |
| CD3 × STEAP1 | AMG509 |
| CD3 × FCRL5 | Cevostamab |
| CD3 × CD123 | APVO436 |
| CD3 × CD123 | JNJ-63709178 |
| CD3 × CD123 | Flotetuzumab (MGD006) |
| CD3 × CD123 | SAR440234 |
| CD3 × CD123 | Vibecotamab (XmAb14045) |
| CD3 × CD20 | Epcoritamab (GEN3013) |
| CD3 × CD20 | IGM-2323 |
| CD3 × CD20 | Odronextamab |
| CD3 × CD20 | Glofitamab (RO7082859) |
| CD3 × CD20 | XmAb13676 |
| CD3 × GPRC5D | Talquetamab (JNJ-64407564) |
| CD3 × CLEC12A | MCLA-117 |
| CD3 × MUC16 | REGN4018 |
| CD3 × CEA | RO6958688 (RG7802) |
| CD3 × GD2 | Nivatrotamab (Hu3F8-BsAb) |
| CD3 × MSLN | ZW171 |
| CD3 × MSLN | CT-95 |

### 6.7. Targeting Moiety Formats

In certain aspects, a targeting moiety (e.g., a TAA targeting moiety, an IL2Rβ targeting moiety, an IL2Rγ targeting moiety, or a TCR complex targeting moiety) can be any type of antibody or fragment thereof that retains specific binding to an antigenic determinant. In one embodiment the antigen binding domain is a full-length antibody. In one embodiment the antigen binding domain is an immunoglobulin molecule, particularly an IgG class immunoglobulin molecule, more particularly an IgG1 or IgG4 immunoglobulin molecule. In another embodiment, the antigen binding domain is a single domain antibody. Antibody fragments include, but are not limited to, VH (or VH) fragments, VL (or VL) fragments, Fab fragments, F(ab′)2 fragments, scFv fragments, Fv fragments, VHH domains, minibodies, diabodies, triabodies, and tetrabodies.

In some embodiments, the TAA targeting moiety of the tumor-targeted IL2Rβ binding molecule and the TAA targeting moiety of the tumor-targeted IL2Rγ binding molecule share the same format (e.g., Fab, scFv or sdAb). In another embodiment, the TAA targeting moiety of the tumor-targeted IL2Rβ binding molecule and the TAA targeting moiety of the tumor-targeted IL2Rγ binding molecule do not share the same format.

In some embodiments the TAA targeting moieties of the tumor-targeted IL2Rβ binding molecule and the tumor-targeted IL2Rγ binding molecule are Fabs. In other embodiments, the TAA targeting moieties of the tumor-targeted IL2Rβ binding molecule and the tumor-targeted IL2Rγ binding molecule are scFvs. In yet other embodiments, the TAA targeting moieties of the tumor-targeted IL2Rβ binding molecule and the tumor-targeted IL2Rγ binding molecule are sdAbs.

In some embodiments, where the IL2Rβ and IL2Rγ binding moieties of a tumor-targeted split IL2 receptor agonist are IL2Rβ and IL2Rγ targeting moieties, the IL2Rβ and IL2Rγ targeting moieties share the same format (e.g., Fab, scFv or sdAb). In particular embodiments, the IL2Rβ and IL2Rγ targeting moieties are both sdAbs. In other embodiments, where the IL2Rβ and IL2Rγ binding moieties of a tumor-targeted split IL2 receptor agonist are IL2Rβ and IL2Rγ targeting moieties, the IL2Rβ and IL2Rγ targeting moieties do not share the same format.

In some embodiments the IL2Rβ and IL2Rγ targeting moieties are Fabs. In other embodiments, the IL2Rβ and IL2Rγ targeting moieties are scFvs. In yet other embodiments, the IL2Rβ and IL2Rγ targeting moieties are sdAbs.

In some embodiments, the TAA targeting moieties and the IL2Rβ and IL2Rγ targeting moieties share the same format (e.g., Fab, scFv or sdAb). In other embodiments, the TAA targeting moieties and the IL2Rβ and IL2Rγ targeting moieties do not share the same format (e.g., the TAA targeting moieties are Fabs and the IL2Rβ and IL2Rγ targeting moieties are sdAbs or vice versa).

### 6.7.1. Fabs

Fab domains were traditionally produced by proteolytic cleavage of immunoglobulin molecules using enzymes such as papain. In the tumor-targeted split IL2 receptor agonists of the disclosure, the Fab domains can be recombinantly expressed as part of the tumor-targeted IL2Rβ binding molecule and/or the tumor-targeted IL2Rγ binding molecule.

The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the tumor-target split IL2 receptor agonists of the disclosure, particularly when the light chain is not a common or universal light chain, it is advantageous to use Fab heterodimerization strategies to permit the correct association of Fab domains belonging to the same ABD and minimize aberrant pairing of Fab domains belonging to different ABDs. For example, the Fab heterodimerization strategies shown in Table F below can be used:

TABLE F
Fab Heterodimerization Strategies

| STRATEGY | VH | CH1 | VL | CL | REFERENCE |
|---|---|---|---|---|---|
| CrossMabCH1-CL | WT | CL domain | WT | CH1 domain | Schaefer et al., |
| | | | | | 2011, Cancer Cell |
| | | | | | 2011; 20: 472-86; |
| | | | | | PMID: 22014573. |
| orthogonal Fab | 39K, 62E | H172A, | 1R, 38D, | L135Y, | Lewis et al., 2014, |
| VHVRD1CH1CRD2 - | | F174G | (36F) | S176W | Nat Biotechnol |
| VLVRD1Cλ | | | | | 32: 191-8 |
| CRD2 | | | | | |
| orthogonal Fab | 39Y | WT | 38R | WT | Lewis et al., 2014, |
| VHVRD2CH | | | | | Nat Biotechnol |
| 1 wt - | | | | | 32: 191-8 |
| VLVRD2Cλ | | | | | |
| wt | | | | | |
| TCR CαCβ | 39K | TCR Cα | 38D | TCR Cβ | Wu et al., 2015, |
| | | | | | MAbs 7: 364-76 |
| CR3 | WT | T192E | WT | N137K, | Golay at al., 2016, J |
| | | | | S114A | Immunol 196: 3199- |
| | | | | | 211. |
| MUT4 | WT | L143Q, | WT | V133T, | Golay at al., 2016, J |
| | | S188V | | S176V | Immunol 196: 3199- |
| | | | | | 211. |
| DuetMab | WT | F126C | WT | S121C | Mazor et al., 2015, |
| | | | | | MAbs 7: 377-89; |
| | | | | | Mazor et al., 2015, |
| | | | | | MAbs 7: 461-669. |
| Domain | WT | CH3 + knob | WT | CH3 + hole | Wozniak-Knopp et |
| exchanged | | or hole | | or knob | al., 2018, |
| | | mutation | | mutation | PLoSONE13(4): |
| | | | | | e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the T-cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each Fab VL region of an IL2Rβ or IL2Rγ receptor agonist of the disclosure. In various embodiments, employing a common light chain as described herein reduces the number of inappropriate species of IL2Rβ or IL2Rγ receptor agonists as compared to employing original cognate VLs. In various embodiments, the VL domains of the IL2Rβ or IL2Rγ receptor agonists are identified from monospecific antibodies comprising a common light chain. In various embodiments, the VH regions of the IL2Rβ or IL2Rγ receptor agonists comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human Vκ1-39JK5 sequence or a rearranged human VK3-20JK1 sequence, and include somatically mutated (e.g., affinity matured) versions. See, for example, U.S. Pat. No. 10,412,940.

6.7.2. scFvs

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 6.9.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.5.3 (typically a repeat of a sequence containing the amino acids glycine and serine, such as the amino acid sequence (Gly4˜Ser)₃ (SEQ ID NO: 47), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.7.3. Single Domain Antibodies

In some embodiments, a targeting moiety e.g., a TAA targeting moiety, an IL2Rβ targeting moiety, or an IL2Rγ targeting moiety) is a single-domain antibody. A single-domain antibody (sdAb) describes a single antigen-binding domain capable of binding to a cognate antigen. sdAbs are often derived from heavy-chain only antibodies, however they also include single VH domains capable of binding to their cognate antigen in the absence of an associated light chain. In some embodiments, sdAbs also include single VL domains capable of binding to their cognate antigen in the absence of an associated light chain. Single VH or VL domains may have amino acid changes relative to native VH or VL sequences that stabilize the domains and/or reduce or eliminate aggregation.

Heavy-chain only antibodies lack both light chains and a functional CH1 domain and thus rely exclusively on a heavy chain variable domain for antigen binding. Heavy-chain only antibodies are produced naturally in the Camelidae family (e.g., camels, dromedaries, llamas, vicunas, guanaco, and alpacas) as well as in cartilaginous fish (e.g., sharks). In addition to natural sources, transgenic mammals (e.g., mice) have been engineered to express heavy-chain only antibodies. Such transgenic mammals include, for example, transgenic animals described in U.S. Patent Publications 2015/0289489 A1, 2023/0270086 A1, and 2023/0062964 A1, and 2020/0267951 A1, each of which is incorporated herein by reference.

In some embodiments, an sdAb is generated by immunizing an animal that produces heavy-chain only antibodies, including a natural producer (e.g., camelids, sharks) or an engineered non-human mammal (e.g., a transgenic mouse), to obtain heavy-chain only antibodies. Such antibodies may be screened to identify those having desirable properties (e.g., target affinity). Once produced and identified, the variable region of the antibody heavy chain is cloned to construct a single domain antibody consisting of only one heavy chain variable region.

sdAbs can also be obtained by immunizing animals that generate traditional antibodies (e.g., rabbits) followed by screening for VHs having high binding affinity in the absence of their cognate light chain (see e.g., Shinozaki et al., 2017, Scientific Reports, 7(1):5794).

sdAbs can be humanized by replacing natural (e.g., camelid) framework sequences with human sequences (see, e.g., Vincke, 2009, The Journal of Biological Chemistry, 285(5):3273-3284; Murakami et al., 2022, Antibodies, 11(1):10; and U.S. Patent Publication No. 2016/0237142 A1, incorporated herein by reference).

Fully human sdAbs can also be obtained using human VH single domains (see, e.g., Rouet et al., 2015, The Journal of Biological Chemistry, 290(19):11905-11917).

Additional methods for producing heavy-chain only antibodies and/or sdAbs are recognized in the art and include, for example, those described in Muyldermans, 2021, The FEBS journal, 288(7):2084-2102.

In some cases, an sdAb is engineered to enhance certain properties. For example, in some embodiments, a disulfide bond is introduced within a VHH to increase stability (see e.g., Hagihara et al., 2007, The Journal of Biological Chemistry, 282(50):36489-36495).

### 6.8. Fc Domains

The tumor-targeted IL2Rβ binding molecule and the tumor-targeted IL2Rγ binding molecule include an Fc domain of the tumor-targeted split IL2 receptor agonists of the disclosure each comprises a pair of Fc domains to which the TAA targeting moiety and the IL2Rβ binding moiety (in the case of the tumor-targeted IL2Rβ binding molecule) or the TAA targeting moiety and the IL2Rγ binding moiety (in the case of the tumor-targeted IL2Rγ binding molecule) are operably linked.

In some embodiments, the tumor-targeted IL2Rβ binding molecule comprises an Fc region formed by the association of an Fc pair, one comprising a TAA targeting moiety at its N-terminus and the other comprising an IL2Rβ binding moiety (e.g., an IL2Rβ targeting moiety) at its N-terminus.

In some embodiments, the tumor-targeted IL2Rγ binding molecule comprises an Fc region formed by the association of an Fc pair, one comprising a TAA targeting moiety at its N-terminus and the other comprising an IL2Rγ binding moiety (e.g., an IL2Rγ targeting moiety) at its N-terminus.

In one embodiment the Fc domains of the tumor-targeted IL2Rβ binding molecule and the tumor-targeted IL2Rγ binding molecule are derived from a human Fc domain.

The Fc domains that can be incorporated into a tumor-targeted IL2Rβ binding molecule and/or a tumor-targeted IL2Rγ binding molecule can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domains of both the tumor-targeted IL2Rβ binding molecule and the tumor-targeted IL2Rγ binding molecule are derived from IgG1, IgG2, IgG3 or IgG4. In one embodiment, one or both pairs of Fc domains are derived from IgG1. In one embodiment, one or both pairs of Fc domains are derived from IgG4.

The two Fc domains within the Fc region of the tumor-targeted IL2Rβ binding molecule and/or a tumor-targeted IL2Rγ binding molecule can be the same or different from one another. In a native antibody the Fc domains are typically identical, but for the purpose of producing molecules with different binding domains (e.g., a TAA targeting moiety and an IL2Rβ binding moiety or an IL2Rγ binding moiety, the Fc domains might advantageously be different to allow for heterodimerization, as described in Section 6.8.2 below.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In the tumor-targeted IL2Rβ binding molecules and/or tumor-targeted IL2Rγ binding molecules of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

It will be appreciated that the heavy chain constant domains for use in producing an Fc region for the tumor-targeted IL2Rβ binding molecules and/or tumor-targeted IL2Rγ binding molecules of the present disclosure may include variants of the naturally occurring constant domains described above. Such variants may comprise one or more amino acid variations compared to wild type constant domains. In one example the Fc region of the present disclosure comprises at least one constant domain that varies in sequence from the wild type constant domain. It will be appreciated that the variant constant domains may be longer or shorter than the wild type constant domain. Preferably the variant constant domains are at least 60% identical or similar to a wild type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the tumor-targeted IL2Rβ binding molecules and/or tumor-targeted IL2Rγ binding molecules of the present disclosure do not comprise a tailpiece.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:5, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:5, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:6, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:6, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:7, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:7, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:8, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:8, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:9, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:9, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:10, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:10, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:11, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO: 11, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:12, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:12, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:13, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:13, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:14, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:14, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:15, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:15, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:16, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:16, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:17, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:17, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:18, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:18, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:19, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:19, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:20, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:20, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:21, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:21, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:22, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:22, optionally with one or more mutations that facilitate heterodimerization or purification, e.g., (a) knob or hole mutations and/or (b) star mutations.

In some embodiments, a tumor-targeted IL2Rβ binding molecule and/or tumor-targeted IL2Rγ binding molecule comprises an Fc domain that has at least 95%, at least 96%, at least 97%, at least 98%, at least 99% or at least 99.5% sequence identity to the amino acid sequence of SEQ ID NO:23, optionally comprising one more mutations that facilitate heterodimerization or purification, e.g., (a) knob or hole mutations and/or (b) star mutations. In some embodiments, a tumor-targeted IL2Rβ binding molecule and/or tumor-targeted IL2Rγ binding molecule comprises an Fc domain that has the amino acid sequence of SEQ ID NO:23, optionally with one or more mutations that facilitate heterodimerization or purification, e.g., (a) knob or hole mutations and/or (b) star mutations.

The Fc domains that are incorporated into the tumor-targeted IL2Rβ binding molecules and/or tumor-targeted IL2Rγ binding molecules of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.8.1.

The Fc domains can also be altered to include modifications that improve manufacturability of asymmetric tumor-targeted IL2Rβ binding molecules and/or tumor-targeted IL2Rγ binding molecules, for example by allowing heterodimerization, which is the preferential pairing of non-identical Fc domains over identical Fc domains. Heterodimerization permits the production of tumor-targeted IL2Rβ binding molecules and/or tumor-targeted IL2Rγ binding molecules in which different polypeptide components are connected to one another by an Fc region containing Fc domains that differ in sequence. Examples of heterodimerization strategies are exemplified in Section 6.8.2.

It will be appreciated that any of the modifications mentioned above can be combined in any suitable manner to achieve the desired functional properties and/or combined with other modifications to alter the properties of the tumor-targeted IL2Rβ binding molecules and/or tumor-targeted IL2Rγ binding molecules.

6.8.1. Fc Domains with Altered Effector Function

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain or the Fc region (e.g., one or both Fc domains of a tumor-targeted IL2Rβ binding molecules and/or tumor-targeted IL2Rγ binding molecule that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table C below: In some embodiments, the Fc domain includes only the bolded portion of the sequences shown below:

TABLE C

| | | SEQ |
|---|---|---|
| Fc Domain | Sequence | ID NO |
| SEQ ID NO: 1 of | DKRVESKYGP PCPPCPAPPV AGPSVFLFPP KPKDTLMISR | 16 |
| WO2014/121087 | TPEVTCVVVD VSQEDPEVQF NWYVDGVEVH NAKTKPREEQ | |
| | FNSTYRVVSV LTVLHQDWLN GKEYKCKVSN KGLPSSIEKT | |
| | ISKAKGQPRE PQVYTLPPSQ EEMTKNQVSL TCLVKGFYPS | |
| | DIAVEWESNG QPENNYKTTP PVLDSDGSFF LYSRLTVDKS | |
| | RWQEGNVFSC SVMHEALHNH YTQKSLSLSL GK | |
| SEQ ID NO: 2 of | DKKVEPKSCD KTHTCPPCPA PPVAGPSVFL FPPKPKDTLM | 17 |
| WO2014/121087 | ISRTPEVTCV VVDVSQEDPE VQFNWYVDGV EVHNAKTKPR | |
| | EEQFNSTYRV VSVLTVLHQD WLNGKEYKCK VSNKGLPSSI | |
| | EKTISKAKGQ PREPQVYTLP PSRDELTKNQ VSLTCLVKGF | |
| | YPSDIAVEWE SNGQPENNYK TTPPVLDSDG SFFLYSKLTV | |
| | DKSRWQQGNV FSCSVMHEAL HNHYTQKSLS LSPGK | |
| SEQ ID NO: 30 | ASTKGPSVFP LAPSSKSTSG GTAALGCLVK DYFPEPVTVS | 18 |
| of | WNSGALTSGV HTFPAVLQSS GLYSLSSVVT VPSSSLGTQT | |
| WO2014/121087 | YICNVNHKPS NTKVDKKVEP KSCDKTHTCP PCPAPPVAGP | |
| | SVFLFPPKPK DTLMISRTPE VTCVVVDVSQ EDPEVQFNWY | |
| | VDGVEVHNAK TKPREEQFNS TYRVVSVLTV LHQDWLNGKE | |
| | YKCKVSNKGL PSSIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPGK | |
| SEQ ID NO: 31 | ASTKGPSVFP LAPCSRSTSE STAALGCLVK DYFPEPVTVS | 19 |
| of | WNSGALTSGV HTFPAVLQSS GLYSLSSVVT VPSSSLGTKT | |
| WO2014/121087 | YTCNVDHKPS NTKVDKRVES KYGPPCPPCP APPVAGPSVF | |
| | LFPPKPKDTL MISRTPEVTC VVVDVSQEDP EVQFNWYVDG | |
| | VEVHNAKTKP REEQFNSTYR VVSVLTVLHQ DWLNGKEYKC | |
| | KVSNKGLPSS IEKTISKAKG QPREPQVYTL PPSQEEMTKN | |
| | QVSLTCLVKG FYPSDIAVEW ESNGQPENNY KTTPPVLDSD | |
| | GSFFLYSRLT VDKSRWQEGN VFSCSVMHEA LHNHYTQKSL | |
| | SLSLGK | |
| SEQ ID NO: 37 | ASTKGPSVFP LAPSSKSTSG GTAALGCLVK DYFPEPVTVS | 20 |
| of | WNSGALTSGV HTFPAVLQSS GLYSLSSVVT VPSSSLGTQT | |
| WO2014/121087 | YICNVNHKPS NTKVDKKVEP KSCDKTHTCP PCPAPPVAGP | |
| | SVFLFPPKPK DTLMISRTPE VTCVVVDVSQ EDPEVQFNWY | |
| | VDGVEVHNAK TKPREEQFNS TYRVVSVLTV LHQDWLNGKE | |
| | YKCKVSNKGL VKGFYPSDIA AKGQPREPQV YTLPPSRDEL | |
| | TKNQVSLTCL PSSIEKTISK VEWESNGQPE NNYKTTPPVL | |
| | DSDGSFFLYS KLTVDKSRWQ QGNVFSCSVM HEALHNRFTQ | |
| | KSLSLSPGK | |
| SEQ ID NO: 38 | ASTKGPSVFP LAPCSRSTSE STAALGCLVK DYFPEPVTVS | 21 |
| of | WNSGALTSGV HTFPAVLQSS GLYSLSSVVT VPSSSLGTKT | |
| WO2014/121087 | YTCNVDHKPS NTKVDKRVES KYGPPCPPCP APPVAGPSVF | |
| | LFPPKPKDTL MISRTPEVTC VVVDVSQEDP EVQFNWYVDG | |
| | VEVHNAKTKP REEQFNSTYR VVSVLTVLHQ DWLNGKEYKC | |
| | KVSNKGLPSS IEKTISKAKG QPREPQVYTL PPSQEEMTKN | |
| | QVSLTCLVKG FYPSDIAVEW ESNGQPENNY KTTPPVLDSD | |
| | GSFFLYSRLT VDKSRWQEGN VFSCSVMHEA LHNRFTQKSL | |
| | SLSLGK | |

In a particular embodiment, the IgG4 with reduced effector function comprises the bolded portion of the amino acid sequence of SEQ ID NO:19 (SEQ ID NO:31 of WO2014/121087), sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:18 (SEQ ID NO:30 of WO2014/121087) (or the bolded portion thereof) and an Fc domain comprising the amino acid sequence of SEQ ID NO:20 (SEQ ID NO:37 of WO2014/121087) (or the bolded portion thereof) or an Fc region comprising an Fc domain comprising the amino acid sequence of SEQ ID NO:19 (SEQ ID NO:31 of WO2014/121087) (or the bolded portion thereof) and an Fc domain comprising the amino acid sequence of SEQ ID NO:21 (SEQ ID NO:38 of WO2014/121087) (or the bolded portion thereof).

### 6.8.2. Fc Heterodimerization Variants

Certain tumor-targeted IL2Rβ binding molecules and/or tumor-targeted IL2Rγ binding molecules entail dimerization between two Fc domains that, unlike a native immunoglobulin, are operably linked to non-identical N-terminal regions, e.g., one Fc domain connected to a TAA targeting moiety and the other Fc domain connected to an IL2Rβ or IL2Rγ binding moiety. Inadequate heterodimerization of two Fc domains to form an Fc region can be an obstacle for increasing the yield of desired heterodimeric molecules and represents challenges for purification. A variety of approaches available in the art can be used in for enhancing dimerization of Fc domains that might be present in the tumor-targeted IL2Rβ binding molecules and/or tumor-targeted IL2Rγ binding molecules of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

The present disclosure provides tumor-targeted IL2Rβ binding molecules and/or tumor-targeted IL2Rγ binding molecules comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Heterodimerization of the two different heavy chains at CH3 domains give rise to the desired tumor-targeted IL2Rβ binding molecule and/or tumor-targeted IL2Rγ binding molecule, while homodimerization of identical heavy chains will reduce yield of the desired tumor-targeted IL2Rβ binding molecule and/or tumor-targeted IL2Rγ binding molecule. Thus, in a preferred embodiment, the polypeptides that associate to form a tumor-targeted IL2Rβ binding molecule and/or tumor-targeted IL2Rγ binding molecule of the disclosure will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the IL2Rβ and/or IL2Rγ receptor agonists comprise a first CH3 domain and a second Ig CH3 domain, wherein the first and second Ig CH3 domains differ from one another by at least one amino acid, and wherein at least one amino acid difference reduces binding of the tumor-targeted IL2Rβ binding molecule and/or tumor-targeted IL2Rγ binding molecule to Protein A as compared to a corresponding tumor-targeted IL2Rβ binding molecule and/or tumor-targeted IL2Rγ binding molecule lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.9. Linkers

In certain aspects, the present disclosure provides tumor-targeted split IL2 receptor agonists in which two or more components of a tumor-targeted IL2Rβ binding molecule and/or a tumor-targeted IL2Rγ binding molecule are connected to one another by a peptide linker. By way of example and not limitation, linkers can be used to connect (a) an IL2Rβ binding moiety or a IL2Rγ binding moiety (e.g., an anti-IL2Rβ or anti-IL2Rγ Fab, scFv, or sdAb) and an Fc domain; (b) a tumor targeting moiety and an Fc domain; or (c) different domains within a tumor targeting moiety (e.g., the VH and VL domains in a scFv).

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible linkers are particularly preferred.

Examples of flexible linkers that can be used in the tumor-targeted split IL2 receptor agonists of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GnS (SEQ ID NO: 33) or SGn (SEQ ID NO:24), where n is an integer from 1 to 10, e.g., 1, 2, 3, 4, 5, 6, 7, 8, 9 or 10. In one embodiment, the linker is or comprises a monomer or multimer of repeat of G4S (SEQ ID NO: 25), e.g., (GGGGS)n (SEQ ID NO: 26).

Polyglycine linkers can suitably be used in the tumor-targeted split IL2 receptor agonists of the disclosure. In some embodiments, a peptide linker comprises two consecutive glycines (2Gly), three consecutive glycines (3Gly), four consecutive glycines (4Gly) (SEQ ID NO: 27), five consecutive glycines (5Gly) (SEQ ID NO: 28), six consecutive glycines (6Gly) (SEQ ID NO: 29), seven consecutive glycines (7Gly) (SEQ ID NO: 30), eight consecutive glycines (8Gly) (SEQ ID NO: 31) or nine consecutive glycines (9Gly) (SEQ ID NO: 32).

### 6.9.1. Hinge Sequences

In other embodiments, the tumor-targeted split IL2 receptor agonists of the disclosure comprise a linker that is a hinge region. In particular, where a tumor-targeted IL2Rβ binding molecule and/or a tumor-targeted IL2Rγ of a tumor-targeted split IL2 receptor agonist contains an immunoglobulin-based targeting moiety, the hinge can be used to connect the targeting moiety, e.g., a Fab domain, to a multimerization domain, e.g., an Fc domain. The hinge region can be a native or a modified hinge region. Hinge regions are typically found at the N-termini of Fc regions. The term “hinge region”, unless the context dictates otherwise, refers to a naturally or non-naturally occurring hinge sequence that in the context of a single or monomeric polypeptide chain is a monomeric hinge domain and in the context of a dimeric polypeptide (e.g., a heterodimeric tumor-targeted IL2Rβ binding molecule or a tumor-targeted IL2Rγ binding molecule formed by the association of two Fc domains) can comprise two associated hinge sequences on separate polypeptide chains.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, a tumor-targeted IL2Rβ binding molecule of the disclosure comprises an Fc region in which one or both Fc domains possesses an intact hinge region at its N-terminus.

In one embodiment, a tumor-targeted IL2Rγ binding molecule of the disclosure comprises an Fc region in which one or both Fc domains possesses an intact hinge region at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the tumor-targeted IL2Rβ binding molecules and/or the tumor-targeted IL2Rγ binding molecules of the disclosure comprise a modified hinge region that reduces binding affinity for an Fcγ receptor relative to a wild-type hinge region of the same isotype (e.g., human IgG1 or human IgG4).

In one embodiment, the tumor-targeted IL2Rβ binding molecules and/or the tumor-targeted IL2Rγ binding molecules of the disclosure comprise an Fc region in which each Fc domain possesses an intact hinge region at its N-terminus, where each Fc domain and hinge region is derived from IgG4 and each hinge region comprise the modified sequence CPPC (SEQ ID NO: 40). The core hinge region of human IgG4 contains the sequence CPSC (SEQ ID NO: 41) compared to IgG1 that contains the sequence CPPC (SEQ ID NO: 40). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angal et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.9.1.1. Chimeric Hinge Sequences

The hinge region can be a chimeric hinge region.

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO: 34) (previously disclosed as SEQ ID NO:8 of WO2014/121087, which is incorporated by reference in its entirety herein) or ESKYGPPCPPCPAPPVA (SEQ ID NO: 35) (previously disclosed as SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.6.1.1).

6.9.1.2. Hinge Sequences with Reduced Effector Function

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG- -, G- - - or - - - - with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C, respectively.

Exemplary hinge regions have residues 226-236, sometimes referred to as middle (or core) and lower hinge, occupied by the modified hinge sequences designated GGG-(233-236), GG- -(233-236), G- - -(233-236) and no G(233-236). Optionally, the hinge domain amino acid sequence comprises CPPCPAPGGG-GPSVF (SEQ ID NO: 36) (previously disclosed as SEQ ID NO:1 of WO2016161010A2), CPPCPAPGG- -GPSVF (SEQ ID NO: 37) (previously disclosed as SEQ ID NO:2 of WO2016161010A2), CPPCPAPG- - -GPSVF (SEQ ID NO: 38) (previously disclosed as SEQ ID NO:3 of WO2016161010A2), or CPPCPAP- - - -GPSVF (SEQ ID NO: 39) (previously disclosed as SEQ ID NO:4 of WO2016161010A2).

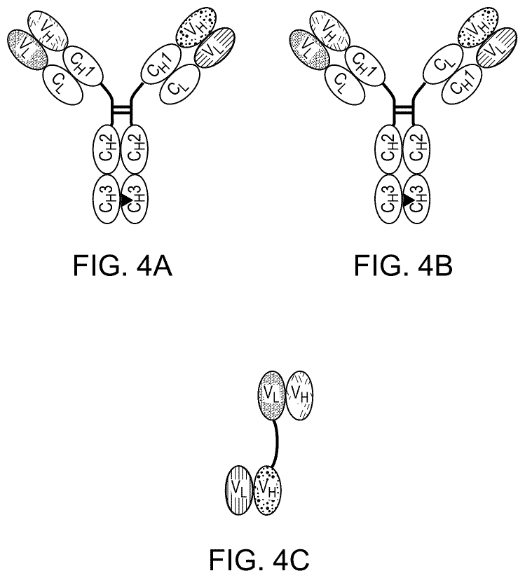

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 6.6.1.1).

The linkers useful in the tumor-targeted split IL2 receptor agonists of the disclosure are typically non-cleavable linkers. A non-cleavable linker is one whose amino acid sequences lacks a (canonical) substrate sequence for a protease, for example a substrate as set forth in Table B on pages 45-49 of international patent publication no. WO2024040249A1 and/or a protease as set forth in Table A on pages 43-44 of international application publication no. WO2024040249A1. The contents of Tables A and B of WO2024040249A1 are incorporated by reference herein.

### 6.10. Nucleic Acids and Host Cells

In another aspect, the disclosure provides nucleic acids encoding the tumor-targeted split IL2 receptor agonists of the disclosure and/or their individual components (the tumor-targeted IL2Rβ binding molecules and the tumor-targeted IL2Rγ binding molecules). In some embodiments, the tumor-targeted split IL2 receptor agonists, the tumor-targeted IL2Rβ binding molecules and/or the tumor-targeted IL2Rγ binding molecules are encoded by a single nucleic acid. In other embodiments, the tumor-targeted IL2Rβ binding molecules and the tumor-targeted IL2Rγ binding molecules are encoded by separate nucleic acids. In other embodiments, for example in the case of a heterodimeric tumor-targeted IL2Rβ binding molecules and/or tumor-targeted IL2Rγ binding molecule, one or both of the tumor-targeted IL2Rβ binding molecules and/or the tumor-targeted IL2Rγ binding molecules, e.g., when comprising an Fc heterodimer or a targeting moiety composed of more than one polypeptide chain, the tumor-targeted IL2Rβ binding molecules and/or the tumor-targeted IL2Rγ binding molecule are encoded by a plurality of (e.g., two, three, four or more) nucleic acids.

A single nucleic acid can encode a tumor-targeted IL2Rβ binding molecule and/or a tumor-targeted IL2Rγ binding molecule that comprises a single polypeptide chain, a tumor-targeted IL2Rβ binding molecules and/or a tumor-targeted IL2Rγ binding molecule that comprises two or more polypeptide chains, or a portion of a tumor-targeted IL2Rβ binding molecule and/or a tumor-targeted IL2Rγ binding molecule that comprises more than two polypeptide chains (for example, a single nucleic acid can encode two polypeptide chains of a tumor-targeted IL2Rβ binding molecules and/or a tumor-targeted IL2Rγ binding molecule comprising three, four or more polypeptide chains, or three polypeptide chains of a tumor-targeted IL2Rβ binding molecule and/or a tumor-targeted IL2Rγ binding molecule comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a tumor-targeted IL2Rβ binding molecule and/or a tumor-targeted IL2Rγ binding molecule comprising two or more polypeptide chains is encoded by two or more nucleic acids. The number of nucleic acids encoding a tumor-targeted split IL2 receptor agonist can be equal to or less than the number of polypeptide chains in the tumor-targeted split IL2 receptor agonist (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.10.1. Vectors

The disclosure provides vectors comprising nucleotide sequences encoding a tumor-targeted IL2Rβ binding molecule and/or a tumor-targeted IL2Rγ binding molecule or component thereof described herein, for example one or two of the polypeptide chains of a tumor-targeted IL2Rβ binding molecules and/or a tumor-targeted IL2Rγ binding molecule. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.10.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.11. Pharmaceutical Compositions

The tumor-targeted split IL2 receptor agonists of the disclosure may be in the form of compositions comprising the tumor-targeted IL2Rβ binding molecules and/or the tumor-targeted IL2Rγ binding molecules and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the tumor-targeted split IL2 receptor agonist and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of a tumor-targeted IL2Rβ receptor agonist and/or tumor-targeted IL2Rγ receptor agonist per dose. The quantity of the tumor-targeted IL2Rβ receptor agonist and/or tumor-targeted IL2Rγ receptor agonist included in a unit dose will depend on the disease being treated, as well as other factors as are well known in the art. Such unit dosages may be in the form of a lyophilized dry powder containing an amount of the tumor-targeted IL2Rβ receptor agonist and/or tumor-targeted IL2Rγ receptor agonist suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of the tumor-targeted IL2Rβ receptor agonist and/or tumor-targeted IL2Rγ receptor agonist suitable for a single administration.

The pharmaceutical compositions may also be supplied in bulk from containing quantities of the tumor-targeted IL2Rβ binding molecule and/or the tumor-targeted IL2Rγ binding molecule suitable for multiple administrations.

When formulated into a single formulation, the tumor-targeted IL2Rβ receptor agonist and tumor-targeted IL2Rγ receptor agonist can be used in approximately equimolar quantities.

Pharmaceutical compositions may be prepared for storage as lyophilized formulations or aqueous solutions by mixing a tumor-targeted IL2Rβ binding molecule and/or a tumor-targeted IL2Rγ binding molecule having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10% w/w of tumor-targeted IL2Rβ and/or IL2Rγ receptor agonist.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### 6.12. Therapeutic Indications and Methods of Treatment

The present disclosure provides methods for using and applications for the tumor-targeted split IL2 receptor agonists of the disclosure.

Tumor-targeted split IL2 receptor agonists of the disclosure are useful in treating disease states where stimulation of the immune system of the host is beneficial, in particular conditions where an enhanced cellular immune response is desirable. These may include disease states where the host immune response is insufficient or deficient.

Disease states for which the tumor-targeted split IL2 receptor agonists of the disclosure can be administered comprise, for example, a tumor or infection where a cellular immune response would be a critical mechanism for specific immunity. Specific disease states for which tumor-targeted split IL2 receptor agonists of the present disclosure can be employed include cancer, including breast cancer, prostate cancer, and colorectal cancer. In some embodiments, tumor-targeted split IL2 receptor agonists of the present disclosure are employed for treatment of a solid tumor. The tumor-targeted split IL2 receptor agonists of the disclosure may be administered per se or in any suitable pharmaceutical composition.

In various embodiments, the tumor-targeted split IL2 receptor agonists of the disclosure are useful for the treatment of cancer, for the prevention or treatment of metastasis, for stimulating the formation, stability and/or activity of a cytotoxic immune synapse, for inducing tumor cytolysis, for inducing anti-tumor cytotoxicity, for stimulating an immune response against a tumor, or any combination of two or more of the foregoing uses.

In one aspect, tumor-targeted split IL2 receptor agonists of the disclosure for use as a medicament are provided. In further aspects, tumor-targeted split IL2 receptor agonists of the disclosure for use in treating a disease are provided. In certain embodiments, tumor-targeted split IL2 receptor agonists of the disclosure for use in a method of treatment are provided. In one embodiment, the disclosure provides a tumor-targeted split IL2 receptor agonist as described herein for use in the treatment of a disease in a subject in need thereof. In certain embodiments, the disclosure provides a tumor-targeted split IL2 receptor agonist for use in a method of treating a subject having a disease comprising administering to the individual a therapeutically effective amount of the tumor-targeted split IL2 receptor agonist. In certain embodiments the disease to be treated is a proliferative disorder. In a preferred embodiment the disease is cancer. In certain embodiments the method further comprises administering to the individual a therapeutically effective amount of at least one additional therapeutic agent, e.g., an anti-cancer agent if the disease to be treated is cancer. In further embodiments, the disclosure provides a tumor-targeted split IL2 receptor agonist for use in stimulating the immune system. In certain embodiments, the disclosure provides a tumor-targeted split IL2 receptor agonist for use in a method of stimulating the immune system in a subject comprising administering to the individual an effective amount of the tumor-targeted split IL2 receptor agonist to stimulate the immune system. An “individual” according to any of the above embodiments is a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL2 receptors, an increase in T-cell responsiveness, an increase in natural killer cell activity or lymphokine-activated killer (LAK) cell activity, and the like.

In a further aspect, the disclosure provides for the use of a tumor-targeted split IL2 receptor agonist of the disclosure in the manufacture or preparation of a medicament for the treatment of a disease in a subject in need thereof. In one embodiment, the medicament is for use in a method of treating a disease comprising administering to a subject having the disease a therapeutically effective amount of the medicament. In certain embodiments the disease to be treated is a proliferative disorder. In a preferred embodiment the disease is cancer. In one such embodiment, the method further comprises administering to the individual a therapeutically effective amount of at least one additional therapeutic agent, e.g., an anti-cancer agent if the disease to be treated is cancer. In a further embodiment, the medicament is for stimulating the immune system. In a further embodiment, the medicament is for use in a method of stimulating the immune system in a subject comprising administering to the individual an amount effective of the medicament to stimulate the immune system. An “individual” according to any of the above embodiments may be a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL2 receptors, an increase in T-cell responsiveness, an increase in natural killer cell activity or lymphokine-activated killer (LAK) cell activity, and the like.

In a further aspect, the disclosure provides a method for treating a disease in a subject, comprising administering to said individual a therapeutically effective amount of a tumor-targeted split IL2 receptor agonist of the disclosure (with the tumor-targeted IL2Rβ binding molecule and the tumor-targeted IL2Rγ binding molecule in separate pharmaceutical preparations or the same pharmaceutical preparation. In one embodiment, one or two compositions comprising a tumor-targeted IL2Rβ binding molecule and a tumor-targeted IL2Rγ binding molecule in a pharmaceutically acceptable form, e.g., in equimolar amounts, are administered to said individual. In certain embodiments, the disease to be treated is a proliferative disorder. In a preferred embodiment, the disease is cancer. In a particular embodiment, the cancer is a solid tumor. In certain embodiments, the method further comprises administering to the individual a therapeutically effective amount of at least one additional therapeutic agent, e.g., an anti-cancer agent if the disease to be treated is cancer. In a further aspect, the disclosure provides a method for stimulating the immune system in a subject, comprising administering to the individual an effective amount of a tumor-targeted split IL2 receptor agonist to stimulate the immune system. An “individual” according to any of the above embodiments may be a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL2 receptors, an increase in T-cell responsiveness, an increase in natural killer cell activity or lymphokine-activated killer (LAK) cell activity, and the like.

In certain aspects, the disclosure provides a method of treating cancer (e.g., a solid tumor), comprising administering to a subject in need thereof a tumor-targeted split IL2 receptor agonist or (a) pharmaceutical composition(s) comprising the tumor-targeted IL2Rβ binding molecule and the tumor-targeted IL2Rγ binding molecule as described herein.

In some embodiments, the disclosure provides a method of treating cancer with a tumor-targeted split IL2 receptor agonist that is targeted to cancer tissue, comprising administering to a subject in need thereof a tumor-targeted split IL2 receptor agonist or (a) pharmaceutical composition(s) comprising the tumor-targeted IL2Rβ binding molecule and the tumor-targeted IL2Rγ binding molecule as described herein, with the tumor-targeted IL2Rβ binding molecule and the tumor-targeted IL2Rγ binding molecule each comprising a targeting moiety that recognizes a target molecule that is expressed on the cancer cells.

The present disclosure further provides a method of localized delivery of a tumor-targeted split IL2 receptor agonist, comprising administering to a subject a tumor-targeted split IL2 receptor agonist or (a) pharmaceutical composition(s) comprising the tumor-targeted IL2Rβ binding molecule and the tumor-targeted IL2Rγ binding molecule as described herein, where the tumor-targeted IL2Rβ binding molecule and the tumor-targeted IL2Rγ binding molecule comprise a targeting moiety that recognizes a target molecule that is expressed by a tissue to which the tumor-targeted split IL2 receptor agonist is to be locally delivered. As used herein, the term “locally delivered” does not require local administration but rather indicates that the tumor-targeted split IL2 receptor agonist be selectively localized to a tissue of interest following administration.

The present disclosure further provides a method of administering to the subject IL2 therapy (i.e., therapy comprising activation of IL2 signaling in a subject, e.g., an IL2 receptor agonist therapy) with reduced systemic exposure and/or reduced systemic toxicity and/or an improved therapeutic index, comprising administering to a subject the IL2 therapy (e.g., IL2 receptor agonist therapy) in the form of a tumor-targeted split IL2 receptor agonist or (a) pharmaceutical composition(s) comprising the tumor-targeted IL2Rβ binding molecule and the tumor-targeted IL2Rγ binding molecule as described herein. Accordingly, the foregoing methods permit IL2 therapy (e.g., IL2 receptor agonist therapy) with reduced off-target side effects by virtue of preferential targeting of an IL2 receptor agonist to a particular target tissue and/or improved anti-tumor cytotoxicity at the site of intended activity. Reduced off-target side effects may be measured, for example, by a reduction in body weight and/or a reduction in systemic T cell expansion as compared to, for example, wild type IL2, an isotype control, and/or a bispecific antibody comprising only a IL2Rβ binding moiety and IL2Rγ binding moiety.

The present disclosure further provides method of locally inducing an immune response in a target tissue, comprising administering to a subject a tumor-targeted split IL2 receptor agonist or (a) pharmaceutical composition(s) comprising the tumor-targeted IL2Rβ binding molecule and the tumor-targeted IL2Rγ binding molecule as described herein, where the tumor-targeted IL2Rβ binding molecule and the tumor-targeted IL2Rγ binding molecule each comprise a targeting moiety capable of binding a target molecule expressed in the target tissue. The tumor-targeted split IL2 receptor agonist can then induce the immune response against at least one cell type in the target tissue. In some embodiments, the target tissue is cancer tissue.

In some embodiments, the administration is not local to the tissue. For example, when the target tissue is cancer tissue, the administration can be systemic or subcutaneous.

In certain embodiments, the disease to be treated is a proliferative disorder, preferably cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. In particular embodiments, the cancer is a solid tumor. Other cell proliferation disorders that can be treated using a tumor-targeted split IL2 receptor agonist of the present disclosure include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments, the cancer is chosen from the group consisting of renal cell cancer, skin cancer, lung cancer, colorectal cancer, breast cancer, brain cancer, head and neck cancer. Similarly, other cell proliferation disorders can also be treated by the IL2 receptor agonists of the present disclosure. Examples of such cell proliferation disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other cell proliferation disease, besides neoplasia, located in an organ system listed above.

Table I below shows exemplary indications for which tumor-targeted split IL2 receptor agonists targeting particular target molecules can be used.

TABLE I
Examples of Target Molecule Indications

| Target | Exemplary Indication(s) |
|---|---|
| ADRB3 | Ewing sarcoma |
| ALK | NSCLC, ALCL, IMT, neuroblastoma |
| B7H3 | melanoma, osteosarcoma, leukemia, breast, prostate, ovarian, pancreatic, |
| | colorectal cancers |
| BCMA | multiple myeloma, leukemia (e.g., acute lymphoblastic leukemia (“ALL”), |
| | acute myeloid leukemia (“AML”), chronic lymphocytic leukemia (“CLL”), |
| | chronic myeloid leukemia (“CML”) and hairy cell leukemia (“HCL”)); |
| | lymphoma (e.g., Hodgkin's lymphoma, non-Hodgkin's lymphoma, including |
| | diffuse large B-cell lymphoma (“DLBCL”)) |
| Cadherin 17 | gastric, pancreatic, and colorectal adenocarcinomas |
| CAIX | clear-cell renal cell carcinoma, hypoxic solid tumors, head and neck |
| | squamous carcinoma |
| CD123 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD171 | neuroblastoma, paraganglioma |
| CD179a | B cell malignancies |
| CD19 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD20 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD22 | leukemia (e.g., ALL, CLL, AML, CML, HCL); lymphoma (e.g., Hodgkin's |
| | lymphoma, non-Hodgkin's lymphoma, e.g., DLBCL); multiple myeloma; |
| | lung cancer |
| CD24 | ovarian, breast, prostate, bladder, renal, non-small cell carcinomas |
| CD30 | anaplastic large cell lymphoma, embryonal carcinoma, Hodgkin Lymphoma |
| CD32b | B cell malignancies, gastric, pancreatic, esophageal, glioblastoma, breast, |
| | colorectal |
| CD33 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD38 | leukemia (e.g., ALL, CLL, AML, CML, HCL); lymphoma (e.g., Hodgkin's |
| | lymphoma, non-Hodgkin's lymphoma, e.g., DLBCL); multiple myeloma |
| CD44v6 | colon cancer, head and neck small cell carcinoma |
| CD97 | B cell malignancies, gastric, pancreatic, esophageal, glioblastoma, breast, |
| | colorectal |
| CEA | colorectal carcinoma, gastric carcinoma, pancreatic carcinoma, lung |
| (CEACAM5) | cancer, breast cancer, medullary thyroid carcinoma |
| CLDN6 | ovarian, breast, lung cancer |
| CLL-1 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CS1 (SLAMF7) | multiple myeloma |
| EGFR | squamous cell carcinoma of lung, anal cancer, glioblastoma, epithelial |
| | tumors of head and neck, colon cancer |
| EGFRvIII | Glioblastoma |
| EPCAM | gastrointestestinal carcinoma, colorectal cancer |
| EphA2 | kaposi's sarcoma, glioblastoma, solid tumors, glioma |
| Ephrin B2 | thyroid cancer, breast cancer, malignant melanoma |
| ERBB2 | breast, ovarian, gastric cancers, lung adenocarcinoma, non-small cell lung |
| (Her2/neu) | cancer, uterine cancer, uterine serous endometrial carcinoma, salivary duct |
| | carcinoma |
| FAP | pancreatic cancer, colorectal cancer, metastasis, epithelial cancers, soft |
| | tissue sarcomas |
| FCRL5 | multiple myeloma |
| FLT3 | leukemia (e.g., ALL, CLL, AML, CML, HCL), lymphoma (e.g., Hodgkin's |
| | lymphoma, non-Hodgkin's lymphoma, e.g., DLBCL), multiple myeloma |
| Folate receptor | ovarian, breast, renal, lung, colorectal, brain cancers |
| alpha | |
| Folate receptor | ovarian cancer |
| beta | |
| Fucosyl GM1 | AML, myeloma |
| GD2 | malignant melanoma, neuroblastoma |
| GD3 | Melanoma |
| GloboH | ovarian, gastric, prostate, lung, breast, and pancreatic cancers |
| gp100 | Melanoma |
| GPNMB | breast cancer, head and neck cancers |
| GPR20 | GIST |
| GPR64 | Ewing sarcoma, prostate, kidney and lung sarcomas |
| GPRC5D | multiple myeloma |
| HAVCR1 | renal cancer |
| HER2 | HER-2 (+) adenocarcinoma of gastroesophageal junction, HER-2 positive |
| | gastric adenocarcinoma, HER2 positive carcinoma of breast |
| HER3 | colon and gastric cancers |
| HMWMAA | melanoma, glioblastoma, breast cancer |
| IGF-I receptor | breast, prostate, lung cancers |
| IL11Rα | papillary thyroid cancer, osteosarcoma, colorectal adenocarcinoma, |
| | lymphocytic leukemia |
| IL13Rα2 | renal cell carcinoma, prostate cancer, gliomas, head and neck cancer, |
| | astrocytoma |
| KIT | myeloid leukemia, kaposi's sarcoma, erythroleukemia, gastrointestinal |
| | stromal tumors |
| KLRG2 | breast cancers, lung cancers and ovarian cancers. |
| LewisY | squamous cell lung carcinoma, lung adenocarcinoma, ovarian carcinoma, |
| | and colorectal adenocarcinoma |
| LMP2 | prostate cancer, Hodgkin's lymphoma, nasopharyngeal carcinoma |
| LRP6 | breast cancer |
| LY6K | breast, lung, ovarian, and cervical cancer |
| LYPD8 | colorectal and gastric cancers |
| Mesothelin | mesothelioma, pancreatic cancer, ovarian cancer, stomach cancer, lung |
| | cancer, endometrial cancer |
| MUC1 | breast and ovarian cancers, lung, stomach, pancreatic, prostate cancers |
| NCAM | melanoma, Wilms' tumor, small cell lung cancer, neuroblastoma, myeloma, |
| | paraganglioma, pancreatic acinar cell carcinoma, myeloid leukemia |
| NY-BR-1 | breast cancer |
| o-acetyl GD2 | neuroblastoma, melanoma |
| OR51E2 | prostate cancer |
| PANX3 | Osteosarcoma |
| PLAC1 | hepatocellular carcinoma |
| Polysialic acid | small cell lung cancer |
| PDGFR-beta | myelomonocytic leukemia, chronic myeloid leukemia, acute myelogenous |
| | leukemia, acute lymphoblastic leukemia |
| PRSS21 | colon cancer, testicular cancer, ovarian cancer |
| PSCA | prostate cancer, gastric and bladder cancers |
| PSMA | prostate cancer |
| ROR1 | metastatic cancers, chronic lymphocytic leukemia, solid tumors in lung, |
| | breast, ovarian, colon, pancreatic, sarcoma |
| SLC34A2 | bladder cancer |
| SLC39A6 | breast cancer, esophageal cancer |
| SLITRK6 | breast cancer, urothelial cancer, lung cancer |
| SSEA-4 | breast cancer, cancer stem cells, epithelial ovarian carcinoma |
| STEAP1 | prostate cancer |
| STEAP2 | prostate cancer (including castrate-resistant prostate cancer), bladder |
| | cancer, cervical cancer, lung cancer, colon cancer, kidney cancer, breast |
| | cancer, pancreatic cancer, stomach cancer, uterine cancer, ovarian |
| | cancer, preferably prostate cancer |
| TACSTD2 | carcinomas, e.g., non-small-cell lung cancer |
| TAG72 | ovarian, breast, colon, lung, pancreatic cancers, gastric cancer |
| TEM1/CD248 | colorectal cancer |
| TEM7R | colorectal cancer |
| Tn | colorectal, breast cancers, cervical, lung, stomach cancers |
| TSHR | thyroid cancer, multiple myeloma |
| Tyrosinase | prostate cancer, melanoma |
| UPK2 | bladder cancer |
| VEGFR2 | ovarian and pancreatic cancers, renal cell carcinoma, colorectal cancer, |
| | medullary thyroid carcinoma |

Additional target molecules and corresponding indications are disclosed in, e.g., Hafeez et al., 2020, Molecules 25:4764, doi:10.3390/molecules25204764, particularly in Table 1. Table 1 is incorporated by reference in its entirety here.

A skilled artisan readily recognizes that in many cases the tumor-targeted split IL2 receptor agonists may not provide a cure but may only provide partial benefit. In some embodiments, a physiological change having some benefit is also considered therapeutically beneficial. Thus, in some embodiments, an amount of tumor-targeted split IL2 receptor agonist that provides a physiological change is considered an “effective amount” or a “therapeutically effective amount”.

The subject, patient, or individual in need of treatment is typically a mammal, more specifically a human.

The appropriate dosage of a tumor-targeted split IL2 receptor agonist of the disclosure (when used alone or in combination with one or more other additional therapeutic agents, e.g., a multispecific T-cell engager) will depend on the type of disease to be treated, the route of administration, the body weight of the patient, the particular tumor-targeted IL2Rβ binding molecule and tumor-targeted IL2Rγ binding molecule in the tumor-targeted split IL2 receptor agonist, the severity and course of the disease, whether the antibody is administered for preventive or therapeutic purposes, previous or concurrent therapeutic interventions, the patient's clinical history and response to the tumor-targeted split IL2 receptor agonist, and the discretion of the attending physician. In some embodiments, the tumor-targeted IL2Rβ binding molecule and tumor-targeted IL2Rγ are administered concurrently and/or in equimolar amounts. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

A therapeutically effective amount of a tumor-targeted split IL2 receptor agonist may comprise only a single administration or many administrations over a period of time. Thus, the tumor-targeted split IL2 receptor agonist is suitably administered to the patient at one time or over a series of treatments, each comprising administration of both a tumor-targeted IL2Rβ binding molecule and a tumor-targeted IL2Rγ binding molecule. Depending on the type and severity of the disease, about 1 μg/kg to 15 mg/kg (e.g., 0.1 mg/kg-10 mg/kg) of each of the tumor-targeted IL2Rβ binding molecule and tumor-targeted IL2Rγ binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the tumor-targeted split IL2 receptor agonist would be in the range from about 0.005 mg/kg to about 10 mg/kg. In other non-limiting examples, a dose may also comprise from about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 μg/kg/body weight to about 500 mg/kg/body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g., every week or every three weeks (e.g., such that the patient receives from about two to about twenty, or e.g., about six doses of the tumor-targeted split IL2 receptor agonist). An initial higher loading dose, followed by one or more lower doses may be administered. However, other dosage regimens may be useful. The progress of this therapy is easily monitored by conventional techniques and assays.

For systemic administration, a therapeutically effective dose can be estimated initially from in vitro assays, such as cell culture assays. A dose can then be formulated in animal models to achieve a circulating concentration range that includes the EC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the tumor-targeted IL2Rβ binding molecule and tumor-targeted IL2Rγ binding molecule which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by ELISA HPLC.

In cases of local administration or selective uptake, the effective local concentration of the tumor-targeted IL2Rβ binding molecule and tumor-targeted IL2Rγ binding molecule may not be related to plasma concentration. One having skill in the art will be able to optimize therapeutically effective local dosages without undue experimentation.

Due to lower toxicity, the tumor-targeted split IL2 receptor agonists of the disclosure can have higher maximum therapeutic doses than wild type IL2, although, the tumor-targeted split IL2 receptor agonists are typically administered at lower doses than wild type IL2 due to the prolonged half-lives.

### 6.13. Combination Therapy

The tumor-targeted split IL2 receptor agonists disclosed herein may be administered in combination with one or more other agents in therapy. For instance, a tumor-targeted split IL2 receptor agonist of the disclosure may be co-administered with at least one additional therapeutic agent. The term “therapeutic agent” encompasses any agent administered to treat a symptom or disease in a subject in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent. In particular embodiments, the additional therapeutic is a multispecific T-cell engager as described in Section 6.6, including but not limited to the multispecific T-cell engagers set forth in Table K.

Such other agents are suitably present in combination in amounts that are effective for the purpose intended. The effective amount of such other agents depends on the amount of tumor-targeted split IL2 receptor agonists used, the type of disorder or treatment, and other factors discussed above. The tumor-targeted split IL2 receptor agonists are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the tumor-targeted split IL2 receptor agonists can occur prior to, simultaneously, and/or following, administration of the additional therapeutic agent and/or adjuvant. Tumor-targeted split IL2 receptor agonists of the disclosure can also be used in combination with radiation therapy.

### 7. SPECIFIC EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.

1. A combination comprising:
(a) a tumor-targeted IL2Rβ binding molecule comprising:
   (i) a first tumor-targeting moiety (e.g., a first tumor-associated antigen (TAA) targeting moiety);
   (ii) an IL2Rβ binding moiety; and
(b) a tumor-targeted IL2Rγ binding molecule comprising:
   (i) a second tumor-targeting moiety (e.g., a second TAA targeting moiety);
   (ii) a IL2Rγ binding moiety; and
for use as a combination therapy, optionally or use as a combination therapy for the treatment of cancer, for use as a combination therapy for the prevention or treatment of metastasis, for use as combination therapy for stimulating the formation, stability and/or activity of a cytotoxic immune synapse, for use as combination therapy for clustering of IL2Rβ and IL2Rγ receptor subunits in a lymphocyte, for eliciting signaling through the IL2 receptor and/or IL15 receptor in a lymphocyte, for use as combination therapy for inducing tumor cytolysis, for use as a combination therapy for inducing anti-tumor cytotoxicity, for use as a combination therapy for stimulating an immune response against a tumor, for use in improving the safety of IL2 agonist cancer treatment, for use in improving the therapeutic window of IL2 agonist cancer treatment, for use as an IL2 receptor agonist therapy with reduced systemic exposure, for use as an IL2 receptor agonist therapy with reduced systemic toxicity, for use as an IL2 receptor agonist therapy with an improved therapeutic index, or any combination of two or more of the foregoing uses.
   2. The combination of embodiment 1, for use as a combination therapy for the treatment of cancer, optionally a solid tumor.
   3. The combination of embodiment 1 or embodiment 2, for use as a combination therapy for the prevention or treatment of metastasis.
   4. The combination of any one of embodiments 1 to 3, for use as combination therapy for stimulating the formation, stability and/or activity of a cytotoxic immune synapse.
   5. The combination of any one of embodiments 1 to 4, for use as combination therapy for clustering of IL2Rβ and IL2Rγ receptor subunits in a lymphocyte.
   6. The combination of any one of embodiments 1 to 5, for use as combination therapy for eliciting signaling through the IL2 receptor and/or IL15 receptor in a lymphocyte.
   7. The combination of any one of embodiments 1 to 6, for use as a combination therapy for inducing tumor cytolysis.
   8. The combination of any one of embodiments 1 to 7, for use as a combination therapy for inducing anti-tumor cytotoxicity.
   9. The combination of any one of embodiments 1 to 8, for use in improving the safety of IL2 agonist cancer treatment.
   10. The combination of any one of embodiments 1 to 9, for use in improving the therapeutic window of IL2 agonist cancer treatment.
   11. The combination of any one of embodiments 1 to 10, for use as an IL2 receptor agonist therapy with reduced systemic exposure.
   12. The combination of any one of embodiments 1 to 11, for use as an IL2 receptor agonist therapy with reduced systemic toxicity.
   13. The combination of any one of embodiments 1 to 12, for use as an IL2 receptor agonist therapy with an improved therapeutic index.
   14. The combination of any one of embodiments 1 to 13, for use as a combination therapy for stimulating an immune response against a tumor.
   15. A method comprising administering to a subject in need thereof a combination comprising:
(a) a tumor-targeted IL2Rβ binding molecule (“R1 agonist”) comprising:
   (i) a first tumor-targeting moiety; and
   (ii) an IL2Rβ binding moiety; and
(b) a tumor-targeted IL2Rγ binding molecule (“R2 agonist”) comprising:
   (i) a second tumor-targeting moiety; and
   (ii) a IL2Rγ binding moiety,
optionally wherein the method is a method of combination therapy for the treatment of cancer, a method of combination therapy for the prevention or treatment of metastasis, a method of combination therapy for stimulating the formation, stability and/or activity of a cytotoxic immune synapse, a method for clustering of IL2Rβ and IL2Rγ receptor subunits in a lymphocyte, a method for eliciting signaling through the IL2 receptor and/or IL15 receptor in a lymphocyte, a method of combination therapy for inducing tumor cytolysis, a method of combination therapy for inducing anti-tumor cytotoxicity, a method of combination therapy for stimulating an immune response against a tumor, a method for improving the safety of IL2 agonist cancer treatment, a method for improving the therapeutic window of IL2 agonist cancer treatment, a method of administering IL2 receptor agonist therapy with reduced systemic exposure, a method of administering IL2 receptor agonist therapy with reduced systemic toxicity, a method of administering IL2 receptor agonist therapy with an improved therapeutic index, or a combination of any two or more of the foregoing methods.
   16. The method of embodiment 15, which is a method for the treatment of cancer, optionally a solid tumor.
   17. The method of embodiment 15 or embodiment 16, which is a method for the prevention or treatment of metastasis.
   18. The method of any one of embodiments 15 to 17, which is a method for stimulating the formation, stability and/or activity of a cytotoxic immune synapse.
   19. The method of any one of embodiments 15 to 18, which is a method for clustering of IL2Rβ and IL2Rγ receptor subunits in a lymphocyte.
   20. The method of any one of embodiments 15 to 19, which is a method for eliciting signaling through the IL2 receptor and/or IL15 receptor in a lymphocyte.
   21. The method of any one of embodiments 15 to 20, which is a method for inducing tumor cytolysis.
   22. The method of any one of embodiments 15 to 21, which is a method for inducing anti-tumor cytotoxicity.
   23. The method of any one of embodiments 15 to 22, a method for improving the safety of IL2 agonist cancer treatment.
   24. The method of any one of embodiments 15 to 23, a method for improving the therapeutic window of IL2 agonist cancer treatment.
   25. The method of any one of embodiments 15 to 24, which is a method for IL2 (e.g., IL2 agonist) therapy with reduced systemic exposure.
   26. The method of any one of embodiments 15 to 25, which is a method for IL2 (e.g., IL2 agonist) therapy with reduced systemic toxicity.
   27. The method of any one of embodiments 15 to 26, which is a method for IL2 (e.g., IL2 agonist) therapy with an improved therapeutic index.
   28. The method of any one of embodiments 15 to 27, which is a method for stimulating an immune response against a tumor.
   29. The combination of any one of embodiments 1 to 14 or the method of any one of embodiments 15 to 28, wherein the IL2Rβ binding moiety and the IL2Rγ binding moiety each comprises or consists of an antigen binding domain of an antibody.
   30. The combination or method of embodiment 29, wherein the IL2Rβ binding moiety and the IL2Rγ binding moiety are Fabs.
   31. The combination or method of embodiment 29, wherein the IL2Rβ binding moiety and the IL2Rγ binding moiety are scFvs.
   32. The combination or method of embodiment 29, wherein the IL2Rβ binding moiety and the IL2Rγ binding moiety are sdAbs.
   33. The combination of any one of embodiments 1 to 14 and 29 to 32 or the method of any one of embodiments 15 to 32, wherein the first tumor-targeting moiety binds to a first tumor-associated antigen and the second tumor-targeting moiety binds to a second tumor-associated antigen.
   34. The combination or method of embodiment 33, wherein the first tumor-associated antigen and the second tumor-associated antigen are expressed on the same tumor cell.
   35. The combination or method of embodiment 33 or embodiment 34, wherein the first tumor-associated antigen and the second tumor-associated antigen are different.
   36. The combination or method of embodiment 33 or embodiment 34, wherein the first tumor-associated antigen and the second tumor-associated antigen are the same.
   37. The combination or method of embodiment 36, wherein the first tumor-targeting moiety and the second tumor-targeting moiety are the same.
   38. The combination or method of embodiment 36, wherein the first tumor-targeting moiety and the second tumor-targeting moiety are different, e.g., bind to different epitopes.
   39. The combination or method of embodiment 36 or embodiment 38, wherein the first tumor-targeting moiety and the second tumor-targeting moiety do not compete for binding to the tumor-associated antigen.
   40. The combination or method of any one of embodiments 36, 38, or 39, wherein the first tumor-targeting moiety and the second tumor-targeting moiety are non-competing targeting moieties as determined as determined using an antibody cross-competition assay as described in Section 8.1.6.
   41. The combination or method of any one of embodiments 33 to 40, wherein the first tumor-targeting moiety and/or the second tumor-targeting moiety are Fabs.
   42. The combination or method of any one of embodiments 33 to 40, wherein the first tumor-targeting moiety and/or the second tumor-targeting moiety are scFvs.
   43. The combination or method of any one of embodiments 33 to 40, wherein the first tumor-targeting moiety and/or the second tumor-targeting moiety are sdAbs.
   44. The combination or method of any one of embodiments 33 to 43, wherein the first tumor-targeting moiety and/or second tumor-targeting moiety bind(s) to (or wherein the first TAA and/or the second TAA is) melanotransferrin (MELTF or CD228), Fibroblast Activation Protein (FAP), the A1 domain of Tenascin-C(TNC A1), the A2 domain of Tenascin-C(TNC A2), the Extra Domain B of Fibronectin (EDB), the Melanoma-associated Chondroitin Sulfate Proteoglycan (MCSP), MART-1/Melan-A, gp100, Dipeptidyl peptidase IV (DPPIV), adenosine deaminase-binding protein (ADAbp), cyclophilin b, colorectal associated antigen (CRC)-C017-1A/GA733, Carcinoembryonic Antigen (CEA) and its immunogenic epitopes CAP-1 and CAP-2, etv6, aml1, prostate-specific membrane antigen (PSMA), T-cell receptor/CD3-zeta chain, GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, Her3, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD20, MCSP, PDGFβR (β-platelet-derived growth factor receptor), ErbB2 epithelial cell adhesion molecule (EpCAM), EGFR variant III (EGFRvIII), CD19, disialoganglioside GD2, ductal-epithelial mucine, gp36, TAG-72, glioma-associated antigen, β-human chorionic gonadotropin, alphafetoprotein (AFP), lectin-reactive AFP, thyroglobulin, MN-CA IX, human telomerase reverse transcriptase, RU1, RU2 (AS), intestinal carboxyl esterase, mut hsp70-2, M-CSF, PAP, LAGA-1a, prostein, survivin and telomerase, prostate-carcinoma tumor antigen-1 (PCTA-1), ELF2M, neutrophil elastase, ephrin B2, insulin growth factor (IGF1)-I, IGF-II, IGFI receptor, 5T4, ROR1, Nkp30, NKG2D, tumor stromal antigens, CA166-9, the extra domain A (EDA) of fibronectin, or the A1 domain of tenascin-C (TnC A1).
   45. The combination or method of any one of embodiments 23 to 33, wherein the first tumor-targeting moiety and/or second tumor-targeting moiety bind(s) to EGFR (e.g., human EGFR).
   46. The combination or method of any one of embodiments 23 to 33, wherein the first TAA and/or second TAA is EGFR.
   47. The combination or method of embodiment 45 or embodiment 46, wherein the first tumor-targeting moiety and/or second tumor-targeting moiety competes with an antibody set forth in Table 1 for binding to EGFR.
   48. The combination or method of embodiment 45 or embodiment 46, wherein the first tumor-targeting moiety and/or second tumor-targeting moiety comprises CDRs having CDR sequences of an anti-EGFR antibody set forth in Table E1.
   49. The combination or method of embodiment 45 or embodiment 46, wherein the first tumor-targeting moiety and/or second tumor-targeting moiety comprises all 6 CDR sequences of an anti-EGFR antibody set forth in Table E1.
   50. The combination or method of embodiment 45 or embodiment 46, wherein the first tumor-targeting moiety and/or second tumor-targeting moiety comprises at least the heavy chain CDR sequences (CDR-H1, CDR-H2, CDR-H3) of an anti-EGFR antibody set forth in Table 1 and the light chain CDR sequences of a universal light chain.
   51. The combination or method of embodiment 45 or embodiment 46, wherein the first tumor-targeting moiety and/or second tumor-targeting moiety comprises (a) a VH comprising the amino acid sequence of the VH of an anti-EGFR antibody set forth in Table 1 and/or (b) a VL comprising the amino acid sequence of the VL of an anti-EGFR antibody set forth in Table E1.
   52. The combination or method of embodiment 45 or embodiment 46, wherein the first tumor-targeting moiety and/or second tumor-targeting moiety competes with an antibody set forth in Table B2 for binding to EGFR.
   53. The combination or method of embodiment 45 or embodiment 46, wherein the first tumor-targeting moiety and/or second tumor-targeting moiety comprises CDRs having CDR sequences of an anti-EGFR antibody set forth in Table B2.
   54. The combination or method of embodiment 45 or embodiment 46, wherein the first tumor-targeting moiety and/or second tumor-targeting moiety comprises all 6 CDR sequences of an anti-EGFR antibody set forth in Table B2.
   55. The combination or method of embodiment 45 or embodiment 46, wherein the first tumor-targeting moiety and/or second tumor-targeting moiety comprises at least the heavy chain CDR sequences (CDR-H1, CDR-H2, CDR-H3) of an anti-EGFR antibody set forth in Table B2 and the light chain CDR sequences of a universal light chain.
   56. The combination or method of embodiment 45 or embodiment 46, wherein the first tumor-targeting moiety and/or second tumor-targeting moiety comprises (a) a VH comprising the amino acid sequence of the VH of an anti-EGFR antibody set forth in Table B2 and/or (b) a VL comprising the amino acid sequence of the VL of an anti-EGFR antibody set forth in Table B2.
   57. The combination or method of embodiment 45 or embodiment 46, wherein the first tumor-targeting moiety and/or second tumor-targeting moiety competes with an antibody set forth in Table B3 for binding to EGFR.
   58. The combination or method of embodiment 45 or embodiment 46, wherein the first tumor-targeting moiety and/or second tumor-targeting moiety comprises CDRs having CDR sequences of an anti-EGFR antibody set forth in Table B3.
   59. The combination or method of embodiment 45 or embodiment 46, wherein the first tumor-targeting moiety and/or second tumor-targeting moiety comprises the CDR3 sequence of an anti-EGFR sdAb set forth in Table B3.
   60. The combination or method of embodiment 45 or embodiment 46, wherein the first tumor-targeting moiety and/or second tumor-targeting moiety comprises all 3 CDR sequences of an anti-EGFR sdAb set forth in Table B3.
   61. The combination or method of embodiment 45 or embodiment 46, wherein the first tumor-targeting moiety and/or second tumor-targeting moiety comprises means for binding EGFR.
   62. The combination or method of embodiment 61, wherein the means for binding EGFR is as disclosed in Table 1 or is an equivalent thereof.
   63. The combination or method of embodiment 61, wherein the means for binding EGFR is as disclosed in Table B2 or is an equivalent thereof.
   64. The combination or method of any one of embodiments 61 to 63, wherein the first tumor-targeting moiety and/or second tumor-targeting moiety is an scFv comprising means for binding EGFR.
   65. The combination or method of any one of embodiments 61 to 63, wherein the first tumor-targeting moiety and/or second tumor-targeting moiety is a Fab comprising means for binding EGFR.
   66. The combination or method of embodiment 61, wherein the means for binding EGFR is as disclosed in Table B3 or is an equivalent thereof.
   67. The combination or method of embodiment 61 or 66, wherein the first tumor-targeting moiety and/or second tumor-targeting moiety is a single domain antibody comprising the means for binding EGFR.
   68. The combination or method of any one of embodiments 33 to 44, wherein the first tumor-targeting moiety and/or second tumor-targeting moiety bind(s) to PSMA (e.g., human PSMA).
   69. The combination or method of any one of embodiments 33 to 44, wherein the first TAA and/or second TAA is PSMA (e.g., human PSMA).
   70. The combination or method of embodiment 68 or embodiment 69, wherein the first tumor-targeting moiety and/or second tumor-targeting moiety competes with an antibody set forth in Table P1 for binding to PSMA.
   71. The combination or method of embodiment 68 or embodiment 69, wherein the first tumor-targeting moiety and/or second tumor-targeting moiety comprises CDRs having CDR sequences of an anti-PSMA antibody set forth in Table P1.
   72. The combination or method of embodiment 68 or embodiment 69, wherein the first tumor-targeting moiety and/or second tumor-targeting moiety comprises all 6 CDR sequences of an anti-PSMA antibody set forth in Table P1.
   73. The combination or method of embodiment 68 or embodiment 69, wherein the first tumor-targeting moiety and/or second tumor-targeting moiety comprises at least the heavy chain CDR sequences (CDR-H1, CDR-H2, CDR-H3) of an anti-PSMA antibody set forth in Table P1 and the light chain CDR sequences of a universal light chain.
   74. The combination or method of embodiment 68 or embodiment 69, wherein the first tumor-targeting moiety and/or second tumor-targeting moiety comprises (a) a VH comprising the amino acid sequence of the VH of an anti-PSMA antibody set forth in Table P1 and/or (b) a VL comprising the amino acid sequence of the VL of an anti-PSMA antibody set forth in Table P1.
   75. The combination or method of embodiment 68 or embodiment 69, wherein the first tumor-targeting moiety and/or second tumor-targeting moiety competes with an antibody set forth in Table P2 for binding to PSMA.
   76. The combination or method of embodiment 68 or embodiment 69, wherein the first tumor-targeting moiety and/or second tumor-targeting moiety comprises CDRs having CDR sequences of an anti-PSMA antibody set forth in Table P2.
   77. The combination or method of embodiment 68 or embodiment 69, wherein the first tumor-targeting moiety and/or second tumor-targeting moiety comprises all 6 CDR sequences of an anti-PSMA antibody set forth in Table P2.
   78. The combination or method of embodiment 68 or embodiment 69, wherein the first tumor-targeting moiety and/or second tumor-targeting moiety comprises at least the heavy chain CDR sequences (CDR-H1, CDR-H2, CDR-H3) of an anti-PSMA antibody set forth in Table P2 and the light chain CDR sequences of a universal light chain.
   79. The combination or method of embodiment 68 or embodiment 69, wherein the first tumor-targeting moiety and/or second tumor-targeting moiety comprises (a) a VH comprising the amino acid sequence of the VH of an anti-PSMA antibody set forth in Table P2 and/or (b) a VL comprising the amino acid sequence of the VL of an anti-PSMA antibody set forth in Table P2.
   80. The combination or method of embodiment 68 or embodiment 69, wherein the first tumor-targeting moiety and/or second tumor-targeting moiety competes with an antibody set forth in Table P3 for binding to PSMA.
   81. The combination or method of embodiment 68 or embodiment 69, wherein the first tumor-targeting moiety and/or second tumor-targeting moiety comprises CDRs having CDR sequences of an anti-PSMA antibody set forth in Table P3.
   82. The combination or method of embodiment 68 or embodiment 69, wherein the first tumor-targeting moiety and/or second tumor-targeting moiety comprises the CDR3 sequence of an anti-PSMA sdAb set forth in Table P3.
   83. The combination or method of embodiment 68 or embodiment 69, wherein the first tumor-targeting moiety and/or second tumor-targeting moiety comprises all 3 CDR sequences of an anti-PSMA sdAb set forth in Table P3.
   84. The combination or method of embodiment 68 or embodiment 69, wherein the first tumor-targeting moiety and/or second tumor-targeting moiety comprises means for binding PSMA.
   85. The combination or method of embodiment 84, wherein the means for binding PSMA is as disclosed in Table P1 or is an equivalent thereof.
   86. The combination or method of embodiment 84, wherein the means for binding PSMA is as disclosed in Table P2 or is an equivalent thereof.
   87. The combination or method of any one of embodiments 84 to 86, wherein the first tumor-targeting moiety and/or second tumor-targeting moiety is an scFv comprising means for binding PSMA.
   88. The combination or method of any one of embodiments 84 to 86, wherein the first tumor-targeting moiety and/or second tumor-targeting moiety is a Fab comprising means for binding PSMA.
   89. The combination or method of embodiment 84, wherein the means for binding PSMA is as disclosed in Table P3 or is an equivalent thereof.
   90. The combination or method of embodiment 84 or 89, wherein the first tumor-targeting moiety and/or second tumor-targeting moiety is a single domain antibody comprising the means for binding PSMA.
   91. The combination or method of any one of embodiments 33 to 44, wherein the first tumor-targeting moiety and/or second tumor-targeting moiety bind(s) to MUC16 (e.g., human MUC16).
   92. The combination or method of any one of embodiments 33 to 44, wherein the first TAA and/or TAA is MUC16 (e.g., human MUC16).
   93. The combination or method of embodiment 91 or embodiment 92, wherein the first tumor-targeting moiety and/or second tumor-targeting moiety competes with an antibody set forth in Table M1 for binding to MUC16.
   94. The combination or method of embodiment 91 or embodiment 92, wherein the first tumor-targeting moiety and/or second tumor-targeting moiety comprises CDRs having CDR sequences of an anti-MUC16 antibody set forth in Table M1.
   95. The combination or method of embodiment 91 or embodiment 92, wherein the first tumor-targeting moiety and/or second tumor-targeting moiety comprises all 6 CDR sequences of an anti-MUC16 antibody set forth in Table M1.
   96. The combination or method of embodiment 91 or embodiment 92, wherein the first tumor-targeting moiety and/or second tumor-targeting moiety comprises at least the heavy chain CDR sequences (CDR-H1, CDR-H2, CDR-H3) of an anti-MUC16 antibody set forth in Table M1 and the light chain CDR sequences of a universal light chain.
   97. The combination or method of embodiment 91 or embodiment 92, wherein the first tumor-targeting moiety and/or second tumor-targeting moiety comprises (a) a VH comprising the amino acid sequence of the VH of an anti-MUC16 antibody set forth in Table M1 and/or (b) a VL comprising the amino acid sequence of the VL of an anti-MUC16 antibody set forth in Table M1.
   98. The combination or method of embodiment 91 or embodiment 92, wherein the first tumor-targeting moiety and/or second tumor-targeting moiety competes with an antibody set forth in Table M2 for binding to MUC16.
   99. The combination or method of embodiment 91 or embodiment 92, wherein the first tumor-targeting moiety and/or second tumor-targeting moiety comprises CDRs having CDR sequences of an anti-MUC16 antibody set forth in Table M2.
   100. The combination or method of embodiment 91 or embodiment 92, wherein the first tumor-targeting moiety and/or second tumor-targeting moiety comprises all 6 CDR sequences of an anti-MUC16 antibody set forth in Table M2.
   101. The combination or method of embodiment 91 or embodiment 92, wherein the first tumor-targeting moiety and/or second tumor-targeting moiety comprises at least the heavy chain CDR sequences (CDR-H1, CDR-H2, CDR-H3) of an anti-MUC16 antibody set forth in Table M2 and the light chain CDR sequences of a universal light chain.
   102. The combination or method of embodiment 91 or embodiment 92, wherein the first tumor-targeting moiety and/or second tumor-targeting moiety comprises (a) a VH comprising the amino acid sequence of the VH of an anti-MUC16 antibody set forth in Table M2 and/or (b) a VL comprising the amino acid sequence of the VL of an anti-MUC16 antibody set forth in Table M2.
   103. The combination or method of embodiment 91 or embodiment 92, wherein the first tumor-targeting moiety and/or second tumor-targeting moiety competes with an antibody set forth in Table M3 for binding to MUC16.
   104. The combination or method of embodiment 91 or embodiment 92, wherein the first tumor-targeting moiety and/or second tumor-targeting moiety comprises CDRs having CDR sequences of an anti-MUC16 antibody set forth in Table M3.
   105. The combination or method of embodiment 91 or embodiment 92, wherein the first tumor-targeting moiety and/or second tumor-targeting moiety comprises the CDR3 sequence of an anti-MUC16 sdAb set forth in Table M3.
   106. The combination or method of embodiment 91 or embodiment 92, wherein the first tumor-targeting moiety and/or second tumor-targeting moiety comprises all 3 CDR sequences of an anti-MUC16 sdAb set forth in Table M3.
   107. The combination or method of embodiment 91 or embodiment 92, wherein the first tumor-targeting moiety and/or second tumor-targeting moiety comprises means for binding MUC16.
   108. The combination or method of embodiment 107, wherein the means for binding MUC16 is as disclosed in Table M1 or is an equivalent thereof.
   109. The combination or method of embodiment 107, wherein the means for binding MUC16 is as disclosed in Table M2 or is an equivalent thereof.
   110. The combination or method of any one of embodiments 107 to 109, wherein the first tumor-targeting moiety and/or second tumor-targeting moiety is an scFv comprising means for binding MUC16.
   111. The combination or method of any one of embodiments 107 to 109, wherein the first tumor-targeting moiety and/or second tumor-targeting moiety is a Fab comprising means for binding MUC16.
   112. The combination or method of embodiment 107, wherein the means for binding MUC16 is as disclosed in Table M3 or is an equivalent thereof.
   113. The combination or method of embodiment 107 or 112, wherein the first tumor-targeting moiety and/or second tumor-targeting moiety is a single domain antibody comprising the means for binding MUC16.
   114. The combination or method of any one of embodiments 33 to 44, wherein the first tumor-targeting moiety and/or second tumor-targeting moiety bind(s) to CA9 (e.g., human CA9).
   115. The combination or method of any one of embodiments 33 to 44, wherein the first TAA and/or second TAA is CA9 (e.g., human CA9).
   116. The combination or method of any one of embodiments 33 to 44, wherein the first tumor-targeting moiety and/or second tumor-targeting moiety bind(s) to MSLN (e.g., human MSLN).
   117. The combination or method of any one of embodiments 33 to 44, wherein the first TAA and/or second TAA bind(s) to MSLN (e.g., human MSLN).
   118. The combination or method of embodiment 116 or embodiment 117, wherein the first tumor-targeting moiety and/or second tumor-targeting moiety competes with an antibody set forth in Table L1 for binding to MSLN.
   119. The combination or method of embodiment 116 or embodiment 117, wherein the first tumor-targeting moiety and/or second tumor-targeting moiety comprises CDRs having CDR sequences of an anti-MSLN antibody set forth in Table L1.
   120. The combination or method of embodiment 116 or embodiment 117, wherein the first tumor-targeting moiety and/or second tumor-targeting moiety comprises all 6 CDR sequences of an anti-MSLN antibody set forth in Table L1.
   121. The combination or method of embodiment 116 or embodiment 117, wherein the first tumor-targeting moiety and/or second tumor-targeting moiety comprises at least the heavy chain CDR sequences (CDR-H1, CDR-H2, CDR-H3) of an anti-MSLN antibody set forth in Table L1 and the light chain CDR sequences of a universal light chain.
   122. The combination or method of embodiment 116 or embodiment 117, wherein the first tumor-targeting moiety and/or second tumor-targeting moiety comprises (a) a VH comprising the amino acid sequence of the VH of an anti-MSLN antibody set forth in Table L1 and/or (b) a VL comprising the amino acid sequence of the VL of an anti-MSLN antibody set forth in Table L1.
   123. The combination or method of embodiment 116 or embodiment 117, wherein the first tumor-targeting moiety and/or second tumor-targeting moiety competes with an antibody set forth in Table L2 for binding to MSLN.
   124. The combination or method of embodiment 116 or embodiment 117, wherein the first tumor-targeting moiety and/or second tumor-targeting moiety comprises CDRs having CDR sequences of an anti-MSLN antibody set forth in Table L2.
   125. The combination or method of embodiment 116 or embodiment 117, wherein the first tumor-targeting moiety and/or second tumor-targeting moiety comprises the CDR3 sequence of an anti-MSLN sdAb set forth in Table L2.
   126. The combination or method of embodiment 116 or embodiment 117, wherein the first tumor-targeting moiety and/or second tumor-targeting moiety comprises all 3 CDR sequences of an anti-MSLN sdAb set forth in Table L2.
   127. The combination or method of embodiment 116 or embodiment 117, wherein the first tumor-targeting moiety and/or second tumor-targeting moiety comprises means for binding MSLN.
   128. The combination or method of embodiment 127, wherein the means for binding MSLN is as disclosed in Table L1 or is an equivalent thereof.
   129. The combination or method of embodiment 127 or 128, wherein the first tumor-targeting moiety and/or second tumor-targeting moiety is an scFv comprising means for binding MSLN.
   130. The combination or method of embodiment 127 or 128, wherein the first tumor-targeting moiety and/or second tumor-targeting moiety is a Fab comprising means for binding MSLN.
   131. The combination or method of embodiment 127, wherein the means for binding MSLN is as disclosed in Table L2 or is an equivalent thereof.
   132. The combination or method of embodiment 127 or 131, wherein the first tumor-targeting moiety and/or second tumor-targeting moiety is a single domain antibody comprising the means for binding MSLN.
   133. The combination or method of any one of embodiments 33 to 44, wherein the first tumor-targeting moiety and/or second tumor-targeting moiety bind(s) to EPCAM (e.g., human EPCAM).
   134. The combination or method of any one of embodiments 33 to 44, wherein the first TAA and/or second TAA is EPCAM (e.g., human EPCAM).
   135. The combination or method of any one of embodiments 33 to 44, wherein the first tumor-targeting moiety and/or second tumor-targeting moiety bind(s) to B7H3 (e.g., human B7H3).
   136. The combination or method of any one of embodiments 33 to 44, wherein the first TAA and/or second TAA is B7H3 (e.g., human B7H3).
   137. The combination or method of any one of embodiments 33 to 44, wherein the first tumor-targeting moiety and/or second tumor-targeting moiety bind(s) to HER2/HER3 (e.g., human HER2).
   138. The combination or method of any one of embodiments 33 to 44, wherein the first TAA and/or second TAA is HER2/HER3 (e.g., human HER2).
   139. The combination or method of embodiment 137 or embodiment 138, wherein the first tumor-targeting moiety and/or second tumor-targeting moiety competes with an antibody set forth in Table H1 for binding to HER2.
   140. The combination or method of embodiment 137 or embodiment 138, wherein the first tumor-targeting moiety and/or second tumor-targeting moiety comprises CDRs having CDR sequences of an anti-HER2 antibody set forth in Table H1.
   141. The combination or method of embodiment 137 or embodiment 138, wherein the first tumor-targeting moiety and/or second tumor-targeting moiety comprises all 6 CDR sequences of an anti-HER2 antibody set forth in Table H1.
   142. The combination or method of embodiment 137 or embodiment 138, wherein the first tumor-targeting moiety and/or second tumor-targeting moiety comprises at least the heavy chain CDR sequences (CDR-H1, CDR-H2, CDR-H3) of an anti-HER2 antibody set forth in Table H1 and the light chain CDR sequences of a universal light chain.
   143. The combination or method of embodiment 137 or embodiment 138, wherein the first tumor-targeting moiety and/or second tumor-targeting moiety comprises (a) a VH comprising the amino acid sequence of the VH of an anti-HER2 antibody set forth in Table H1 and/or (b) a VL comprising the amino acid sequence of the VL of an anti-HER2 antibody set forth in Table H1.
   144. The combination or method of embodiment 137 or embodiment 138, wherein the first tumor-targeting moiety and/or second tumor-targeting moiety competes with an antibody set forth in Table H2 for binding to HER2.
   145. The combination or method of embodiment 137 or embodiment 138, wherein the first tumor-targeting moiety and/or second tumor-targeting moiety comprises CDRs having CDR sequences of an anti-HER2 antibody set forth in Table H2.
   146. The combination or method of embodiment 137 or embodiment 138, wherein the first tumor-targeting moiety and/or second tumor-targeting moiety comprises all 6 CDR sequences of an anti-HER2 antibody set forth in Table H2.
   147. The combination or method of embodiment 137 or embodiment 138, wherein the first tumor-targeting moiety and/or second tumor-targeting moiety comprises at least the heavy chain CDR sequences (CDR-H1, CDR-H2, CDR-H3) of an anti-HER2 antibody set forth in Table H2 and the light chain CDR sequences of a universal light chain.
   148. The combination or method of embodiment 137 or embodiment 138, wherein the first tumor-targeting moiety and/or second tumor-targeting moiety comprises (a) a VH comprising the amino acid sequence of the VH of an anti-HER2 antibody set forth in Table H2 and/or (b) a VL comprising the amino acid sequence of the VL of an anti-HER2 antibody set forth in Table H2.
   149. The combination or method of embodiment 137 or embodiment 138, wherein the first tumor-targeting moiety and/or second tumor-targeting moiety competes with an antibody set forth in Table H3 for binding to HER2.
   150. The combination or method of embodiment 137 or embodiment 138, wherein the first tumor-targeting moiety and/or second tumor-targeting moiety comprises CDRs having CDR sequences of an anti-HER2 antibody set forth in Table H3.
   151. The combination or method of embodiment 137 or embodiment 138, wherein the first tumor-targeting moiety and/or second tumor-targeting moiety comprises the CDR3 sequence of an anti-HER2 sdAb set forth in Table H3.
   152. The combination or method of embodiment 137 or embodiment 138, wherein the first tumor-targeting moiety and/or second tumor-targeting moiety comprises all 3 CDR sequences of an anti-HER2 sdAb set forth in Table H3.
   153. The combination or method of embodiment 137 or embodiment 138, wherein the first tumor-targeting moiety and/or second tumor-targeting moiety comprises means for binding HER2.
   154. The combination or method of embodiment 153, wherein the means for binding HER2 is as disclosed in Table H1 or is an equivalent thereof.
   155. The combination or method of embodiment 153, wherein the means for binding HER2 is as disclosed in Table H2 or is an equivalent thereof.
   156. The combination or method of any one of embodiments 153 to 155, wherein the first tumor-targeting moiety and/or second tumor-targeting moiety is an scFv comprising means for binding HER2.
   157. The combination or method of any one of embodiments 153 to 155, wherein the first tumor-targeting moiety and/or second tumor-targeting moiety is a Fab comprising means for binding HER2.
   158. The combination or method of embodiment 153, wherein the means for binding HER2 is as disclosed in Table H3 or is an equivalent thereof.
   159. The combination or method of embodiment 153 or 158, wherein the first tumor-targeting moiety and/or second tumor-targeting moiety is a single domain antibody comprising the means for binding HER2.
   160. The combination or method of any one of embodiments 33 to 44, wherein the first tumor-targeting moiety and/or second tumor-targeting moiety bind(s) to STEAP1 (e.g., human STEAP1).
   161. The combination or method of any one of embodiments 33 to 44, wherein the first TAA and/or second TAA is STEAP1 (e.g., human STEAP1).
   162. The combination or method of embodiment 160 or embodiment 161, wherein the first tumor-targeting moiety and/or second tumor-targeting moiety competes with an antibody set forth in Table A1 for binding to STEAP1.
   163. The combination or method of embodiment 160 or embodiment 161, wherein the first tumor-targeting moiety and/or second tumor-targeting moiety comprises CDRs having CDR sequences of an anti-STEAP1 antibody set forth in Table A1.
   164. The combination or method of embodiment 160 or embodiment 161, wherein the first tumor-targeting moiety and/or second tumor-targeting moiety comprises all 6 CDR sequences of an anti-STEAP1 antibody set forth in Table A1.
   165. The combination or method of embodiment 160 or embodiment 161, wherein the first tumor-targeting moiety and/or second tumor-targeting moiety comprises at least the heavy chain CDR sequences (CDR-H1, CDR-H2, CDR-H3) of an anti-STEAP1 antibody set forth in Table A1 and the light chain CDR sequences of a universal light chain.
   166. The combination or method of embodiment 160 or embodiment 161, wherein the first tumor-targeting moiety and/or second tumor-targeting moiety comprises (a) a VH comprising the amino acid sequence of the VH of an anti-STEAP1 antibody set forth in Table A1 or (b) a VL comprising the amino acid sequence of the VL of an anti-STEAP1 antibody set forth in Table A1.
   167. The combination or method of embodiment 160 or embodiment 161, wherein the first tumor-targeting moiety and/or second tumor-targeting moiety comprises means for binding STEAP1.
   168. The combination or method of embodiment 167, wherein the means for binding STEAP1 is as disclosed in Table A1 or is an equivalent thereof.
   169. The combination or method of embodiment 167 or 168, wherein the first tumor-targeting moiety and/or second tumor-targeting moiety is an scFv comprising means for binding STEAP1.
   170. The combination or method of embodiment 167 or 168, wherein the first tumor-targeting moiety and/or second tumor-targeting moiety is a Fab comprising means for binding STEAP1.
   171. The combination or method of any one of embodiments 33 to 44, wherein the first tumor-targeting moiety and/or second tumor-targeting moiety bind(s) to CEACAM5 (e.g., human CEACAM5).
   172. The combination or method of any one of embodiments 33 to 44, wherein the first TAA and/or second TAA is CEACAM5 (e.g., human CEACAM5).
   173. The combination of any one of embodiments 1 to 14 and 29 to 172 or the method of any one of embodiments 15 to 172, wherein the tumor-targeted IL2Rβ binding molecule comprises
(a) a first polypeptide chain comprising, in N- to C-terminal orientation:
   (i) the first tumor-targeting moiety or component thereof (or a component thereof, e.g., a VH-CH1 or VL-CL), optionally associated with another component thereof on a separate polypeptide chain (or a component thereof, e.g., a VL-CL or VH-CH1);
   (ii) optionally, a linker (a “TAA-Fc linker”); and
   (iii) a first Fc domain; and
(b) a second polypeptide chain comprising, in N- to C-terminal orientation:
   (i) the IL2Rβ binding moiety or component thereof (or a component thereof, e.g., a VH-CH1 or VL-CL), optionally associated with another component thereof on a separate polypeptide chain (or a component thereof, e.g., a VL-CL or VH-CH1);
   (ii) optionally, a linker; and
   (iii) a second Fc domain associated with the first Fc domain.
      174. The combination or method of embodiment 173, wherein the first and second Fc domains are both IgG1 Fc domains, IgG2 Fc domains, IgG3 Fc domains or IgG4 Fc domains.
      175. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:5, optionally comprising knob/hole substitutions and/or star mutation(s).
      176. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:6, optionally comprising knob/hole substitutions and/or star mutation(s).
      177. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:7, optionally comprising knob/hole substitutions and/or star mutation(s).
      178. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:8, optionally comprising knob/hole substitutions and/or star mutation(s).
      179. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:9, optionally comprising knob/hole substitutions and/or star mutation(s).
      180. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:10, optionally comprising knob/hole substitutions and/or star mutation(s).
      181. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:11, optionally comprising knob/hole substitutions and/or star mutation(s).
      182. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:12, optionally comprising knob/hole substitutions and/or star mutation(s).
      183. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:13, optionally comprising knob/hole substitutions and/or star mutation(s).
      184. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:14, optionally comprising knob/hole substitutions and/or star mutation(s).
      185. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:15, optionally comprising knob/hole substitutions and/or star mutation(s).
      186. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:16, optionally comprising knob/hole substitutions and/or star mutation(s).
      187. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:17, optionally comprising knob/hole substitutions and/or star mutation(s).
      188. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:18, optionally comprising knob/hole substitutions and/or star mutation(s).
      189. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:19, optionally comprising knob/hole substitutions and/or star mutation(s).
      190. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:20, optionally comprising knob/hole substitutions and/or star mutation(s).
      191. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:21, optionally comprising knob/hole substitutions and/or star mutation(s).
      192. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:22, optionally comprising knob/hole substitutions and/or star mutation(s).
      193. The combination or method of embodiment 173 or embodiment 174, wherein the first Fc domain and second Fc domain each comprises an amino acid sequence having at least 98%, at least 99% or at least 99.5% sequence identity to the amino acid sequence of SEQ ID NO:23, optionally comprising knob/hole substitutions and/or star mutation(s).
      194. The combination or method of any one of embodiments 173 to 193, wherein the first Fc domain and second Fc domain each comprises a chimeric hinge domain.
      195. The combination or method of any one of one of embodiments 173 to 194, wherein the first Fc domain and second Fc domain each has reduced effector function.
      196. The combination or method of any one of embodiments 173 to 195, wherein the first Fc domain and second Fc domain form an Fc heterodimer.
      197. The combination of any one of embodiments 1 to 14 and 29 to 196 or the method of any one of embodiments 15 to 196, wherein the tumor-targeted IL2Rγ binding molecule comprises
(a) a third polypeptide chain comprising, in N- to C-terminal orientation:
   (i) the second tumor-targeting moiety or component thereof (or a component thereof, e.g., a VH-CH1 or VL-CL), optionally associated with another component thereof on a separate polypeptide chain (or a component thereof, e.g., a VL-CL or VH-CH1);
   (ii) optionally, a linker (a “TAA-Fc linker”); and
   (iii) a third Fc domain; and
(b) a third polypeptide chain comprising, in N- to C-terminal orientation:
   (i) the IL2Rγ binding moiety or component thereof (or a component thereof, e.g., a VH-CH1 or VL-CL), optionally associated with another component thereof on a separate polypeptide chain (or a component thereof, e.g., a VL-CL or VH-CH1);
   (ii) optionally, a linker; and
   (iii) a fourth Fc domain associated with the third Fc domain.
      198. The combination or method of embodiment 197, wherein the third and fourth Fc domains are IgG1, IgG2, IgG3 or IgG4 Fc domains.
      199. The combination or method of embodiment 197 or embodiment 198, wherein the third Fc domain and fourth Fc domain each comprises an amino acid sequence having at least 95% sequence identity to the amino acid sequence of SEQ ID NO:11.
      200. The combination or method of embodiment 197 or embodiment 198, wherein the third Fc domain and fourth Fc domain each comprises an amino acid sequence having at least 95% sequence identity to the amino acid sequence of SEQ ID NO:12.
      201. The combination or method of embodiment 197 or embodiment 198, wherein the third Fc domain and fourth Fc domain each comprises an amino acid sequence having at least 95% sequence identity to the amino acid sequence of SEQ ID NO:13.
      202. The combination or method of any one of embodiments 197 to 201, wherein the third Fc domain and fourth Fc domain each comprises a chimeric hinge domain.
      203. The combination or method of any one of embodiments 197 to 202, wherein the third Fc domain and fourth Fc domain each has reduced effector function.
      204. The combination or method of any one of one of embodiments 197 to 203, wherein the third Fc domain and fourth Fc domain form an Fc heterodimer.
      205. The combination of any one of embodiments 1 to 14 and 29 to 204 or the method of any one of embodiments 15 to 204, wherein the tumor-targeted IL2Rβ binding molecule is monovalent for the first tumor-targeting moiety.
      206. The combination of any one of embodiments 1 to 14 and 29 to 205 or the method of any one of embodiments 15 to 205, wherein the tumor-targeted IL2Rβ binding molecule is monovalent for the IL2Rβ binding moiety.
      207. The combination of any one of embodiments 1 to 14 and 29 to 206 or the method of any one of embodiments 15 to 206, wherein the tumor-targeted IL2Rγ binding molecule is monovalent for the second tumor-targeting moiety.
      208. The combination of any one of embodiments 1 to 14 and 29 to 207 or the method of any one of embodiments 15 to 207, wherein the tumor-targeted IL2Rγ binding molecule is monovalent for the IL2Rγ binding moiety.
      209. The combination of any one of embodiments 1 to 14 and 29 to 208 or the method of any one of embodiments 15 to 208, wherein the tumor-targeted IL2Rβ binding molecule and the tumor-targeted IL2Rγ binding molecule are both in the form of a pharmaceutical composition comprising the molecule and an excipient.
      210. The combination or method of embodiment 209, wherein the tumor-targeted IL2Rβ binding molecule and the tumor-targeted IL2Rγ binding molecule are in the same pharmaceutical composition.
      211. The combination or method of embodiment 209, wherein the tumor-targeted IL2Rβ binding molecule and the tumor-targeted IL2Rγ binding molecule are in different pharmaceutical compositions.
      212. The combination of any one of embodiments 1 to 14 and 29 to 211 or the method of any one of embodiments 15 to 211, wherein tumor-targeted IL2Rβ binding molecule is configured as illustrated in FIG. 2A.
      213. The combination of any one of embodiments 1 to 14 and 29 to 211 or the method of any one of embodiments 15 to 211, wherein tumor-targeted IL2Rβ binding molecule is configured as illustrated in FIG. 2B.
      214. The combination of any one of embodiments 1 to 14 and 29 to 211 or the method of any one of embodiments 15 to 211, wherein tumor-targeted IL2Rβ binding molecule is configured as illustrated in FIG. 2C.
      215. The combination of any one of embodiments 1 to 14 and 29 to 211 or the method of any one of embodiments 15 to 211, wherein tumor-targeted IL2Rβ binding molecule is configured as illustrated in FIG. 2D.
      216. The combination of any one of embodiments 1 to 14 and 29 to 211 or the method of any one of embodiments 15 to 211, wherein tumor-targeted IL2Rβ binding molecule is configured as illustrated in FIG. 2E.
      217. The combination of any one of embodiments 1 to 14 and 29 to 211 or the method of any one of embodiments 15 to 211, wherein tumor-targeted IL2Rβ binding molecule is configured as illustrated in FIG. 2F.
      218. The combination of any one of embodiments 1 to 14 and 29 to 211 or the method of any one of embodiments 15 to 211, wherein tumor-targeted IL2Rβ binding molecule is configured as illustrated in FIG. 2G.
      219. The combination of any one of embodiments 1 to 14 and 29 to 211 or the method of any one of embodiments 15 to 211, wherein tumor-targeted IL2Rβ binding molecule is configured as illustrated in FIG. 2H.
      220. The combination of any one of embodiments 1 to 14 and 29 to 219 or the method of any one of embodiments 15 to 219, wherein tumor-targeted IL2Rγ binding molecule is configured as illustrated in FIG. 3A.
      221. The combination of any one of embodiments 1 to 14 and 29 to 219 or the method of any one of embodiments 15 to 219, wherein tumor-targeted IL2Rγ binding molecule is configured as illustrated in FIG. 3B.
      222. The combination of any one of embodiments 1 to 14 and 29 to 219 or the method of any one of embodiments 15 to 219, wherein tumor-targeted IL2Rγ binding molecule is configured as illustrated in FIG. 3C.
      223. The combination of any one of embodiments 1 to 14 and 29 to 219 or the method of any one of embodiments 15 to 219, wherein tumor-targeted IL2Rγ binding molecule is configured as illustrated in FIG. 3D.
      224. The combination of any one of embodiments 1 to 14 and 29 to 219 or the method of any one of embodiments 15 to 219, wherein tumor-targeted IL2Rγ binding molecule is configured as illustrated in FIG. 3E.
      225. The combination of any one of embodiments 1 to 14 and 29 to 219 or the method of any one of embodiments 15 to 219, wherein tumor-targeted IL2Rγ binding molecule is configured as illustrated in FIG. 3F.
      226. The combination of any one of embodiments 1 to 14 and 29 to 219 or the method of any one of embodiments 15 to 219, wherein tumor-targeted IL2Rγ binding molecule is configured as illustrated in FIG. 3G.
      227. The combination of any one of embodiments 1 to 14 and 29 to 219 or the method of any one of embodiments 15 to 219, wherein tumor-targeted IL2Rγ binding molecule is configured as illustrated in FIG. 3H.
      228. The combination of any one of embodiments 1 to 14 and 29 to 227 or the method of any one of embodiments 15 to 227, wherein the combination further comprises or the method further comprises administering, a multispecific T-cell engager (e.g., simultaneously, sequentially or separately).
      229. The combination or method of embodiment 228, wherein the multispecific T-cell engager is a bispecific T-cell engager.
      230. The combination or method of embodiment 228 or embodiment 229, wherein the multispecific T-cell engager comprises a TAA targeting moiety and a CD3 targeting moiety.
      231. The combination or method of embodiment 230, wherein the TAA targeting moiety of the multispecific T-cell engager targets the same TAA as the TAA targeted by the tumor-targeted IL2Rβ binding molecule and/or the tumor-targeted IL2Rγ binding molecule.
      232. The combination or method of embodiment 230, wherein the TAA targeting moiety of the multispecific T-cell engager targets a TAA that is different from the TAA targeted by the tumor-targeted IL2Rβ binding molecule and/or the tumor-targeted IL2Rγ binding molecule.
      233. The combination or method of embodiment 232, wherein the targeting moiety of the multispecific T-cell engager targets MSLN (e.g., human MSLN).
      234. The combination or method of embodiment 232 or embodiment 233, wherein the TAA targeted by the tumor-targeted IL2Rβ binding molecule and/or the tumor-targeted IL2Rγ binding molecule is MUC16 (e.g., human MUC16).
      235. The combination of any one of embodiment 1 to 14 and 29 to 234 or the method of any one of embodiments 15 to 234, which is suitable for or results in improving the safety of IL2 receptor agonist cancer treatment.
      236. The combination of any one of embodiment 1 to 14 and 29 to 235 or the method of any one of embodiments 15 to 235, which is suitable for or results in improving the therapeutic window of IL2 receptor agonist cancer treatment.
      237. The combination of any one of embodiment 1 to 14 and 29 to 236 or the method of any one of embodiments 15 to 236, which has or results in improved anti-tumor activity compared to an isotype control, optionally wherein anti-tumor activity is determined by a reduction in post-implantation tumor radiance.
      238. The combination of any one of embodiment 1 to 14 and 29 to 237 or the method of any one of embodiments 15 to 237, which has or results in reduced adverse effects compared to a bispecific antibody comprising the IL2Rβ binding moiety and the IL2Rγ binding moiety, optionally wherein the adverse side effects are reduction in body weight and/or increase in systemic T cell expansion.
      239. The combination of any one of embodiment 1 to 14 and 29 to 238 or the method of any one of embodiments 15 to 238, wherein the IL2Rβ binding moiety and IL2Rγ binding moiety are agonistic binders.
      240. The combination of any one of embodiment 1 to 14 and 29 to 239 or the method of any one of embodiments 15 to 239, wherein the binding of the IL2Rβ binding moiety and IL2Rγ binding moiety to their respective targets on an IL2 receptor-expressing cell elicits receptor signaling.
      241. The combination of any one of embodiment 1 to 14 and 29 to 240 or the method of any one of embodiments 15 to 240, wherein the binding of the IL2Rβ binding moiety and IL2Rγ binding moiety to their respective targets on an IL2 receptor-expressing cell elicits receptor signaling in a STAT5 reporter assay, e.g., as described in Section 8.1.2.
      242. The combination of any one of embodiment 1 to 14 and 29 to 241 or the method of any one of embodiments 15 to 241, wherein the binding of the IL2Rβ binding moiety and IL2Rγ binding moiety to their respective targets on an IL2 receptor-expressing cell elicits receptor signaling in a pSTAT5 assay, e.g., as described in Section 8.1.3.
      243. A combination comprising:
(a) a tumor-targeted IL2Rβ binding molecule comprising:
   (i) a first polypeptide chain comprising:
      (1) a first tumor-targeting moiety that binds to a first tumor-associated antigen, or component thereof (e.g., VH) associated with another component (e.g., VL) on a separate polypeptide chain; and
      (2) a first Fc domain having one or more amino acid substitutions that reduce binding to an Fc receptor and/or effector function; and
   (ii) a second polypeptide chain comprising:
      (1) an IL2Rβ binding moiety, optionally in the form of a single domain antibody (sdAb), scFv domain or Fab domain; and
      (2) a second Fc domain that is capable of heterodimerizing with the first Fc domain and having one or more amino acid substitutions that reduce binding to an Fc receptor and/or effector function; and
(b) a tumor-targeted IL2Rγ binding molecule comprising:
   (i) a third polypeptide chain comprising:
      (1) a second tumor-targeting moiety that binds to a second tumor-associated antigen expressed on the same tumor cell as the first tumor-associated antigen, or component thereof (e.g., VH) associated with another component (e.g., VL) on a separate polypeptide chain; and
      (2) a third Fc domain having one or more amino acid substitutions that reduce binding to an Fc receptor and/or effector function; and
   (ii) a fourth polypeptide chain comprising:
      (1) an IL2Rγ binding moiety, optionally in the form of a single domain antibody (sdAb), scFv domain or Fab domain; and
      (2) a fourth Fc domain that is capable of heterodimerizing with the third Fc domain and having one or more amino acid substitutions that reduce binding to an Fc receptor and/or effector function.
         244. The combination of embodiment 243, wherein the IL2Rβ binding moiety and IL2Rγ binding moiety share the same format.
         245. The combination of embodiment 243, wherein the IL2Rβ binding moiety and IL2Rγ binding moiety are sdAbs.
         246. The combination of embodiment 243, wherein the IL2Rβ binding moiety and IL2Rγ binding moiety are scFv domains.
         247. The combination of embodiment 243, wherein the wherein the IL2Rβ binding moiety and IL2Rγ binding moiety are Fab domains.
         248. The combination of embodiment 243, wherein the IL2Rβ binding moiety and IL2Rγ binding moiety have different formats.
         249. The combination of embodiment 243, wherein one of the IL2Rβ binding moiety and IL2Rγ binding moiety is a sdAb and the other is a Fab domain.
         250. The combination of any one of embodiments 243 to 249, wherein the IL2Rβ binding moiety and IL2Rγ binding moiety are agonistic binders.
         251. The combination of any one of embodiments 243 to 250, wherein the binding of the IL2Rβ binding moiety and IL2Rγ binding moiety to their respective targets on an IL2 receptor-expressing cell elicits receptor signaling.
         252. The combination of any one of embodiments 243 to 250, wherein the binding of the IL2Rβ binding moiety and IL2Rγ binding moiety to their respective targets on an IL2 receptor-expressing cell elicits receptor signaling in a STAT5 reporter assay, e.g., as described in Section 8.1.2.
         253. The combination of any one of embodiments 243 to 250, wherein the binding of the IL2Rβ binding moiety and IL2Rγ binding moiety to their respective targets on an IL2 receptor-expressing cell elicits receptor signaling in a pSTAT5 assay, e.g., as described in Section 8.1.3.
         254. The combination of any one of embodiments 243 to 253, wherein the first tumor-associated antigen and the second tumor-associated antigen are different.
         255. The combination of any one of embodiments 243 to 253, wherein the first tumor-associated antigen and the second tumor-associated antigen are the same.
         256. The combination of embodiment 255, wherein the first tumor-targeting moiety and the second tumor-targeting moiety are the same.
         257. The combination of embodiment 255, wherein the first tumor-targeting moiety and the second tumor-targeting moiety are different, e.g., bind to different epitopes.
         258. The combination embodiment 255 or embodiment 257, wherein the first tumor-targeting moiety and the second tumor-targeting moiety do not compete for binding to the tumor-associated antigen (e.g., are non-competing tumor-targeting moieties as determined using an antibody cross-competition assay as described in Section 8.1.6).
         259. The combination or method of any one of embodiments 243 to 258, wherein the first tumor-targeting moiety and/or the second tumor-targeting moiety are Fabs.
         260. The combination or method of any one of embodiments 243 to 258, wherein the first tumor-targeting moiety and/or the second tumor-targeting moiety are scFvs.
         261. The combination or method of any one of embodiments 243 to 258, wherein the first tumor-targeting moiety and/or the second tumor-targeting moiety are sdAbs.
         262. The combination or method of any one of embodiments 243 to 261, wherein the first tumor-targeting moiety and/or second tumor-targeting moiety bind(s) to EGFR.
         263. The combination or method of any one of embodiments 243 to 261, wherein the first tumor-targeting moiety and/or second tumor-targeting moiety bind(s) to PSMA.
         264. The combination or method of any one of embodiments 243 to 261, wherein the first tumor-targeting moiety and/or second tumor-targeting moiety bind(s) to MUC16.
         265. The combination or method of any one of embodiments 243 to 261, wherein the first tumor-targeting moiety and/or second tumor-targeting moiety bind(s) to HER2.
         266. The combination or method of any one of embodiments 243 to 261, wherein the first tumor-targeting moiety and/or second tumor-targeting moiety bind(s) to STEAP1.
         267. The combination or method of any one of embodiments 243 to 261, wherein the first tumor-targeting moiety and/or second tumor-targeting moiety bind(s) to CEACAM5.
         268. The combination of any one of embodiments 243 to 267, wherein the first and second Fc domains are IgG1, IgG2 or IgG4 Fc domains.
         269. The combination of any one of embodiments 243 to 267, wherein the first Fc domain and second Fc domain each comprises an amino acid sequence having at least 98% sequence identity to the amino acid sequence of SEQ ID NO:5, optionally comprising knob/hole substitutions and/or star mutation(s).
         270. The combination of any one of embodiments 243 to 267, wherein the first Fc domain and second Fc domain each comprises an amino acid sequence having at least 98% sequence identity to the amino acid sequence of SEQ ID NO:4, optionally comprising knob/hole substitutions and/or star mutation(s).
         271. The combination of any one of embodiments 243 to 267, wherein the first Fc domain and second Fc domain each comprises an amino acid sequence having at least 98% sequence identity to the amino acid sequence of SEQ ID NO:10, optionally comprising knob/hole substitutions and/or star mutation(s).
         272. The combination of any one of embodiments 243 to 271, wherein the first and second Fc domains have chimeric hinge domains.
         273. The combination of any one of embodiments 243 to 272, wherein the third and fourth Fc domains are IgG1, IgG2 or IgG4 Fc domains.
         274. The combination of any one of embodiments 243 to 273, wherein the third Fc domain and fourth Fc domain each comprises an amino acid sequence having at least 98% sequence identity to the amino acid sequence of SEQ ID NO:5, optionally comprising knob/hole substitutions and/or star mutation(s).
         275. The combination of any one of embodiments 243 to 273, wherein the third Fc domain and fourth Fc domain each comprises an amino acid sequence having at least 98% sequence identity to the amino acid sequence of SEQ ID NO:8, optionally comprising knob/hole substitutions and/or star mutation(s).
         276. The combination of any one of embodiments 243 to 273, wherein the third Fc domain and fourth Fc domain each comprises an amino acid sequence having at least 98% sequence identity to the amino acid sequence of SEQ ID NO:10, optionally comprising knob/hole substitutions and/or star mutation(s).
         277. The combination of any one of embodiments 243 to 276, wherein the third and fourth Fc domains have chimeric hinge domains.
         278. The combination of any one of embodiments 243 to 276, wherein the tumor-targeted IL2Rβ binding molecule and the tumor-targeted IL2Rγ binding molecule are in the same pharmaceutical composition.
         279. The combination of any one of embodiments 243 to 276, wherein the tumor-targeted IL2Rβ binding molecule and the tumor-targeted IL2Rγ binding molecule are in different pharmaceutical compositions.
         280. The combination of any one of embodiments 243 to 279, wherein the combination further comprises a multispecific T-cell engager.
         281. The combination of embodiment 280, wherein the multispecific T-cell engager is a bispecific T-cell engager.
         282. The combination or method of embodiment 280 or embodiment 281, wherein the multispecific T-cell engager comprises a TAA targeting moiety and a CD3 targeting moiety.
         283. The combination or method of embodiment 282, wherein the TAA targeting moiety of the multispecific T-cell engager targets the same TAA as the TAA targeted by the tumor-targeted IL2Rβ binding molecule and/or the tumor-targeted IL2Rγ binding molecule.
         284. The combination or method of embodiment 282, wherein the TAA targeting moiety of the multispecific T-cell engager targets a TAA that is different from the TAA targeted by the tumor-targeted IL2Rβ binding molecule and/or the tumor-targeted IL2Rγ binding molecule.
         285. The combination of any one of embodiments 282 to 284, wherein the TAA targeting moiety of the multispecific T-cell engager binds to MSLN.
         286. The combination of any one of embodiments 243 to 285, which is suitable for the treatment of cancer, optionally wherein the cancer is a solid tumor.
         287. The combination of any one of embodiments 243 to 286, which is suitable for the prevention or treatment of metastasis.
         288. The combination of any one of embodiments 243 to 287, which is suitable for stimulating the formation, stability and/or activity of a cytotoxic immune synapse.
         289. The combination of any one of embodiments 243 to 288, which is suitable for clustering of IL2Rβ and IL2Rγ receptor subunits in a lymphocyte.
         290. The combination of any one of embodiments 243 to 289, which is suitable for eliciting signaling through the IL2 receptor and/or IL15 receptor in a lymphocyte.
         291. The combination of any one of embodiments 243 to 290, which is suitable for inducing tumor cytolysis.
         292. The combination of any one of embodiments 243 to 291, which is suitable for inducing anti-tumor cytotoxicity.
         293. The combination of any one of embodiments 243 to 292, which is suitable for stimulating an immune response against a tumor.
         294. The combination of any one of embodiments 243 to 293, which is suitable for improving the safety of IL2 agonist cancer treatment.
         295. The combination of any one of embodiments 243 to 294, which is suitable for improving the therapeutic window of IL2 agonist cancer treatment.
         296. The combination of any one of embodiments 243 to 295, which has improved anti-tumor activity compared to an isotype control, optionally wherein anti-tumor activity is determined by (a) a reduction in post-implantation tumor radiance in a non-human animal (e.g., murine) model or (b) a reduction in tumor burden.
         297. The combination of any one of embodiments 243 to 296, which has reduced adverse effects compared to a bispecific antibody comprising the IL2Rβ binding moiety and the IL2Rγ binding moiety, optionally wherein the adverse side effects are reduction in body weight and/or increased systemic T cell expansion.
         298. The combination of any one of embodiments 243 to 297 for use as a medicament.
         299. The combination for use of embodiment 298 for use in a method for the treatment of cancer.
         300. A tumor-targeted IL2Rβ binding molecule for use in a method for the treatment of cancer, wherein:
(a) the tumor-targeted IL2Rβ binding molecule is defined as in any one of embodiments 243 to 299, and
(b) the method comprises administering to a subject in need thereof the tumor-targeted IL2Rβ binding molecule and a tumor-targeted IL2Rγ binding molecule as defined in any one of embodiments 243 to 299.
   301. A tumor-targeted IL2Rγ binding molecule for use in a method for the treatment of cancer, wherein:
(a) the tumor-targeted IL2Rγ binding molecule is defined as in any one of embodiments 243 to 299, and
(b) the method comprises administering to a subject in need thereof the tumor-targeted IL2Rγ binding molecule and a tumor-targeted IL2Rβ binding molecule as defined in any one of embodiments 243 to 299.
   302. A method comprising administering to a subject a combination as defined in any one of embodiments 243 to 301.
   303. The method of embodiment 302, wherein the method is a method of combination therapy for the treatment of cancer.
   304. The combination for use of embodiment 298 or embodiment 299, the tumor-targeted IL2Rβ binding molecule for use of embodiment 300, the tumor-targeted IL2Rγ binding molecule for use of embodiment 301, of the method of embodiment 302 or embodiment 303, wherein the method is a method of combination therapy:
(a) for the prevention or treatment of metastasis;
(b) by stimulating the formation, stability and/or activity of a cytotoxic immune synapse;
(c) by clustering of IL2Rβ and IL2Rγ receptor subunits in a lymphocyte;
(d) by eliciting signaling through the IL2 receptor in a lymphocyte;
(e) by inducing tumor cytolysis;
(f) by inducing anti-tumor cytotoxicity;
(g) by stimulating an immune response against a tumor;
(h) for improving the safety of IL2 agonist cancer treatment;
(i) for improving the therapeutic window of IL2 agonist cancer treatment; or
(j) a combination of any two or more of the foregoing uses.
   305. The (a) combination for use of embodiment 298, embodiment 299 or embodiment 304; (b) tumor-targeted IL2Rβ binding molecule for use of embodiment 300 or embodiment 304; (c) tumor-targeted IL2Rγ binding molecule for use of embodiment 301 or embodiment 304; or (d) method of any one of embodiments 302 to 304, wherein the method comprises administering a multispecific T-cell engager (e.g., simultaneously, sequentially or separately).
   306. The (a) combination for use of embodiment 305, (b) tumor-targeted IL2Rβ binding molecule for use of embodiment 305; (c) tumor-targeted IL2Rγ binding molecule for use of embodiment 305; or (d) method of embodiment 305, wherein the multispecific T-cell engager is as defined in any one of embodiments 280 to 285.
   307. A method comprising administering to a subject the combination of any one of embodiments 243 to 306.
   308. The method of embodiment 307, wherein the subject has cancer.
   309. The method of embodiment 308, wherein the cancer is a solid tumor.
   310. The method of any one of embodiments 307 to 309, wherein the administration results in eliciting IL2 signaling in tumor lymphocytes.
   311. The method of any one of embodiments 307 to 310, wherein the administration results in prevention or treatment of metastasis.
   312. The method of any one of embodiments 307 to 311, wherein the administration results in stimulating the formation, stability and/or activity of a cytotoxic immune synapse.
   313. The method of any one of embodiments 307 to 312, wherein the administration results in clustering of IL2Rβ and IL2Rγ receptor subunits in a lymphocyte.
   314. The method of any one of embodiments 307 to 313, wherein the administration results in eliciting signaling through the IL2 receptor in a lymphocyte.
   315. The method of any one of embodiments 307 to 314, wherein the administration results in inducing tumor cytolysis.
   316. The method of any one of embodiments 307 to 315, wherein the administration results in inducing anti-tumor cytotoxicity.
   317. The method of any one of embodiments 307 to 316, wherein the administration results in stimulating an immune response against a tumor.
   318. The method of any one of embodiments 307 to 317, wherein the administration results in improving the safety of IL2 agonist cancer treatment.
   319. The method of any one of embodiments 307 to 318, wherein the administration results in improving the therapeutic window of IL2 agonist cancer treatment.
   320. The method of any one of embodiments 307 to 319, wherein the administration results in improved anti-tumor activity compared to an isotype control.
   321. The method of any one of embodiments 307 to 320, wherein the administration results in reduced adverse effects compared to a bispecific antibody comprising the IL2Rβ binding moiety and the IL2Rγ binding moiety.
   322. A method comprising administering to a subject:
(a) a tumor-targeted IL2Rβ binding molecule comprising:
   (i) a first polypeptide chain comprising:
   (1) a first tumor-targeting moiety that binds to a first tumor-associated antigen (TAA), or component thereof (e.g., VH) associated with another component (e.g., VL) on a separate polypeptide chain; and
   (2) a first Fc domain having one or more mutations that reduce effector function; and
   (ii) a second polypeptide chain comprising:
   (1) a single domain antibody (sdAb) that binds to IL2Rβ; and
   (2) a second Fc domain that is capable of heterodimerizing with the first Fc domain and having one or more mutations that reduce effector function; and
(b) a tumor-targeted IL2Rγ binding molecule comprising:
   (i) a third polypeptide chain comprising:
   (1) a second tumor-targeting moiety that binds to a second tumor-associated antigen (TAA) expressed on the same tumor cell as the first tumor-associated antigen, or component thereof (e.g., VH) associated with another component (e.g., VL) on a separate polypeptide chain; and
   (2) a third Fc domain having one or more mutations that reduce effector function; and
   (ii) a fourth polypeptide chain comprising:
   (1) a single domain antibody (sdAb) that binds to IL2Rγ; and
   (2) a fourth Fc domain that is capable of heterodimerizing with the third Fc domain and having one or more mutations that reduce effector function.
      323. The method of embodiment 322, wherein the first tumor-associated antigen and the second tumor-associated antigen are different.
      324. The method of embodiment 322, wherein the first tumor-associated antigen and the second tumor-associated antigen are the same.
      325. The method of embodiment 324, wherein the first tumor-targeting moiety and the second tumor-targeting moiety are the same.
      326. The method of embodiment 324, wherein the first tumor-targeting moiety and the second tumor-targeting moiety are different, e.g., bind to different epitopes.
      327. The method of embodiment 324 or embodiment 326, wherein the first tumor-targeting moiety and the second tumor-targeting moiety are non-competing tumor-targeting moieties (e.g., as determined using an antibody cross-competition assay as described in Section 8.1.6).
      328. The method of any one of embodiments 322 to 327, wherein the first tumor-targeting moiety and/or the second tumor-targeting moiety are Fabs.
      329. The method of any one of embodiments 322 to 327, wherein the first tumor-targeting moiety and/or the second tumor-targeting moiety are scFvs.
      330. The method of any one of embodiments 322 to 327, wherein the first tumor-targeting moiety and/or the second tumor-targeting moiety are sdAbs.
      331. The method of any one of embodiments 322 to 330, wherein the first TAA and/or second TAA is EGFR.
      332. The method of any one of embodiments 322 to 330, wherein the first TAA and/or second TAA is PSMA.
      333. The method of any one of embodiments 322 to 330, wherein the first TAA and/or second TAA is MUC16.
      334. The method of any one of embodiments 322 to 330, wherein the first TAA and/or second TAA is HER2.
      335. The method of any one of embodiments 322 to 330, wherein the first TAA and/or second TAA is STEAP1.
      336. The method of any one of embodiments 322 to 330, wherein the first TAA and/or second TAA is CEACAM5.
      337. The method of any one of embodiments 322 to 336, wherein both the first and second Fc domains are IgG1 Fc domains, IgG2 Fc domains or IgG4 Fc domains.
      338. The method of any one of embodiments 322 to 336, wherein the first Fc domain and second Fc domain each comprises an amino acid sequence having at least 98% sequence identity to the amino acid sequence of SEQ ID NO:5, optionally comprising knob/hole substitutions and/or star mutation(s).
      339. The method of any one of embodiments 322 to 336, wherein the first Fc domain and second Fc domain each comprises an amino acid sequence having at least 98% sequence identity to the amino acid sequence of SEQ ID NO:4, optionally comprising knob/hole substitutions and/or star mutation(s).
      340. The method of any one of embodiments 322 to 336, wherein the first Fc domain and second Fc domain each comprises an amino acid sequence having at least 98% sequence identity to the amino acid sequence of SEQ ID NO:10, optionally comprising knob/hole substitutions and/or star mutation(s).
      341. The method of any one of embodiments 322 to 340, wherein the first and second Fc domains have chimeric hinge domains.
      342. The method of any one of embodiments 322 to 341, wherein the third and fourth Fc domains are IgG1, IgG2 or IgG4 Fc domains.
      343. The method of any one of embodiments 322 to 342, wherein the third Fc domain and fourth Fc domain each comprises an amino acid sequence having at least 98% sequence identity to the amino acid sequence of SEQ ID NO:5, optionally comprising knob/hole substitutions and/or star mutation(s).
      344. The method of any one of embodiments 322 to 342, wherein the third Fc domain and fourth Fc domain each comprises an amino acid sequence having at least 98% sequence identity to the amino acid sequence of SEQ ID NO:8, optionally comprising knob/hole substitutions and/or star mutation(s).
      345. The method of any one of embodiments 322 to 342, wherein the third Fc domain and fourth Fc domain each comprises an amino acid sequence having at least 98% sequence identity to the amino acid sequence of SEQ ID NO:10, optionally comprising knob/hole substitutions and/or star mutation(s).
      346. The method of any one of embodiments 322 to 345, wherein the third and fourth Fc domains have chimeric hinge domains.
      347. The method of any one of embodiments 322 to 346, wherein the tumor-targeted IL2Rβ binding molecule and the tumor-targeted IL2Rγ binding molecule are in the same pharmaceutical composition.
      348. The method of any one of embodiments 322 to 346, wherein the tumor-targeted IL2Rβ binding molecule and the tumor-targeted IL2Rγ binding molecule are in different pharmaceutical compositions.
      349. The method of any one of embodiments 322 to 348, which further comprises administering to the subject a multispecific T-cell engager.
      350. The method of embodiment 349, wherein the multispecific T-cell engager is a bispecific T-cell engager.
      351. The method of embodiment 349 or embodiment 350, wherein the multispecific T-cell engager comprises a TAA targeting moiety and a CD3 targeting moiety.
      352. The method of embodiment 351, wherein the TAA targeting moiety of the multispecific T-cell engager targets the same TAA as the TAA targeted by the tumor-targeted IL2Rβ binding molecule and/or the tumor-targeted IL2Rγ binding molecule.
      353. The method of embodiment 351, wherein the TAA targeting moiety of the multispecific T-cell engager targets a TAA that is different from the TAA targeted by the tumor-targeted IL2Rβ binding molecule and/or the tumor-targeted IL2Rγ binding molecule.
      354. The method of any one of embodiments 351 to 353, wherein the TAA targeting moiety binds to MSLN.
      355. The method of any one of embodiments 322 to 354, wherein the subject has cancer.
      356. The method of embodiment 355, wherein the cancer is a solid tumor.
      357. The method of any one of embodiments 322 to 356, wherein the administration results in:
(a) prevention or treatment of metastasis;
(b) stimulating the formation, stability and/or activity of a cytotoxic immune synapse;
(c) clustering of IL2Rβ and IL2Rγ receptor subunits in a lymphocyte;
(d) eliciting signaling through the IL2 receptor in a lymphocyte;
(e) inducing tumor cytolysis;
(f) inducing anti-tumor cytotoxicity;
(g) stimulating an immune response against a tumor;
(h) improving the safety of IL2 receptor agonist cancer treatment;
(i) improving the therapeutic window of IL2 receptor agonist cancer treatment; or
(j) any two or more of (a) through (j).

### 8. EXAMPLES

### 8.1. Materials and Methods

### 8.1.1. Design and Production of Tumor-targeted IL2Rβ and IL2Rγ Binding Molecule Constructs

Exemplary tumor-targeted IL2Rβ binding molecules as depicted in FIG. 2B were designed to comprise two polypeptides herein referred to as the first and second polypeptides, the first polypeptide comprising from N- to C-terminal, a first tumor targeting moiety in Fab format, a linker, and a first Fc domain which enables dimerization; and the second polypeptide comprising from N- to C-terminal, an IL2Rβ binding moiety comprising a single domain anti- IL2Rβ antibody, a linker, and a second Fc domain that forms a dimer with the first Fc domain.

Similarly, exemplary tumor-targeted IL2Rγ binding molecules as depicted in FIG. 3B were designed to comprise two polypeptides herein referred to as the third and fourth polypeptides, the third polypeptide comprising from N- to C-terminal, a second tumor targeting moiety in Fab format, a linker, and a third Fc domain which enables dimerization; and the fourth polypeptide comprising from N- to C-terminal, a single domain anti-IL2Rγ antibody, a linker, and a fourth Fc domain that forms a dimer with the third Fc domain.

The details of exemplary tumor-targeted IL2Rβ and IL2Rγ binding molecule constructs produced are provided in Table E1 below.

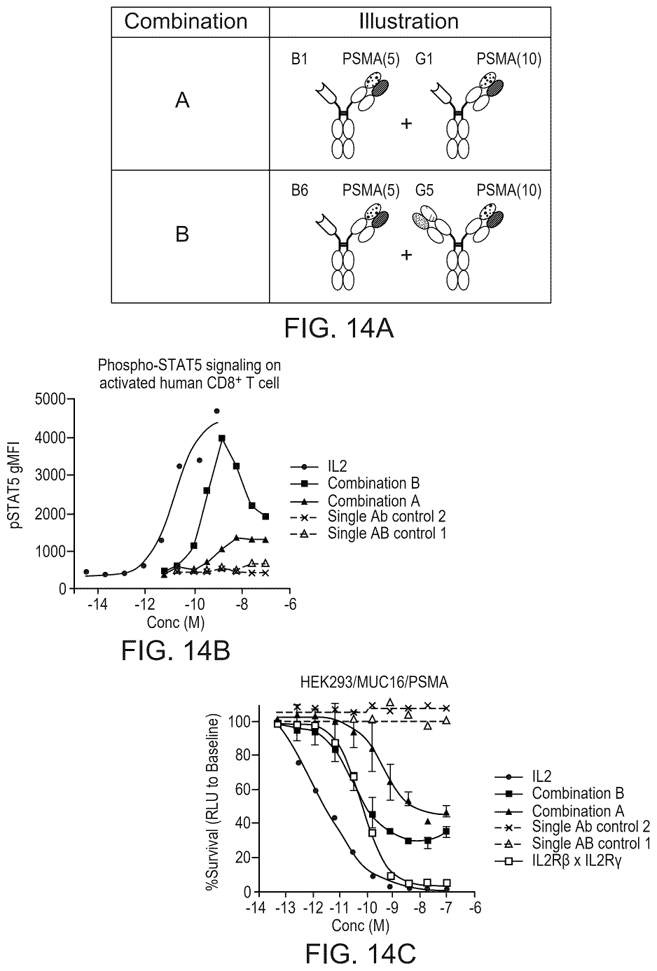

TABLE E1

| Construct Name/ | |
|---|---|
| Components | Sequence |
| B1 x PSMA (5) | Chain 1 - B1_IL2Rb HC hole star - |
| | QVQLQESGPGLVKPSGTLSLTCAVSGGSISSSDWWSWVRQPPGKGLEWIGEI |
| | DHSGSTNYNPSLMSRVTISVDKSKNQFSLKLSSVTAADTAVYFCGRGSWELSD |
| | AFDIRGQGTLVTVSSESKYGPPCPPCPAPPVAGPSVFLFPPKPKDTLMISRTP |
| | EVTCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQV |
| | SLSCAVKGFYPSDIAVEWESNGQPENNYKTTPPVLDSDGSFFLVSRLTVDKSR |
| | WQEGNVFSCSVMHEALHNRFTQKSLSLSPGK (SEQ ID NO: 48) |
| | Chain 2 - anti-PSMA(5) Fab HC knob |
| | [Anti-PSMA(5) VH] - [IgG4 CH1] - |
| | ESKYGPPCPPCPAPPVAGPSVFLFPPKPKDTLMISRTPEVTCVVVDVSQEDPE |
| | VQFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIA |
| | VEWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| G1 x PSMA (8) | Chain 1 - G1_IL2Rg HC hole star - |
| | QVQLVESGGGLVKPGGSLRLSCAASGFTFSDYYMSWIRQAPGKGLEWVSSISS |
| | SGDTIYYADSVQGRFTLSRDNAENSLFLQMNSLRAEDTAVYYCARGDAVSITGD |
| | YRGQGTLVTVSSESKYGPPCPPCPAPPVAGPSVFLFPPKPKDTLMISRTPEVTC |
| | VVVDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLSC |
| | AVKGFYPSDIAVEWESNGQPENNYKTTPPVLDSDGSFFLVSRLTVDKSRWQEG |
| | NVFSCSVMHEALHNRFTQKSLSLSPGK (SEQ ID NO: 50) |
| | Chain 2 - anti-PSMA(8)Fab HC knob |
| | [Anti-PSMA(8) VH] - [IgG4 CH1] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| B2 x PSMA (3) | Chain 1 - B2_IL2Rb HC hole star |
| | QVQLQESGPGLVKSSETLSLTCTVSGGSISSSDWWSWVRQPPGKGLEWIGEID |
| | HSGSTNYNPSLMSRVTISVDKSKNQFSLKLSSVTAADTAVYFCARGSWELTDAF |
| | DIRGQGTLVTVSSESKYGPPCPPCPAPPVAGPSVFLFPPKPKDTLMISRTPEVT |
| | CVVVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | SCAVKGFYPSDIAVEWESNGQPENNYKTTPPVLDSDGSFFLVSRLTVDKSRWQ |
| | EGNVFSCSVMHEALHNRFTQKSLSLSPGK (SEQ ID NO: 51) |
| | Chain 2 - anti-PSMA(3) Fab HC knob |
| | [Anti-PSMA(3) VH] - [IgG4 CH1] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| G4 x PSMA (8) | Chain 1 - G4_IL2Rg HC hole star - |
| | QVQLVESGGDLVKPGGSLRLSCAASGFTFSDYYMSWLRQAPGKELEWVSHIS |
| | SSGTTTYYADSVEGRFTITRDNAKNSLYLQMNSLRAEDTAVYYCARGAAVAPG |
| | FDSRGQGTLVTVSSESKYGPPCPPCPAPPVAGPSVFLFPPKPKDTLMISRTPEV |
| | TCVVVDVSQEDPEVQFNWYVDGVEVHNAKTKPREEQFNSTYRVVSVLTVLHQ |
| | DWLNGKEYKCKVSNKGLPSSIEKTISKAKGQPREPQVYTLPPSQEEMTKNQVS |
| | LSCAVKGFYPSDIAVEWESNGQPENNYKTTPPVLDSDGSFFLVSRLTVDKSRW |
| | QEGNVFSCSVMHEALHNRFTQKSLSLSPGK (SEQ ID NO: 52) |
| | Chain 2 - anti-PSMA(8) Fab HC knob |
| | [Anti-PSMA(8) VH] - [IgG4 CH1] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| B2 x PSMA (5) | Chain 1 - B2_IL2Rb HC hole star - |
| | QVQLQESGPGLVKSSETLSLTCTVSGGSISSSDWWSWVRQPPGKGLEWIGEID |
| | HSGSTNYNPSLMSRVTISVDKSKNQFSLKLSSVTAADTAVYFCARGSWELTDAF |
| | DIRGQGTLVTVSSESKYGPPCPPCPAPPVAGPSVFLFPPKPKDTLMISRTPEVT |
| | CVVVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | SCAVKGFYPSDIAVEWESNGQPENNYKTTPPVLDSDGSFFLVSRLTVDKSRWQ |
| | EGNVFSCSVMHEALHNRFTQKSLSLSPGK (SEQ ID NO: 51) |
| | Chain 2 - anti-PSMA(5) Fab HC knob - |
| | [Anti-PSMA(5) VH] - [IgG4 CH1] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| G2 x PSMA (8) | Chain 1 - G2_IL2Rg HC hole star - |
| | QVQLVESGGGLVKPGGSLRLSCAASGFTFSDYYMSWIRQAPGKGLEWVSYISS |
| | SGSTIYYADSVKGRFTISRDNAKNSLYLQMNSLRAEDTAVYYCARGDAVSITGD |
| | YRGQGTLVTVSSESKYGPPCPPCPAPPVAGPSVFLFPPKPKDTLMISRTPEVTC |
| | VVVDVSQEDPEVQFNWYVDGVEVHNAKTKPREEQFNSTYRVVSVLTVLHQDWA |
| | LNGKEYKCKVSNKGLPSSIEKTISKAKGQPREPQVYTLPPSQEEMTKNQVSLSC |
| | VKGFYPSDIAVEWESNGQPENNYKTTPPVLDSDGSFFLVSRLTVDKSRWQEG |
| | NVFSCSVMHEALHNRFTQKSLSLSPGK (SEQ ID NO: 53) |
| | Chain 2 - anti-PSMA(8) Fab HC knob - |
| | [Anti-PSMA(8) VH] - [IgG4 CH1] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| G3 x PSMA (8) | Chain 1 - G3_IL2Rg HC hole star - |
| | QVQLVESGGGLVKPGGSLRLSCAASGFTFNDYYMSWIRQAPGKGLEWVSHISS |
| | SGSTIYYADSVKGRFTVSRDNANNSLYLQMHSLRAEDTAVYYCARGDAVSITGD |
| | YRGQGTLVTVSSESKYGPPCPPCPAPPVAGPSVFLFPPKPKDTLMISRTPEVTC |
| | VVVDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLSC |
| | AVKGFYPSDIAVEWESNGQPENNYKTTPPVLDSDGSFFLVSRLTVDKSRWQEG |
| | NVFSCSVMHEALHNRFTQKSLSLSPGK (SEQ ID NO: 54) |
| | Chain 2 - anti-PSMA(8) Fab HC knob |
| | [Anti-PSMA(8) VH] - [IgG4 CH1] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| G4 x PSMA (8) | Chain 1 - G4_IL2Rg HC hole star - |
| | QVQLVESGGDLVKPGGSLRLSCAASGFTFSDYYMSWLRQAPGKELEWVSHIS |
| | SSGTTTYYADSVEGRFTITRDNAKNSLYLQMNSLRAEDTAVYYCARGAAVAPG |
| | FDSRGQGTLVTVSSESKYGPPCPPCPAPPVAGPSVFLFPPKPKDTLMISRTPEV |
| | TCVVVDVSQEDPEVQFNWYVDGVEVHNAKTKPREEQFNSTYRVVSVLTVLHQ |
| | DWLNGKEYKCKVSNKGLPSSIEKTISKAKGQPREPQVYTLPPSQEEMTKNQVS |
| | LSCAVKGFYPSDIAVEWESNGQPENNYKTTPPVLDSDGSFFLVSRLTVDKSRW |
| | QEGNVFSCSVMHEALHNRFTQKSLSLSPGK (SEQ ID NO: 52) |
| | Chain 2 - anti-PSMA(8) Fab HC knob - |
| | [Anti-PSMA(8) VH] - [IgG4 CH1] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| B1 x MUC16 (4) | Chain 1 - B1_IL2Rb HC hole star - |
| | QVQLQESGPGLVKPSGTLSLTCAVSGGSISSSDWWSWVRQPPGKGLEWIGEI |
| | DHSGSTNYNPSLMSRVTISVDKSKNQFSLKLSSVTAADTAVYFCGRGSWELSD |
| | AFDIRGQGTLVTVSSESKYGPPCPPCPAPPVAGPSVFLFPPKPKDTLMISRTPE |
| | VTCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQV |
| | SLSCAVKGFYPSDIAVEWESNGQPENNYKTTPPVLDSDGSFFLVSRLTVDKSR |
| | WQEGNVFSCSVMHEALHNRFTQKSLSLSPGK (SEQ ID NO: 48) |
| | Chain 2 - anti-MUC16(4) Fab HC knob - |
| | [Anti-MUC16(4) VH] - [IgG4 CH1] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| G1 x MUC16 (9) | Chain 1 - G1_IL2Rg HC hole star - |
| | QVQLVESGGGLVKPGGSLRLSCAASGFTFSDYYMSWIRQAPGKGLEWVSSISS |
| | SGDTIYYADSVQGRFTLSRDNAENSLFLQMNSLRAEDTAVYYCARGDAVSITGD |
| | YRGQGTLVTVSSESKYGPPCPPCPAPPVAGPSVFLFPPKPKDTLMISRTPEVTC |
| | VVVDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLSC |
| | AVKGFYPSDIAVEWESNGQPENNYKTTPPVLDSDGSFFLVSRLTVDKSRWQEG |
| | NVFSCSVMHEALHNRFTQKSLSLSPGK (SEQ ID NO: 50) |
| | Chain 2 - anti-MUC16(9) Fab HC knob - |
| | [Anti-MUC16(9) VH] - [IgG4 CH1] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHEAL |
| | HNHYTQKSLSLSLGK (SEQ ID NO: 9) |
| | Chain 3 - Universal light chain |
| B(6) x PSMA (5) | Chain 1 - B(6)_IL2Rb HC hole star - |
| Combination B in | [Anti-IL2Rß(6)) VHH]- |
| FIG. 14 | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLSCAVKGFYPSDIAV |
| | EWESNGQPENNYKTTPPVLDSDGSFFLVSRLTVDKSRWQEGNVFSCSVMHEA |
| | LHNRFTQKSLSLSPGK (SEQ ID NO: 257) |
| | Chain 2 - anti-PSMA(5) Fab HC knob |
| | [Anti-PSMA(5) VH] - [IgG4 CH1] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| G5 x PSMA (10) | Chain 1 - G5_IL2Rg HC hole star - |
| Combination B in | [Anti-IL2Ry(5) VH] - [IgG4 CH1] - |
| FIG. 14 | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLSCAVKGFYPSDIAV |
| | EWESNGQPENNYKTTPPVLDSDGSFFLVSRLTVDKSRWQEGNVFSCSVMHEA |
| | LHNRFTQKSLSLSPGK (SEQ ID NO: 257) |
| | Chain 2 - anti-PSMA(10) Fab HC knob |
| | [Anti-PSMA(10) VH] - [IgG4 CH1] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| B1 x PSMAscfv | Chain 1 - B1_IL2Rb HC hole star - |
| (5) | QVQLQESGPGLVKPSGTLSLTCAVSGGSISSSDWWSWVRQPPGKGLEWIGEI |
| | DHSGSTNYNPSLMSRVTISVDKSKNQFSLKLSSVTAADTAVYFCGRGSWELSD |
| | AFDIRGQGTLVTVSSESKYGPPCPPCPAPPVAGPSVFLFPPKPKDTLMISRTPE |
| | VTCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQV |
| | SLSCAVKGFYPSDIAVEWESNGQPENNYKTTPPVLDSDGSFFLVSRLTVDKSR |
| | WQEGNVFSCSVMHEALHNRFTQKSLSLSPGK (SEQ ID NO: 48) |
| | Chain 2 - anti-PSMAscFv(5) HC knob |
| | [Anti-PSMA scFv(B)] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |
| G1 x PSMAscfv | Chain 1 - G1_IL2Rg HC hole star - |
| (10) | QVQLVESGGGLVKPGGSLRLSCAASGFTFSDYYMSWIRQAPGKGLEWVSSISS |
| | SGDTIYYADSVQGRFTLSRDNAENSLFLQMNSLRAEDTAVYYCARGDAVSITGD |
| | YRGQGTLVTVSSESKYGPPCPPCPAPPVAGPSVFLFPPKPKDTLMISRTPEVTC |
| | WVVDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLSC |
| | AVKGFYPSDIAVEWESNGQPENNYKTTPPVLDSDGSFFLVSRLTVDKSRWQEG |
| | NVFSCSVMHEALHNRFTQKSLSLSPGK (SEQ ID NO: 50) |
| | Chain 2 - anti-PSMA (10) HC knob |
| | [Anti-PSMA scFv(A)] - |
| | ESKYGPPCPPCPAPPVAGPSVFLFPPKPKDTLMISRTPEVTCVVVDVSQEDPEV |
| | QFNWYVDGVEVHNAKTKPREEQFNSTYRVVSVLTVLHQDWLNGKEYKCKVSN |
| | KGLPSSIEKTISKAKGQPREPQVYTLPPSQEEMTKNQVSLWCLVKGFYPSDIAV |
| | EWESNGQPENNYKTTPPVLDSDGSFFLYSRLTVDKSRWQEGNVFSCSVMHEA |
| | LHNHYTQKSLSLSLGK (SEQ ID NO: 49) |
| | Chain 3 - Universal light chain |

The constructs were expressed in Expi293F™ cells by transient transfection following the manufacturer's protocol (Thermo Fisher Scientific). Proteins in Expi293F™ supernatant were purified using the ProteinMaker system (Protein BioSolutions, Gaithersburg, MD) with either HiTrap™ Protein or MabSelect SuRe columns (Cytiva). After single step elution, the constructs were neutralized, dialyzed into a final buffer of phosphate buffered saline (PBS) with 5% glycerol, aliquoted and stored at −80° C. until use.

### 8.1.2. STAT5 Reporter Assay

A Signal Transducer and Activator of Transcription 5 (STAT5)-driven luciferase-based reporter assay was used to evaluate the ability of tumor-targeted IL2Rβ binding molecules and tumor-targeted IL2Rγ binding molecules to activate STAT5-mediated transcription in STAT5-Luc reporter cells.

A day prior to screening, YT reporter cells were diluted to 5×10⁵ cells/mL in Iscove's media supplemented with 2 mM L-Glutamine/Penicillin/Streptomycin+20% FBS. On the day of the assay, cells were spun down, resuspended in assay medium (RPMI1640 media supplemented with 2 mM L-Glutamine/Penicillin/Streptomycin+10% FBS), plated at 2.5×10⁴ reporter cells/well in 96-well white flat bottom plates, and incubated with recombinant IL2, tumor-targeted IL2Rβ and IL2Rγ binding molecules, or bispecific IL2Rβ×IL2Rγ control antibodies. In instances where TAA-expressing cells were used for measuring TAA-targeted agonism, we used HEK293 or Raji cells engineered to express hPSMA, or endogenous PSMA-expressing LNCaP or 22Rv1 cells, or endogenous MUC16-expressing OVCAR3 cells. TAA-expressing cells were suspended in assay medium, plated at 2.5×10⁴ cells/well, and co-incubated with titrated molecules for 10 mins prior to adding YT reporter cells. Each construct was serially diluted (1:5) over an 11-point titration range (50 nM to 5.12 fM) and a 12^{th} point containing no protein. After plates were incubated for 4 hours and 30 minutes at 37° C. and 5% CO₂, 100 μL ONE-Glo™ (Promega) reagent was added to the wells to lyse the cells and detect luciferase activity. The emitted light was measured in RLU on an Envision multilabel plate reader (Revvity). All serial dilutions were tested in duplicates.

8.1.3. pSTAT5 Assay

Human peripheral blood mononuclear cells (PBMCs) were thawed and rested in assay media (RPMI+10% FBS+2 mM L-glutamine/Pen/Strep) overnight to get naive unstimulated PBMCs. Separately, human PBMCs were pre-activated by culturing for 72 hours in assay medium (RPMI+10% FBS+2 mM L-glutamine/Pen/Strep) in the presence of CD3/CD28 Dynabeads™ (Thermo/11132D) at a 1:1 (beads:PBMC) ratio and in the presence of 30 U/mL human IL2 (Proleukin). Beads were removed and activated PBMCs were rested in assay medium overnight at 37° C. C4-2 tumor cells were spun down, resuspended in assay medium and added to plates at 5×10⁴ cells/well. Tumor-targeted IL2Rβ and IL2Rγ binding molecules were serially diluted (range: 100 nM to 47.7fM) alone or in equal molar combination and added to C4-2 cells, followed by addition of either naïve or activated PBMCs at 5×10⁴ cells/well. For the activated PBMC assay, a 200 pM constant Signal1 (STEAP1×CD3) was also added. Plates were incubated for 1 hour at 37° C. before fixation with Cytofix buffer (BD/554655) for 12 minutes at 37° C. Cells were then permeabilization with pre-chilled Perm Buffer III (BD/558050) for 10 mins on ice and washed 2 times. Cells were then stained with aCD3 (BD/563918), aCD8 (BD/563256), aCD4 (BD/562843), aCD25 (BD/562442), aFOXP3 (BD/560047) and pSTAT5 (BD/562076) for 1 hour. Cells were washed twice before data was acquired using a BD Fortessa ×20 flow cytometer.

8.1.4. In vitro Target Cell Killing and IFNγ Release Assay

Human peripheral blood mononuclear cells (PBMCs) were isolated from healthy donor leukocyte packs using the EasySep™ Direct Human PBMC Isolation Kit and following the manufacturers recommended protocol. Subsequently, CD3+ T-cells were isolated using an EasySep™ Human CD8+ T Cell Isolation Kit from StemCell Technologies and following the manufacturer's recommended instructions. Media used for maintaining CD3+ T-cells and conducting experiments consisted of X-VIVO 15 cell culture media supplemented with 10% FBS, HEPES, NaPyr, NEAA, and 0.01 mM BME. CD3+ T-cells were pre-activated with CD3/CD28 Dynabeads™ (Thermo/11132D) and human IL2 (Proleukin) for 3 days. Target cells for this assay were HEK293 cells engineered to express hPSMA and hMUC16, which also express a luminescent tag containing a caspase cleavable domain, such that when caspases are active luminescence is lost. Thus, as target cells die the RLU signal is reduced. Activated T-cells and target cells were incubated together with 6 pM constant dose of MUC16×CD3 and a serial dilution (10-point, 4-fold titration starting at 100 nM of hPSMA-targeted IL2Rβ and IL2Rγ binding molecules alone or in equal molar combination. The lowest point on the curve contains no titrated IL2R or Isotype control molecules. Assay was readout after 3 days at 37° C. 5% CO₂. Prior to addition of Nano-Glo to sample wells for detecting luminescence, supernatant was collected for assessing IFNγ release. For the luminescent readout emitted light was measured in RLU on a multilabel plate reader Envision (Revvity). For assessing IFNγ release, 5 μL from the day 3 collected supernatant was tested using IFNγ alphaLISA (Revvity) for each well. All serial dilutions were tested in duplicates. EC50 values of the antibodies were determined using GraphPad Prism™ software from a four-parameter logistic equation over a 10-point dose-response curve.

8.1.5. In vivo Administration of Constructs

Female NSG mice were injected with 2.5×106 PBMC cells intraperitoneally two weeks before 1.5×10⁶ ascites cells from the OVCAR-3/Luc cell line, which were previously passaged in vivo, were administered intraperitoneally (day 0). On Day 4 after administration, mice were checked for T cell engraftment by flow cytometry and then assigned to treatment groups based on BLI imaging to ensure similar tumor burden (n=4-5 mice per group). On the day of randomization (Day 4) and on Days 7 and 11, mice were injected intraperitoneally with the assigned constructs at the predetermined amounts presented in Table E2. Blood samples were collected on Day14 for flow cytometry analysis. Tumor burden (assessed via BLI imaging) and body weight changes were measured twice per week throughout the study.

TABLE E2

| Group | Treatments | N |
|---|---|---|
| 1 | Isotype (2 mg/kg) | 5 |
| 2 | MSLN × CD3 (0.5 mg/kg) | 5 |
| 3 | IL2Rβ × MUC16 (2 mg/kg) + | 4 |
| | IL2Rγ × MUC16 (2 mg/kg) | |
| 4 | MSLN × CD3 (0.5 mg/kg) + | 5 |
| | IL2Rβ × MUC16 (0.5 mg/kg) + | |
| | IL2Rγ × MUC16 (0.5 mg/kg) | |
| 5 | MSLN × CD3 (0.5 mg/kg) + | 5 |
| | IL2Rβ × IL2Rγ (0.5 mg/kg) | |

### 8.1.6. Antibody Cross-Competition Assessment

Competition of antibodies for binding to a target molecule was determined using a real time, label-free bio-layer interferometry assay on the Octet HTX biosensor platform (Pall ForteBio Corp.). The assay was performed at 25° C. in a buffer of 10 mM HEPES, 150 mM NaCl, 3 mM EDTA, 1 mg/mL BSA, 0.05% v/v Surfactant Tween-20, pH 7.4 (HBS-EBT buffer) with the plate shaking at the speed of 1000 rpm. To assess whether two test antibodies compete with one another for binding to their respective epitopes on their specific target antigen, the target antigen with a His tag was first captured on Octet biosensor tips coated with an antibody against the His-tag. The antigen captured biosensor tips were then saturated with a first test antibody by dipping into wells containing a solution of the first test antibody. The biosensor tips were then subsequently dipped into wells containing a solution of a second test antibody. The biosensor tips were washed in HBS-EBT buffer in between every step of the assay. The real-time binding response was monitored during the entire course of the assay, the binding response at the end of every step was recorded and the response of the second test antibody binding to the target antigen pre-complexed with the first test antibody was assessed. Target-specific antibodies having a response of 0.2 or less are considered to be “competing” antibodies, those having a response of 0.6 or greater are considered to be “non-competing” antibodies, and those having a response greater than 0.2 and less than 0.6 are considered to be “partially-competing” antibodies.

### 8.2. Example 1: Activation of STAT5-Signaling by Tumor-Targeted IL2Rβ and IL2Rγ Binding Molecules

Tumor-targeted IL2Rβ and IL2Rγ binding molecules were designed and produced as described in Section 8.1.1. Tumor targeting moieties of the tumor-targeted IL2Rβ and IL2Rγ binding molecule constructs comprised anti-PSMA Fabs. The ability of a combination of tumor-targeted IL2Rβ and IL2Rγ binding molecules to activate STAT5-signaling was assessed in a STAT5-reporter cell-based assay as described in Section 8.1.2. in the presence and absence of PSMA-expressing 293 or Raji cells.

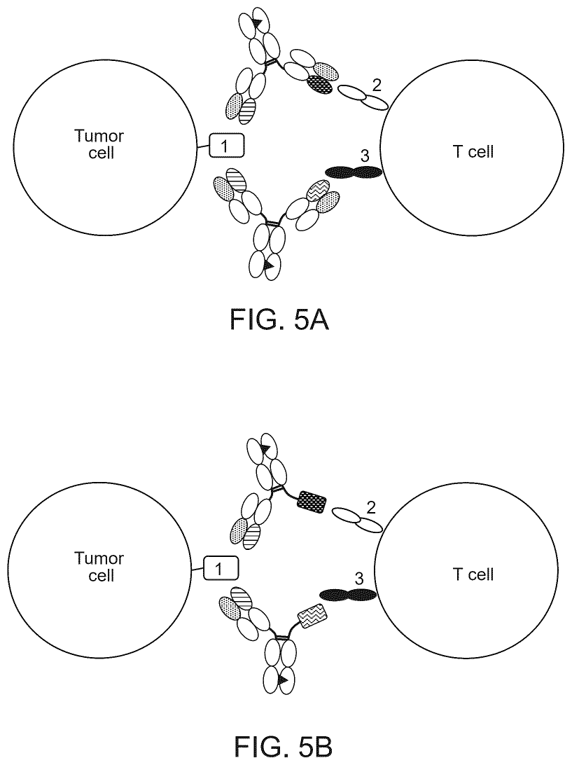

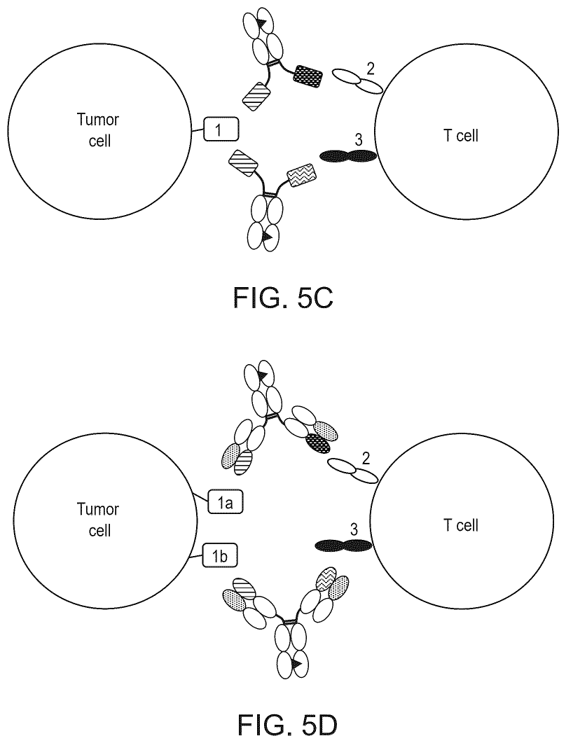

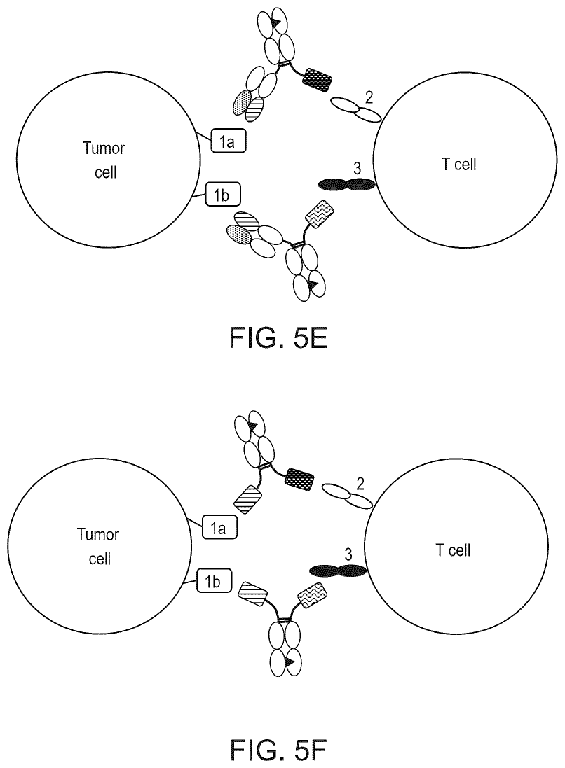

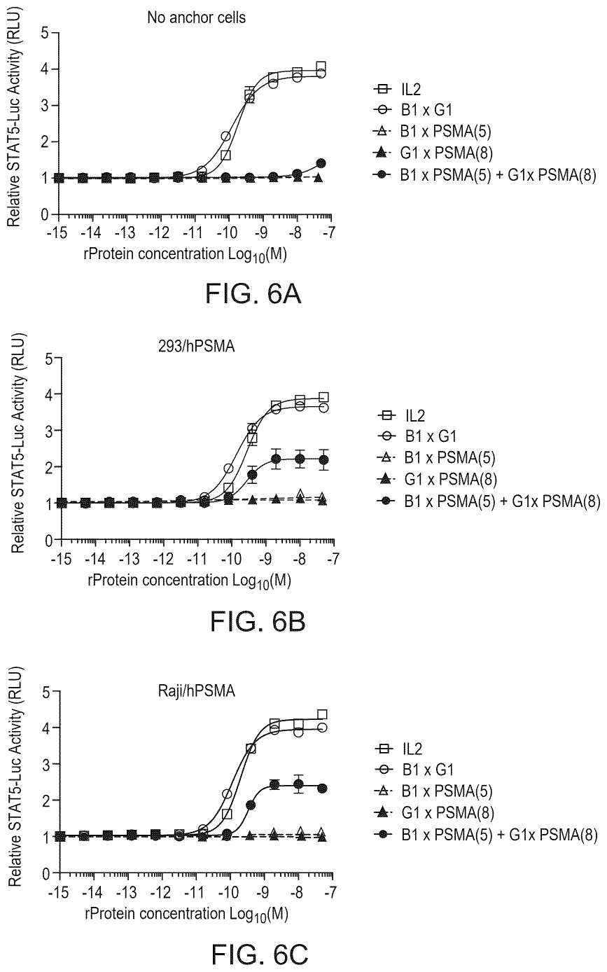

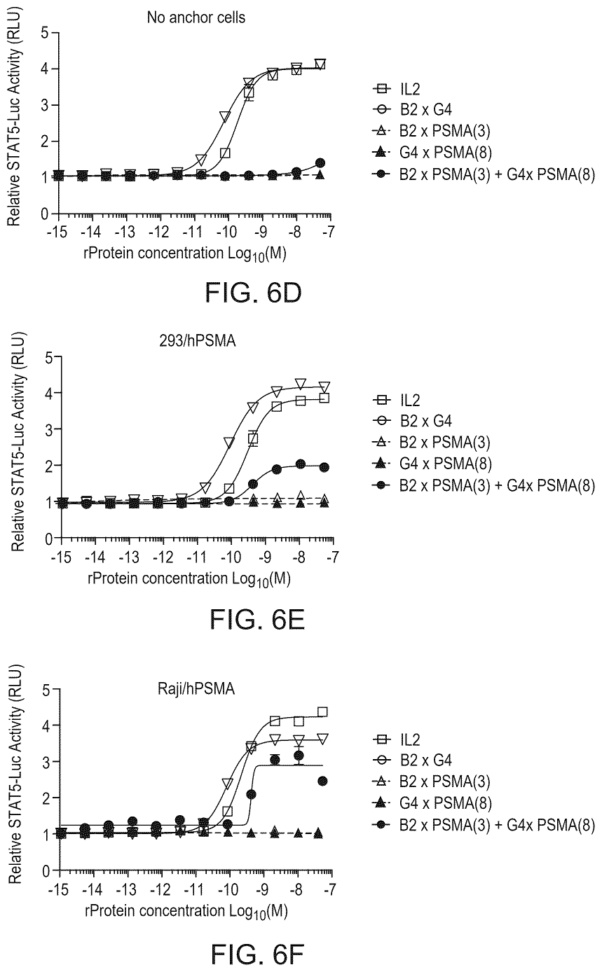

First, tumor-targeted IL2Rβ binding molecule construct B1×PSMA(5), IL2Rγ binding molecule construct G1×PSMA(8) were evaluated alone or in combination. A bispecific IL2Rβ×IL2Rγ antibody B1×G1 was used as a control. When no PSMA-expressing cells were present, tumor-targeted IL2Rβ and IL2Rγ binding molecules failed to activate STAT5 signaling neither alone nor in combination, except at the highest concentration (FIG. 6A). In the presence of PSMA-expressing 293 or Raji cells, the combination of the tumor-targeted IL2Rβ and IL2Rγ binding molecule constructs activated STAT5 signaling (FIGS. 5B and 5C).

Next, tumor-targeted IL2Rβ binding molecule construct B2×PSMA(3), IL2Rγ binding molecule construct G2×PSMA(8) were evaluated alone or in combination and a bispecific IL2Rβ×IL2Rγ antibody B2×G2 was used as a control. Similarly, tumor-targeted IL2Rβ and IL2Rγ binding molecules did not activate STAT5 signaling alone or in combination (FIG. 6D) when no PSMA-expressing cells were present, and the combination of the tumor-targeted IL2Rβ and IL2Rγ binding molecule constructs activated STAT5 signaling in the presence of PSMA-expressing 293 or Raji cells (FIGS. 6E and 6F).

### 8.3. Example 2: Activation of STAT5-Signaling by Tumor-Targeted IL2Rβ and IL2Rγ Binding Molecules in the Presence of Endogenous PSMA Expressing Cells

Tumor-targeted IL2Rβ and IL2Rγ binding molecules were designed and produced as described in Section 8.1.1. Tumor targeting moieties of the tumor-targeted IL2Rβ and IL2Rγ binding molecule constructs comprised anti-PSMA or anti-MUC16 Fabs. The ability of a combination of tumor-targeted IL2Rβ and IL2Rγ binding molecules to activate STAT5-signaling was assessed in a STAT5-reporter cell-based assay as described in Section 8.1.2. in the presence or absence of endogenous PSMA-expressing cells 22Rv1 and LNCaP, or MUC16-expressing OVCAR cells.

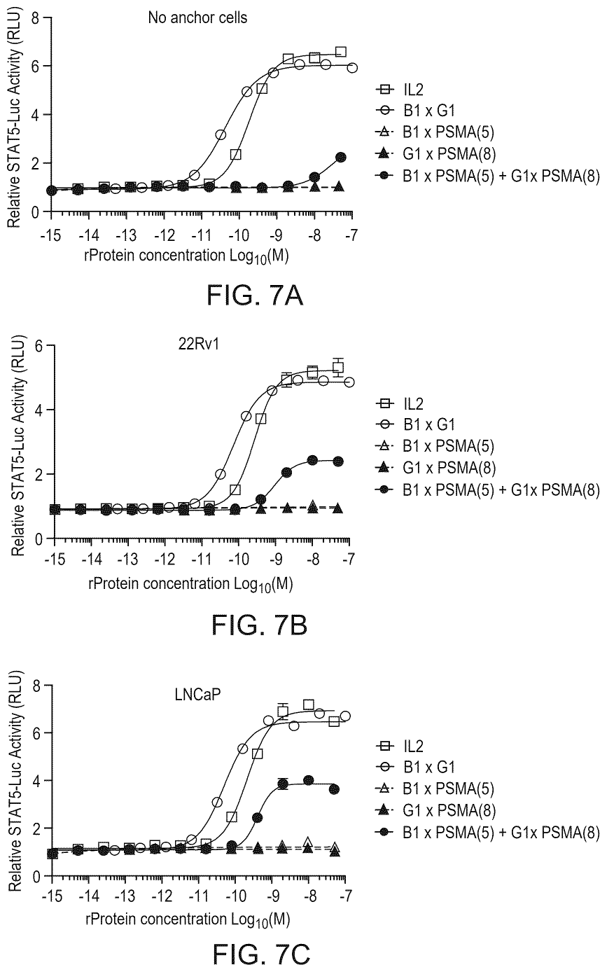

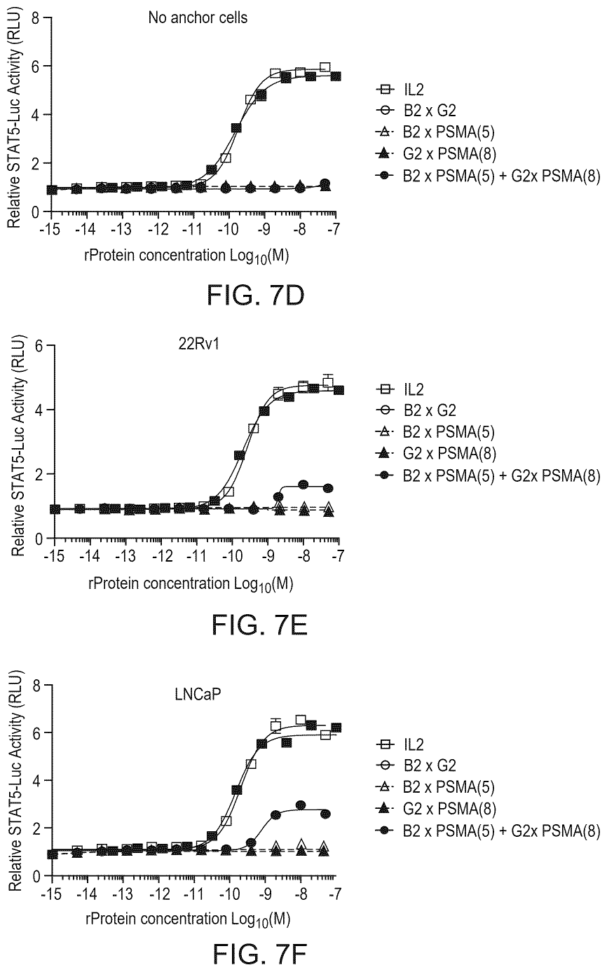

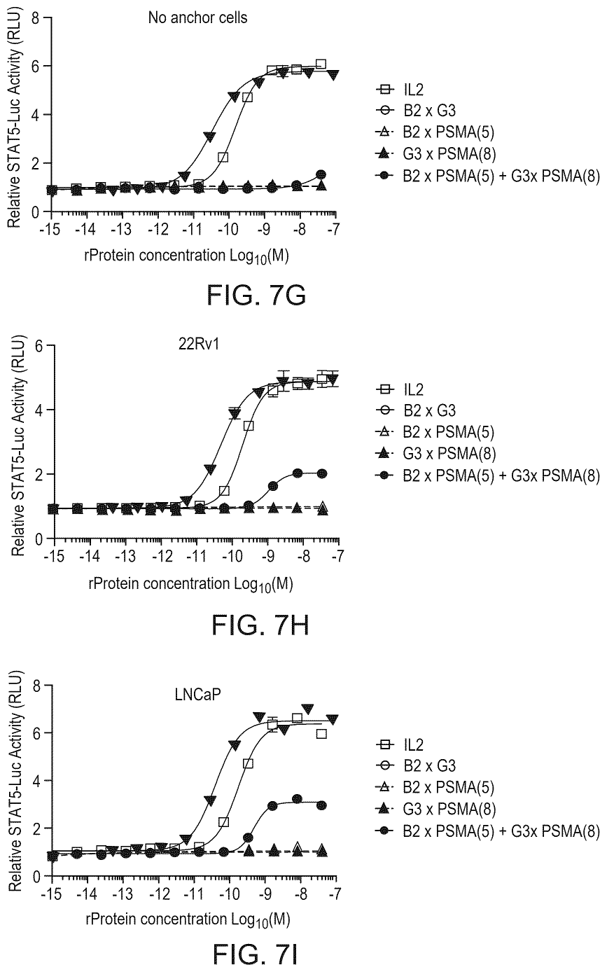

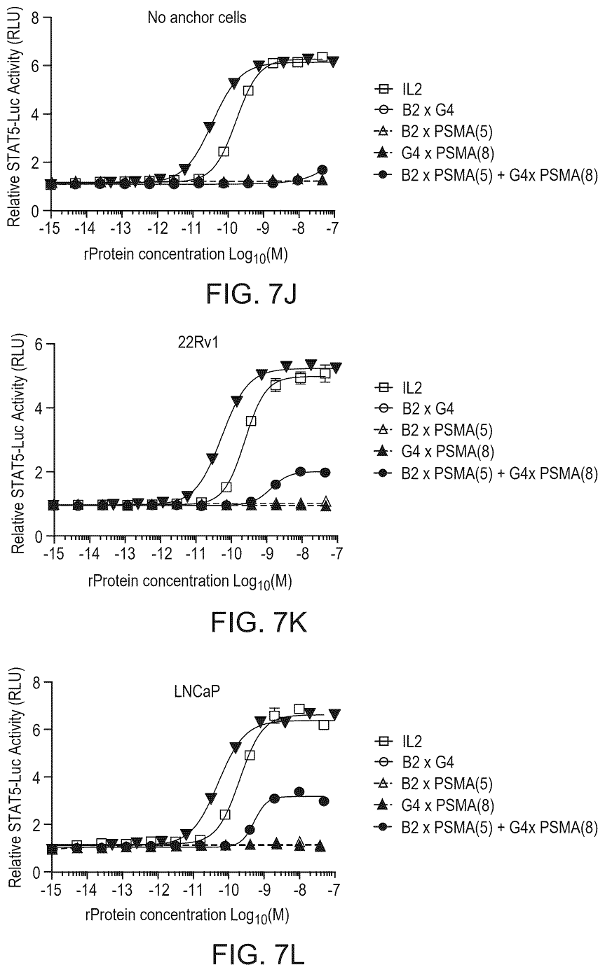

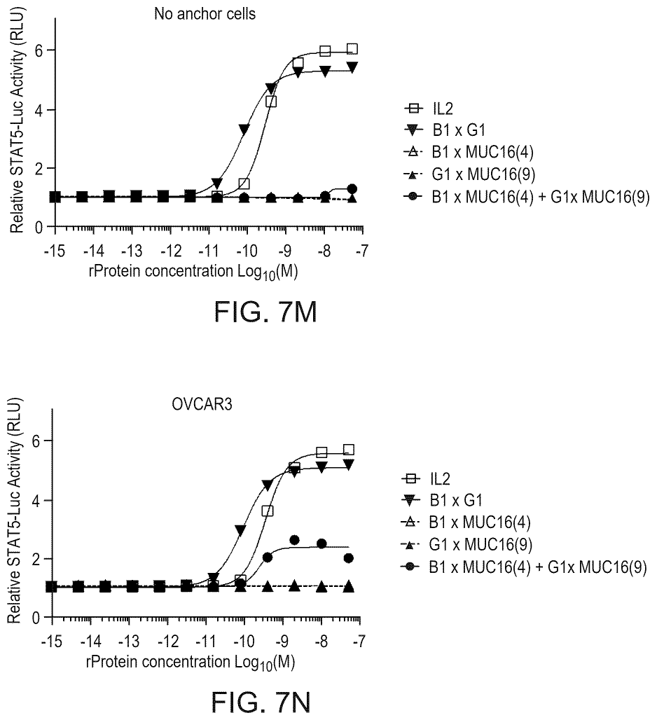

In the first set of assessments, tumor-targeted IL2Rβ binding molecule construct B1×PSMA(5), IL2Rγ binding molecule construct G1×PSMA(8) were evaluated alone or in combination. A bispecific IL2Rβ×IL2Rγ antibody B1×G1 was used as a control. When no PSMA-expressing cells were present, tumor-targeted IL2Rβ and IL2Rγ binding molecules minimally activated STAT5 signaling (FIG. 7A). In the presence of PSMA-expressing 22Rv1 or LNCaP cells, the combination of the tumor-targeted IL2Rβ and IL2Rγ binding molecule constructs activated STAT5 signaling (FIGS. 7B and 7C).

In the second set of assessments, tumor-targeted IL2Rβ binding molecule construct B2×PSMA(5), IL2Rγ binding molecule construct G2×PSMA(8) were evaluated alone or in combination. A bispecific IL2Rβ×IL2Rγ antibody B2×G2 was used as a control. When no PSMA-expressing cells were present, tumor-targeted IL2Rβ and IL2Rγ binding molecules failed to activate STAT5 signaling neither alone nor in combination (FIG. 7D). In the presence of PSMA-expressing 22Rv1 or LNCaP cells, the combination of the tumor-targeted IL2Rβ and IL2Rγ binding molecule constructs activated STAT5 signaling (FIGS. 7E and 7F).

In the third set of assessments, tumor-targeted IL2Rβ binding molecule construct B2×PSMA(5), IL2Rγ binding molecule construct G3×PSMA(8) were evaluated alone or in combination. A bispecific IL2Rβ×IL2Rγ antibody B2×G3 was used as a control. When no PSMA-expressing cells were present, tumor-targeted IL2Rβ and IL2Rγ binding molecules failed to activate STAT5 signaling neither alone nor in combination, except at the highest concentration (FIG. 7G). In the presence of PSMA-expressing 22Rv1 or LNCaP cells, the combination of the tumor-targeted IL2Rβ and IL2Rγ binding molecule constructs activated STAT5 signaling (FIGS. 7H and 7I).

In the fourth set of assessments, tumor-targeted IL2Rβ binding molecule construct B2×PSMA(5), IL2Rγ binding molecule construct G4×PSMA(8) were evaluated alone or in combination. A bispecific IL2Rβ×IL2Rγ antibody B2×G4 was used as a control. When no PMSA-expressing cells were present, tumor-targeted IL2Rβ and IL2Rγ binding molecules failed to activate STAT5 signaling neither alone nor in combination, except at the highest concentration (FIG. 7J). In the presence of PSMA-expressing 22Rv1 or LNCaP cells, the combination of the tumor-targeted IL2Rβ and IL2Rγ binding molecule constructs activated STAT5 signaling (FIGS. 7K and 7L).

Finally, in the fifth set of assessments, tumor-targeted IL2Rβ binding molecule construct B1×MUC16(4), IL2Rγ binding molecule construct G1×MUC16(9) were evaluated alone or in combination. A bispecific IL2Rβ×IL2Rγ antibody B1×G1 was used as a control. When no MUC16-expressing cells were present, tumor-targeted IL2Rβ and IL2Rγ binding molecules failed to activate STAT5 signaling neither alone nor in combination, except at the highest concentration (FIG. 7M). In the presence of MUC16-expressing OVCAR3 cells, the combination of the tumor-targeted IL2Rβ and IL2Rγ binding molecule constructs activated STAT5 signaling (FIG. 7N).

### 8.4. Example 3: STAT5-Phosphorylation Induced by Tumor-Targeted IL2Rβ and IL2Rγ Binding Molecules

The ability of a combination of tumor-targeted IL2Rβ and IL2Rγ binding molecules to induce STAT5-phosphorylation in different human T cell populations was assessed with a pSTAT5 assay as described in Section 8.1.3.

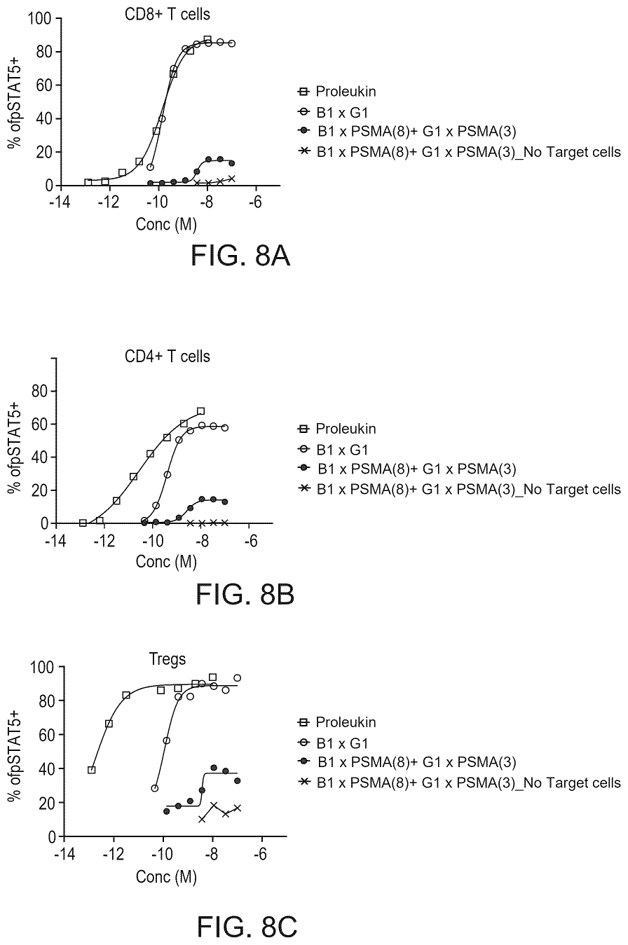

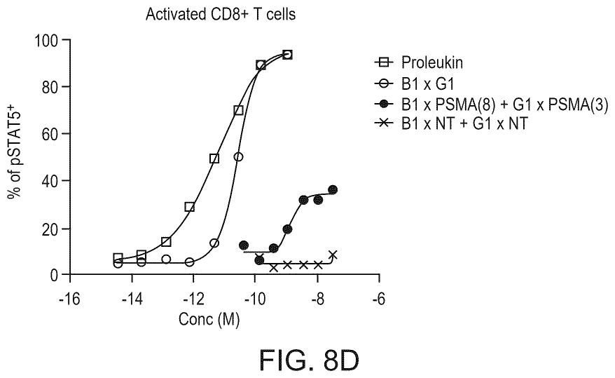

In the presence of C4-2 tumor cells, a combination of tumor-targeted IL2Rβ and IL2Rγ binding molecules led to STAT5-phosphorylation in unstimulated CD8+ T-cells, CD4+ T-cells and Tregs, respectively (FIGS. 8A-8D). However, when no tumor cells were present, the same combination of tumor-targeted IL2Rβ and IL2Rγ binding molecules failed to induce STAT5-phosphorylation in these T cell populations (FIGS. 8A-8D). In the presence of C4-2 tumor cells, a combination of tumor-targeted IL2Rβ and IL2Rγ binding molecules led to STAT5-phosphorylation in activated CD8+ T-cells (FIG. 8D), whereas a combination of non-targeted controls bearing the same IL2Rβ and IL2Rγ binding domains failed to induce STAT5-phosphorylation.

### 8.5. Example 4: Target Cell Killing and IFNγ Release by Tumor-Targeted IL2Rβ and IL2Rγ Binding Molecules

The ability of a combination of tumor-targeted IL2Rβ and IL2Rγ binding molecules to elicit target cell killing was assessed with an in vitro target cell killing assay as described in Section 8.1.4. IFNγ release induced by tumor-targeted IL2Rβ and IL2Rγ binding molecules was assessed with the IFNγ release assay described in Section 8.1.4.

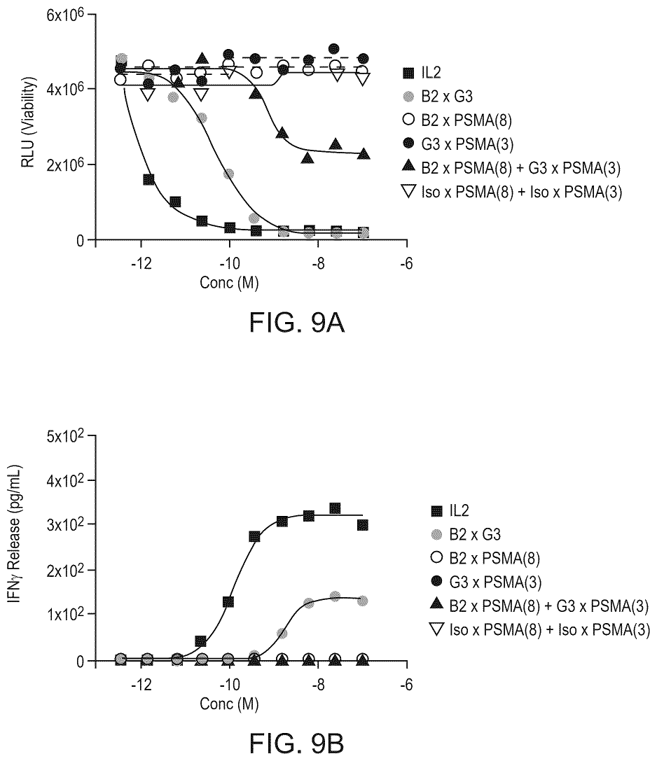

A tumor-targeted IL2Rβ binding molecule (B2×PSMA(8)) alone, a tumor-targeted IL2Rγ binding molecule (G3×PSMA(3)) alone, and a combination of both tumor-targeted IL2Rβ and IL2Rγ binding molecules (B2×PSMA(8) and G3×PSMA(3)) were evaluated in comparison to the bispecific antibody B2×G3, and human IL2 (Proleukin), using activated CD8+ T-cells. In this case, the tumor-targeted IL2Rβ binding molecule and the tumor-targeted IL2Rγ binding molecule alone did not induce target cell killing; however, the combination of the tumor-targeted IL2Rβ and IL2Rγ binding molecules induced target cell killing (FIG. 9A). The combination of the tumor-targeted IL2Rβ and IL2Rγ binding molecules did not induce IFNγ release (FIG. 9B).

### 8.6. Example 5: The Effect of Tumor Target Expression Levels on STAT5-Signaling Induced by Tumor-Targeted IL2Rβ and IL2Rγ Binding Molecules

Tumor-targeted IL2Rβ and IL2Rγ binding molecules were designed and produced as described in Section 8.1.1. Tumor targeting moieties of the tumor-targeted IL2Rβ and IL2Rγ binding molecule constructs comprised anti-HER2 Fabs. The ability of a combination of tumor-targeted IL2Rβ and IL2Rγ binding molecules to activate STAT5-signaling was assessed in a STAT5-reporter cell-based assay as described in Section 8.1.2. in the presence of endogenous HER2-expressing cells NCI-N87 cells, JIMT-1 cells, or NCI-H292 cells.

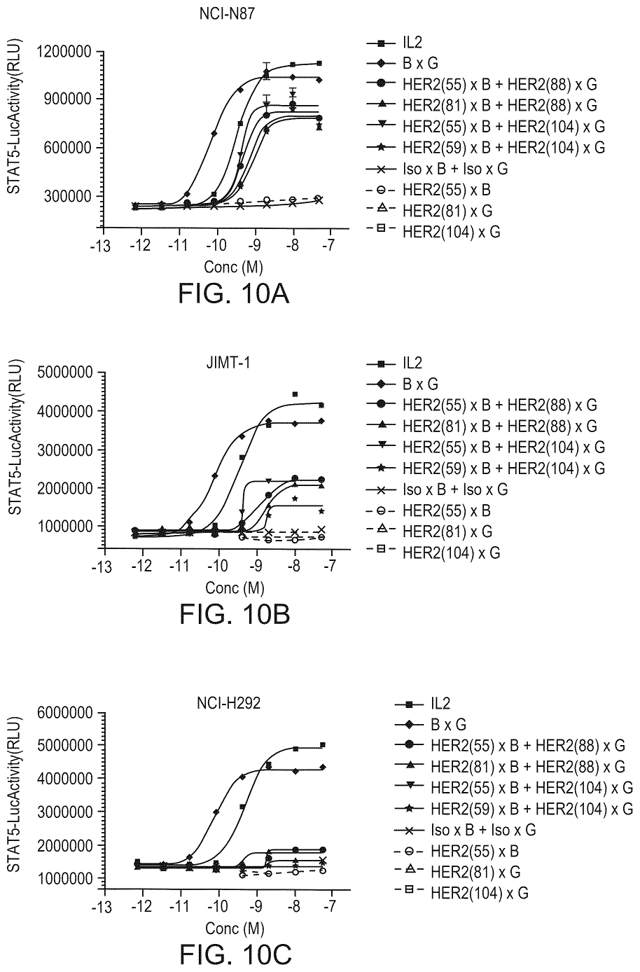

NCI-N87 cells express high levels of HER2 relative to JIMT-1 cells, whereas NCI-H292 cells express relatively low levels of HER2. In the presence of NCI-N87 cells, the combinations of the HER2-targeted IL2Rβ and IL2Rγ binding molecule constructs were associated with robust activation of STAT5 signaling (FIG. 10A). A less robust activation of STAT5 signaling by HER2-targeted IL2Rβ and IL2Rγ binding molecule constructs was observed in the presence of JIMT-1 cells (FIG. 10B). Low levels of STAT5 signaling by HER2-targeted IL2Rβ and IL2Rγ binding molecule constructs was observed in the presence of NCI-H292 cells (FIG. 10C).

### 8.7. Example 6: STAT5-Signaling Induced by Tumor-Targeted IL2Rβ and IL2Rγ Binding Molecules with Different Tumor Targeting Moieties

Tumor-targeted IL2Rβ and IL2Rγ binding molecules were designed and produced as described in Section 8.1.1. Tumor targeting moieties of the tumor-targeted IL2Rβ molecule constructs comprised anti-EGFR Fabs and IL2Rγ binding molecule constructs comprised either anti-EGFR Fabs or anti-HER2 Fabs. The ability of a combination of tumor-targeted IL2Rβ and IL2Rγ binding molecules to activate STAT5-signaling was assessed in a STAT5-reporter cell-based assay as described in Section 8.1.2. in the presence of endogenous EGFR- and HER2-expressing cells JIMT-1 cells or NCI-H292 cells.

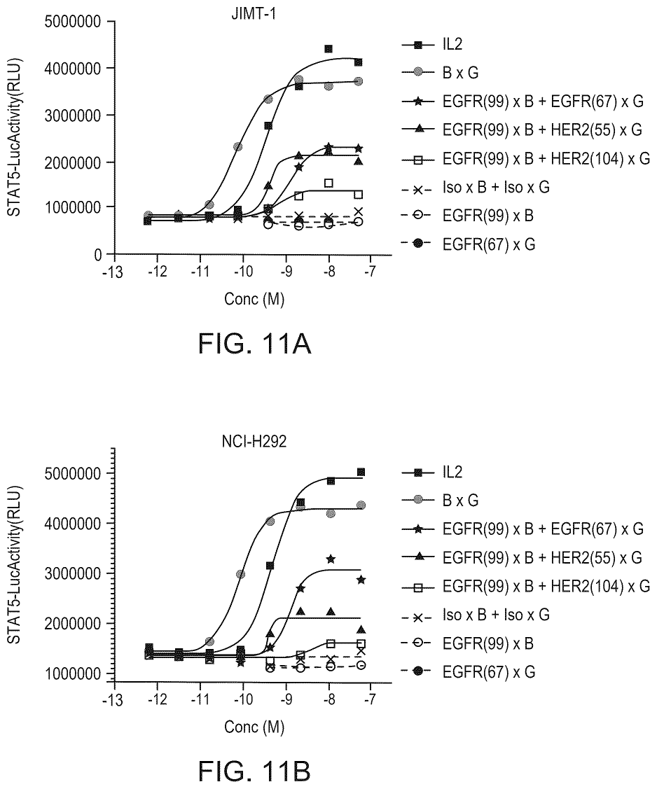

In the presence of JIMT-1 cells, the combinations EGFR(99)×B+EGFR(67)×G and EGFR(99)×B+HER2 (55)×G were associated with comparable levels of STAT5-reporter activity (FIG. 11A). Using NCI-H292 cells, which express higher levels of EGFR and lower levels of HER2 relative to JIMT1 cells, instead of JIMT-1 cells improved the performance of the combination EGFR(99)×B+EGFR(67)×G but not the other combinations EGFR(99)×B+HER2 (55)×G and EGFR(99)×B+HER2 (104)×G (FIG. 11B).

### 8.8. Example 7: Effect of the Tumor Targeting Moiety Format on STAT5-Signaling Induced by Tumor-Targeted IL2Rβ and IL2Rγ Binding Molecules

Tumor-targeted IL2Rβ and IL2Rγ binding molecules were designed and produced as described in Section 8.1.1. Tumor targeting moieties of the tumor-targeted IL2Rβ and IL2Rγ molecule constructs comprised either anti-PSMA Fabs or anti-PSMA scFvs. The ability of a combination of tumor-targeted IL2Rβ and IL2Rγ binding molecules to activate STAT5-signaling was assessed in a STAT5-reporter cell-based assay as described in Section 8.1.2. The following constructs were evaluated alone or in combinations: PSMA(1)×B, which comprises a PSMA-targeting moiety comprising a PSMA(5) Fab and an IL2Rβ-binding moiety comprising a B1 sdAb; PSMA(2)×G, which comprises a PSMA-targeting moiety comprising a PSMA(8) Fab and an IL2Rβ-binding moiety comprising a G1 sdAb; PSMA(1)scFv×B, which comprises a PSMA-targeting moiety comprising a PSMA(5) scFv and an IL2Rβ-binding moiety comprising a B1 sdAb; and PSMA(2)scFv×G, which comprises a PSMA-targeting moiety comprising a PSMA(8) scFv and an IL2Rβ-binding moiety comprising a G1 sdAb. IL2 (Proleukin) and B×G (B1×G1 bispecific antibody) were used as controls.

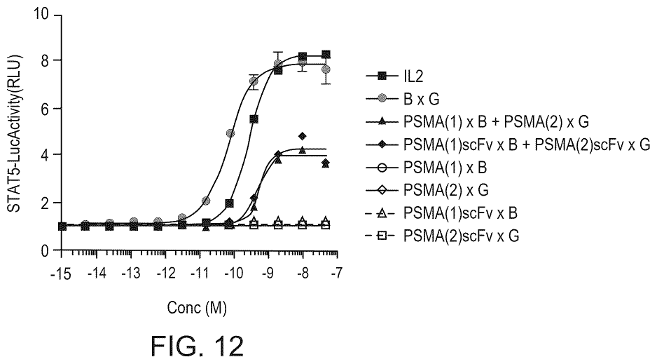

PSMA(1)Fab×B+ and PSMA(2)Fab×G and PSMA(1)scFv×B+ and PSMA(2)scFv×G were associated with comparable STAT5-reporter activity (FIG. 12).

### 8.9. Example 8: STAT5-Phosphorylation Induced by Tumor-Targeted IL2Rβ and IL2Rγ Binding Molecules with Different Tumor Targeting Moieties

Tumor-targeted IL2Rβ and IL2Rγ binding molecules were designed and produced as described in Section 8.1.1. Tumor targeting moieties of the tumor-targeted IL2Rβ and IL2Rγ molecule constructs comprised anti-EGFR, anti-MSLN, or anti-STEAP1 Fabs. The ability of a combination of tumor-targeted IL2Rβ and IL2Rγ binding molecules to activate STAT5-signaling was assessed in a STAT5-reporter cell-based assay as described in Section 8.1.2. in the presence of endogenous EGFR-expressing A431 cells (for anti-EGFR constructs), MSLN-expressing PEO1 cells (for anti-MSLN constructs), or STEAP1-expressing C4-2 cells (for anti-STEAP1 constructs).

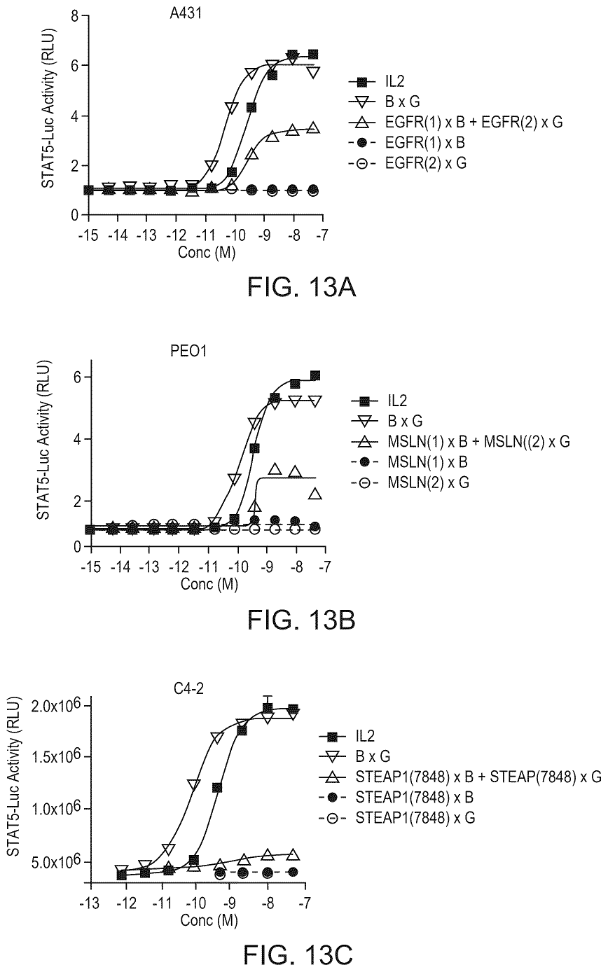

In the presence of A431 cells, the combination of the EGFR-targeted IL2Rβ and IL2Rγ binding molecule constructs was associated with robust activation of STAT5 signaling, while the EGFR-targeted IL2Rβ binding molecule or the EGFR-targeted IL2Rγ binding molecule alone did not activate STAT5 signaling (FIG. 13A). Similarly, the combination of MSLN-targeted IL2Rβ and IL2Rγ binding molecule constructs activated STAT5 signaling in PEO1 cells, while the individual molecules alone did not activate STAT5 signaling (FIG. 13B). The combination of STEAP1-targeted IL2Rβ and IL2Rγ binding molecule constructs activated STAT5 signaling in C4-2 cells, while the individual molecules alone showed no STAT5 activation (FIG. 13C).

### 8.10. Example 9: Activation of STAT5 Phosphorylation and Target Cell Killing by Additional Tumor-Targeted IL2Rβ and IL2Rγ Binding Molecules with Different Formats

Additional tumor targeted IL2Rβ and IL2Rγ binding molecules were designed. First, a new tumor-targeted IL2Rγ binding molecule was designed as depicted in FIG. 3A to comprise an anti-PSMA Fab and an anti-IL2Rγ Fab. This anti-PSMA targeted IL2Rγ binding molecule was used in combination with a new anti-PSMA targeted IL2Rβ binding molecule with single domain anti-IL2Rβ antibody arms (Combination B in FIG. 14A).

The ability of combinations of tumor-targeted IL2Rβ and IL2Rγ binding molecules with different formats to activate STAT5 phosphorylation was assessed in a pSTAT5 assay as described in Section 8.1.3 in the presence of C4-2 tumor cells. The ability of a combination of tumor-targeted IL2Rβ and IL2Rγ binding molecules to elicit target cell killing was assessed with an in vitro target cell killing assay as described in Section 8.1.4. The evaluated combinations are depicted in FIG. 14A, which are:
Combination A: anti-PSMA(5) Fab×anti-IL2Rβ (B1) sdAb+anti-PSMA(10) Fab×anti-IL2Rγ (G1) sdAb; and
Combination B: anti-PSMA(5) Fab×anti-IL2Rβ (B6) sdAb+anti-PSMA(10) Fab×anti-IL2Rγ (G5) Fab

STAT5 signaling and target cell killing was evaluated. Combination B and Combination A were both associated with higher pSTAT5 levels than was achieved with either single molecule alone (FIG. 14B). Further, both combinations reduced the percentage of surviving cells (FIG. 14C).

### 8.11. Example 10: In Vivo Assessments of Tumor-Targeted IL2Rβ and IL2Rγ Binding Molecules

Human PBMC pre-engrafted mice were inoculated with tumor ascites cells, randomized into treatment groups and i.p. injected with assigned IL2Rβ and IL2Rγ binding molecules or control constructs either together with or without an MSLN×CD3 bispecific antibody as described in Section 8.1.5. Tumor burden and body weight change were assessed twice per week for the duration of assessments. T cell expansion was evaluated in blood samples collected on Day 14.

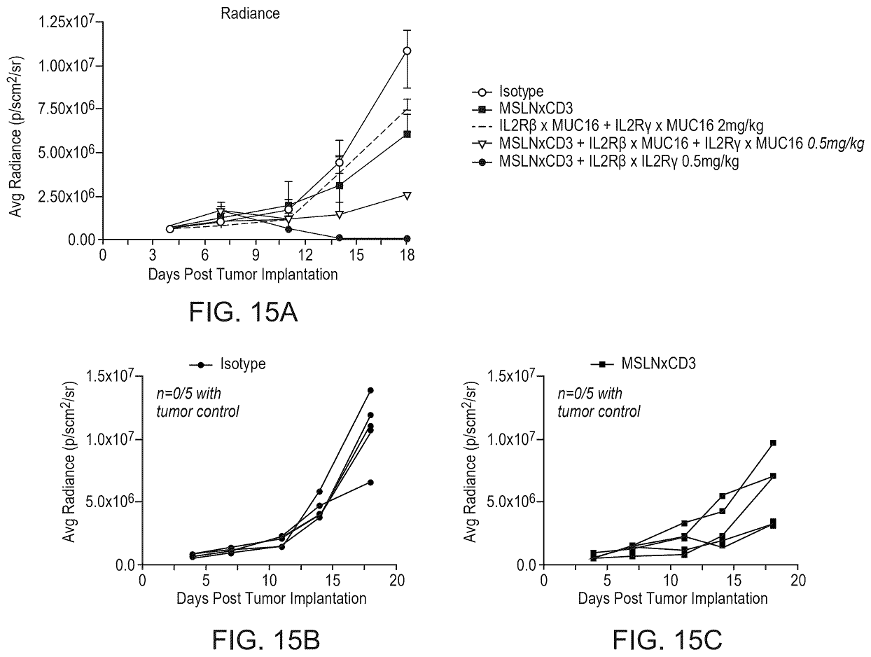

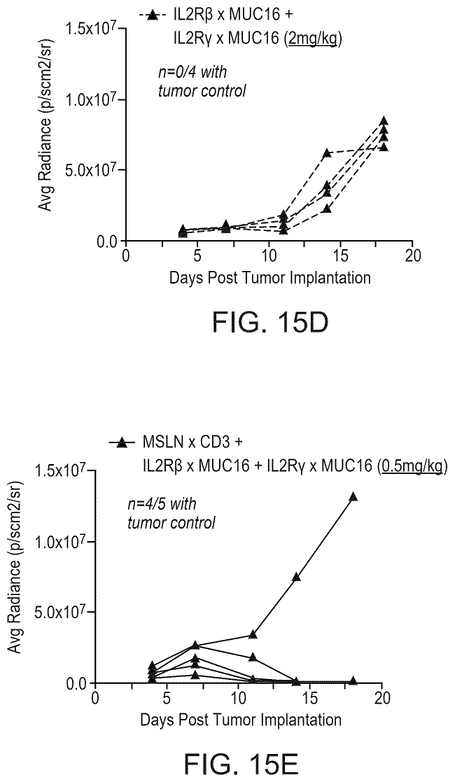

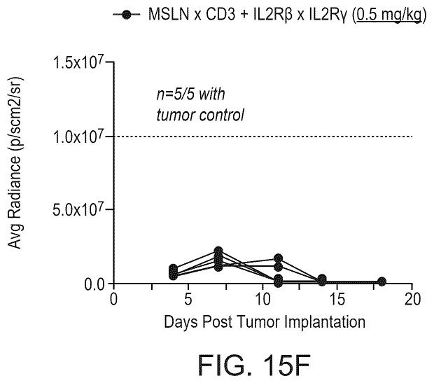

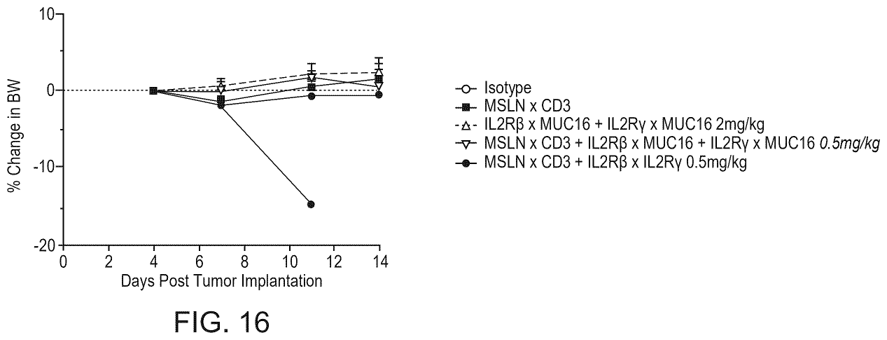

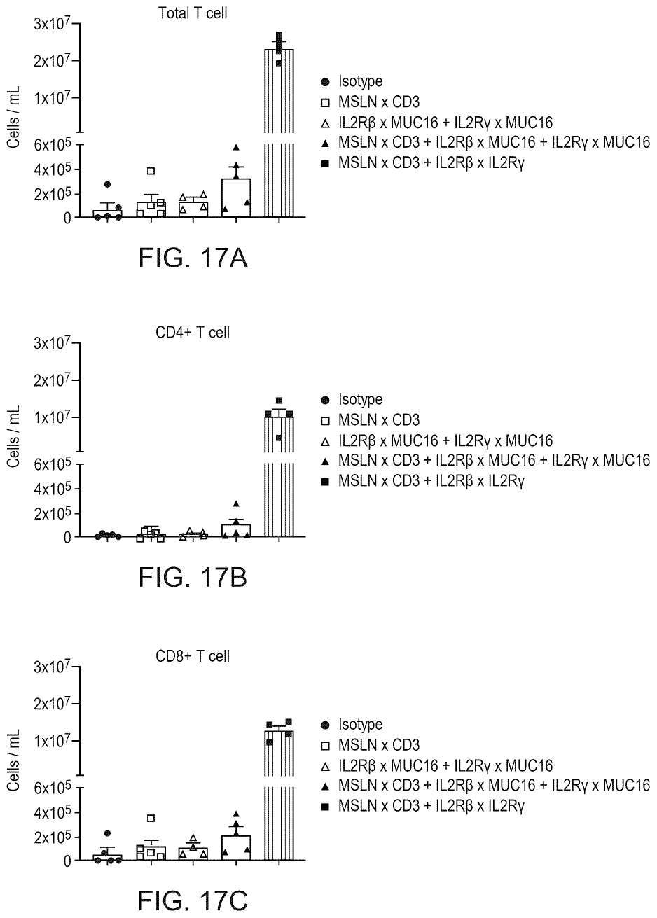

Combinations of tumor-targeted IL2Rβ and IL2Rγ binding molecules were evaluated against control treatments (FIGS. 15A-15F). MSLN×CD3 bispecific antibody alone and the combination of IL2Rβ×MUC16 and IL2Rγ×MUC16 without MSLN×CD3 bispecific antibody were each associated with little to no anti-tumor activity in this model (FIGS. 15A, 15C, and 15D). Treatment of mice with IL2Rβ and IL2Rγ binding molecules together with MSLN×CD3 bispecific antibody was associated with anti-tumor activity (FIG. 15E). None of the treatments (except MSLN×CD3 combined with IL2Rβ×IL2Rγ) led to acute loss of body weight and mortality (FIG. 16) or massive systemic T cell expansion (FIGS. 17A-17C). By contrast, treatment with MSLN×CD3 combined with a bispecific IL2Rβ×IL2Rγ antibody led to significant weight loss and mortality (FIG. 16) and systemic T cell expansion (FIGS. 17A-17C), highlighting the significantly improved safety profile of tumor-targeted split IL2 receptor agonists relative to a systemic IL2 agonist.

### 8.12. Example 11: Assessment of Cross-Competition of PSMA-Targeting Antibodies

Cross-competition of PSMA-binding antibodies was assessed with the Octet assay described in Section 8.1.6. The results of this assessment are set forth in Table E3 below which shows responses for each combination of antibody (indicated in italics), where the “first test antibody” is indicated in the first column and the “second test antibody” is indicated in the second row. Target-specific antibodies having a response of less than 0.2 were determined to be “competing” antibodies, while those having a response of 0.6 or greater were determined to be “non-competing” antibodies.

The results indicated the following:
PSMA(3) and PSMA(5) were determined to be competing binders;
PSMA(8) and PSMA(10) were determined to be competing binders;
PSMA(3) and PSMA(8) were determined to be non-competing binders;
PSMA(3) and PSMA(10) were determined to be non-competing binders;
PSMA(5) and PSMA(8) were determined to be non-competing binders; and
PSMA(5) and PSMA(10) were determined to be non-competing binders.

TABLE E3
Response to 50 μg/mL of second test antibody competing
with first test antibody bound to hPSMA.mmh

| Anti- | hPSMA.mmh | 50 μg/mL | | | | | |
|---|---|---|---|---|---|---|---|
| PSMA | Capture | first test | | | | | Isotype |
| Antibody | Level (nm) | antibody | PMSA(8) | PMSA(10) | PMSA(5) | PMSA(3) | control |
| PMSA(8) | 0.49 ± 0.05 | 0.53 ± 0.02 | 0 | 0.1 | 0.7 | 0.7 | 0 |
| PMSA(10) | 0.48 ± 0.05 | 0.50 ± 0.02 | 0 | 0 | 0.6 | 0.7 | 0.02 |
| PMSA(5) | 0.52 ± 0.05 | 0.73 ± 0.02 | 0.6 | 0.7 | 0 | 0 | 0 |
| PMSA(3) | 0.50 ± 0.04 | 0.68 ± 0.03 | 0.6 | 0.69 | 0 | 0 | 0.03 |
| Isotype | 0.47 ± 0.05 | 0.01 ± 0.01 | 0.5 | 0.53 | 0.7 | 0.6 | 0 |
| control | | | | | | | |

### 8.13. Example 12: Activation of STAT5-Signaling by Combinations of IL2Rβ and IL2Rγ Binding Molecules Comprising Competing or Non-Competing Anti-PSMA Fabs

To determine the effect of competing or non-competing PSMA targeting moieties in combinations of tumor-targeted IL2Rβ and IL2Rγ binding molecules, STAT5-signaling was assessed in a STAT5-reporter cell-based assay as described in Section 8.1.2.

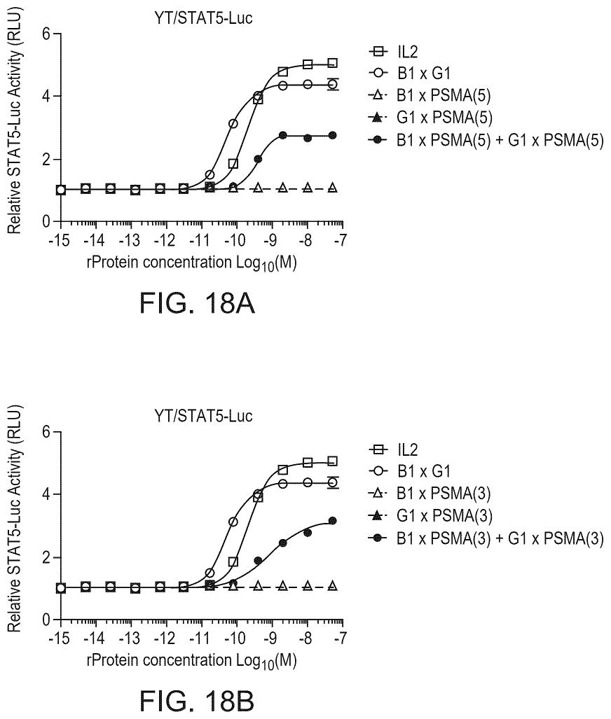

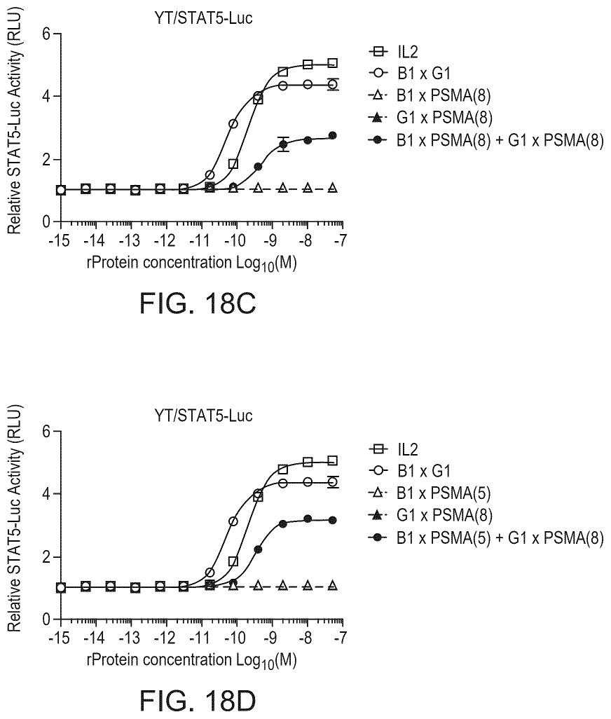

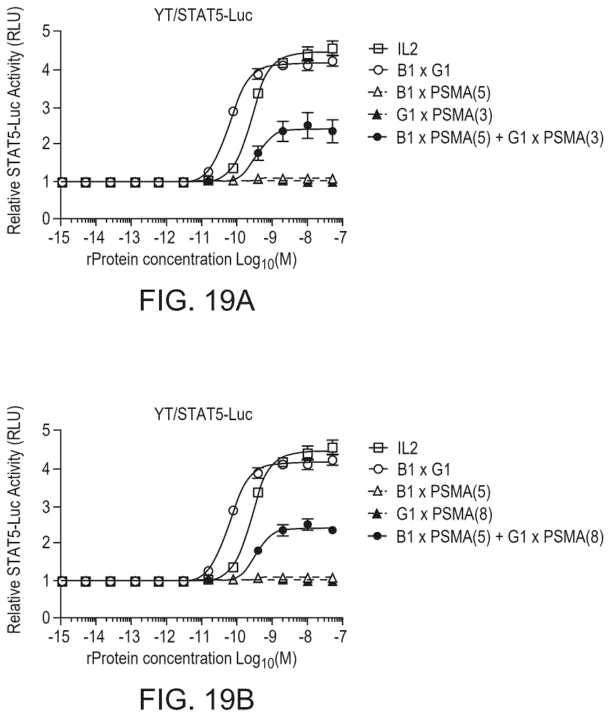

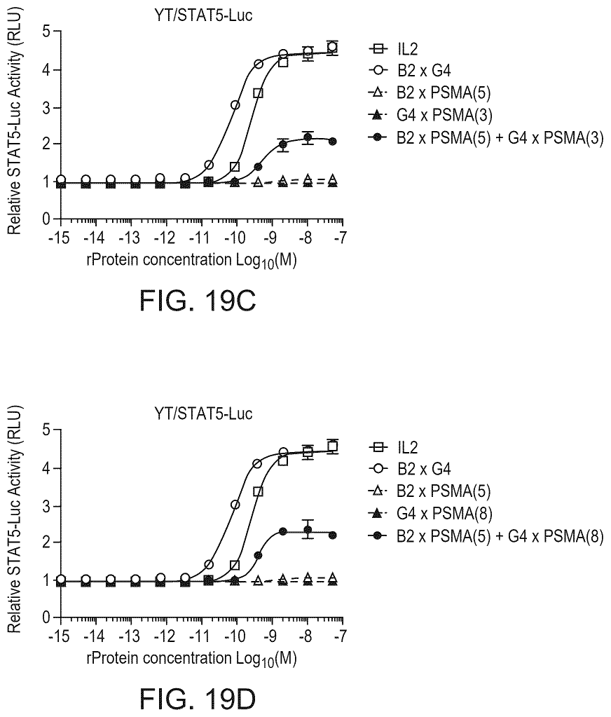

Tumor-targeted IL2Rβ and IL2Rγ binding molecules comprising the same PSMA targeting moieties were evaluated alone and in combination. No STAT5 activation was observed for each tumor-targeted IL2Rβ and IL2Rγ binding molecule when administered alone. Activation of STAT5 signaling was observed for IL2Rβ binding molecule construct B1×PSMA(5) and IL2Rγ binding molecule construct G1×PSMA(5) (FIG. 18A); IL2Rβ binding molecule construct B1×PSMA(3) and IL2Rγ binding molecule construct G1×PSMA(3) (FIG. 18B); and IL2Rβ binding molecule construct B1×PSMA(8) and IL2Rγ binding molecule construct G1×PSMA(8) (FIG. 18C). Next, the combination of tumor-targeted IL2Rβ and IL2Rγ binding molecules comprising non-competing PSMA targeting moieties was evaluated. Activation of STAT5 signaling was also observed for the binding molecules comprising the non-competing PSMA targeting moieties (FIG. 18D). STAT5 signaling activation of combinations of tumor-targeted IL2Rβ and IL2Rγ binding molecules (combination of B1 and G1 or combination of B2 and G4), comprising competing PSMA targeting moieties (FIGS. 19A and 19C), was compared to their counterparts comprising non-competing PSMA targeting moieties (FIGS. 19B and 19D). STAT5 signaling activation was observed in all combinations tested. Table E4, below, provides EC50, Min, and Max values for certain results depicted in FIGS. 19C and 19D.

TABLE E4

| | | | | Fold |
|---|---|---|---|---|
| | | | | change |
| | | | | (Max/Min |
| | EC50 | Min | Max | signal) |
| IL2 | 2.566E−10 | 3.596E+05 | 1.685E+06 | 4.7 |
| B2 × G4 | 6.720E−11 | 3.774E+05 | 1.715E+06 | 4.5 |
| B2 × PSMA(5) + | 3.936E−10 | 3.678E+05 | 9.098E+05 | 2.5 |
| G4 × PSMA(8) | | | | |
| B2 × PSMA(5) + | 5.708E−10 | 3.502E+05 | 7.701E+05 | 2.2 |
| G4 × PSMA(3) | | | | |

### 9. SEQUENCE LISTING

Exemplary sequences of the present disclosure are provided in Table S below (with the column “SEQ” indicating the SEQ ID NO:).

TABLE S

| SEQ | DESCRIPTION | SEQUENCE |
|---|---|---|
| 1 | WT full length | MDSYLLMWGLLTFIMVPGCQAELCDDDPPEIPHATFKAMAYKEG |
| | human IL2Ra | TMLNCECKRGFRRIKSGSLYMLCTGNSSHSSWDNQCQCTSSAT |
| | Uniprot Accession | RNTTKQVTPQPEEQKERKTTEMQSPMQPVDQASLPGHCREPPP |
| | no. P01589 | WENEATERIYHFVVGQMVYYQCVQGYRALHRGPAESVCKMTHG |
| | (extracellular | KTRWTQPQLICTGEMETSQFPGEEKPQASPEGRPESETSCLVTT |
| | domain | TDFQIQTEMAATMETSIFTTEYQVAVAGCVFLLISVLLLSGLTW |
| | corresponds to | QRRQRKSRRTI |
| | amino acids 22-272 | |
| 2 | WT full length | MAAPALSWRLPLLILLLPLATSWASAAVNGTSQFTCFYNSRANIS |
| | human IL2Rβ | CVWSQDGALQDTSCQVHAWPDRRRWNQTCELLPVSQASWACNL |
| | Uniprot Accession | ILGAPDSQKLTTVDIVTLRVLCREGVRWRVMAIQDFKPFENLRLM |
| | no. P14784 | APISLQVVHVETHRCNISWEISQASHYFERHLEFEARTLSPGHTW |
| | (extracellular | EEAPLLTLKQKQEWICLETLTPDTQYEFQVRVKPLQGEFTTWSP |
| | domain | WSQPLAFRTKPAALGKDTIPWLGHLLVGLSGAFGFIILVYLLINC |
| | corresponds to | RNTGPWLKKVLKCNTPDPSKFFSQLSSEHGGDVQKWLSSPFPSSS |
| | amino acids 24- | FSPGGLAPEISPLEVLERDKVTQLLLQQDKVPEPASLSSNHSLTS |
| | 240) | CFTNQGYFFFHLPDALEIEACQVYFTYDPYSEEDPDEGVAGAPTG |
| | | SSPQPLQPLSGEDDAYCTFPSRDDLLLFSPSLLGGPSPPSTAPG |
| | | GSGAGEERMPPSLQERVPRDWDPQPLGPPTPGVPDLVDFQPPP |
| | | ELVLREAGEEVPDAGPREGVSFPWSRPPGQGEFRALNARLPLNT |
| | | DAYLSLQELQGQDPTHLV |
| 3 | WT full length | MLKPSLPFTSLLFLQLPLLGVGLNTTILTPNGNEDTTADFFLTTM |
| | human IL2Rγ | PTDSLSVSTLPLPEVQCFVFNVEYMNCTWNSSSEPQPTNLTLHYWY |
| | Uniprot Accession | KNSDNDKVQKCSHYLFSEEITSGCQLQKKEIHLYQTFVVQLQDPR |
| | no. P31785 | EPRRQATQMLKLQNLVIPWAPENLTLHKLSESQLELNWNNRFLN |
| | (extracellular | HCLEHLVQYRTDWDHSWTEQSVDYRHKFSLPSVDGQKRYTFRV |
| | domain | RSRFNPLCGSAQHWSEWSHPIHWGSNTSKENPFLFALEAVVISV |
| | corresponds to | GSMGLIISLLCVYFWLERTMPRIPTLKNLEDLVTEYHGNFSAWSG |
| | amino acids 23- | VSKGLAESLQPDYSERLCLVSEIPPKGGALGEGPGASPCNQHSP |
| | 262) | YWAPPCYTLKPET |
| 4 | WT mature human | APTSSSTKKTQLQLEHLLLDLQMILNGINNYKNPKLTRMLTFKFYM |
| | IL2 | PKKATELKHLQCLEEELKPLEEVLNLAQSKNFHLRPRDLISNINVI |
| | | VLELKGSETTFMCEYADETATIVEFLNRWITFCQSIISTLT |
| 5 | hIgG1 Fc | EPKSCDKTHTCPPCPAPELLGGPSVFLFPPKPKDTLMISRTPEVT |
| | (amino acids 99- | CVVVDVSHEDPEVKFNWYVDGVEVHNAKTKPREEQYNSTYRVV |
| | 330 of UniprotKB | SVLTVLHQDWLNGKEYKCKVSNKALPAPIEKTISKAKGQPREPQV |
| | P01857-1) | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGK |
| 6 | hIgG2 Fc | ERKCCVECPPCPAPPVAGPSVFLFPPKPKDTLMISRTPEVTCVVV |
| | (amino acids 99- | DVSHEDPEVQFNWYVDGVEVHNAKTKPREEQFNSTFRVVSVLTV |
| | 326 of UniprotKB | VHQDWLNGKEYKCKVSNKGLPAPIEKTISKTKGQPREPQVYTLPP |
| | P01859-1) | SREEMTKNQVSLTCLVKGFYPSDISVEWESNGQPENNYKTTPPM |
| | | LDSDGSFFLYSKLTVDKSRWQQGNVFSCSVMHEALHNHYTQKSL |
| | | SLSPGK |
| 7 | hIgG3 Fc | ELKTPLGDTTHTCPRCPEPKSCDTPPPCPRCPEPKSCDTPPPCP |
| | (amino acids 99- | RCPEPKSCDTPPPCPRCPAPELLGGPSVFLFPPKPKDTLMISRTP |
| | 377 of UniprotKB | EVTCVVVDVSHEDPEVQFKWYVDGVEVHNAKTKPREEQYNSTF |
| | P01860-1) | RVVSVLTVLHQDWLNGKEYKCKVSNKALPAPIEKTISKTKGQPRE |
| | | PQVYTLPPSREEMTKNQVSLTCLVKGFYPSDIAVEWESSGQPEN |
| | | NYNTTPPMLDSDGSFFLYSKLTVDKSRWQQGNIFSCSVMHEALH |
| | | NRFTQKSLSLSPGK |
| 8 | hIgG4 Fc | ESKYGPPCPSCPAPEFLGGPSVFLFPPKPKDTLMISRTPEVTCVV |
| | (amino acids 99- | VDVSQEDPEVQFNWYVDGVEVHNAKTKPREEQFNSTYRVVSVL |
| | 327 of UniprotKB | TVLHQDWLNGKEYKCKVSNKGLPSSIEKTISKAKGQPREPQVYTL |
| | P01861-1) | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQ |
| | | KSLSLSLGK |
| 9 | Human IgG4s Fc | ESKYGPPCPPCPAPPVAGPSVFLFPPKPKDTLMISRTPEVTCVVV |
| | Variant hIgG4 | DVSQEDPEVQFNWYVDGVEVHNAKTKPREEQFNSTYRVVSVLT |
| | hinge and Fc, with | VLHQDWLNGKEYKCKVSNKGLPSSIEKTISKAKGQPREPQVYTLP |
| | IgG2-based hinge | PSQEEMTKNQVSLTCLVKGFYPSDIAVEWESNGQPENNYKTTPP |
| | region with S108P | VLDSDGSFFLYSRLTVDKSRWQEGNVFSCSVMHEALHNHYTQK |
| | mutation (S228P by | SLSLSLGK |
| | EU numbering), | |
| | and IgG1 CH2 and | |
| | CH3 | |
| 10 | human IgG1 PVA | EPKSCDKTHTCPPCPAPPVAGPSVFLFPPKPKDTLMISRTPEVT |
| | Variant hIgG1 | CVVVDVSHEDPEVKFNWYVDGVEVHNAKTKPREEQYNSTYRVVS |
| | hinge and Fc, with | VLTVLHQDWLNGKEYKCKVSNKALPAPIEKTISKAKGQPREPQVY |
| | IgG2-based hinge | TLPPSRDELTKNQVSLTCLVKGFYPSDIAVEWESNGQPENNYKTT |
| | region and IgG1 | PPVLDSDGSFFLYSKLTVDKSRWQQGNVFSCSVMHEALHNHYT |
| | CH2 and CH3 | QKSLSLSPGK |
| 11 | hIgG4us Fc | ESKYGPPCPPCPAPGGGGPSVFLFPPKPKDTLMISRTPEVTCVV |
| | | VDVSQEDPEVQFNWYVDGVEVHNAKTKPREEQFNSTYRVVSVL |
| | | TVLHQDWLNGKEYKCKVSNKGLPSSIEKTISKAKGQPREPQVYTL |
| | | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQ |
| | | KSLSLSLGK |
| 12 | hIgG1s Fc | DKKVEPKSCDKTHTCPPCPAPPVAGPSVFLFPPKPKDTLMISRTP |
| | | EVTCVVVDVSQEDPEVQFNWYVDGVEVHNAKTKPREEQFNSTY |
| | | RVVSVLTVLHQDWLNGKEYKCKVSNKGLPSSIEKTISKAKGQPRE |
| | | PQVYTLPPSRDELTKNQVSLTCLVKGFYPSDIAVEWESNGQPEN |
| | | NYKTTPPVLDSDGSFFLYSKLTVDKSRWQQGNVFSCSVMHEALH |
| | | NHYTQKSLSLSPGK |
| 13 | hIgG1us Fc | DKKVEPKSCDKTHTCPPCPAPGGGGPSVFLFPPKPKDTLMISRT |
| | | PEVTCVVVDVSQEDPEVQFNWYVDGVEVHNAKTKPREEQFNST |
| | | YRVVSVLTVLHQDWLNGKEYKCKVSNKGLPSSIEKTISKAKGQPR |
| | | EPQVYTLPPSRDELTKNQVSLTCLVKGFYPSDIAVEWESNGQPE |
| | | NNYKTTPPVLDSDGSFFLYSKLTVDKSRWQQGNVFSCSVMHEAL |
| | | HNHYTQKSLSLSPGK |
| 14 | hIgG1 PVA/P329A | EPKSCDKTHTCPPCPAPPVAGPSVFLFPPKPKDTLMISRTPEVT |
| | Fc | CVVVDVSHEDPEVKFNWYVDGVEVHNAKTKPREEQYNSTYRVVS |
| | | VLTVLHQDWLNGKEYKCKVSNKALAAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| 15 | hIgG1 LALAPG Fc | EPKSCDKTHTCPPCPAPEAAGGPSVFLFPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGK |
| 16 | Fc domain (SEQ ID | DKRVESKYGP PCPPCPAPPV AGPSVFLFPP KPKDTLMISR |
| | NO: 1 of | TPEVTCVVVD VSQEDPEVQF NWYVDGVEVH NAKTKPREEQ |
| | WO2014/121087) | FNSTYRVVSV LTVLHQDWLN GKEYKCKVSN KGLPSSIEKT |
| | | ISKAKGQPRE PQVYTLPPSQ EEMTKNQVSL TCLVKGFYPS |
| | | DIAVEWESNG QPENNYKTTP PVLDSDGSFF LYSRLTVDKS |
| | | RWQEGNVFSC SVMHEALHNH YTQKSLSLSL GK |
| 17 | Fc domain (SEQ ID | DKKVEPKSCD KTHTCPPCPA PPVAGPSVFL FPPKPKDTLM |
| | NO: 2 of | ISRTPEVTCV VVDVSQEDPE VQFNWYVDGV EVHNAKTKPR |
| | WO2014/121087) | EEQFNSTYRV VSVLTVLHQD WLNGKEYKCK VSNKGLPSSI |
| | | EKTISKAKGQ PREPQVYTLP PSRDELTKNQ VSLTCLVKGF |
| | | YPSDIAVEWE SNGQPENNYK TTPPVLDSDG SFFLYSKLTV |
| | | DKSRWQQGNV FSCSVMHEAL HNHYTQKSLS LSPGK |
| 18 | Fc domain (SEQ ID | ASTKGPSVFP LAPSSKSTSG GTAALGCLVK DYFPEPVTVS |
| | NO: 30 of | WNSGALTSGV HTFPAVLQSS GLYSLSSVVT VPSSSLGTQT |
| | WO2014/121087) | YICNVNHKPS NTKVDKKVEP KSCDKTHTCP PCPAPPVAGP |
| | | SVFLFPPKPK DTLMISRTPE VTCVVVDVSQ EDPEVQFNWY |
| | | VDGVEVHNAK TKPREEQFNS TYRVVSVLTV LHQDWLNGKE |
| | | YKCKVSNKGL PSSIEKTISK AKGQPREPQV YTLPPSRDEL |
| | | TKNQVSLTCL VKGFYPSDIA VEWESNGQPE NNYKTTPPVL |
| | | DSDGSFFLYS KLTVDKSRWQ QGNVFSCSVM HEALHNHYTQ |
| | | KSLSLSPGK |
| 19 | Fc domain (SEQ ID | ASTKGPSVFP LAPCSRSTSE STAALGCLVK DYFPEPVTVS |
| | NO: 31 of | WNSGALTSGV HTFPAVLQSS GLYSLSSVVT VPSSSLGTKT |
| | WO2014/121087) | YTCNVDHKPS NTKVDKRVES KYGPPCPPCP APPVAGPSVF |
| | | LFPPKPKDTL MISRTPEVTC VVVDVSQEDP EVQFNWYVDG |
| | | VEVHNAKTKP REEQFNSTYR VVSVLTVLHQ DWLNGKEYKC |
| | | KVSNKGLPSS IEKTISKAKG QPREPQVYTL PPSQEEMTKN |
| | | QVSLTCLVKG FYPSDIAVEW ESNGQPENNY KTTPPVLDSD |
| | | GSFFLYSRLT VDKSRWQEGN VFSCSVMHEA LHNHYTQKSL |
| | | SLSLGK |
| 20 | Fc domain (SEQ ID | ASTKGPSVFP LAPSSKSTSG GTAALGCLVK DYFPEPVTVS |
| | NO: 37 of | WNSGALTSGV HTFPAVLQSS GLYSLSSVVT VPSSSLGTQT |
| | WO2014/121087) | YICNVNHKPS NTKVDKKVEP KSCDKTHTCP PCPAPPVAGP |
| | | SVFLFPPKPK DTLMISRTPE VTCVVVDVSQ EDPEVQFNWY |
| | | VDGVEVHNAK TKPREEQFNS TYRVVSVLTV LHQDWLNGKE |
| | | YKCKVSNKGL PSSIEKTISK AKGQPREPQV YTLPPSRDEL |
| | | TKNQVSLTCL VKGFYPSDIA VEWESNGQPE NNYKTTPPVL |
| | | DSDGSFFLYS KLTVDKSRWQ QGNVFSCSVM HEALHNRFTQ |
| | | KSLSLSPGK |
| 21 | Fc domain (SEQ ID | ASTKGPSVFP LAPCSRSTSE STAALGCLVK DYFPEPVTVS |
| | NO: 38 of | WNSGALTSGV HTFPAVLQSS GLYSLSSVVT VPSSSLGTKT |
| | WO2014/121087) | YTCNVDHKPS NTKVDKRVES KYGPPCPPCP APPVAGPSVF |
| | | LFPPKPKDTL MISRTPEVTC VVVDVSQEDP EVQFNWYVDG |
| | | VEVHNAKTKP REEQFNSTYR VVSVLTVLHQ DWLNGKEYKC |
| | | KVSNKGLPSS IEKTISKAKG QPREPQVYTL PPSQEEMTKN |
| | | QVSLTCLVKG FYPSDIAVEW ESNGQPENNY KTTPPVLDSD |
| | | GSFFLYSRLT VDKSRWQEGN VFSCSVMHEA LHNRFTQKSL |
| | | SLSLGK |
| 22 | hIgG1 N180G, also | EPKSCDKTHTCPPCPAPELLGGPSVFLFPPKPKDTLMISRTPEV |
| | referred to as | TCVVVDVSHEDPEVKFNWYVDGVEVHNAKTKPREEQYGSTYRVV |
| | N297G | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGK |
| 23 | hIgG4 S108P | ESKYGPPCPPCPAPEFLGGPSVFLFPPKPKDTLMISRTPEVTCV |
| | | VVDVSQEDPEVQFNWYVDGVEVHNAKTKPREEQFNSTYRVVSVL |
| | | TVLHQDWLNGKEYKCKVSNKGLPSSIEKTISKAKGQPREPQVYTL |
| | | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQ |
| | | KSLSLSLGK |
| 24 | Linker | SGn |
| 25 | Linker | G4S |
| 26 | Linker | (GGGGS)n |
| 27 | Linker | GGGG |
| 28 | Linker | GGGGG |
| 29 | Linker | GGGGGG |
| 30 | Linker | GGGGGGG |
| 31 | Linker | GGGGGGGG |
| 32 | Linker | GGGGGGGGG |
| 33 | Linker | GnS |
| 34 | Chimeric hinge | EPKSCDKTHTCPPCPAPPVA |
| | region (SEQ ID | |
| | NO: 8 of | |
| | WO2014/121087) | |
| 35 | Chimeric hinge | ESKYGPPCPPCPAPPVA |
| | region (SEQ ID | |
| | NO: 9 of | |
| | WO2014/121087) | |
| 36 | Modified hinge | CPPCPAPGGG-GPSVF |
| 37 | Modified hinge | CPPCPAPGG--GPSVF |
| 38 | Modified hinge | CPPCPAPG---GPSVF |
| 39 | Modified hinge | CPPCPAP----GPSVF |
| 40 | Hinge core | CPPC |
| 41 | Hinge core | CPSC |

What is claimed is:

1. A method comprising administering to a subject:
(a) a tumor-targeted IL2Rβ binding molecule comprising:
(i) a first polypeptide chain comprising:
(1) a first tumor-targeting moiety that binds to a first tumor-associated antigen (TAA), or component thereof (e.g., VH) associated with another component (e.g., VL) on a separate polypeptide chain; and
(2) a first Fc domain having one or more mutations that reduce effector function; and
(ii) a second polypeptide chain comprising:
(1) a single domain antibody (sdAb) that binds to IL2Rβ; and
(2) a second Fc domain that is capable of heterodimerizing with the first Fc domain and having one or more mutations that reduce effector function; and
(b) a tumor-targeted IL2Rγ binding molecule comprising:
(i) a third polypeptide chain comprising:
(1) a second tumor-targeting moiety that binds to a second tumor-associated antigen (TAA) expressed on the same tumor cell as the first tumor-associated antigen, or component thereof (e.g., VH) associated with another component (e.g., VL) on a separate polypeptide chain; and
(2) a third Fc domain having one or more mutations that reduce effector function; and
(ii) a fourth polypeptide chain comprising:
(1) a single domain antibody (sdAb) that binds to IL2Rγ; and
(2) a fourth Fc domain that is capable of heterodimerizing with the third Fc domain and having one or more mutations that reduce effector function.

2. The method of claim 1, wherein the first TAA and the second TAA are different.

3. The method of claim 1, wherein the first TAA and the second TAA are the same.

4. The method of claim 3, wherein the first tumor-targeting moiety and the second tumor-targeting moiety are the same.

5. The method of claim 3, wherein the first tumor-targeting moiety and the second tumor-targeting moiety are different, e.g., bind to different epitopes.

6. The method of claim 3 or claim 5, wherein the first tumor-targeting moiety and the second tumor-targeting moiety are non-competing tumor-targeting moieties (e.g., as determined using an antibody cross-competition assay as described in Section 8.1.6).

7. The method of any one of claims 1 to 6, wherein the first tumor-targeting moiety and/or the second tumor-targeting moiety are Fabs.

8. The method of any one of claims 1 to 6, wherein the first tumor-targeting moiety and/or the second tumor-targeting moiety are scFvs.

9. The method of any one of claims 1 to 6, wherein the first tumor-targeting moiety and/or the second tumor-targeting moiety are sdAbs.

10. The method of any one of claims 1 to 9, wherein the first TAA and/or second TAA is EGFR.

11. The method of any one of claims 1 to 9, wherein the first TAA and/or second TAA is PSMA.

12. The method of any one of claims 1 to 9, wherein the first TAA and/or second TAA is MUC16.

13. The method of any one of claims 1 to 9, wherein the first TAA and/or second TAA is HER2.

14. The method of any one of claims 1 to 9, wherein the first TAA and/or second TAA is STEAP1.

15. The method of any one of claims 1 to 9, wherein the first TAA and/or second TAA is CEACAM5.

16. The method of any one of claims 1 to 15, wherein both the first and second Fc domains are IgG1 Fc domains, IgG2 Fc domains or IgG4 Fc domains.

17. The method of any one of claims 1 to 15, wherein the first Fc domain and second Fc domain each comprises an amino acid sequence having at least 98% sequence identity to the amino acid sequence of SEQ ID NO:5, optionally comprising knob/hole substitutions and/or star mutation(s).

18. The method of any one of claims 1 to 15, wherein the first Fc domain and second Fc domain each comprises an amino acid sequence having at least 98% sequence identity to the amino acid sequence of SEQ ID NO:4, optionally comprising knob/hole substitutions and/or star mutation(s).

19. The method of any one of claims 1 to 15, wherein the first Fc domain and second Fc domain each comprises an amino acid sequence having at least 98% sequence identity to the amino acid sequence of SEQ ID NO:10, optionally comprising knob/hole substitutions and/or star mutation(s).

20. The method of any one of claims 1 to 19, wherein the first and second Fc domains have chimeric hinge domains.

21. The method of any one of claims 1 to 20, wherein the third and fourth Fc domains are IgG1, IgG2 or IgG4 Fc domains.

22. The method of any one of claims 1 to 21, wherein the third Fc domain and fourth Fc domain each comprises an amino acid sequence having at least 98% sequence identity to the amino acid sequence of SEQ ID NO:5, optionally comprising knob/hole substitutions and/or star mutation(s).

23. The method of any one of claims 1 to 21, wherein the third Fc domain and fourth Fc domain each comprises an amino acid sequence having at least 98% sequence identity to the amino acid sequence of SEQ ID NO:8, optionally comprising knob/hole substitutions and/or star mutation(s).

24. The method of any one of claims 1 to 21, wherein the third Fc domain and fourth Fc domain each comprises an amino acid sequence having at least 98% sequence identity to the amino acid sequence of SEQ ID NO:10, optionally comprising knob/hole substitutions and/or star mutation(s).

25. The method of any one of claims 1 to 24, wherein the third and fourth Fc domains have chimeric hinge domains.

26. The method of any one of claims 1 to 24, wherein the tumor-targeted IL2Rβ binding molecule and the tumor-targeted IL2Rγ binding molecule are in the same pharmaceutical composition.

27. The method of any one of claims 1 to 24, wherein the tumor-targeted IL2Rβ binding molecule and the tumor-targeted IL2Rγ binding molecule are in different pharmaceutical compositions.

28. The method of any one of claims 1 to 27, which further comprises administering to the subject a multispecific T-cell engager.

29. The method of claim 28, wherein the multispecific T-cell engager is a bispecific T-cell engager.

30. The method of claim 28 or claim 29, wherein the multispecific T-cell engager comprises a TAA targeting moiety and a CD3 targeting moiety.

31. The method of claim 30, wherein the TAA targeting moiety of the multispecific T-cell engager targets the same TAA as the TAA targeted by the tumor-targeted IL2Rβ binding molecule and/or the tumor-targeted IL2Rγ binding molecule.

32. The method of claim 30, wherein the TAA targeting moiety of the multispecific T-cell engager targets a TAA that is different from the TAA targeted by the tumor-targeted IL2Rβ binding molecule and/or the tumor-targeted IL2Rγ binding molecule.

33. The method of any one of claims 30 to 32, wherein the TAA targeting moiety binds to MSLN.

34. The method of any one of claims 1 to 33, wherein the subject has cancer.

35. The method of claim 34, wherein the cancer is a solid tumor.

36. The method of any one of claims 1 to 35, wherein the administration results in:
(a) prevention or treatment of metastasis;
(b) stimulating the formation, stability and/or activity of a cytotoxic immune synapse;
(c) clustering of IL2Rβ and IL2Rγ receptor subunits in a lymphocyte;
(d) eliciting signaling through the IL2 receptor in a lymphocyte;
(e) inducing tumor cytolysis;
(f) inducing anti-tumor cytotoxicity;
(g) stimulating an immune response against a tumor;
(h) improving the safety of IL2 receptor agonist cancer treatment;
(i) improving the therapeutic window of IL2 receptor agonist cancer treatment; or
(j) any two or more of (a) through (j).


SEQUENCE LISTING (257 sequences)

SEQ ID NO: 1 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 2 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 4 - protein, 133 aa, Homo sapiens [sequence omitted]

SEQ ID NO: 5 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 7 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 8 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 228 aa, Homo sapiens
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 231 aa, Homo sapiens
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 228 aa, Homo sapiens
ESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 235 aa, Homo sapiens
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 235 aa, Homo sapiens
DKKVEPKSCDKTHTCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 231 aa, Homo sapiens
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 232 aa, Homo sapiens
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 232 aa, Homo sapiens
DKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 235 aa, Homo sapiens; identical to SEQ ID NO: 12

SEQ ID NO: 18 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 21 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 22 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 11 aa, synthetic construct
  VARIANT 2..11: note = This region may encompass 1-10 residues
SGGGGGGGGGG

SEQ ID NO: 25 - protein, 5 aa, synthetic construct
GGGGS

SEQ ID NO: 26 - protein, 5 aa, synthetic construct
GGGGS

SEQ ID NO: 27 - protein, 4 aa, synthetic construct
GGGG

SEQ ID NO: 28 - protein, 5 aa, synthetic construct
GGGGG

SEQ ID NO: 29 - protein, 6 aa, synthetic construct
GGGGGG

SEQ ID NO: 30 - protein, 7 aa, synthetic construct
GGGGGGG

SEQ ID NO: 31 - protein, 8 aa, synthetic construct
GGGGGGGG

SEQ ID NO: 32 - protein, 9 aa, synthetic construct
GGGGGGGGG

SEQ ID NO: 33 - protein, 11 aa, synthetic construct
  VARIANT 1..10: note = This region may encompass 1-10 residues
GGGGGGGGGGS

SEQ ID NO: 34 - protein, 20 aa, synthetic construct
EPKSCDKTHTCPPCPAPPVA

SEQ ID NO: 35 - protein, 17 aa, synthetic construct
ESKYGPPCPPCPAPPVA

SEQ ID NO: 36 - protein, 15 aa, synthetic construct
CPPCPAPGGGGPSVF

SEQ ID NO: 37 - protein, 14 aa, synthetic construct
CPPCPAPGGGPSVF

SEQ ID NO: 38 - protein, 13 aa, synthetic construct
CPPCPAPGGPSVF

SEQ ID NO: 39 - protein, 12 aa, synthetic construct
CPPCPAPGPSVF

SEQ ID NO: 40 - protein, 4 aa, synthetic construct
CPPC

SEQ ID NO: 41 - protein, 4 aa, Homo sapiens
CPSC

SEQ ID NO: 42 - protein, 124 aa, synthetic construct
EVQLVESGGGLVQPGRSLRLSCAASGFTFADYTMHWVRQAPGKGLEWVSDISWNSGSIAYADSVKGRFTISRDNAKNSLYLQMNSLRTEDTAFYYCAKDSRGYGHYKYLGLDVWGQGTTVTVSS

SEQ ID NO: 43 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 44 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 45 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 46 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 47 - protein, 15 aa, synthetic construct
GGGGSGGGGSGGGGS

SEQ ID NO: 48 - protein, 348 aa, synthetic construct
QVQLQESGPGLVKPSGTLSLTCAVSGGSISSSDWWSWVRQPPGKGLEWIGEIDHSGSTNYNPSLMSRVTISVDKSKNQFSLKLSSVTAADTAVYFCGRGSWELSDAFDIRGQGTLVTVSSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 49 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 347 aa, synthetic construct
QVQLVESGGGLVKPGGSLRLSCAASGFTFSDYYMSWIRQAPGKGLEWVSSISSSGDTIYYADSVQGRFTLSRDNAENSLFLQMNSLRAEDTAVYYCARGDAVSITGDYRGQGTLVTVSSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 51 - protein, 348 aa, synthetic construct
QVQLQESGPGLVKSSETLSLTCTVSGGSISSSDWWSWVRQPPGKGLEWIGEIDHSGSTNYNPSLMSRVTISVDKSKNQFSLKLSSVTAADTAVYFCARGSWELTDAFDIRGQGTLVTVSSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 52 - protein, 347 aa, synthetic construct
QVQLVESGGDLVKPGGSLRLSCAASGFTFSDYYMSWLRQAPGKELEWVSHISSSGTTTYYADSVEGRFTITRDNAKNSLYLQMNSLRAEDTAVYYCARGAAVAPGFDSRGQGTLVTVSSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 53 - protein, 347 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 54 - protein, 347 aa, synthetic construct
QVQLVESGGGLVKPGGSLRLSCAASGFTFNDYYMSWIRQAPGKGLEWVSHISSSGSTIYYADSVKGRFTVSRDNANNSLYLQMHSLRAEDTAVYYCARGDAVSITGDYRGQGTLVTVSSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 55 - protein, 120 aa, synthetic construct
QVQLQESGPGLVKPSGTLSLTCAVSGGSISSSDWWSWVRQPPGKGLEWIGEIDHSGSTNYNPSLMSRVTISVDKSKNQFSLKLSSVTAADTAVYFCGRGSWELSDAFDIRGQGTLVTVSS

SEQ ID NO: 56 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 57 - protein, 120 aa, synthetic construct
QVQLQESGPGLVKSSETLSLTCTVSGGSISSSDWWSWVRQPPGKGLEWIGEIDHSGSTNYNPSLMSRVTISVDKSKNQFSLKLSSVTAADTAVYFCARGSWELTDAFDIRGQGTLVTVSS

SEQ ID NO: 58 - protein, 120 aa, synthetic construct
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSS

SEQ ID NO: 59 - protein, 126 aa, synthetic construct
QVQLVESGGGVVQPGRSLRLSCAASGFTFSSYGMHWVRQAPGKEREWVAVISYDGSNKYYTDSVKGRFTISRDNSKNTLYLEMNSLRAEDTAVYYCARDLDYDVLTGDPVGGFDIWGQGTLVTVSS

SEQ ID NO: 60 - protein, 121 aa, synthetic construct
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSS

SEQ ID NO: 61 - protein, 108 aa, synthetic construct
QSALTQPASVSGSPGQSIAISCTGTSSDIGHYDFVSWYQQHPGTAPKLIIYDINNRPSGISNRFSGSKSDNMASLTISGLQPEDEADYYCSAYTSSDTLVFGGGTKLT

SEQ ID NO: 62 - protein, 119 aa, synthetic construct
EVQLVQSGTEVKKPGASVKVSCKASGYTFTTYAMHWVRQAPGQSLEWMGWINTGNGNTKYSQNFQGRVTMTRDTSISTAYMELSRLRSDDTAVYYCARDLGQLERLYFWGQGTLVTVSS

SEQ ID NO: 63 - protein, 107 aa, synthetic construct
DIQMTQSPSTLSASVGDRVTLSCRAGQAISSWLAWYQQKPGKAPKLLIYKASNLESGVPSRFSGGGSGAEFTLTISSLQPDDFATYYCQQYQSYPYTFGQGTKLEIR

SEQ ID NO: 64 - protein, 114 aa, synthetic construct
HVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCARDLGDYWGQGTLVTVSS

SEQ ID NO: 65 - protein, 107 aa, synthetic construct
DIQLTQSPSSLSASVGDRVTITCQASQDIGNYLNWYQLKPGKAPKLLIYDASNLETGVPSRFSGSGSGTDFTFTISSLQPEDIATYYCLQLYDYPLTFGGGTKVEIK

SEQ ID NO: 66 - protein, 117 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSSSGDAFDIWGQGTMVTVSS

SEQ ID NO: 67 - protein, 110 aa, synthetic construct
NFMLTQPHSVSESPGKTVTISCTRSSGSIASNYVQWYQQRPGSSPTTVIFDDNQRPTGVPDRFSAAIDTSSSSASLTISGLTAEDEADYYCQSSHSTAVVFGGGTKLTVL

SEQ ID NO: 68 - protein, 108 aa, synthetic construct
QSALTQPASVSGSPGQSIAISCTGTSSDIGDYDFVSWYQQHPGTAPKLIIYDINNRPSGISNRFSGSKSDNMASLTISGLQPEDEADYYCSAYTSSDTLVFGGGTKLT

SEQ ID NO: 69 - protein, 121 aa, synthetic construct
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYNWGQGTLVTVSS

SEQ ID NO: 70 - protein, 121 aa, synthetic construct
EVQLVQSGAEVKKPGSSVKVSCKASGYTFTNYYIHWVRQAPGQGLEWMGAIMPSRGGTSYPQKFQGRVTMTGDTSTSTVYMELSSLRSEDTAVYYCARGEYYYDSSGYYYWGQGTLVTVSS

SEQ ID NO: 71 - protein, 108 aa, synthetic construct
QSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLT

SEQ ID NO: 72 - protein, 108 aa, synthetic construct
QSALTQPASVSGSPGQSIAISCTGTSSDIGHYDFVSWYQQHPGTAPKLIIYDINNRASGISNRFSGSKSDNMASLTISGLQPEDEADYYCSAYTSSDTVVFGGGTKLT

SEQ ID NO: 73 - protein, 108 aa, synthetic construct
QSALTQPASVSGSPGQSIAISCTGTSSDIGHYDFVSWYQQHPGTAPKLIIYDNNNRPSGISNRFSGSKSDNMASLTISGLQPEDEADYYCSAYTSSDTLVFGGGTKLT

SEQ ID NO: 74 - protein, 108 aa, synthetic construct
QSALTQPASVSGSPGQSIAISCTGTSSDIGHYDFVSWYQQHPGTAPKLIIYDINNRPSGISNRFSGSKSDDMASLTISGLQPEDEADYYCSAYTSSDTVVFGGGTKLT

SEQ ID NO: 75 - protein, 121 aa, synthetic construct
EVQLVQSGAEVKKPGSSVKVSCKASGYTFTNYYMHWVRQPPGQGLEWMGAIMPSRGGTSYPQKFQGRVTMTGDTSTSTVYMELSSLRSEDTAVYYCARGEYYYDSSGYYYWGQGTLVTVSS

SEQ ID NO: 76 - protein, 108 aa, synthetic construct
QSALTQPASVSGSPGQSITISCTGTSSDIGHYDFVSWYQQHPGTAPKLIIYDINNRPSGISNRFSGSKSDNMASLTISGLQPEDEADYYCSAYTSSDTVVFGGGTKLT

SEQ ID NO: 77 - protein, 121 aa, synthetic construct
EVQLVQSGAEVKKPGSTVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSS

SEQ ID NO: 78 - protein, 108 aa, synthetic construct
QSALTQPASVSGSPGQSIAISCTGTSSDIGHYDFISWYQQHPGTAPKLIIYDFNNRPSGISNRFSGSKSDNMASLTISGLQPEDEADYYCSAYTSSDTLVFGGGTKLT

SEQ ID NO: 79 - protein, 108 aa, synthetic construct
QSALTQPASVSGSPGQSIAISCTGTSSDIGHYDFVSWYQQHPGTAPKLIIYDNNNRPSGISNRFSGSKSDNMASLTISGLQPEDEADYYCSAYTSSDTVVFGGGTKLT

SEQ ID NO: 80 - protein, 108 aa, synthetic construct
QSALTQPASVSGSPGQSIVISCTGTSSDIGHYDFVSWYQQHPGTAPKLIIYDINNRPSGISNRFSGSKSDNMASLTISGLQPEDEADYYCSAYTSSDTVVFGGGTKLT

SEQ ID NO: 81 - protein, 108 aa, synthetic construct
QSALTQPASVSGSPGQSIAISCTGTSSDIGDYDFVSWYQQHPGTAPKLIIYDINNRPSGISNRFSGSKSDNMASLIISGLQPEDEADYYCSAYTSSDTLVFGGGTKLT

SEQ ID NO: 82 - protein, 108 aa, synthetic construct
QSALTQPASVSGNPGQSIAISCTGTSSDIGHYDFVSWYQQHPGTAPKLIIYDINNRPSGISNRFSGSKSDNMASLTISGLQPEDEADYYCSAYTSSDTVVFGGGTKLT

SEQ ID NO: 83 - protein, 121 aa, synthetic construct
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAMYYCARGEYYYDSSGYYYWGQGTLVTVSS

SEQ ID NO: 84 - protein, 108 aa, synthetic construct
QSTLTQPASVSGSPGQSITISCTGTSSDIGHYDFVSWYQQHPGTAPKLIIYDINNRPSGISNRFSGSKSDNMASLTISGLQPEDEADYYCSAYTSSDTVVFGGGTKLT

SEQ ID NO: 85 - protein, 121 aa, synthetic construct
EVQLVQSGAEVKKPGSSVKVSCKASGYTFTNYYMHWVRQAPGQGLEWMGAIMPSRGGTSYPQKFQGRVTMTGDTSTSTVYMELSSLRTEDTAVYYCARGEYYYDSSGYYYWGQGTLVTVSS

SEQ ID NO: 86 - protein, 121 aa, synthetic construct
EVQLVQSGAEVKKPGSSVKVSCKASGYTFTNYYMHWVRQAPGQGLEWMGAIMPSRGGTSYPQKFQGRVTMTGDTSTSTVYMELSNLRSEDTAVYYCARGEYYYDSSGYYYWGQGTLVTVSS

SEQ ID NO: 87 - protein, 121 aa, synthetic construct
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVNVSS

SEQ ID NO: 88 - protein, 121 aa, synthetic construct
EVQLVQSGAEVKKPGSSVKVSCKASGYTFSNYYMHWVRQAPGQGLEWIGAIMPSRGGTSYPQKFQGRVTMTGDTSTSTVYMELSSLRSEDTAVYYCARGEYYYDSSGYYYWGQGTLVTVSS

SEQ ID NO: 89 - protein, 121 aa, synthetic construct
EVQLVQSGAEVKKPGSSVKVSCKATGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSS

SEQ ID NO: 90 - protein, 121 aa, synthetic construct
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTPVTVSS

SEQ ID NO: 91 - protein, 108 aa, synthetic construct
QSALTQPASVSGSPGQSIAISCTGTSSDIGDYDFVSWYQQHPGTAPKLIIYDINNRPSGISNRFSGSKSDNMASLTISGLQPEDEADYYCSAYTSSDTVVFGGGTKLT

SEQ ID NO: 92 - protein, 121 aa, synthetic construct
EVQLVQSGAEVKKPGASVKVSCKASGYTFTNYYMHWVRQAPGQGLEWMGAIMPSRGGTSYPQKFQGRVTITADKSTSTAYMELSSLRSEDTAVYYCARGEYYYDSSGYYYWGQGTLVTVSS

SEQ ID NO: 93 - protein, 108 aa, synthetic construct
QSVLTQPPSVSGAPGQRVTISCTGTSSDIGHYDFVSWYQQLPGTAPKLLIYDINNRPSGVPDRFSGSKSGTSASLAITGLQAEDEADYYCSAYTSSDTLVFGGGTKLT

SEQ ID NO: 94 - protein, 119 aa, synthetic construct
EVQLVESGGGLVQTGGSLRLSCAASGSQFINDVMGWYRQVPGKQRELVADMDDTGSTEYADSVKGRFTILRDSVKNTAYLQMSNLKPEDTGVYYCKAGLWIKGRHFDYWGQGTQVTVSS

SEQ ID NO: 95 - protein, 126 aa, synthetic construct
QVQLVESGGGSVQPGGSLRLSCAASGFTFSNYAMSWVRQAPGKGLEWVASITGFGRGTDYADSVKGRFTISRDNAEDTLYLQMNSLKPEDTAVYYCAKYSSSTYYPPTPARGRDYRGQGTQVTVSS

SEQ ID NO: 96 - protein, 123 aa, synthetic construct
EVQLVESGGGLVQAGGSLRLSCAASGRAIENYPVGWFRQAPGKEREFVAAITWISGSTLYADSVKGRFTISRDNAKNTVYLQMSSLKPEDTALYYCAAALKTITRGQNDYSYWGQGTQVTVSS

SEQ ID NO: 97 - protein, 116 aa, synthetic construct
QVQLQESGGGLVQAGGSLRLSCVASGSVSSINGMAWYRQGADNQRVLVAAISRVGNTAYGDSVKGRFTISRQNARNTVYLQMNSLKPEDTAVYYCNADSWGGDDYWGQGTQVTVSS

SEQ ID NO: 98 - protein, 125 aa, synthetic construct
QVQLVESGGGLVQPGGSLRLSCAISGGTLDSYGIGWVRQAPGKQREGVSCMSRSDDRTYYADSVKGRFTISKDSAKNTVYLQMTSLKPEDTAVYYCAAVDAYGCSLVQPTTYDFWGLGTQVTVSS

SEQ ID NO: 99 - protein, 127 aa, synthetic construct
EVQLVESGGGLVQTGGSLRLSCAASGGTFSRDAMAWFRQVPGKEREFVALISWSGATTNYADSVKGRFAISRDNGKNTVYLQMNRLKPADTAIYYCAADRRPMGSRSYFEPTEYDDWGQGTQVTVSS

SEQ ID NO: 100 - protein, 123 aa, synthetic construct
EVQLVESGGGLVQAGGSLRLSCAASGRDFSSYAMGWFRQAPGKEREFVVAITWTKRSTDFPDSVKGRFTISRDNAKNTVYLDMNSLKPEDTAVYYCASARGLPVTPLGDIIYWGEGTLVTVSS

SEQ ID NO: 101 - protein, 126 aa, synthetic construct
EVQLVESGGGLVQAGGSLRLSCAASGRTFSINAMGWFRQAPGKEREFVAAISRSGGSTVYVDGVKGRFTISRDNAKNTVYLQMNSLEPEDTAVYYCAATMAVGWTTRWRTADFDSWGQGTQVTVSS

SEQ ID NO: 102 - protein, 126 aa, synthetic construct
EVQLVESGGGLVQAGGSLRLSCAASGSIFSINAMAWFRQVPGMERELVAAISRDGGASVYRDSVKGRFTISRDNSKNTVYLQMNTLKPEDTAIYVCAATRAIGWTARWITTDFDFWGQGTQVTVSS

SEQ ID NO: 103 - protein, 123 aa, synthetic construct
QVQLVESGGGLVQAGGSLRLSCAVSGDVFVRYTMAWFRQAPGKEREFVASVTDSGRTTDYVHSVKGRFTVSRDNAKNTVYLQMNNLKPEDTAVYYCAANTDYFQIKSLDANTWGQGTQVTVSS

SEQ ID NO: 104 - protein, 126 aa, synthetic construct
QVQLVESGGELVQGGASLRLSCAASGRTFSNANMAWFRQAPEKEREFVALITWSSGSTLYADSVKGRFTISRDNARKMVYLQMNSLKPEDTAVYYCAADGPPYSGTYYRYDTYDYWGQGTQVTVSS

SEQ ID NO: 105 - protein, 123 aa, synthetic construct
QVQLVESGGGLVQTGDSLRLSCAASGRSLDTTYIAWFRQAPGKERDFLAYISPRFSHTWYADSVKGRFTISRNIAKRTVDLEMNSLEPEDTAVYYCAAREHSGSTAWEHYDHWGQGTQVTVSS

SEQ ID NO: 106 - protein, 123 aa, synthetic construct
QVQLQESGGGLVQAGGSLRLSCAASGDVFVRYTMAWFRQAPGKEREFVASVTDSGRTTEYVDSVKGRFTVSRDNAKNTAYLQMNNLKPEDTAIYYCAANTDYFQIRSLDLNTWGQGTQVTVSS

SEQ ID NO: 107 - protein, 126 aa, synthetic construct
QVQLQESGGGSVQAGGSLRLSCVTSGYTYSSANMAWFRQAPGKEREGVAIITPSGRATTYADSVKGRFTISRDNAANTLYLQMNSLKPEDTAMYYCAADTPPYSGLWYAERTYNYWGQGTQVTVSS

SEQ ID NO: 108 - protein, 128 aa, synthetic construct
QVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSS

SEQ ID NO: 109 - protein, 120 aa, synthetic construct
QVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSS

SEQ ID NO: 110 - protein, 128 aa, synthetic construct
QVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 111 - protein, 119 aa, synthetic construct
QVQLVESGGGLVKPGGSLRLSCAASGFTFSDYYMSWIRQAPGKGLEWVSSISSSGDTIYYADSVQGRFTLSRDNAENSLFLQMNSLRAEDTAVYYCARGDAVSITGDYRGQGTLVTVSS

SEQ ID NO: 112 - protein, 119 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSS

SEQ ID NO: 113 - protein, 119 aa, synthetic construct
QVQLVESGGGLVKPGGSLRLSCAASGFTFNDYYMSWIRQAPGKGLEWVSHISSSGSTIYYADSVKGRFTVSRDNANNSLYLQMHSLRAEDTAVYYCARGDAVSITGDYRGQGTLVTVSS

SEQ ID NO: 114 - protein, 119 aa, synthetic construct
QVQLVESGGDLVKPGGSLRLSCAASGFTFSDYYMSWLRQAPGKELEWVSHISSSGTTTYYADSVEGRFTITRDNAKNSLYLQMNSLRAEDTAVYYCARGAAVAPGFDSRGQGTLVTVSS

SEQ ID NO: 115 - protein, 120 aa, synthetic construct
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSS

SEQ ID NO: 116 - protein, 112 aa, synthetic construct
DVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 117 - protein, 120 aa, synthetic construct
QVQLQQWGAGLLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSS

SEQ ID NO: 118 - protein, 120 aa, synthetic construct
QVQLQQWGAGMLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSS

SEQ ID NO: 119 - protein, 120 aa, synthetic construct
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAGDILTGYALDYWGQGTLVTVSS

SEQ ID NO: 120 - protein, 108 aa, synthetic construct
SYELTQPPSMSVSPGQTARITCSGDALPKQFAFWYQQKPGQAPVLVIYKDTERPSGIPERFSGSSSGTTVTLTITGVQAEDEADYYCQSPDSSGTVEVFGGGTKLTVL

SEQ ID NO: 121 - protein, 112 aa, synthetic construct
DVVMTQSPLSLPVTPGESVSISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 122 - protein, 120 aa, synthetic construct
EVQLVESGGGLVQPGGSLRLSCAASGGSFSGYYWSWVRQAPGKGLEWVSEINHSGSTNYNPSLKSRFTISRDNSKNTLYLQMNSLRAEDTAVYYCARSPGGYSGGYFQHWGQGTLVTVSS

SEQ ID NO: 123 - protein, 112 aa, synthetic construct
DIQMTQSPSSLSASVGDRVTITCRSSQSLLHSNGYNYLDWYQQKPGKAPKLLIYLGSNRDSGVPSRFSGSGSGTDFTLTISSLQPEDFATYYCMQGTHWPWTFGQGTKVEIK

SEQ ID NO: 124 - protein, 118 aa, synthetic construct
QVQLVESGGGLVQAGGSLTLSCAAPGRTFGTDVVGWFRQAPGKEREFVASISRSGDGIYYDDSVKGRFTISRNNAWNTVNLQMNSLKVEDTAVYYCAAGDGWSTYDYWGQGTQVTVSS

SEQ ID NO: 125 - protein, 126 aa, synthetic construct
QVQLVESGGGLVQAGGSLRLSCAASGRTLSRYAMGWFRQAPGKEREFVTANSWGGDTYYADSVQGRFTFSRDNAKNTVYLQMNSLQPEDTAVYYCAAAPTSFATTAYSGSNSYAYWGQGTQVTVSS

SEQ ID NO: 126 - protein, 126 aa, synthetic construct
QVQLVESGGGLVQAGGSLRLACVASGLTFDNYYMGWFRQAPGKEREFVAGIIWNGDHTAYADSIKGRFTISRDNAKNTAYLRMNSLKPEDTAVYYCAATFWIERATTPDIGQYAYWGQGTQVTVSS

SEQ ID NO: 127 - protein, 126 aa, synthetic construct
EVQLVESGGGWVQDGGSLRLSCALSGRTFVRGIMGWFRQAPGKEREFVARIIWHINSTRYADSVKGRFTISRDSAKNTMYLQMDSLRPEDTAVYYCAARDRYGSGNSLSPSAYDYWGQGTQVTVSS

SEQ ID NO: 128 - protein, 121 aa, synthetic construct
QVQLVESGGGLVQAGGSLRLSCTGYGGAFTGYALGWFRQAPGKEREFVARINWSGSFTYYASSVKGRFTISRDNAKNTMYLQMNNLKPEDTAVYYCAADNPSTLATDYDNWGQGTQVTVSS

SEQ ID NO: 129 - protein, 129 aa, synthetic construct
QVQLVESGGGLVQAGGSLRLSCAASGRTFGSTAVGWFRQVPGKEREFVSAINRSGSATTYADSVKGRFTISRDNAKNTVYLQMNSLTPEDTGVYYCAADSLPYGRPYYFQRSAGEYDYWGQGTQVTVSS

SEQ ID NO: 130 - protein, 129 aa, synthetic construct
QLQLVESGGGLVQAGGSLRLSCAASGPTFSRVAVGWFRQAPGKEREFVAAVNRPATMTKYADSVKGRFTVSRDNAKNTVDLQMNSMKPEDTAVYYCAADSVPYGRPYYWQTSAGDYDYWGQGTQVTVSS

SEQ ID NO: 131 - protein, 126 aa, synthetic construct
QVQLVESGGGLVQAGSSLRLSCAASGRTLSRLAMGWFRQAPGKEREFVAVNSWGGDTFYADSVEGRFTYSRDNAKSAVYLQMNSLQPEDTAVYYCAAAPTSFATTAYSSSNSYAYWGQGAQVTVSS

SEQ ID NO: 132 - protein, 126 aa, synthetic construct
QVQLQESGGGLVQGGGSLRLSCAASGGIFSSYAMGWFRQAPGKEREFVAAISRSGRSTNYADSVKGRFTISRDNAKSTVYLQMNSLKPEETAVYYCAAGRYYNSAYDPSPGDFGSWGHGTQVTVSS

SEQ ID NO: 133 - protein, 126 aa, synthetic construct
QVQLVESGGGLVQAGGSLRLSCAASGRTLSRYAMGWFRQAPGSEREFVAASSWGGDTFYADSVEGRFTFSRDNAKNAVYLQMNSLQPEDTAAYYCAAAPTSFPTTAYSSSNSYAYWGQGTQVTVSS

SEQ ID NO: 134 - protein, 126 aa, synthetic construct
QVQLVESGGGLVQAGGSLRLSCAASGRTLSRYAMGWFRQAPGKEREYVAIDSWGGDTFYADSVEGRFTFSRDNAKNEVYLQMNSLQPEDTAVYYCAGAPTSFATTAYSSSNSYRYWGQGTQVTVSS

SEQ ID NO: 135 - protein, 126 aa, synthetic construct
QVQLVESGGGLVQAGGSLRLSCAASGRSLSRDAMGWFRQAPGKEREFVAVMSWGGDTFYTDSVEGRFTFSRDNAKNAVYLEMNDLQPEDTAVYYCAAAPTSFATTAYSSSNSYSYWGRGTQVTVSS

SEQ ID NO: 136 - protein, 129 aa, synthetic construct
QVQLQESGGGSVQAGGSLRLSCAASGYTYSKNWYMGWFRQTPGKEREGVAVIAYDDWPTYADSVKGRFTISKDNTKNTLYLQMNSLKPEDTAMYYCAARQLGGDYCYFPNLSRFCYNYWGQGTQVTVSS

SEQ ID NO: 137 - protein, 118 aa, synthetic construct
QVQLQESGGGSVQAGGSLRLSCTASGFTFNEANHMGWYRQAPGNECELVSTISSDGTTYYPDSVKGRFTISQDNAKKTAFLQMNSLKPEDTAVYYCAADQSRRGSLCLGQGTQVTVSS

SEQ ID NO: 138 - protein, 133 aa, synthetic construct
QVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSS

SEQ ID NO: 139 - protein, 122 aa, synthetic construct
HVQLVESGGGLVQPGRSLRLSCAASGFTFSSYWMYWVRQTPGKGLEWVSTINRDGSATWYADSVKGRFTISRDNAKNTGYLQMNSLEPDDTAVYYCVSDPDNYSSDEMVPYWGQGTQVTVSS

SEQ ID NO: 140 - protein, 122 aa, synthetic construct
QVQLVESGGGLVQPGGSLRLSCAASGFTFSSYWMYWVRQTPGKGLEWVSTINRDGSATWYADSVKGRFTISRDNAKNTGYLQMNSLKPDDTAVYYCVSDPDNYSSDEMVPYWGQGTQVTVSS

SEQ ID NO: 141 - protein, 122 aa, synthetic construct
  VARIANT 1: note = Any amino acid
  VARIANT 16: note = Any amino acid
  VARIANT 87: note = Any amino acid
XVQLVESGGGLVQPGXSLRLSCAASGFTFSSYWMYWVRQTPGKGLEWVSTINRDGSATWYADSVKGRFTISRDNAKNTGYLQMNSLXPDDTAVYYCVSDPDNYSSDEMVPYWGQGTQVTVSS

SEQ ID NO: 142 - protein, 130 aa, synthetic construct
QVQLVESGGGLVQAGGSLRLSCAASGFTFDDWAIGWFRQAPGKEREGVSCISKRHGTTHYADSVKGRFTISSDNAKNTVYLRMNGLKPEDTAVYYCAASSWGSCTVATMRDVDRYDYDYWGQGTQVTVSS

SEQ ID NO: 143 - protein, 123 aa, synthetic construct [sequence omitted]

SEQ ID NO: 144 - protein, 129 aa, synthetic construct
DVQLVESGGGSVQSGGSLRLSCAASGYTYRRYYMGWFRQAPGEQREGVAVINNDGRTNYADSVKGRFRISRDNAENTLHLEMNSLKPEDTAMYYCAATGNILPPMTAVPPLGRQWYPYWGRGTLVTVSS

SEQ ID NO: 145 - protein, 130 aa, synthetic construct
HVQLVESGGGSVQSGGSLRLSCAASGYAVKNCMGWFRQAPGKEREGVAVINRNGITTYADSVKGRFTISQDKDKNTLDLQMNSLKPEDTAMYYCAATPTLLTIPARFLCDVRNPSGFTDWGQGTLVTVSS

SEQ ID NO: 146 - protein, 128 aa, synthetic construct
QVQLVESGGGSVQAGGSLRLSCVVSAYSAYTYKTMCMGWFRQAPGKEREGVAAIYRGGLNTYYADSVKGRFIISRDNAESTMYLQMNSLKPEDTAMYYCAADWLRGDDCNIGANFDYWGQGTQVTVSS

SEQ ID NO: 147 - protein, 132 aa, synthetic construct
QVQLVESGGGSVQAGGSLRLSCVATGFTISRKCMGWFREAPGKKREVIATINTGSSSPYYADGVKGRFTISQDNAKNTVYLQMNSLKPEDTAMYYCAATKGVVVGTGYCGGPYVERPNSAYWGQGTQVTVSS

SEQ ID NO: 148 - protein, 126 aa, synthetic construct
DVQLVESGGGSVQAGRSLRLSCELSDYTWSTVCMGWFRQAPGKEREGVAVIYTRSGGTTYADSAKGRFTISRDNAKDTLYLQMDSLKPEDTAMYYCAAGPLYDGRCTYRSPAFHYWGQGTQVTVSS

SEQ ID NO: 149 - protein, 122 aa, synthetic construct
DVQLVESGGGSAQAGGSLRLSCAASGPTSSLRTMGWFRQASGKERERVAVIWDGRTTDYDDSVQDRFTISQDNAKSTVYLQMNTLKPEDTAMYYCAASPRIVPFASTYFQHWGQGTQVTVSS

SEQ ID NO: 150 - protein, 127 aa, synthetic construct
HVQLVESGGGSVQAGGSLKLSCAASGSIFSGSIFSRCGMRWYRQAPGKERELVSSTSKDGFTSYTDSVKGRFTISQDNANNTLYLQMSSLKTEDTAVYSCAAICAVGGYSLSTYTYWGQGTQVTVSS

SEQ ID NO: 151 - protein, 124 aa, synthetic construct
EVQLVESGGDSVQAGGSLRLSCAASGYSPGSYCMGWFRQAPGKERERVAIIESRGTVTYVDSVKGRFTISKDNAKNTLYLQMNSLKPEDTAMYYCAASRPWSGVRCLHDKYDYWGQGTQVTVSS

SEQ ID NO: 152 - protein, 129 aa, synthetic construct
HVQLVESGGGSVQSGGSLRLSCAVSGYAYSSLAWFRQAPGKEREGVAALLTAIGGPTRTTYADSVKGRLAISQDHAKNTLYLQMSSLKPEDTAMYYCAAGRPAGTPRWLLLAPRDYNYWGQGTQVTVSS

SEQ ID NO: 153 - protein, 115 aa, synthetic construct
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 154 - protein, 117 aa, synthetic construct
QVQLQESGGGLVQPGGSLRLSCAASGFIFSNDAMTWVRQAPGKGLEWVSSINWSGTHTNYADSVKGRFTISRDNAKRTLYLQMNSLKDEDTALYYCVTGYGVTKTPTGQGTQVTVSS

SEQ ID NO: 155 - protein, 127 aa, synthetic construct
QVQLVQSGGGLVQAGGSLSLSCAFSGRTFSMYTMGWFRQAPGKEREFVAANRGRGLSPDIADSVNGRFTISRDNAKNTLYLQMDSLKPEDTAVYYCAADLQYGSSWPQRSSAEYDYWGQGTTVTVSS

SEQ ID NO: 156 - protein, 116 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVS

SEQ ID NO: 157 - protein, 117 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 158 - protein, 120 aa, synthetic construct
QVQLQESGGGLVQAGGSLRLSCAASGRTVSSLFMGWFRQAPGKERELVAAISRYSLYTYYADSVKGRFTISADNAKNAVYLQMNSLKPEDTAVYYCASKLEYTSNDYDSWGQGTQVTVSS

SEQ ID NO: 159 - protein, 120 aa, synthetic construct
QVQLQESGGGLVQAGDSLRLSCAASGRAVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 160 - protein, 120 aa, synthetic construct
QVQLQESGGGLVQAGDSLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 161 - protein, 116 aa, synthetic construct
QVQLQESGGGLVQPGDSMRLSCAAEGDSLDGYVVGWFRQAPGKERQGVSSISGDGSMRYVADSVKGRFTISRDNAKNTVYLQMIDLKPEDTGVYYCAADPPTWDYWGQGTQVTVSS

SEQ ID NO: 162 - protein, 120 aa, synthetic construct
QVQLQESGGGLVQPGGSLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 163 - protein, 120 aa, synthetic construct
QVQLQESGGGLVQAGESLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 164 - protein, 111 aa, synthetic construct
EVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSS

SEQ ID NO: 165 - protein, 125 aa, synthetic construct
QLQLVESGGGLVHAGGSLRLSCAASGSTFSINAIGWYRQAPGKQRELVAALSSGGSKNYADSVKGRFTISRDNAKNTVYLQMNRLKPEDTAVYYCNAEIYYSDGVDDGYRGMDYWGKGTQVTVSS

SEQ ID NO: 166 - protein, 129 aa, synthetic construct
EVQVVESGGGLVQTGGSLRLSCAASGPPLSSYAVAWFRQTPGKEREFVAAISWSGSNTYYADSVKGRFTISKDNAKNTVLVYLQMNSLKPEDTAVYYCAADRRGGPLSDYEWEDEYADWGQGTQVTVSS

SEQ ID NO: 167 - protein, 116 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 168 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQDANDPRTFGQGTKVEIK

SEQ ID NO: 169 - protein, 109 aa, synthetic construct
DIQMTQSPSSLSASVGDRVTITCQASQDIGTYLNWYQQKPGKAPKLLIYEASTLETGVPSRFSGSGSGTDFTFTISSLQPEDIATYYCQQDGARDDYATFGQGTKVEIK

SEQ ID NO: 170 - protein, 107 aa, synthetic construct
DIQMTQSPSSLSASVGDRVTITCQASQDIDDYLNWYQQKPGKAPKLLIYDASNLQSGVPSRFSGSGSGTDFTLTISSLQPEDFATYYCQQDSMDPRTFGQGTKVEIK

SEQ ID NO: 171 - protein, 109 aa, synthetic construct
DIQMTQSPSSLSASVGDRVTITCRASQSIDEYLNWYQQKPGKAPKLLIYEASKLQSGVPSRFSGSGSGTDFTLTISSLQPEDFATYYCQQDGAMDTYATFGQGTKVEIK

SEQ ID NO: 172 - protein, 107 aa, synthetic construct
EIVLTQSPATLSLSPGERATLSCRASQSVDEYLAWYQQKPGQAPRLLIYDASERATGIPARFSGSGSGTDFTLTISSLEPEDFAVYYCQQDATDPRTFGQGTKVEIK

SEQ ID NO: 173 - protein, 119 aa, synthetic construct
QVQLQESGGGSVQAGGSLRLSCVGSGYTYDTSDMSWYRQAPGKEREFVSDIDSGDWAAYADAVKGRFTISRDNAKKTVYLQMNSLEPEDTAMYYCKASYWKWGKLNNFWGPGTQVTVSS

SEQ ID NO: 174 - protein, 116 aa, synthetic construct
QVQLQESGGGLVQPGGSLRLSCVASGFTFSNYWIFWVRQAAGKGLEWLSTSNTGGDTTKYADSVKGRFTISRDSAKNTEYLQMNSLKPEDTAVYYCETGRCARSGGYQGTQVTVSS

SEQ ID NO: 175 - protein, 122 aa, synthetic construct
QVQLQESGGGLVQPGGSLKLSCAASGFRFSNYGMSWVRQAPGEGLEWVSYINGDGSRTHYADSVKGRFTISRDNAKNTLYLQLNSLKTEDTAMYYCEKGLSRDGWSLSAASRGQGTQVTVSS

SEQ ID NO: 176 - protein, 125 aa, synthetic construct
QVQLQESGGGSVQTGGSLRLSCAVSGYTTYSFNYMGWFRQAPGKEREGVAVIYTGGGSTLYADSVKGRFTISQDNAKNTVYLQMNSLKPEDTAMYYCAADDQRFASPLYAYFGYWGQGTQVTVSS

SEQ ID NO: 177 - protein, 124 aa, synthetic construct
QVQLQESGGGSVQVGGSLRLSCATSGDTKSIRCMGWFRQTPGKEREGIAAIDREGFATYADSVYDRFTIAQDNAQNTLYLEMNALKPEDTAMYYCAAQNMCRVVRGAMTGVDYWGKGTQVTVSS

SEQ ID NO: 178 - protein, 124 aa, synthetic construct
QVQLQESGGGSVQAGGSLRLSCAASEYTASRYCMAWFRQAPGKEREGVAAIHPGGGTTYYADSVKGRFSISQDSADNTLYLQMNSLKPEDTAMYYCAAGSLWVPFGDRCAANYWGQGTQVTVSS

SEQ ID NO: 179 - protein, 122 aa, synthetic construct
QVQLQESGGGSVQAGGSLRLSCAASGYEYCRIHMTWYRQGPGKEREFVSSIGSDGRKTYANSVTGRFTISRDNANHTVYLQMNSLSPEDTAMYYCKTEYLYGLGCPDGSAYWGQGTQVTVSS

SEQ ID NO: 180 - protein, 125 aa, synthetic construct
QVQLQESGGGSVQVGGSLKLSCAASGYTYSSYYCMGWFRQAPGKEREGVAAIDSDGSTSYADSVKGRFTISQDDAKNTLYLQMNSLKPEDTAMYYCAASYEVVDCYPSGYGQDYWGKGTQVTVSS

SEQ ID NO: 181 - protein, 123 aa, synthetic construct
QVQLVQSGAEVKKPGASVRVSCKASGYTFTDYDIHWVRQAPGHGLEWMGWINPNSGGTNYAQKFQGRVTMTRDTSISTVYMDLSRLRSDDTAVYYCARADYSSSYYYYGMDVWGQGTTVTVSS

SEQ ID NO: 182 - protein, 113 aa, synthetic construct
DIVMTQSPDSLAVSLGERATINCKSSQSVLYSSKNKNYLSWYQQKPGQPPKLLIYWASTREFGVPDRFSGRGSGTDFTLTISSLQAEDVAVYYCQQYYTTPYTFGQGTKLEIK

SEQ ID NO: 183 - protein, 121 aa, synthetic construct
QVQLVESGGGVVQPGRSLRLSCTASGFTFRSYDMYWVRQAPGKGLEWVSVITYDGNNKYYADSVKGRFTISRDNSKNTLFLQMSSLRPEDTAVYYCAKRGLIWVGESFDYWGQGTLVTVSS

SEQ ID NO: 184 - protein, 107 aa, synthetic construct
DIQMTQSPSTLSASVGDRVTITCRASQSINSWLAWYQQKPGKAPNLLIYKASSLESGVPSRFSGSGSGTEFTLTISSLQPDDFATYYCQQYKSYSWTFGQGTKVEIK

SEQ ID NO: 185 - protein, 120 aa, synthetic construct
QVQLVESGGGVVQPGRSLRLSCAASGFNFRNFGMHWVRQAPGKGLEWVAGILYDGSSKYYADSVKDRFTISRDNSKNTLFLQMNSLRAEDTAMYYCAKEEDTAMVPFDSWGPGTLVTVSS

SEQ ID NO: 186 - protein, 107 aa, synthetic construct
DIQLTQSPSFLSASVGDRVTITCWASQGISSYLAWYQQKPGKAPTLLIYAASTLQSGVPSRFSGSGSGTEFTLTISSLQPEDFASYYCQQLKSYPLTFGGGTKVEIK

SEQ ID NO: 187 - protein, 122 aa, synthetic construct
QVQLQESGPGLVKPSQTLSLTCTVSGGSISSGGYYWSWIRQHPGKGLEWIGFIYYSGKTYYNPSLKSRLTISVDTSKSQFSLKLRSVTAADTAVYYCARLGYTNSAGWFDPWGQGTLVTVSS

SEQ ID NO: 188 - protein, 107 aa, synthetic construct
DIQMTQSPSSLSASVGDRVTITCRASQSISSYLNWYQQKPGKAPNLLIYAASSLQSGVPSRFSGSGSGTDFTLTISSLQPEDLATYYCQQSYTTPFTFGPGTKVDIK

SEQ ID NO: 189 - protein, 125 aa, synthetic construct
EVQLVESGGGLVKPGGSLRLSCAASGFTFSTAWMSWVRQSPGRGLEWVGRMKSKTDGGTTFYAAPVKGRFTISRDDSKNTLYLQMNSLKTEDTAVYYCTTGLVPAFYKYYGVDVWGQGTTVTVSS

SEQ ID NO: 190 - protein, 106 aa, synthetic construct
DIQMTQSPSSLSASVGDRITITCQASQDITNYLNWYQQKPGKAPNLLIYDASNLVTGVPSRFSGSGSGTDFTFTILSLQPEDIATYYCQQYDSLLTFGPGTKVDIK

SEQ ID NO: 191 - protein, 123 aa, synthetic construct
EVQLVESGGGLVQPGGSLRLSCAASGFTFNNYAMHWVRQAPGKGLEYVSSISSSGGSTYYEDSVKGRFTISRDNSKNTLYLQMGSLRAEDMAVYYCARSFYGSGTYYDTFDMWGQGTMVTVSS

SEQ ID NO: 192 - protein, 107 aa, synthetic construct
DIQMTQSPSSLSASIGDRVTITCRASQSISRYLNWYQQKPGKAPKLLIYAASSLQSGVPSRFSASGSGTDFTLTISSLQPEDFATYYCQQSYSTPFTFGQGTKLEIK

SEQ ID NO: 193 - protein, 114 aa, synthetic construct
QVQLVESGGDLVKPGGSLRLSCATSGFTFSDFYMTWIRQAPGKGLEWISYISNSGSIVKYADSVKGRFTISRDNAKNSLYLQMNSLRAEDTAIYYCARFYGDRWGQGTLVTVSS

SEQ ID NO: 194 - protein, 107 aa, synthetic construct
DIQLTQSPSFLSASVGDRVTITCWASQGISTFLAWYQQKPGKAPKLLIYAASTLQSGVPSRFSGSGSGTDFTLTISSLQPEDFATYHCQQLNNYPWTFGQGTKVEIK

SEQ ID NO: 195 - protein, 114 aa, synthetic construct
QVQLVESGGGLVKPGGSLRLSCEASGFTFNDFYMTWIRQAPGKGLEWIAYISKSGDKMRYADSVKGRFSTSRDNAKNSLSLQMNSLRAEDTAVYYCARFYGDIWGQGTLVTVSS

SEQ ID NO: 196 - protein, 107 aa, synthetic construct
DIQLTQSPSFLSASVGDRVTITCWASQDISSFLVWYQQKPGKAPNLLIYAASALQSGVPSRFSGSGSGTEFTLTISSLQPEDFASYYCEQLNNYPWTFGQGTKVEIK

SEQ ID NO: 197 - protein, 125 aa, synthetic construct
EVQLVESGGRLVQPGGSLRLSCEASGFTFSNYGMTWVRQAPGKGLEWVSVISGSDNRKYYAESVKGRFTISRDNSKNTLYLQMNSLRAEDTAVYYCAKLGYSRSSKDFYYGMDVWGQGTTVTVSS

SEQ ID NO: 198 - protein, 113 aa, synthetic construct
DIVMTQSPDSLAVSLGERATINCKSSQSVLYNSNNRNYLVWYQQKPGQSPKLLIYWASTRESGVPDRFSGSGSGTDFTLTISSLQAEDVAVYYCQQYYNVPYTFGQGTKLEIK

SEQ ID NO: 199 - protein, 116 aa, synthetic construct
EVQLVESGGGVVRPGGSLRLSCAASGFTFDDYGMSWVRQAPGKGLEWISSINRNGGSADYADSVKGRFTISRDNAKNSLFLQMSSLRAEDTALYHCASGEFRFDYWGQGTLVTVSS

SEQ ID NO: 200 - protein, 116 aa, synthetic construct
EVQLVESGGGLVQPGRSLRLSCAASGFTLEDYAMHWVRQAPGKGLEWVSGISWNRGSTGYADSVKGRFTISRDNAKNSLYLQMTSLRAEDTALYYCAKGFYSMDVWGQGTTVTVSS

SEQ ID NO: 201 - protein, 121 aa, synthetic construct
QVQLQQSGPGLVKPSQTLSLTCAISGDSVSSNIAAWNWIRLSPSRGLEWLGRTFFRSTWFYDYSLSVKGRITINPDTSKNQFSLHLNSVTPEDAAVYYCARTGRRWSLDYWGQGTLVTVSS

SEQ ID NO: 202 - protein, 116 aa, synthetic construct
EVQLVESGGGVVRPGGSLRLSCATSGFTFDDYGMSWVRQVPGKGLEWVSSVNRNGGTTDYADSVKGRFTISRDNAKRSLFLQMNSLRAEDTALYHCATGELFFDYWGQGTLVTVSS

SEQ ID NO: 203 - protein, 119 aa, synthetic construct
QVQLVQSGAEVKKPGASVKVSCKASGYTFTGHYMHWVRQAPGQGLEWMGWIYPHSGHTNYAKRFQGRVTMTRDTSITTAYMELIRLRSDDTAVYYCARRSGRSWYFDLWGRGTLVTVSS

SEQ ID NO: 204 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 205 - protein, 117 aa, synthetic construct
EVQLVESGGGLVQPGGSLGLSCAASGFTFSNYAMSWVRQAPGKGLEWVSAVSGGGGGTYYADSVKGRFTISRDNSKNTVLLQMNSLRAEDTAVYYCARGRTGGLDYWGPGTLVTVSS

SEQ ID NO: 206 - protein, 112 aa, synthetic construct
DVVMTQSPLSLPVIFGQPASISCRSSQSLVDSDGNTYLNWLQQRPGQSPRRLIYEVSNRDSGVPDRFSGSGSGTDFTLTISRVEAEDVGIYYCMQGTRWPPTFGGGTKVEIK

SEQ ID NO: 207 - protein, 122 aa, synthetic construct
EVQLVESGGGVVRPGGSLRLSCAASGFIFDDYDMSWVRQPPGRGLEWVSGIDWFGGTRGYADSMKGRFTISRDNAKNSLYLQMNSLRVEDTAFYYCARGGAIVGAVTPFDYWGQGTLVTVSS

SEQ ID NO: 208 - protein, 107 aa, synthetic construct
DIQMTQSPSSLSASVGNRVTLSCRASQSINTYLSWYQQRPGKAPKLLIYAASSLQSGVPSRFSGSGAGTDFTLTISSLQPEDFATYYCQQSYSAPLTFGGGTKVEIK

SEQ ID NO: 209 - protein, 122 aa, synthetic construct
QLQLQESGPGLVKPSETLSLTCTVSGGSISIKNYYWGWIRQPPGKGLEWIGSIYYSGTTYYNPSLKSRVTISVDTSKNQFSLKLSSVTAADTAVYHCARHGYSYGHGWFDPWGQGTLVTVSS

SEQ ID NO: 210 - protein, 122 aa, synthetic construct
QVQLQQSGPGLVKPSQTLSLTCDISGDSVSSNIATWNWIRQSPSRGLEWLGRTYYRSKWYKDYAVSVKSRITINPDTSKNQFSLQVNSVTPEDTAVYYCARMTGPRYYFEYWGQGTLVTVSS

SEQ ID NO: 211 - protein, 122 aa, synthetic construct
EVQLVESGGGVVRPGGSLRLSCAASGFTFDDFDMSWVRQGPGKGLEWVSGINWHGSSTGYADSVKGRFTISRDNAKNSLYLQMSSLRAEDTALYHCVRGGTIVGATTPLDYWGQGTLVTVSS

SEQ ID NO: 212 - protein, 107 aa, synthetic construct
DIQMTQSPSSLSASVGDRVTMTCRASRTISSYLSWYQQKSGKVPNLLIFGASSLQSGVPSRFSASGSGTDFTLIISSLQPEDFATYYCQQSYSSPLTFGGGTKVEIK

SEQ ID NO: 213 - protein, 117 aa, synthetic construct
EVQLVESGGDLVQPGGSLRLSCTASGFIFRNYAMNWVRQAPGKGLEWLSGILGSNDNTYYVDSVKGRFTISRDNSRNTLYLQMNSLRAEDSAVYYCAKGDAGGFDYWGQGTLVTVSS

SEQ ID NO: 214 - protein, 112 aa, synthetic construct
DVVMTQSPLSLPVILGQPASISCRSSQSLVSSDGNTYLNWFQQRPGQSPRRLIYKVSNRDSGVPDRFSGSGSGTDFTLKISRVEAEDVGAYYCMQGSYWPPTFGQGTKLEIK

SEQ ID NO: 215 - protein, 121 aa, synthetic construct
QVQLVESGGGVVKPGGSLRLSCAASGFTFSNSGIHWVRQAPGKGLEWVALISYAGSNKYYADSVKGRFTISRDNSKNTLSLQMNSLRAEDTAVYYCAKEVWTGTYDSFDMWGRGTMVTVSS

SEQ ID NO: 216 - protein, 121 aa, synthetic construct
EVQLVESGGGLVQPGGSLRLSCAASGFIFSSYEMHWVRQAPGKGLEWISYISSSGTTIYYADSVKGRFTISRDNAKNSLYLHMNSLRAEDTAVYYCTRARITGTFDVFDIWGQGTMVTVSS

SEQ ID NO: 217 - protein, 107 aa, synthetic construct
DIQMTQSPSSLSASVGDRVTITCRASQSISSYLNWYQQKPGKAPKLLIFAASNLQSGVPSRFSGSRSGTDFTLTISSLQPEDFATYYCQQNYNIPYTFGQGTKLEIK

SEQ ID NO: 218 - protein, 122 aa, synthetic construct
QVQLQESGPGLVKPSQTLSLTCTVSGGSITSGGYYWSWIRQYPGQGLEWIGYIYYSGKTYYNPSFTSRITISVDTSKKQFSLKMSSVTAADTAVYYCARAGFTSSNGWFDPWGQGTLVTVSS

SEQ ID NO: 219 - protein, 107 aa, synthetic construct
DIQMTQSPSSLSASVGDRVTITCRASQNIRSYLNWYQQKPGKAPKLLIYSASSLQSGVPSRFSGSGSGTDFTLTISSLQPEDFPTYYCQQTYSSPWTFGPGTKVEIK

SEQ ID NO: 220 - protein, 109 aa, synthetic construct
DIQMTQSPSSLSASVGDRVTITCRASQSIYYYLNWYQQKPGKAPKLLIYDASALQSGVPSRFSGSGSGTDFTLTISSLQPEDFATYYCQQIDFTAGSITFGQGTKVEIK

SEQ ID NO: 221 - protein, 108 aa, synthetic construct
DIQLTQSPSFLSASVGDRVTITCRASQTIDAPLRWYQQKPGKAPKLLIYLTSSLQSGVPSRFSGSGSGTEFTLTISSLQPEDFATYYCQQGYAAGPSTFGQGTKVEIK

SEQ ID NO: 222 - protein, 107 aa, synthetic construct
DIQMTQSPSTLSASVGDTVTITCRASHYITTWLAWYQQKPGKAPKLLIYDVSSLESGVPSRFRGRGSGTEFTLTISSLQPDDFATYYCQQYESYSPTFGQGTKVEIK

SEQ ID NO: 223 - protein, 108 aa, synthetic construct
DIQMTQSPSTLSASVGDRVTITCRASQTIYGPLNWYQQKPGKAPKLLIYSTSYLESGVPSRFSGSGSGTEFTLTISSLQPDDFATYYCQQAGYASAPTFGQGTKVEIK

SEQ ID NO: 224 - protein, 112 aa, synthetic construct
DIVMTQSPDSLAVSLGERATINCKSSQSVLYSEVAYTALAWYQQKPGQPPKLLIYATSTRESGVPDRFSGSGSGTDFTLTISSLQAEDVAVYYCQQGYGHPTFGQGTKVEIK

SEQ ID NO: 225 - protein, 114 aa, synthetic construct
DIVMTQSPDSLAVSLGERATINCKSSQSVLYDDFGNANLAWYQQKPGQPPKLLIYYGSYRESGVPDRFSGSGSGTDFTLTISSLQAEDVAVYYCQQVDVGLAITFGQGTKVEIK

SEQ ID NO: 226 - protein, 106 aa, synthetic construct
DIQLTQSPSFLSASVGDRVTITCRASQDIGIELAWYQQKPGKAPKLLIYFESHLQSGVPSRFSGSGSGTEFTLTISSLQPEDFATYYCQQIRIDPTFGQGTKVEIK

SEQ ID NO: 227 - protein, 109 aa, synthetic construct
EIVLTQSPGTLSLSPGERATLSCRASQDVATRGLAWYQQKPGQAPRLLIYGASSRATGIPDRFSGSGSGTDFTLTISRLEPEDFAVYYCQQYELEHPATFGQGTKVEIK

SEQ ID NO: 228 - protein, 107 aa, synthetic construct
DIQMTQSPSSLSASVGDRVTITCQASQDIAGYLNWYQQKPGKAPKLLIYTASTLETGVPSRFSGSGSGTDFTFTISSLQPEDIATYYCQQWAFGPVTFGQGTKVEIK

SEQ ID NO: 229 - protein, 107 aa, synthetic construct
EIVLTQSPATLSLSPGERATLSCRASQSVFANLNWYQQKPGQAPRLLIYDSSGRATGIPARFSGSGSGTDFTLTISSLEPEDFAVYYCQQGFGPSLTFGQGTKVEIK

SEQ ID NO: 230 - protein, 108 aa, synthetic construct
EIVLTQSPGTLSLSPGERATLSCRASQNVNHNFLTWYQQKPGQAPRLLIYSASARATGIPDRFSGSGSGTDFTLTISRLEPEDFAVYYCQQFNYAPLTFGQGTKVEIK

SEQ ID NO: 231 - protein, 121 aa, synthetic construct
QVQLQESGGGSVQAGGSLRLSCAASGFTFDDSDMGWYRQAPGNECDLVSTISSDGSTYYADSVKGRFTISQDNAKNTVYLQMDSVKPEDTAVYYCAADFMIAIQAPGAGCWGQGTQVTVSS

SEQ ID NO: 232 - protein, 117 aa, synthetic construct
QVQLQESGGGSVPAGGSLKLSCAASGFSFSSYPMTWARQAPGKGLEWVSTIASDGGSTAYAASVEGRFTISRDNAKSTLYLQLNSLKTEDTAMYYCTKGYGDGTPAPGQGTQVTVSS

SEQ ID NO: 233 - protein, 121 aa, synthetic construct
QVQLQESGGGSVQTGGSLRLSCTASGFTFDDREMNWYRQAPGNECELVSTISSDGSTYYADSVKGRFTISQDNAKNTVYLQMDSVKPEDTAVYYCAADFMIAIQAPGAGCWGQGTQVTVSS

SEQ ID NO: 234 - protein, 125 aa, synthetic construct
QVQLQESGGGSVQAGGSLRLSCTASGFTFDDSDMGWYRQAPGNECELVSTISSDGNTYYTDSVKGRFTISQDNAKNTVYLQMNSLGPEDTAVYYCAAEPRGYYSNYGGRRECNYWGQGTQVTVSS

SEQ ID NO: 235 - protein, 117 aa, synthetic construct
QVQLQESGGGSVQAGGSLRLSCAASGFSFSSYPMTWARQAPGKGLEWVSTIASDGGSTAYAASVEGRFTISRDNAKSTLYLQLNSLKTEDTAMYYCTKGYGDGTPAPGQGTQVTVSS

SEQ ID NO: 236 - protein, 120 aa, synthetic construct
QVQLQESGGGAVQAGGSLRLSCAASGFTFSNAHMSWVRQAPGKGREWISSIYSGGSTWYADSVKGRFTISRDNSKNTLYLQLNSLKTEDTAMYYCAENRLHYYSDDDSLRGQGTQVTVSS

SEQ ID NO: 237 - protein, 121 aa, synthetic construct
QVQLQESGGGLVQPGGSLRLSCAASGFTFDDREMNWYRQAPGNECELVSTISSDGSTYYADSVKGRFTISQDNAKNTVYLQMDSVKPEDTAVYYCAADFMIAIQAPGAGCWGQGTQVTVSS

SEQ ID NO: 238 - protein, 127 aa, synthetic construct
QVQLQESGGGSVQAGGSLRLSCVASGYTFSSYCMGWFRQAPGKEREGVAALGGGSTYYADSVKGRFTISQDNAKNTLYLQMNSLKPEDTAMYYCAAAWVACLEFGGSWYDLARYKHWGQGTQVTVSS

SEQ ID NO: 239 - protein, 124 aa, synthetic construct
QVQLQESGGGSVQAGGSLRLSCTASGFTFDDSDMGWYRQAPGGECELVTISSDGSTYYADSVKGRFTISQDNAKNTVYLQMNSLKPEDTAVYYCAAEPRGYYSNYGGRRECNYWGQGTQVTVSS

SEQ ID NO: 240 - protein, 127 aa, synthetic construct
QVQLQESGGGSVQAGGSLRLSCAASGSIYSSAYIGWFRQAPGKKREGVAGIYTRDGSTAYADSVKGRFTISQDSAKKTVYLQMNSLKPEDTAMYYCAAGRRTKSYVYIFRPEEYNYWGQGTQVTVSS

SEQ ID NO: 241 - protein, 120 aa, synthetic construct
QVQLQESGGGSVQAGGSLRLSCAASGFTFSSAHMSWVRQAPGKGREWIASIYSGGGTFYADSVKGRFTISRDNAKNTLYLQLNSLKTEDTAMYYCATNRLHYYSDDDSLRGQGTQVTVSS

SEQ ID NO: 242 - protein, 120 aa, synthetic construct
QVQLQESGGGSVQAGGSLRLSCAASGFTFSNAHMSWVRQAPGKGREWISSIYSGGSTWYADSVKGRFTISRDNSKNTLYLQLNSLKTEDTAMYYCAENRLHYYSDDDSLRGQGTQVTVSS

SEQ ID NO: 243 - protein, 125 aa, synthetic construct
QVQLQESGGGSVQAGGSLRLSCTASRFIFDDSDMGWYRQAPGNECELVSTISSDGSTYYADSVKGRFTISRDNAKNTVYLQMNSLKPEDTAVYYCAAEPRGYYSNYGGRRECNYWGQGTQVTVSS

SEQ ID NO: 244 - protein, 124 aa, synthetic construct
QVQLQESGGGSVQAGGSLKLSCTVSGFTADDSDMGWYRQGPGNECELVTISSDGSTYYADSVKGRFTISQDNAKNTVYLQMNSLKPEDTAVYYCAAEPRGYYSNYGGRRECNYWGQGTQVTVSS

SEQ ID NO: 245 - protein, 120 aa, synthetic construct
QVQLQESGGGLVQPGGSLRLSCAASGFTFSSAHMSWVRQAPGKGREWIASIYSGGGTFYADSVKGRFTISRDNAKNTLYLQLNSLKAEDTAMYYCATNRLHYYSDDDSLRGQGTQVTVSS

SEQ ID NO: 246 - protein, 120 aa, synthetic construct
QVQLQESGGGLVQPGGSLRLSCVASGFTFSNAHMSWVRQAPGKGREWISSIYSGGSTWYADSVKGRFTISRDNSKNTLYLQLNSLKTEDTAMYYCAENRLHYYSDDDSLRGQGTQVTVSS

SEQ ID NO: 247 - protein, 120 aa, synthetic construct
QVQLQESGGGLVQPGGSLRLSCAASGFTFSNAHMSWVRQAPGKGREWISSIYSGGSTWYADSVKGRFTISRDNSKNTLYLQLNSLKTEDTAMYYCAENRLHYYSDDDSLRGQGTQVTVSS

SEQ ID NO: 248 - protein, 117 aa, synthetic construct
QVQLQESGGGLVQPGGSLRLSCAASGFTFSSYPMTWARQAPGKGLEWVSTIASDGGSTAYAASVEGRFTISRDNAKSTLYLQLNSLKTEDTAMYYCTKGYGDGTPAPGQGTQVTVSS

SEQ ID NO: 249 - protein, 121 aa, synthetic construct
QVQLQESGGGSVQAGGSLRLSCTASGFTFDDREMNWYRQAPGNECELVSTISSDGSTYYADSVKGRFTISQDNAKNTVYLQMDSVKPEDTAVYYCAADFMIAIQAPGAGCWGQGTQVTVSS

SEQ ID NO: 250 - protein, 125 aa, synthetic construct
QVQLQESGGGSVQAGGSLRLSCTASGFTFDDSDMGWYRQAPGNECELVSTISSDGSTYYADSVKGRFTISQDNAKNTVYLQMNSLKPEDTAVYYCAAEPRGYYSNYGGRRECNYWGQGTQVTVSS

SEQ ID NO: 251 - protein, 119 aa, synthetic construct
QVQLQESGGGSVQAGGSLRLSCVASGYTSCMGWFRQAPGKEREAVATIYTRGRSIYYADSVKGRFTISQDNAKNTLYLQMNSLKPEDIAMYSCAAGGYSWSAGCEFNYWGQGTQVTVSS

SEQ ID NO: 252 - protein, 117 aa, synthetic construct
QVQLQESGGGLVQPGGSLRLSCTASGFSFSSYPMTWARQAPGKGLEWVSTIASDGGSTAYAASVEGRFTISRDNAKSTLYLQLNSLKTEDTAMYYCTKGYGDGTPAPGQGTQVTVSS

SEQ ID NO: 253 - protein, 117 aa, synthetic construct
QVQLQESGGGLVQPGGSLRLSCAASGFSFSSYPMTWARQAPGKGLEWVSTIASDGGSTAYAASVEGRFTISRDNAKSTLYLQLNSLKTEDTAMYYCTKGYGDGTPAPGQGTQVTVSS

SEQ ID NO: 254 - protein, 124 aa, synthetic construct
QVQLVESGGGVVQPGRSLRLSCAASGFTFSSYGMHWVRQAPGKGLEWVAFMSYDGSNKFYSDSVKGRFTISRDNSRKMLFLQMNNLRAEDTAVYYCARDQYYDFLTDHGVFDYWGQGTLVTVSS

SEQ ID NO: 255 - protein, 124 aa, synthetic construct
QVQLVESGGGVVQPGRSLRLSCAASGFTFSSYGMHWVRQAPGKGLEWVAVISYAGNNKYYADSVKGRFTVSRDNSKKTLYLQMNSLRSEDTAVYYCAKDSYYDFLTDPDVLDIWGQGTMVTVSS

SEQ ID NO: 256 - protein, 94 aa, synthetic construct
EVQLVESGGGLVQPGGSLTLSCAASRFMISEYSMHWVRQAPGKGLEWVSTINPAGTTDYAESVKGRFTISRDNAKNTLYLQMNSLKPEDTAVYY

SEQ ID NO: 257 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK